(19) United States
(12) Patent Application Publication
(10) Pub. No.: US 2025/0313556 A1
(43) Pub. Date: Oct. 9, 2025

(54) CERTAIN CHEMICAL ENTITIES, COMPOSITIONS, AND METHODS

(71) Applicant: MRJA THERAPEUTICS, INC., Encinitas, CA (US)

(72) Inventor: Xiangping (Shawn) QIAN, Foster City, CA (US)

(21) Appl. No.: 18/860,464

(22) PCT Filed: Apr. 26, 2023
(86) PCT No.: PCT/US2023/066259

§ 371 Date: Oct. 25, 2024

Related U.S. Application Data
(60) Provisional application No. 63/335,687, filed on Apr. 27, 2022.
(60) Provisional application No. 63/403,661, filed on Sep. 2, 2022.

(51) Int. Cl. C07D 471/04; A61K 31/4439; A61K 31/4545; A61K 31/496; A61K 31/5377

(52) U.S. Cl. CPC C07D 471/04; A61K 31/4439; A61K 31/4545; A61K 31/496; A61K 31/5377

(57) ABSTRACT

Provided herein are inhibitors of c-KIT kinase, pharmaceutical compositions comprising said inhibitory compounds, and methods for using said c-KIT kinase inhibitory compounds for the treatment of cancer, inflammatory, allergic, or autoimmune diseases, and other disorders characterized by aberrant c-KIT pathway signaling.

### CROSS-REFERENCE TO RELATED APPLICATION

This application claims the benefit of U.S. Patent Application No. 63/403,661, filed on Sep. 2, 2022; and U.S. Patent Application No. 63/335,687, filed on Apr. 27, 2022; each of which is hereby incorporated by reference in its entirety.

### BACKGROUND

The receptor tyrosine kinase c-KIT is involved in multiple signaling pathways crucial for cell proliferation and differentiation. In many types of tumors, c-KIT is overexpressed or mutated, and inhibition of c-KIT has become a validated treatment modality. Accordingly, therapies that target c-KIT kinase activity are desired for use in the treatment of cancer, inflammatory, allergic, and autoimmune diseases, and other disorders characterized by aberrant c-KIT pathway signaling.

### BRIEF SUMMARY OF THE INVENTION

Provided herein are inhibitors of c-KIT kinase, pharmaceutical compositions comprising said inhibitory compounds, and methods for using said inhibitory compounds for the treatment of disease.

One embodiment provides a compound, or a pharmaceutically acceptable salt or solvate thereof, having the structure of Formula (I):

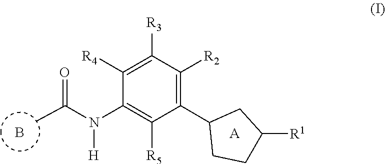

wherein,
Ring A is an optionally substituted 5-membered nitrogen-containing heteroaryl;
Ring B is an optionally substituted 9- or 10-membered bicyclic nitrogen-containing heteroaryl;
R¹ is optionally substituted alkyl, optionally substituted carbocyclyl, optionally substituted carbocyclylalkyl, optionally substituted heterocyclyl, optionally substituted heterocyclylalkyl, optionally substituted aryl, optionally substituted aralkyl, optionally substituted heteroaryl, or optionally substituted heteroaralkyl.
R² is hydrogen, halo, cyano, or optionally substituted C1-C6 alkyl;
R³ is halo;
R⁴ is optionally substituted C1-C6 alkyl; and
R⁵ is hydrogen, halo, cyano, or optionally substituted C1-C6 alkyl.

One embodiment provides a pharmaceutical composition comprising a compound of Formula (I), or pharmaceutically acceptable salt or solvate thereof, and at least one pharmaceutically acceptable excipient.

One embodiment provides a method of treating a disease or disorder in a patient in need thereof comprising administering to the patient a compound of Formula (I), or pharmaceutically acceptable salt or solvate thereof. Another embodiment provides the method wherein the disease or disorder is cancer, inflammatory, allergic, or autoimmune disease, and other disorder characterized by aberrant c-KIT pathway signaling.

### INCORPORATION BY REFERENCE

All publications, patents, and patent applications mentioned in this specification are herein incorporated by reference for the specific purposes identified herein.

### DETAILED DESCRIPTION OF THE INVENTION

As used herein and in the appended claims, the singular forms “a,” “and,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth. When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range, in some instances, will vary between 1% and 15% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, “consist of” or “consist essentially of” the described features.

### Definitions

As used in the specification and appended claims, unless specified to the contrary, the following terms have the meaning indicated below.

“Amino” refers to the —NH₂ radical.

“Cyano” refers to the —CN radical.

“Nitro” refers to the —NO₂ radical.

“Oxa” refers to the —O— radical.

“Oxo” refers to the ═O radical.

“Thioxo” refers to the ═S radical.

“Imino” refers to the ═N—H radical.

“Oximo” refers to the ═N—OH radical.

“Hydrazino” refers to the ═N—NH₂ radical.

“Alkyl” refers to a straight or branched hydrocarbon chain radical consisting solely of carbon and hydrogen atoms, containing no unsaturation, having from one to fifteen carbon atoms (e.g., C₁-C₁₅ alkyl). In certain embodiments, an alkyl comprises one to thirteen carbon atoms (e.g., C₁-C₁₃ alkyl). In certain embodiments, an alkyl comprises one to eight carbon atoms (e.g., C₁-C₈ alkyl). In other embodiments, an alkyl comprises one to five carbon atoms (e.g., C₁-C₅ alkyl). In other embodiments, an alkyl comprises one to four carbon atoms (e.g., C₁-C₄ alkyl). In other embodiments, an alkyl comprises one to three carbon atoms (e.g., C₁-C₃ alkyl). In other embodiments, an alkyl comprises one to two carbon atoms (e.g., C₁-C₂ alkyl). In other embodiments, an alkyl comprises one carbon atom (e.g., C₁ alkyl). In other embodiments, an alkyl comprises five to fifteen carbon atoms (e.g., C₅-C₁₅ alkyl). In other embodiments, an alkyl comprises five to eight carbon atoms (e.g., C₅-C₈ alkyl). In other embodiments, an alkyl comprises two to five carbon atoms (e.g., C₂-C₅ alkyl). In other embodiments, an alkyl comprises three to five carbon atoms (e.g., C₃-C₅ alkyl). In other embodiments, the alkyl group is selected from methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), 1-pentyl (n-pentyl). The alkyl is attached to the rest of the molecule by a single bond. Unless stated otherwise specifically in the specification, an alkyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2); where each R^{a} is independently hydrogen, alkyl (optionally substituted with halogen, hydroxy, methoxy, amino or trifluoromethyl), fluoroalkyl, carbocyclyl (optionally substituted with halogen, hydroxy, methoxy, amino or trifluoromethyl), carbocyclylalkyl (optionally substituted with halogen, hydroxy, methoxy, amino or trifluoromethyl), aryl (optionally substituted with halogen, hydroxy, methoxy, amino or trifluoromethyl), aralkyl (optionally substituted with halogen, hydroxy, methoxy, amino or trifluoromethyl), heterocyclyl (optionally substituted with halogen, hydroxy, methoxy, amino or trifluoromethyl), heterocyclylalkyl (optionally substituted with halogen, hydroxy, methoxy, amino or trifluoromethyl), heteroaryl (optionally substituted with halogen, hydroxy, methoxy, amino or trifluoromethyl), or heteroarylalkyl (optionally substituted with halogen, hydroxy, methoxy, amino or trifluoromethyl), or optionally, for a —N(R^{a})₂ group, both R^{a} groups may join to form a nitrogen-containing heterocyclyl. In certain embodiments, an optionally substituted alkyl is a haloalkyl. In other embodiments, an optionally substituted alkyl is a fluoroalkyl. In other embodiments, an optionally substituted alkyl is a —CF₃ group.

“Alkoxy” refers to a radical bonded through an oxygen atom of the formula —O-alkyl, where alkyl is an alkyl chain as defined above.

“Alkenyl” refers to a straight or branched hydrocarbon chain radical group consisting solely of carbon and hydrogen atoms, containing at least one carbon-carbon double bond, and having from two to twelve carbon atoms. In certain embodiments, an alkenyl comprises two to eight carbon atoms. In other embodiments, an alkenyl comprises two to four carbon atoms. The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like. Unless stated otherwise specifically in the specification, an alkenyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, amino or trifluoromethyl), fluoroalkyl, carbocyclyl (optionally substituted with halogen, hydroxy, methoxy, amino or trifluoromethyl), carbocyclylalkyl (optionally substituted with halogen, hydroxy, methoxy, amino or trifluoromethyl), aryl (optionally substituted with halogen, hydroxy, methoxy, amino or trifluoromethyl), aralkyl (optionally substituted with halogen, hydroxy, methoxy, amino or trifluoromethyl), heterocyclyl (optionally substituted with halogen, hydroxy, methoxy, amino or trifluoromethyl), heterocyclylalkyl (optionally substituted with halogen, hydroxy, methoxy, amino or trifluoromethyl), heteroaryl (optionally substituted with halogen, hydroxy, methoxy, amino or trifluoromethyl), or heteroarylalkyl (optionally substituted with halogen, hydroxy, methoxy, amino or trifluoromethyl), or optionally, for a —N(R^{a})₂ group, both R^{a} groups may join to form a nitrogen-containing heterocyclyl.

“Alkynyl” refers to a straight or branched hydrocarbon chain radical group consisting solely of carbon and hydrogen atoms, containing at least one carbon-carbon triple bond, having from two to twelve carbon atoms. In certain embodiments, an alkynyl comprises two to eight carbon atoms. In other embodiments, an alkynyl comprises two to six carbon atoms. In other embodiments, an alkynyl comprises two to four carbon atoms. The alkynyl is attached to the rest of the molecule by a single bond, for example, ethynyl, propynyl, butynyl, pentynyl, hexynyl, and the like. Unless stated otherwise specifically in the specification, an alkynyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, amino or trifluoromethyl), fluoroalkyl, carbocyclyl (optionally substituted with halogen, hydroxy, methoxy, amino or trifluoromethyl), carbocyclylalkyl (optionally substituted with halogen, hydroxy, methoxy, amino or trifluoromethyl), aryl (optionally substituted with halogen, hydroxy, methoxy, amino or trifluoromethyl), aralkyl (optionally substituted with halogen, hydroxy, methoxy, amino or trifluoromethyl), heterocyclyl (optionally substituted with halogen, hydroxy, methoxy, amino or trifluoromethyl), heterocyclylalkyl (optionally substituted with halogen, hydroxy, methoxy, amino or trifluoromethyl), heteroaryl (optionally substituted with halogen, hydroxy, methoxy, amino or trifluoromethyl), or heteroarylalkyl (optionally substituted with halogen, hydroxy, methoxy, amino or trifluoromethyl), or optionally, for a —N(R^{a})₂ group, both R^{a} groups may join to form a nitrogen-containing heterocyclyl.

“Alkylene” or “alkylene chain” refers to a straight or branched divalent hydrocarbon chain linking the rest of the molecule to a radical group, consisting solely of carbon and hydrogen, containing no unsaturation, and having from one to twelve carbon atoms, for example, methylene, ethylene, propylene, n-butylene, and the like. The alkylene chain is attached to the rest of the molecule through a single bond and to the radical group through a single bond. The points of attachment of the alkylene chain to the rest of the molecule and to the radical group are through one carbon in the alkylene chain or through any two carbons within the chain. In certain embodiments, an alkylene comprises one to eight carbon atoms (e.g., C₁-C₈ alkylene). In other embodiments, an alkylene comprises one to five carbon atoms (e.g., C₁-C₅ alkylene). In other embodiments, an alkylene comprises one to four carbon atoms (e.g., C₁-C₄ alkylene). In other embodiments, an alkylene comprises one to three carbon atoms (e.g., C₁-C₃ alkylene). In other embodiments, an alkylene comprises one to two carbon atoms (e.g., C₁-C₂ alkylene). In other embodiments, an alkylene comprises one carbon atom (e.g., C₁ alkylene). In other embodiments, an alkylene comprises five to eight carbon atoms (e.g., C₅-C₈ alkylene). In other embodiments, an alkylene comprises two to five carbon atoms (e.g., C₂-C₅ alkylene). In other embodiments, an alkylene comprises three to five carbon atoms (e.g., C₃-C₅ alkylene). Unless stated otherwise specifically in the specification, an alkylene chain is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, amino or trifluoromethyl), fluoroalkyl, carbocyclyl (optionally substituted with halogen, hydroxy, methoxy, amino or trifluoromethyl), carbocyclylalkyl (optionally substituted with halogen, hydroxy, methoxy, amino or trifluoromethyl), aryl (optionally substituted with halogen, hydroxy, methoxy, amino or trifluoromethyl), aralkyl (optionally substituted with halogen, hydroxy, methoxy, amino or trifluoromethyl), heterocyclyl (optionally substituted with halogen, hydroxy, methoxy, amino or trifluoromethyl), heterocyclylalkyl (optionally substituted with halogen, hydroxy, methoxy, amino or trifluoromethyl), heteroaryl (optionally substituted with halogen, hydroxy, methoxy, amino or trifluoromethyl), or heteroarylalkyl (optionally substituted with halogen, hydroxy, methoxy, amino or trifluoromethyl), or optionally, for a —N(R^{a})₂ group, both R^{a} groups may join to form a nitrogen-containing heterocyclyl.

“Alkenylene” or “alkenylene chain” refers to a straight or branched divalent hydrocarbon chain linking the rest of the molecule to a radical group, consisting solely of carbon and hydrogen, containing at least one carbon-carbon double bond, and having from two to twelve carbon atoms. The alkenylene chain is attached to the rest of the molecule through a single bond and to the radical group through a single bond. In certain embodiments, an alkenylene comprises two to eight carbon atoms (e.g., C₂-C₈ alkenylene). In other embodiments, an alkenylene comprises two to five carbon atoms (e.g., C₂-C₅ alkenylene). In other embodiments, an alkenylene comprises two to four carbon atoms (e.g., C₂-C₄ alkenylene). In other embodiments, an alkenylene comprises two to three carbon atoms (e.g., C₂-C₃ alkenylene). In other embodiments, an alkenylene comprises two carbon atoms (e.g., C₂ alkenylene). In other embodiments, an alkenylene comprises five to eight carbon atoms (e.g., C₅-C₈ alkenylene). In other embodiments, an alkenylene comprises three to five carbon atoms (e.g., C₃-C₅ alkenylene).

Unless stated otherwise specifically in the specification, an alkenylene chain is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, amino or trifluoromethyl), fluoroalkyl, carbocyclyl (optionally substituted with halogen, hydroxy, methoxy, amino or trifluoromethyl), carbocyclylalkyl (optionally substituted with halogen, hydroxy, methoxy, amino or trifluoromethyl), aryl (optionally substituted with halogen, hydroxy, methoxy, amino or trifluoromethyl), aralkyl (optionally substituted with halogen, hydroxy, methoxy, amino or trifluoromethyl), heterocyclyl (optionally substituted with halogen, hydroxy, methoxy, amino or trifluoromethyl), heterocyclylalkyl (optionally substituted with halogen, hydroxy, methoxy, amino or trifluoromethyl), heteroaryl (optionally substituted with halogen, hydroxy, methoxy, amino or trifluoromethyl), or heteroarylalkyl (optionally substituted with halogen, hydroxy, methoxy, amino or trifluoromethyl), or optionally, for a —N(R^{a})₂ group, both R^{a} groups may join to form a nitrogen-containing heterocyclyl.

“Alkynylene” or “alkynylene chain” refers to a straight or branched divalent hydrocarbon chain linking the rest of the molecule to a radical group, consisting solely of carbon and hydrogen, containing at least one carbon-carbon triple bond, and having from two to twelve carbon atoms. The alkynylene chain is attached to the rest of the molecule through a single bond and to the radical group through a single bond. In certain embodiments, an alkynylene comprises two to eight carbon atoms (e.g., C₂-C₈ alkynylene). In other embodiments, an alkynylene comprises two to five carbon atoms (e.g., C₂-C₅ alkynylene). In other embodiments, an alkynylene comprises two to four carbon atoms (e.g., C₂-C₄ alkynylene). In other embodiments, an alkynylene comprises two to three carbon atoms (e.g., C₂-C₃ alkynylene). In other embodiments, an alkynylene comprises two carbon atoms (e.g., C₂ alkynylene). In other embodiments, an alkynylene comprises five to eight carbon atoms (e.g., C₅-C₈ alkynylene). In other embodiments, an alkynylene comprises three to five carbon atoms (e.g., C₃-C₅ alkynylene).

Unless stated otherwise specifically in the specification, an alkynylene chain is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, amino or trifluoromethyl), fluoroalkyl, carbocyclyl (optionally substituted with halogen, hydroxy, methoxy, amino or trifluoromethyl), carbocyclylalkyl (optionally substituted with halogen, hydroxy, methoxy, amino or trifluoromethyl), aryl (optionally substituted with halogen, hydroxy, methoxy, amino or trifluoromethyl), aralkyl (optionally substituted with halogen, hydroxy, methoxy, amino or trifluoromethyl), heterocyclyl (optionally substituted with halogen, hydroxy, methoxy, amino or trifluoromethyl), heterocyclylalkyl (optionally substituted with halogen, hydroxy, methoxy, amino or trifluoromethyl), heteroaryl (optionally substituted with halogen, hydroxy, methoxy, amino or trifluoromethyl), or heteroarylalkyl (optionally substituted with halogen, hydroxy, methoxy, amino or trifluoromethyl), or optionally, for a —N(R^{a})₂ group, both R^{a} groups may join to form a nitrogen-containing heterocyclyl.

“Aryl” refers to a radical derived from an aromatic monocyclic or multicyclic hydrocarbon ring system by removing a hydrogen atom from a ring carbon atom. The aromatic monocyclic or multicyclic hydrocarbon ring system contains only hydrogen and carbon from five to eighteen carbon atoms, where at least one of the rings in the ring system is fully unsaturated, i.e., it contains a cyclic, delocalized (4n+2) π-electron system in accordance with the Hückel theory.

The ring system from which aryl groups are derived include, but are not limited to, groups such as benzene, fluorene, indane, indene, tetralin and naphthalene. Unless stated otherwise specifically in the specification, the term “aryl” or the prefix “ar-” (such as in “aralkyl”) is meant to include aryl radicals optionally substituted by one or more substituents independently selected from optionally substituted alkyl, optionally substituted alkenyl, optionally substituted alkynyl, halo, cyano, nitro, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); where each R^{a} is independently hydrogen, alkyl (optionally substituted with halogen, hydroxy, methoxy, amino or trifluoromethyl), fluoroalkyl, cycloalkyl (optionally substituted with halogen, hydroxy, methoxy, amino or trifluoromethyl), cycloalkylalkyl (optionally substituted with halogen, hydroxy, methoxy, amino or trifluoromethyl), aryl (optionally substituted with halogen, hydroxy, methoxy, amino or trifluoromethyl), aralkyl (optionally substituted with halogen, hydroxy, methoxy, amino or trifluoromethyl), heterocyclyl (optionally substituted with halogen, hydroxy, methoxy, amino or trifluoromethyl), heterocyclylalkyl (optionally substituted with halogen, hydroxy, methoxy, amino or trifluoromethyl), heteroaryl (optionally substituted with halogen, hydroxy, methoxy, amino or trifluoromethyl), or heteroarylalkyl (optionally substituted with halogen, hydroxy, methoxy, amino or trifluoromethyl); or optionally, for a —N(R^{a})₂ group, both R^{a} groups may join to form a nitrogen-containing heterocyclyl; each R^{b} is independently a direct bond or a straight or branched alkylene or alkenylene chain, and R^{c} is a straight or branched alkylene or alkenylene chain, and where each of the R^{a}, R^{b}, or R^{c} substituents is unsubstituted unless otherwise indicated.

“Aralkyl” refers to a radical of the formula —R-aryl where R^{c} is an alkylene chain as defined above, for example, methylene, ethylene, and the like. The alkylene chain part of the aralkyl radical is optionally substituted as described above for an alkylene chain. The aryl part of the aralkyl radical is optionally substituted as described above for an aryl group.

“Aralkenyl” refers to a radical of the formula —R^{d}-aryl where R^{d} is an alkenylene chain as defined above. The aryl part of the aralkenyl radical is optionally substituted as described above for an aryl group. The alkenylene chain part of the aralkenyl radical is optionally substituted as defined above for an alkenylene group.

“Aralkynyl” refers to a radical of the formula —R^{e}-aryl, where R^{e} is an alkynylene chain as defined above. The aryl part of the aralkynyl radical is optionally substituted as described above for an aryl group. The alkynylene chain part of the aralkynyl radical is optionally substituted as defined above for an alkynylene chain.

“Aralkoxy” refers to a radical bonded through an oxygen atom of the formula —O—R^{c}-aryl where R^{c} is an alkylene chain as defined above, for example, methylene, ethylene, and the like. The alkylene chain part of the aralkyl radical is optionally substituted as described above for an alkylene chain. The aryl part of the aralkyl radical is optionally substituted as described above for an aryl group.

“Carbocyclyl” refers to a stable non-aromatic monocyclic or polycyclic hydrocarbon radical consisting solely of carbon and hydrogen atoms, which includes fused or bridged ring systems, having from three to fifteen carbon atoms. In certain embodiments, a carbocyclyl comprises three to ten carbon atoms. In other embodiments, a carbocyclyl comprises five to seven carbon atoms. The carbocyclyl is attached to the rest of the molecule by a single bond. Carbocyclyl is saturated (i.e., containing single C—C bonds only) or unsaturated (i.e., containing one or more double bonds or triple bonds). A fully saturated carbocyclyl radical is also referred to as “cycloalkyl.” Examples of monocyclic cycloalkyls include, e.g., cyclopropyl, cyclobutyl, cyclopentyl, cyclohexyl, cycloheptyl, and cyclooctyl. An unsaturated carbocyclyl is also referred to as “cycloalkenyl.” Examples of monocyclic cycloalkenyls include, e.g., cyclopentenyl, cyclohexenyl, cycloheptenyl, and cyclooctenyl. Polycyclic carbocyclyl radicals include, for example, adamantyl, norbornyl (i.e., bicyclo[2.2.1]heptanyl), norbornenyl, decalinyl, 7,7-dimethyl-bicyclo[2.2.1]heptanyl, and the like. Unless otherwise stated specifically in the specification, the term “carbocyclyl” is meant to include carbocyclyl radicals that are optionally substituted by one or more substituents independently selected from optionally substituted alkyl, optionally substituted alkenyl, optionally substituted alkynyl, halo, oxo, thioxo, cyano, nitro, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); where each R^{a} is independently hydrogen, alkyl (optionally substituted with halogen, hydroxy, methoxy, amino or trifluoromethyl), fluoroalkyl, cycloalkyl (optionally substituted with halogen, hydroxy, methoxy, amino or trifluoromethyl), cycloalkylalkyl (optionally substituted with halogen, hydroxy, methoxy, amino or trifluoromethyl), aryl (optionally substituted with halogen, hydroxy, methoxy, amino or trifluoromethyl), aralkyl (optionally substituted with halogen, hydroxy, methoxy, amino or trifluoromethyl), heterocyclyl (optionally substituted with halogen, hydroxy, methoxy, amino or trifluoromethyl), heterocyclylalkyl (optionally substituted with halogen, hydroxy, methoxy, amino or trifluoromethyl), heteroaryl (optionally substituted with halogen, hydroxy, methoxy, amino or trifluoromethyl), or heteroarylalkyl (optionally substituted with halogen, hydroxy, methoxy, amino or trifluoromethyl); or optionally, for a —N(R^{a})₂ group, both R^{a} groups may join to form a nitrogen-containing heterocyclyl; each R^{b} is independently a direct bond or a straight or branched alkylene or alkenylene chain, and R^{c} is a straight or branched alkylene or alkenylene chain, and where each of the R^{a}, R^{b}, or R^{c} substituents is unsubstituted unless otherwise indicated.

“Carbocyclylalkyl” refers to a radical of the formula —R^{c}-carbocyclyl where R^{c} is an alkylene chain as defined above. The alkylene chain and the carbocyclyl radical is optionally substituted as defined above.

“Carbocyclylalkynyl” refers to a radical of the formula —R^{c}-carbocyclyl where R^{c} is an alkynylene chain as defined above. The alkynylene chain and the carbocyclyl radical is optionally substituted as defined above.

“Carbocyclylalkoxy” refers to a radical bonded through an oxygen atom of the formula —O—R^{c}-carbocyclyl where R^{c} is an alkylene chain as defined above. The alkylene chain and the carbocyclyl radical is optionally substituted as defined above.

“Halo” or “halogen” refers to bromo, chloro, fluoro or iodo substituents.

“Fluoroalkyl” refers to an alkyl radical, as defined above, that is substituted by one or more fluoro radicals, as defined above, for example, trifluoromethyl, difluoromethyl, fluoromethyl, 2,2,2-trifluoroethyl, 1-fluoromethyl-2-fluoroethyl, and the like. In some embodiments, the alkyl part of the fluoroalkyl radical is optionally substituted as defined above for an alkyl group.

“Heterocyclyl” refers to a stable 3- to 18-membered non-aromatic ring radical that comprises two to twelve carbon atoms and from one to six heteroatoms selected from nitrogen, oxygen and sulfur. Unless stated otherwise specifically in the specification, the heterocyclyl radical is a monocyclic, bicyclic, tricyclic, or tetracyclic ring system, which optionally includes fused or bridged ring systems. The heteroatoms in the heterocyclyl radical are optionally oxidized. One or more nitrogen atoms, if present, are optionally quaternized. The heterocyclyl radical is partially or fully saturated. The heterocyclyl is attached to the rest of the molecule through any atom of the ring(s). Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, the term “heterocyclyl” is meant to include heterocyclyl radicals as defined above that are optionally substituted by one or more substituents selected from optionally substituted alkyl, optionally substituted alkenyl, optionally substituted alkynyl, halo, fluoroalkyl, oxo, thioxo, cyano, nitro, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); where each R^{a} is independently hydrogen, alkyl (optionally substituted with halogen, hydroxy, methoxy, amino or trifluoromethyl), fluoroalkyl, cycloalkyl (optionally substituted with halogen, hydroxy, methoxy, amino or trifluoromethyl), cycloalkylalkyl (optionally substituted with halogen, hydroxy, methoxy, amino or trifluoromethyl), aryl (optionally substituted with halogen, hydroxy, methoxy, amino or trifluoromethyl), aralkyl (optionally substituted with halogen, hydroxy, methoxy, amino or trifluoromethyl), heterocyclyl (optionally substituted with halogen, hydroxy, methoxy, amino or trifluoromethyl), heterocyclylalkyl (optionally substituted with halogen, hydroxy, methoxy, amino or trifluoromethyl), heteroaryl (optionally substituted with halogen, hydroxy, methoxy, amino or trifluoromethyl), or heteroarylalkyl (optionally substituted with halogen, hydroxy, methoxy, amino or trifluoromethyl), or optionally, for a —N(R^{a})₂ group, both R^{a} groups may join to form a nitrogen-containing heterocyclyl; each R^{b} is independently a direct bond or a straight or branched alkylene or alkenylene chain; R^{c} is a straight or branched alkylene or alkenylene chain; and where each of the R^{a}, R^{b}, or R^{c} substituents is unsubstituted unless otherwise indicated.

“N-heterocyclyl” or “N-attached heterocyclyl” refers to a heterocyclyl radical as defined above containing at least one nitrogen and where the point of attachment of the heterocyclyl radical to the rest of the molecule is through a nitrogen atom in the heterocyclyl radical. An N-heterocyclyl radical is optionally substituted as described above for heterocyclyl radicals.

Examples of such N-heterocyclyl radicals include, but are not limited to, 1-morpholinyl, 1-piperidinyl, 1-piperazinyl, 1-pyrrolidinyl, pyrazolidinyl, imidazolinyl, and imidazolidinyl.

“C-heterocyclyl” or “C-attached heterocyclyl” refers to a heterocyclyl radical as defined above containing at least one heteroatom and where the point of attachment of the heterocyclyl radical to the rest of the molecule is through a carbon atom in the heterocyclyl radical. A C-heterocyclyl radical is optionally substituted as described above for heterocyclyl radicals. Examples of such C-heterocyclyl radicals include, but are not limited to, 2-morpholinyl, 2- or 3- or 4-piperidinyl, 2-piperazinyl, 2- or 3-pyrrolidinyl, and the like.

“Heterocyclylalkyl” refers to a radical of the formula —R^{c}-heterocyclyl where R^{c} is an alkylene chain as defined above. If the heterocyclyl is a nitrogen-containing heterocyclyl, the heterocyclyl is optionally attached to the alkyl radical at the nitrogen atom. The alkylene chain of the heterocyclylalkyl radical is optionally substituted as defined above for an alkylene chain.

The heterocyclyl part of the heterocyclylalkyl radical is optionally substituted as defined above for a heterocyclyl group.

“Heterocyclylalkoxy” refers to a radical bonded through an oxygen atom of the formula —O—R^{c}-heterocyclyl where R^{c} is an alkylene chain as defined above. If the heterocyclyl is a nitrogen-containing heterocyclyl, the heterocyclyl is optionally attached to the alkyl radical at the nitrogen atom. The alkylene chain of the heterocyclylalkoxy radical is optionally substituted as defined above for an alkylene chain. The heterocyclyl part of the heterocyclylalkoxy radical is optionally substituted as defined above for a heterocyclyl group.

“Heteroaryl” refers to a radical derived from a 3- to 18-membered aromatic ring radical that comprises two to seventeen carbon atoms and from one to six heteroatoms selected from nitrogen, oxygen, and sulfur. As used herein, the heteroaryl radical is a monocyclic, bicyclic, tricyclic, or tetracyclic ring system, wherein at least one of the rings in the ring system is fully unsaturated, i.e., it contains a cyclic, delocalized (4n+2) π-electron system in accordance with the Hückel theory. Heteroaryl includes fused or bridged ring systems. The heteroatom(s) in the heteroaryl radical is optionally oxidized. One or more nitrogen atoms, if present, are optionally quaternized. The heteroaryl is attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, triazolyl, tetrazolyl, triazinyl, thieno[2,3-d]pyrimidinyl, thieno[3,2-d]pyrimidinyl, thieno[2,3-c]pridinyl, and thiophenyl (i.e. thienyl). Unless stated otherwise specifically in the specification, the term “heteroaryl” is meant to include heteroaryl radicals as defined above which are optionally substituted by one or more substituents selected from optionally substituted alkyl, optionally substituted alkenyl, optionally substituted alkynyl, halo, optionally substituted fluoroalkyl, optionally substituted haloalkenyl, optionally substituted haloalkynyl, oxo, thioxo, cyano, nitro, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); where each R^{a} is independently hydrogen, alkyl (optionally substituted with halogen, hydroxy, methoxy, amino or trifluoromethyl), fluoroalkyl, cycloalkyl (optionally substituted with halogen, hydroxy, methoxy, amino or trifluoromethyl), cycloalkylalkyl (optionally substituted with halogen, hydroxy, methoxy, amino or trifluoromethyl), aryl (optionally substituted with halogen, hydroxy, methoxy, amino or trifluoromethyl), aralkyl (optionally substituted with halogen, hydroxy, methoxy, amino or trifluoromethyl), heterocyclyl (optionally substituted with halogen, hydroxy, methoxy, amino or trifluoromethyl), heterocyclylalkyl (optionally substituted with halogen, hydroxy, methoxy, amino or trifluoromethyl), heteroaryl (optionally substituted with halogen, hydroxy, methoxy, amino or trifluoromethyl), or heteroarylalkyl (optionally substituted with halogen, hydroxy, methoxy, amino or trifluoromethyl); or optionally, for a —N(R^{a})₂ group, both R^{a} groups may join to form a nitrogen-containing heterocyclyl; each R^{b} is independently a direct bond or a straight or branched alkylene or alkenylene chain, and R^{c} is a straight or branched alkylene or alkenylene chain, and where each of the R^{a}, R^{b}, or R^{c} substituents is unsubstituted unless otherwise indicated.

“N-heteroaryl” refers to a heteroaryl radical as defined above containing at least one nitrogen and where the point of attachment of the heteroaryl radical to the rest of the molecule is through a nitrogen atom in the heteroaryl radical. An N-heteroaryl radical is optionally substituted as described above for heteroaryl radicals.

“C-heteroaryl” refers to a heteroaryl radical as defined above and where the point of attachment of the heteroaryl radical to the rest of the molecule is through a carbon atom in the heteroaryl radical. A C-heteroaryl radical is optionally substituted as described above for heteroaryl radicals.

“Heteroarylalkyl” refers to a radical of the formula —R^{c}-heteroaryl, where R^{c} is an alkylene chain as defined above. If the heteroaryl is a nitrogen-containing heteroaryl, the heteroaryl is optionally attached to the alkyl radical at the nitrogen atom. The alkylene chain of the heteroarylalkyl radical is optionally substituted as defined above for an alkylene chain. The heteroaryl part of the heteroarylalkyl radical is optionally substituted as defined above for a heteroaryl group.

“Heteroarylalkoxy” refers to a radical bonded through an oxygen atom of the formula —O—R^{c}-heteroaryl, where R^{c} is an alkylene chain as defined above. If the heteroaryl is a nitrogen-containing heteroaryl, the heteroaryl is optionally attached to the alkyl radical at the nitrogen atom. The alkylene chain of the heteroarylalkoxy radical is optionally substituted as defined above for an alkylene chain. The heteroaryl part of the heteroarylalkoxy radical is optionally substituted as defined above for a heteroaryl group.

The compounds disclosed herein, in some embodiments, contain one or more asymmetric centers and thus give rise to enantiomers, diastereomers, and other stereoisomeric forms that are defined, in terms of absolute stereochemistry, as (R)- or (S)-. Unless stated otherwise, it is intended that all stereoisomeric forms of the compounds disclosed herein are contemplated by this disclosure. When the compounds described herein contain alkene double bonds, and unless specified otherwise, it is intended that this disclosure includes both E and Z geometric isomers (e.g., cis or trans.) Likewise, all possible isomers, as well as their racemic and optically pure forms, and all tautomeric forms are also intended to be included. The term “geometric isomer” refers to E or Z geometric isomers (e.g., cis or trans) of an alkene double bond. The term “positional isomer” refers to structural isomers around a central ring, such as ortho-, meta-, and para-isomers around a benzene ring.

A “tautomer” refers to a molecule wherein a proton shift from one atom of a molecule to another atom of the same molecule is possible. The compounds presented herein, in certain embodiments, exist as tautomers. In circumstances where tautomerization is possible, a chemical equilibrium of the tautomers will exist. The exact ratio of the tautomers depends on several factors, including physical state, temperature, solvent, and pH. Some examples of tautomeric equilibrium include:

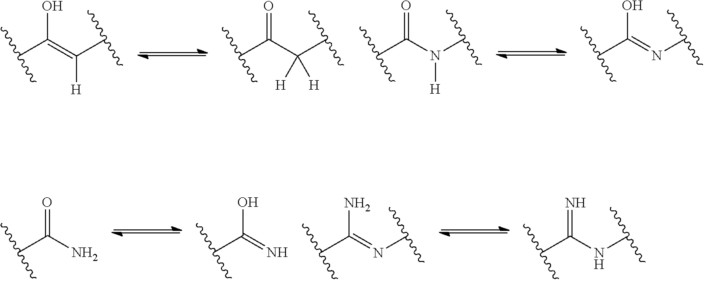

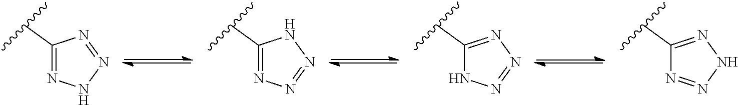 [SMILES: *c1nn[nH]n1 ; *c1nnn[nH]1 ; *c1nnn[nH]1 ; *c1nn[nH]n1]

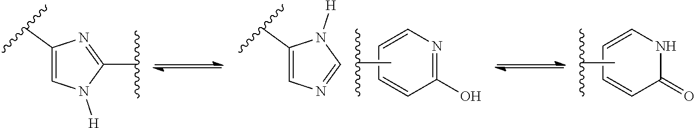

The compounds disclosed herein, in some embodiments, are used in different enriched isotopic forms, e.g., enriched in the content of ²H, ³H, ¹¹C, ¹³C and/or ¹⁴C. In one particular embodiment, the compound is deuterated in at least one position. Such deuterated forms can be made by the procedure described in U.S. Pat. Nos. 5,846,514 and 6,334,997. As described in U.S. Pat. Nos. 5,846,514 and 6,334,997, deuteration can improve the metabolic stability and or efficacy, thus increasing the duration of action of drugs.

Unless otherwise stated, structures depicted herein are intended to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of the present disclosure.

The compounds of the present disclosure optionally contain unnatural proportions of atomic isotopes at one or more atoms that constitute such compounds. For example, the compounds may be labeled with isotopes, such as for example, deuterium (²H), tritium (³H), iodine-125 (¹²⁵I) or carbon-14 (¹⁴C). Isotopic substitution with ²H, ¹¹C, ¹³C, ¹⁴C, ¹⁵C, ¹²N, ¹³N, ¹⁵N, ¹⁶N, ¹⁶O ¹⁷O, ¹⁴F, ¹⁵F, ¹⁶F, ¹⁷F, ¹⁸F, ³³S, ³⁴S, ³⁵S, ³⁶S, ³⁵Cl, ³⁷Cl, ⁷⁹Br, ⁸¹Br, ¹²⁵I are all contemplated. In some embodiments, isotopic substitution with ¹⁸F is contemplated. All isotopic variations of the compounds of the present invention, whether radioactive or not, are encompassed within the scope of the present invention.

In certain embodiments, the compounds disclosed herein have some or all of the ¹H atoms replaced with ²H atoms. The methods of synthesis for deuterium-containing compounds are known in the art and include, by way of non-limiting example only, the following synthetic methods.

Deuterium substituted compounds are synthesized using various methods such as described in: Dean, Dennis C.; Editor. Recent Advances in the Synthesis and Applications of Radiolabeled Compounds for Drug Discovery and Development. [Curr., Pharm. Des., 2000; 6 (10)]2000, 110 pp; George W.; Varma, Rajender S. The Synthesis of Radiolabeled Compounds via Organometallic Intermediates, Tetrahedron, 1989, 45(21), 6601-21; and Evans, E. Anthony. Synthesis of radiolabeled compounds, J. Radioanal. Chem., 1981, 64 (1-2), 9-32.

Deuterated starting materials are readily available and are subjected to the synthetic methods described herein to provide for the synthesis of deuterium-containing compounds. Large numbers of deuterium-containing reagents and building blocks are available commercially from chemical vendors, such as Aldrich Chemical Co.

Deuterium-transfer reagents suitable for use in nucleophilic substitution reactions, such as iodomethane-d₃ (CD₃I), are readily available and may be employed to transfer a deuterium-substituted carbon atom under nucleophilic substitution reaction conditions to the reaction substrate. The use of CD₃I is illustrated, by way of example only, in the reaction schemes below.

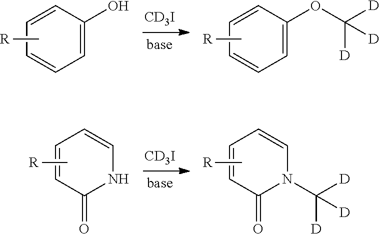

Deuterium-transfer reagents, such as lithium aluminum deuteride (LiAlD₄), are employed to transfer deuterium under reducing conditions to the reaction substrate. The use of LiAlD₄ is illustrated, by way of example only, in the reaction schemes below.

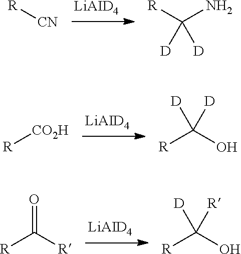

Deuterium gas and palladium catalyst are employed to reduce unsaturated carbon-carbon linkages and to perform a reductive substitution of aryl carbon-halogen bonds as illustrated, by way of example only, in the reaction schemes below.

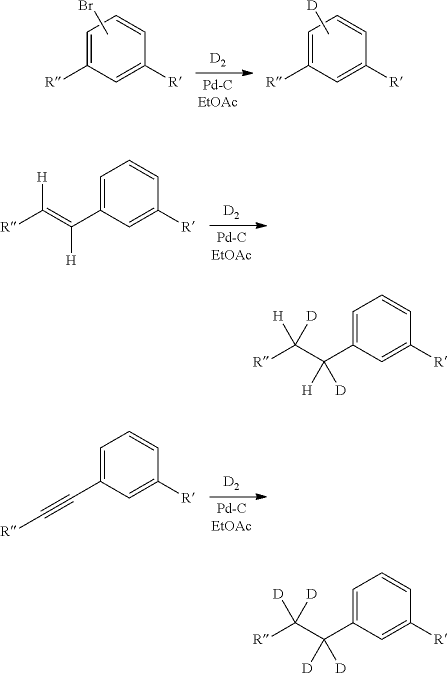

In one embodiment, the compounds disclosed herein contain one deuterium atom. In another embodiment, the compounds disclosed herein contain two deuterium atoms. In another embodiment, the compounds disclosed herein contain three deuterium atoms. In another embodiment, the compounds disclosed herein contain four deuterium atoms. In another embodiment, the compounds disclosed herein contain five deuterium atoms. In another embodiment, the compounds disclosed herein contain six deuterium atoms. In another embodiment, the compounds disclosed herein contain more than six deuterium atoms. In another embodiment, the compound disclosed herein is fully substituted with deuterium atoms and contains no non-exchangeable ¹H hydrogen atoms. In one embodiment, the level of deuterium incorporation is determined by synthetic methods in which a deuterated synthetic building block is used as a starting material.

“Pharmaceutically acceptable salt” includes both acid and base addition salts. A pharmaceutically acceptable salt of any one of the c-KIT kinase inhibitory compounds described herein is intended to encompass any and all pharmaceutically suitable salt forms. Preferred pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and. aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997)). Acid addition salts of basic compounds are, in some embodiments, prepared by contacting the free base forms with a sufficient amount of the desired acid to produce the salt according to methods and techniques with which a skilled artisan is familiar.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. Pharmaceutically acceptable base addition salts are, in some embodiments, formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

“Pharmaceutically acceptable solvate” refers to a composition of matter that is the solvent addition form. In some embodiments, solvates contain either stoichiometric or non-stoichiometric amounts of a solvent, and are formed during the process of making with pharmaceutically acceptable solvents such as water, ethanol, and the like. Hydrates are formed when the solvent is water, or alcoholates are formed when the solvent is alcohol. Solvates of compounds described herein are conveniently prepared or formed during the processes described herein. The compounds provided herein exist in either unsolvated or solvated forms.

The term “subject” or “patient” encompasses mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human.

As used herein, “treatment” or “treating,” or “palliating” or “ameliorating” are used interchangeably. These terms refer to an approach for obtaining beneficial or desired results including but not limited to therapeutic benefit and/or a prophylactic benefit. By “therapeutic benefit” is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient is still afflicted with the underlying disorder. For prophylactic benefit, the compositions are, in some embodiments, administered to a patient at risk of developing a particular disease, or to a patient reporting one or more of the physiological symptoms of a disease, even though a diagnosis of this disease has not been made.

c-KIT Kinase

c-KIT is a cytokine receptor that is classified as a type-III receptor tyrosine kinase. It is encoded by the c-kit gene and expressed on the surfaces of many cell types, including melanocytes, interstitial cells of Cajal, and hematopoietic cells (e.g., stem cells, hematopoietic progenitors, and mast cells). Proto-oncogene c-KIT (also called CD117, mast/stem cell growth factor receptor KIT, p145 c-kit, PBT, piebald trait protein, SFCR, v-kit Hardy Zuckerman 4 feline sarcoma viral oncogene homolog) is located on human chromosome 4q11-4q13. Factors regulating transcription of c-KIT include transcription factor SCL (e.g., transcription factor SCL/Tal1, runt-related transcription factor 1 (RUNX1), and far upstream binding protein 1 (FUBP1). It is expressed as multiple mRNA transcripts. The protein product of this gene is c-KIT, a receptor tyrosine kinase.

Structurally, c-KIT is a glycosylated transmembrane protein, although the protein can also be soluble. c-KIT has a molecular weight of approximately 145 kDa. c-KIT comprises an N-terminal extracellular region with five immunoglobulin-like domains, a hydrophobic transmembrane domain, a juxtamembrane domain, and a C-terminal intracellular tyrosine kinase region that is split into two domains by a hydrophilic insert sequence of approximately 80 amino acids. Four c-KIT isoforms formed by alternative splicing of RNA have been identified in humans. These isoforms are distinguished by (1) the presence of serine residues in the intracellular kinase insert sequence and (2) the presence of a sequence of four amino acids in the extracellular domain. The four isoforms exhibit differing abilities to induce signal transduction and tumorigenesis. Of the four isoforms, the isoform lacking the tetrapeptide exhibits the strongest signal transduction and tumorigenic abilities.

Functionally, c-KIT is involved in multiple signaling pathways crucial for cell survival, proliferation, differentiation, migration, and/or apoptosis. c-KIT is a type-III receptor tyrosine kinase. Signaling with c-KIT is typical of that of other receptor tyrosine kinases. c-KIT's cytokine ligand is called stem cell factor (SCF). Binding of SCF to c-KIT triggers dimerization of c-KIT. Dimerization activates c-KIT towards phosphorylation of multiple intracellular proteins for signal transduction. Four signaling pathways involving c-KIT have been at least partially elucidated: PI3-kinase, Src family kinase, Ras-Erk, and JAK/STAT. Following activation, c-KIT is ubiquitinated and thereby marked for destruction in a lysosome.

c-KIT is thought to be primarily involved in stem-cell differentiation and maintenance, e.g., hematopoiesis; gametogenesis; melanogenesis; and in the development, migration, and function of mast cells. c-KIT is especially expressed in hematopoietic stem cells, which divide asymmetrically. Division of hematopoietic stem cells results in self-renewal or differentiation into myeloid (e.g., monocytes and macrophages, neutrophils, basophils, eosinophils, erythrocytes, megakaryocytes/platelets, and dendritic cells) and lymphoid (e.g., T-, B-, and NK-cells) lineages. During differentiation, self-renewal and stemness are reduced, and expression of c-KIT can be significantly diminished, or even nearly vanished. In germ cells, c-KIT is involved in apoptosis, cell migration, and proliferation. In melanocytes, c-KIT is involved in proliferation and migration from the neuronal crest to the dermis. c-KIT is also involved in the development and function of interstitial cells of Cajal and is implicated in the migration of neural stem cells (neuroproliferative cells) to injured areas of the brain.

SCF (also known as “steel factor”), the c-KIT cytokine ligand, is a glycosylated protein with a molecular weight of 2-35 kDa that stimulates the growth and movement of cells. SCF binds to the second and third immunoglobulin domains of c-KIT. Soluble SCF has a weight of approximately 18.5 kDa and forms a dimer. The gene coding for SCF is located at 12q21-32. SCF is found in normal human blood serum at a concentration of 3.3 ng/mL. SCF mainly exists in fibroblasts and bone marrow stromal cells. SCF is involved in hematopoiesis, spermatogenesis, and melanogenesis. Both soluble and transmembrane SCF must be present for normal hematopoietic function; these disparate forms are produced by alternative splicing of the same RNA transcript. At exon 6, soluble SCF contains a proteolytic cleavage site, cleavage at which triggers release of the extracellular portion of the protein. It has been hypothesized that SCF guides (e.g., by binding KIT) hematopoietic stem cells to their stem cell niches and is involved in maintenance of these cells. Similarly, during development, SCF helps to localize melanocytes by guiding melanoblasts to their terminal locations, and, during spermatogenesis, SCF guides germ cells to their appropriate locations in the body. In addition to SCF, c-KIT interacts with APS, BCR, CD63, CD81, CD9, CRK, CRKL, DOK1, FES, GRB10, Grb2, KITLG, LNK, LYN, MATK, MPDZ, PIK3R11, PTPN11, PTPN6, STAT1, SOCS1, SOCS6, SRC, and TEC.

Because c-KIT is a proto-oncogene, deregulation (e.g., through gain of function, loss of function, overexpression, and point mutations) of this gene can lead to cancer. The precise role of c-KIT in the development and progression of cancer has not been fully elucidated. Mutations in the c-KIT proto-oncogene have been associated with piebaldism, gastrointestinal stromal tumors (GIST), melanoma, systemic mastocytosis, mast cell disease, leukemia (acute myelogenous, core-factor binding, and mast cell), and other cancers, including sinonasal natural killer/T-cell lymphoma (NKTCL), seminoma, lung adenocarcinoma, colon adenocarcinoma, conventional glioblastoma multiforme, and intracranial and ovarian dysgerminoma.

In many types of tumors, c-KIT is overexpressed or mutated. For example, seminomas (a type of testicular germ cell tumors) frequently exhibit mutations at exon 17, amplification, and/or overexpression of c-KIT. While other tumors (e.g., melanoma, thyroid carcinoma, and breast cancer) can be associated with loss-of function mutations of c-KIT, gain-of-function mutations are thought to be the main events leading to cancer progression. Examples of gain-of-function mutations of c-KIT include D816V, located in the juxtamembrane domain, and V560G, located in the tyrosine kinase domain. Point mutations in either of these domains can induce dimerization, and thus also activation, of c-KIT. The role of c-KIT in the aforementioned diseases has inspired the concept that inhibition of c-KIT can be a target for cancer therapy. c-KIT inhibition has shown promising results for the treatment of GIST, acute myeloid leukemia, melanoma, and other cancers. Drugs that target c-KIT include, but are not limited to, Axitinib, Dasatinib, Imatinib, Imetelstat, Midostaurin, Pazopanib, Sorafenib, Sunitinib, and Tasinga (nilotinib). Select publications disclosing c-KIT kinase inhibitors include WO2013033167, WO2013033116, WO2013/033203, and U.S. Pat. No. 9,199,981.

Several challenges are associated with the targeting of c-KIT for the treatment of cancer. Mutations of c-KIT are not always present in tumors, even those tumors most commonly associated with c-KIT mutations. For example, only 26% of testicular seminomas have been found to be associated with c-KIT mutations. Furthermore, for those cancers most associated with c-KIT mutations, these mutations do not appear to be sufficient for tumorigenesis. Additionally, a major concern is the development of resistance towards drugs. Such resistance could originate from mutations that decrease c-KIT binding affinity for a given drug, overexpression of transport proteins that decrease the intracellular concentration of a given drug, or both. Resistance to imatinib, in chronic myelogenous leukemia, has been attributed both to mutations in the kinase domain, which is also specific for drug biding, and to overexpression of Ber-Abl. An additional confounding factor for the development of cancer treatment based on c-KIT inhibitors is the occurrence of c-KIT in normal, noncancerous tissues, such as breast epithelial, vascular endothelial, sweat glands, and retinal astrocytes. Thus, targeting of c-KIT is only effective for the treatment of cancer when c-KIT mutations “drive” the cancer. Given the vast complexity of cancer therapy, novel c-KIT inhibitors are needed to aid in treatment of cancer.

c-KIT Kinase Inhibitory Compounds

In one aspect, provided herein is a c-KIT kinase inhibitory compound.

One embodiment provides a compound, or a pharmaceutically acceptable salt or solvate thereof, having the structure of Formula (I):

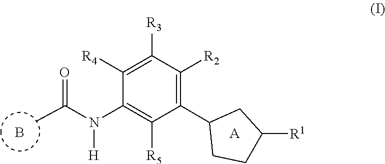

wherein,
Ring A is an optionally substituted 5-membered nitrogen-containing heteroaryl;
Ring B is an optionally substituted 9- or 10-membered bicyclic nitrogen-containing heteroaryl;
R¹ is optionally substituted alkyl, optionally substituted carbocyclyl, optionally substituted carbocyclylalkyl, optionally substituted heterocyclyl, optionally substituted heterocyclylalkyl, optionally substituted aryl, optionally substituted aralkyl, optionally substituted heteroaryl, or optionally substituted heteroaralkyl.
R² is hydrogen, halo, cyano, or optionally substituted C1-C6 alkyl;
R³ is halo;
R⁴ is optionally substituted C1-C6 alkyl; and
R⁵ is hydrogen, halo, cyano, or optionally substituted C1-C6 alkyl.

Another embodiment provides the compound of Formula (I), or a pharmaceutically acceptable salt or solvate thereof, wherein ring A is

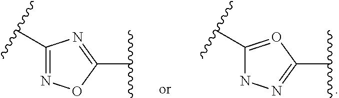

Another embodiment provides the compound of Formula (I), or a pharmaceutically acceptable salt or solvate thereof, wherein ring A is

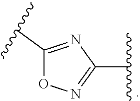 [SMILES: *c1noc(*)n1]

Another embodiment provides the compound of Formula (I), or a pharmaceutically acceptable salt or solvate thereof, wherein ring A is

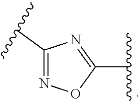 [SMILES: *c1noc(*)n1]

Another embodiment provides the compound of Formula (I), or a pharmaceutically acceptable salt or solvate thereof, wherein ring A is

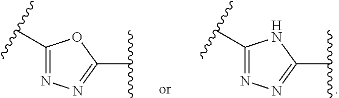 [SMILES: *c1nnc(*)o1 ; *c1nnc(*)[nH]1]

Another embodiment provides the compound of Formula (I), or a pharmaceutically acceptable salt or solvate thereof, wherein ring A is

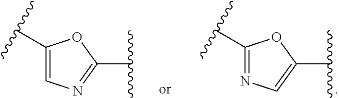 [SMILES: *c1cnc(*)o1 ; *c1cnc(*)o1]

Another embodiment provides the compound of Formula (I), or a pharmaceutically acceptable salt or solvate thereof, wherein ring A is

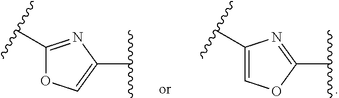 [SMILES: *c1coc(*)n1 ; *c1coc(*)n1]

Another embodiment provides the compound of Formula (I), or a pharmaceutically acceptable salt or solvate thereof, wherein ring B is an optionally substituted 9-membered bicyclic nitrogen-containing heteroaryl.

Another embodiment provides the compound of Formula (I), or a pharmaceutically acceptable salt or solvate thereof, wherein ring B is an optionally substituted 10-membered bicyclic nitrogen-containing heteroaryl.

Another embodiment provides the compound of Formula (I), or a pharmaceutically acceptable salt or solvate thereof, wherein ring B is

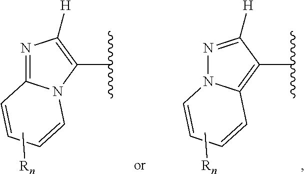

wherein n is 0-4; and each R is independently selected from the group consisting of hydrogen, cyano, halo, hydroxy, azido, amino, nitro, —CO₂H, —S(O)—R¹⁰, —S—R¹⁰, —S(O)₂—R¹⁰, optionally substituted C1-C6 alkoxy, optionally substituted C1-C6 alkyl, optionally substituted C1-C6 alkynyl, optionally substituted carbocyclyl, optionally substituted C1-C6 alkenyl, optionally substituted heterocyclyl, —N(R¹¹)₂, —CO—R¹⁰, —CO₂—R¹⁰, —CON(R¹¹)₂, —NR¹¹CO—R¹⁰, —NR¹¹CO₂—R¹⁰, —SO₂N(R¹¹)₂, —C(═NR¹²)—N(R¹¹)₂, —NR¹¹CO—R¹⁰, —NR¹¹CO₂—R¹⁰, —NR¹¹CO—N(R¹⁰)₂, and —NR¹¹SO₂—N(R¹⁰)₂;
each R¹⁰ is independently selected from the group consisting of optionally substituted C1-C6 alkyl, optionally substituted C3-C8 carbocyclyl, optionally substituted heterocyclyl, optionally substituted aryl, and optionally substituted heteroaryl;
each R¹¹ is independently selected from the group consisting of hydrogen, optionally substituted C1-C6 alkyl, optionally substituted C3-C8 carbocyclyl, optionally substituted heterocyclyl, optionally substituted aryl, and optionally substituted heteroaryl; and R¹² is H or optionally substituted C1-C6 alkyl.

Another embodiment provides the compound of Formula (I), or a pharmaceutically acceptable salt or solvate thereof, wherein ring B is

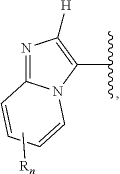

wherein n is 0-4; and each R is independently selected from the group consisting of hydrogen, cyano, halo, hydroxy, azido, amino, nitro, —CO₂H, —S(O)—R¹⁰, —S—R¹⁰, —S(O)₂—R¹⁰, optionally substituted C1-C6 alkoxy, optionally substituted C1-C6 alkyl, optionally substituted C1-C6 alkynyl, optionally substituted carbocyclyl, optionally substituted C1-C6 alkenyl, optionally substituted heterocyclyl, —N(R¹¹)₂, —CO—R¹⁰, —CO₂—R¹⁰, —CON(R¹¹)₂, —NR¹¹CO—R¹⁰, —NR¹¹CO₂—R¹⁰, —SO₂N(R¹¹)₂, —C(═NR¹²)—N(R¹¹)₂, —NR¹¹CO—R¹⁰, —NR¹¹CO₂—R¹⁰, —NR¹¹CO—N(R¹⁰)₂, and —NR¹¹SO₂—N(R¹⁰)₂;
each R¹⁰ is independently selected from the group consisting of optionally substituted C1-C6 alkyl, optionally substituted C3-C8 carbocyclyl, optionally substituted heterocyclyl, optionally substituted aryl, and optionally substituted heteroaryl;
each R¹¹ is independently selected from the group consisting of hydrogen, optionally substituted C1-C6 alkyl, optionally substituted C3-C8 carbocyclyl, optionally substituted heterocyclyl, optionally substituted aryl, and optionally substituted heteroaryl; and
R¹² is H or optionally substituted C1-C6 alkyl.

Another embodiment provides the compound of Formula (I), or pharmaceutically acceptable salt or solvate thereof, wherein R is hydrogen, halo, cyano, —CONH₂, or heterocyclyl. Another embodiment provides the compound of Formula (I), or pharmaceutically acceptable salt or solvate thereof, wherein R is hydrogen or halo. Another embodiment provides the compound of Formula (I), or pharmaceutically acceptable salt or solvate thereof, wherein n is 1 or 2; and each R is independently selected from the group consisting of cyano, halo, hydroxy, —CO₂H, optionally substituted C1-C6 alkoxy, optionally substituted C1-C6 alkyl, optionally substituted heterocyclyl, or —CON(R¹¹)₂. Another embodiment provides the compound of Formula (I), or pharmaceutically acceptable salt or solvate thereof, wherein n is 1; and R is optionally substituted heterocyclyl. Another embodiment provides the compound, or pharmaceutically acceptable salt or solvate thereof, wherein the optionally substituted heterocyclyl is an optionally substituted morpholinyl or piperazinyl.

Another embodiment provides the compound of Formula (I), or a pharmaceutically acceptable salt or solvate thereof, wherein ring B is

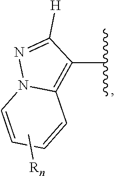

wherein n is 0-4; and each R is independently selected from the group consisting of hydrogen, cyano, halo, hydroxy, azido, amino, nitro, —CO₂H, —S(O)—R¹⁰, —S—R¹⁰, —S(O)₂—R¹⁰, optionally substituted C1-C6 alkoxy, optionally substituted C1-C6 alkyl, optionally substituted C1-C6 alkynyl, optionally substituted carbocyclyl, optionally substituted C1-C6 alkenyl, optionally substituted heterocyclyl, —N(R¹¹)₂, —CO—R¹⁰, —CO₂—R¹⁰, —CON(R¹¹)₂, —NR¹¹CO—R¹⁰, —NR¹¹CO₂—R¹⁰, —SO₂N(R¹¹)₂, —C(═NR¹²)—N(R¹¹)₂, —NR¹¹CO—R¹⁰, —NR¹¹CO₂—R¹⁰, —NR¹¹CO—N(R¹⁰)₂, and —NR¹¹SO₂—N(R¹⁰)₂;
each R¹⁰ is independently selected from the group consisting of optionally substituted C1-C6 alkyl, optionally substituted C3-C8 carbocyclyl, optionally substituted heterocyclyl, optionally substituted aryl, and optionally substituted heteroaryl;
each R¹¹ is independently selected from the group consisting of hydrogen, optionally substituted C1-C6 alkyl, optionally substituted C3-C8 carbocyclyl, optionally substituted heterocyclyl, optionally substituted aryl, and optionally substituted heteroaryl; and
R¹² is H or optionally substituted C1-C6 alkyl.

Another embodiment provides the compound of Formula (I), or pharmaceutically acceptable salt or solvate thereof, wherein R is hydrogen, halo, cyano, —CONH₂, or heterocyclyl. Another embodiment provides the compound of Formula (I), or pharmaceutically acceptable salt or solvate thereof, wherein R is hydrogen or halo. Another embodiment provides the compound of Formula (I), or pharmaceutically acceptable salt or solvate thereof, wherein n is 1 or 2; and each R is independently selected from the group consisting of cyano, halo, hydroxy, —CO₂H, optionally substituted C1-C6 alkoxy, optionally substituted C1-C6 alkyl, optionally substituted heterocyclyl, or —CON(R¹¹)₂. Another embodiment provides the compound of Formula (I), or pharmaceutically acceptable salt or solvate thereof, wherein n is 1; and R is optionally substituted heterocyclyl. Another embodiment provides the compound, or pharmaceutically acceptable salt or solvate thereof, wherein the optionally substituted heterocyclyl is an optionally substituted morpholinyl or piperazinyl.

Another embodiment provides the compound of Formula (I), or a pharmaceutically acceptable salt or solvate thereof, wherein the R group is placed to provide the regioisomer indicated below:

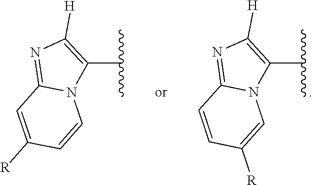

Another embodiment provides the compound of Formula (I), or a pharmaceutically acceptable salt or solvate thereof, wherein ring B is

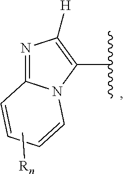

wherein n is 0.

Another embodiment provides the compound of Formula (I), or a pharmaceutically acceptable salt or solvate thereof, wherein ring B is

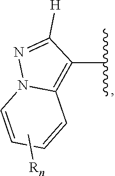

wherein n is 0.

Another embodiment provides the compound of Formula (I), or a pharmaceutically acceptable salt or solvate thereof, wherein R¹ is optionally substituted carbocyclyl. Another embodiment provides the compound, or pharmaceutically acceptable salt or solvate thereof, wherein the optionally substituted carbocyclyl is a 3- or 4-membered optionally substituted carbocyclyl. Another embodiment provides the compound, or pharmaceutically acceptable salt or solvate thereof, wherein the optionally substituted carbocyclyl is substituted with at least a halogen.

Another embodiment provides the compound of Formula (I), or a pharmaceutically acceptable salt or solvate thereof, wherein R¹ is optionally substituted heterocyclyl. Another embodiment provides the compound, or pharmaceutically acceptable salt or solvate thereof, wherein R¹ is an optionally substituted azetidine, or an optionally substituted oxetane. Another embodiment provides the compound, or pharmaceutically acceptable salt or solvate thereof, wherein R¹ is an optionally substituted piperazine, or an optionally substituted morpholine. Another embodiment provides the compound, or pharmaceutically acceptable salt or solvate thereof, wherein R¹ is an optionally substituted pyrrolidine. Another embodiment provides the compound, or pharmaceutically acceptable salt or solvate thereof, wherein the optional substituent is bonded to the nitrogen of the heterocyclyl group.

Another embodiment provides the compound of Formula (I), or a pharmaceutically acceptable salt or solvate thereof, wherein R¹ is an optionally substituted azetidine and the azetidine is optionally substituted with one or more substituents selected from optionally substituted alkyl, halo, fluoroalkyl, oxo, thioxo, cyano, nitro, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); where each R^{a} is independently hydrogen, alkyl (optionally substituted with halogen, hydroxy, methoxy, amino or trifluoromethyl), fluoroalkyl, cycloalkyl (optionally substituted with halogen, hydroxy, methoxy, amino or trifluoromethyl), cycloalkylalkyl (optionally substituted with halogen, hydroxy, methoxy, amino or trifluoromethyl), aryl (optionally substituted with halogen, hydroxy, methoxy, amino or trifluoromethyl), aralkyl (optionally substituted with halogen, hydroxy, methoxy, amino or trifluoromethyl), heterocyclyl (optionally substituted with halogen, hydroxy, methoxy, amino or trifluoromethyl), heterocyclylalkyl (optionally substituted with halogen, hydroxy, methoxy, amino or trifluoromethyl), heteroaryl (optionally substituted with halogen, hydroxy, methoxy, amino or trifluoromethyl), or heteroarylalkyl (optionally substituted with halogen, hydroxy, methoxy, amino or trifluoromethyl), or optionally, for a —N(R^{a})₂ group, both R^{a} groups may join to form a nitrogen-containing heterocyclyl; each R^{b} is independently a direct bond or a straight or branched alkylene or alkenylene chain; R^{c} is a straight or branched alkylene or alkenylene chain; and where each of the R^{a}, R^{b}, or R^{c} substituents is unsubstituted unless otherwise indicated. Another embodiment provides the compound, or pharmaceutically acceptable salt or solvate thereof, wherein the optional substituent is bonded to the nitrogen of the heterocyclyl group.

Another embodiment provides the compound of Formula (I), or a pharmaceutically acceptable salt or solvate thereof, wherein R¹ is an optionally substituted azetidine and the azetidine is optionally substituted with one or more substituents selected from —R^{b}—C(O)OR^{a}, —R^{b}—C(O)N(R^{a})₂; where each R^{a} is independently hydrogen, alkyl (optionally substituted with halogen, hydroxy, methoxy, amino or trifluoromethyl), fluoroalkyl, cycloalkyl (optionally substituted with halogen, hydroxy, methoxy, amino or trifluoromethyl), cycloalkylalkyl (optionally substituted with halogen, hydroxy, methoxy, amino or trifluoromethyl), aryl (optionally substituted with halogen, hydroxy, methoxy, amino or trifluoromethyl), aralkyl (optionally substituted with halogen, hydroxy, methoxy, amino or trifluoromethyl), heterocyclyl (optionally substituted with halogen, hydroxy, methoxy, amino or trifluoromethyl), heterocyclylalkyl (optionally substituted with halogen, hydroxy, methoxy, amino or trifluoromethyl), heteroaryl (optionally substituted with halogen, hydroxy, methoxy, amino or trifluoromethyl), or heteroarylalkyl (optionally substituted with halogen, hydroxy, methoxy, amino or trifluoromethyl), or optionally, for a —N(R^{a})₂ group, both R^{a} groups may join to form a nitrogen-containing heterocyclyl; each R^{b} is independently a direct bond or a straight or branched alkylene or alkenylene chain; and where each of the R^{a} or R^{b} substituents is unsubstituted unless otherwise indicated. Another embodiment provides the compound, or pharmaceutically acceptable salt or solvate thereof, wherein the optional substituent is bonded to the nitrogen of the heterocyclyl group.

Another embodiment provides the compound of Formula (I), or a pharmaceutically acceptable salt or solvate thereof, wherein R¹ is an optionally substituted azetidine and the azetidine is optionally substituted with one or more substituents selected from —C(O)OR^{a}, —C(O)N(R^{a})₂; where each R^{a} is independently hydrogen, alkyl (optionally substituted with halogen, hydroxy, methoxy, amino or trifluoromethyl), or cycloalkyl (optionally substituted with halogen, hydroxy, methoxy, amino or trifluoromethyl). Another embodiment provides the compound of Formula (I), or pharmaceutically acceptable salt or solvate thereof, wherein R¹ is a substituted azetidine substituted with a —C(O)OR^{a}, wherein R^{a} is alkyl (optionally substituted with halogen, hydroxy, methoxy, amino or trifluoromethyl). Another embodiment provides the compound, or pharmaceutically acceptable salt or solvate thereof, wherein the optional substituent is bonded to the nitrogen of the heterocyclyl group.

Another embodiment provides the compound of Formula (I), or a pharmaceutically acceptable salt or solvate thereof, wherein R¹ is

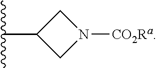

Another embodiment provides the compound of Formula (I), or a pharmaceutically acceptable salt or solvate thereof, wherein R¹ is optionally substituted alkyl, optionally substituted carbocyclylalkyl, optionally substituted heterocyclylalkyl, optionally substituted heteroaryl, or optionally substituted heteroaralkyl.

Another embodiment provides the compound of Formula (I), or a pharmaceutically acceptable salt or solvate thereof, wherein R² is hydrogen, or halo. Another embodiment provides the compound of Formula (I), or pharmaceutically acceptable salt or solvate thereof, wherein R² is hydrogen.

Another embodiment provides the compound of Formula (I), or a pharmaceutically acceptable salt or solvate thereof, wherein R³ is fluoro.

Another embodiment provides the compound of Formula (I), or a pharmaceutically acceptable salt or solvate thereof, wherein R⁴ is optionally substituted C₁-C₂ alkyl. Another embodiment provides the compound of Formula (I), or pharmaceutically acceptable salt or solvate thereof, wherein R⁴ is optionally substituted C₁ alkyl. Another embodiment provides the compound of Formula (I), or pharmaceutically acceptable salt or solvate thereof, wherein R⁴ is CH₃.

Another embodiment provides the compound of Formula (I), or a pharmaceutically acceptable salt or solvate thereof, wherein R⁵ is hydrogen, or halo. Another embodiment provides the compound of Formula (I), or pharmaceutically acceptable salt or solvate thereof, wherein R⁵ is hydrogen. Another embodiment provides the compound of Formula (I), or pharmaceutically acceptable salt or solvate thereof, wherein R⁵ is fluoro.

Another embodiment provides the compound of Formula (I), or a pharmaceutically acceptable salt or solvate thereof, wherein R² is hydrogen, R³ is fluoro, R⁴ is CH₃, and R⁵ is fluoro.

Another embodiment provides the compound of Formula (I), or a pharmaceutically acceptable salt or solvate thereof, wherein ring A is

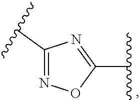 [SMILES: *c1noc(*)n1]

R² is hydrogen, R³ is fluoro, R⁴ is CH₃, and R⁵ is fluoro.

Another embodiment provides the compound of Formula (I), or a pharmaceutically acceptable salt or solvate thereof, wherein ring A is

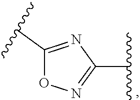 [SMILES: *c1noc(*)n1]

R² is hydrogen, R³ is fluoro, R⁴ is CH₃, and R⁵ is fluoro.

Another embodiment provides the compound of Formula (I), or a pharmaceutically acceptable salt or solvate thereof, wherein ring A is

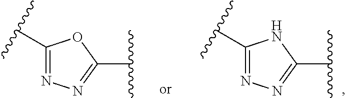 [SMILES: *c1nnc(*)o1 ; *c1nnc(*)[nH]1]

R² is hydrogen, R³ is fluoro, R⁴ is CH₃, and R⁵ is fluoro.

Another embodiment provides the compound of Formula (I), or a pharmaceutically acceptable salt or solvate thereof, wherein ring A is

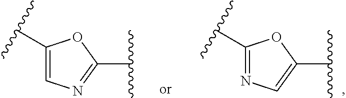 [SMILES: *c1cnc(*)o1 ; *c1cnc(*)o1]

is hydrogen, is fluoro, R⁴ is CH₃, and R⁵ is fluoro.

Another embodiment provides the compound of Formula (I), or a pharmaceutically acceptable salt or solvate thereof, wherein ring A is

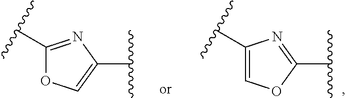 [SMILES: *c1coc(*)n1 ; *c1coc(*)n1]

R² is hydrogen, R³ is fluoro, R⁴ is CH₃, and R⁵ is fluoro.

Another embodiment provides the compound of Formula (I), or a pharmaceutically acceptable salt or solvate thereof, wherein ring B is

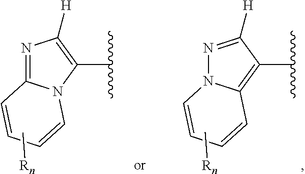

wherein n is 0-4; and each R is independently selected from the group consisting of hydrogen, cyano, halo, hydroxy, azido, amino, nitro, —CO₂H, —S(O)—R¹⁰, —S—R¹⁰, —S(O)₂—R¹⁰, optionally substituted C1-C6 alkoxy, optionally substituted C1-C6 alkyl, optionally substituted C1-C6 alkynyl, optionally substituted carbocyclyl, optionally substituted C1-C6 alkenyl, optionally substituted heterocyclyl, —N(R¹¹)₂, —CO—R¹⁰, —CO₂—R¹⁰, —CON(R¹¹)₂, —NR¹¹CO—R¹⁰, —NR¹¹CO₂—R¹⁰, —SO₂N(R¹¹)₂, —C(═NR¹²)—N(R¹¹)₂NR¹¹CO—R¹⁰, —NR¹¹CO₂—R¹⁰, —NR¹¹CO—N(R¹⁰)₂, and —NR¹¹SO₂—N(R¹⁰)₂;
each R¹⁰ is independently selected from the group consisting of optionally substituted C1-C6 alkyl, optionally substituted C3-C8 carbocyclyl, optionally substituted heterocyclyl, optionally substituted aryl, and optionally substituted heteroaryl;
each R¹¹ is independently selected from the group consisting of hydrogen, optionally substituted C1-C6 alkyl, optionally substituted C3-C8 carbocyclyl, optionally substituted heterocyclyl, optionally substituted aryl, and optionally substituted heteroaryl; and
R¹² is H or optionally substituted C1-C6 alkyl;
R² is hydrogen, R³ is fluoro, R⁴ is CH₃, and R⁵ is fluoro.

Another embodiment provides the compound of Formula (I), or a pharmaceutically acceptable salt or solvate thereof, wherein ring B is

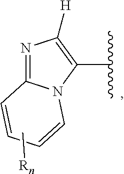

wherein n is 0-4; and each R is independently selected from the group consisting of hydrogen, cyano, halo, hydroxy, azido, amino, nitro, —CO₂H, —S(O)—R¹⁰, —S—R¹⁰, —S(O)₂—R¹⁰, optionally substituted C1-C6 alkoxy, optionally substituted C1-C6 alkyl, optionally substituted C1-C6 alkynyl, optionally substituted carbocyclyl, optionally substituted C1-C6 alkenyl, optionally substituted heterocyclyl, —N(R¹¹)₂, —CO—R¹⁰, —CO₂—R¹⁰, —CON(R¹¹)₂, —NR¹¹CO—R¹⁰, —NR¹¹CO₂—R¹¹, —SO₂N(R¹¹)₂, —C(═NR¹²)—N(R¹¹)₂, —NR¹¹CO—R¹⁰, —NR¹¹CO₂—R¹⁰, —NR¹¹CO—N(R¹⁰)₂, and —NR¹¹SO₂—N(R¹⁰)₂;
each R¹⁰ is independently selected from the group consisting of optionally substituted C1-C6 alkyl, optionally substituted C3-C8 carbocyclyl, optionally substituted heterocyclyl, optionally substituted aryl, and optionally substituted heteroaryl;
each R¹¹ is independently selected from the group consisting of hydrogen, optionally substituted C1-C6 alkyl, optionally substituted C3-C8 carbocyclyl, optionally substituted heterocyclyl, optionally substituted aryl, and optionally substituted heteroaryl; and
R¹² is H or optionally substituted C1-C6 alkyl;
R² is hydrogen, R³ is fluoro, R⁴ is CH₃, and R⁵ is fluoro.

Another embodiment provides the compound of Formula (I), or a pharmaceutically acceptable salt or solvate thereof, wherein ring B is

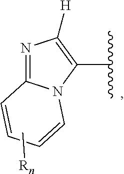

wherein n is 0;
R² is hydrogen, R³ is fluoro, R⁴ is CH₃, and R⁵ is fluoro.

Another embodiment provides the compound of Formula (I), or a pharmaceutically acceptable salt or solvate thereof, wherein the R group is placed to provide the regioisomer indicated below:

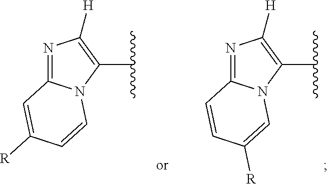

R² is hydrogen, R³ is fluoro, R⁴ is CH₃, and R⁵ is fluoro.

Another embodiment provides the compound of Formula (I), or a pharmaceutically acceptable salt or solvate thereof, wherein R¹ is an optionally substituted azetidine, or an optionally substituted oxetane; R² is hydrogen, R³ is fluoro, R⁴ is CH₃, and R⁵ is fluoro.

Another embodiment provides the compound of Formula (I), or a pharmaceutically acceptable salt or solvate thereof, wherein R¹ is an optionally substituted azetidine; R² is hydrogen, R³ is fluoro, R⁴ is CH₃, and R⁵ is fluoro.

Another embodiment provides the compound of Formula (I), or a pharmaceutically acceptable salt or solvate thereof, wherein ring A is

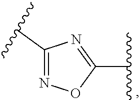 [SMILES: *c1noc(*)n1]

R¹ is a substituted azetidine substituted with a —C(O)OR^{a}, wherein R^{a} is alkyl (optionally substituted with halogen, hydroxy, methoxy, amino or trifluoromethyl), R² is hydrogen, R³ is fluoro, R⁴ is CH₃, and R⁵ is fluoro.

Another embodiment provides the compound of Formula (I), or a pharmaceutically acceptable salt or solvate thereof, wherein ring A is

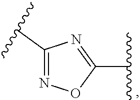 [SMILES: *c1noc(*)n1]

R¹ is a substituted azetidine substituted with a —C(O)OR^{a}, wherein R^{a} is alkyl (optionally substituted with halogen, hydroxy, methoxy, amino or trifluoromethyl), R² is hydrogen, R³ is fluoro, R⁴ is CH₃, and R⁵ is fluoro, and ring B is

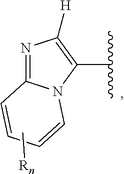

wherein n is 0.

Another embodiment provides the compound of Formula (I), or a pharmaceutically acceptable salt or solvate thereof, wherein ring A is

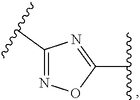 [SMILES: *c1noc(*)n1]

R¹ is a substituted azetidine substituted with a —C(O)OR^{a}, wherein R^{a} is alkyl (optionally substituted with halogen, hydroxy, methoxy, amino or trifluoromethyl), and R³ is fluoro.

One embodiment provides an c-KIT kinase inhibitory compound, or a pharmaceutically acceptable salt or solvate thereof, selected from the group consisting of:
N-(5-(5-(3,3-difluorocyclobutyl)-1,2,4-oxadiazol-3-yl)-3-fluoro-2-methylphenyl)imidazo[1,2-a]pyridine-3-carboxamide;
N-(3-fluoro-5-(5-((1R,2S)-2-fluorocyclopropyl)-1,2,4-oxadiazol-3-yl)-2-methylphenyl)imidazo[1,2-a]pyridine-3-carboxamide;
methyl 3-(3-(3-fluoro-5-(imidazo[1,2-a]pyridine-3-carboxamido)-4-methylphenyl)-1,2,4-oxadiazol-5-yl)azetidine-1-carboxylate;
N-(5-(5-(3,3-difluorocyclobutyl)-1,2,4-oxadiazol-3-yl)-3-fluoro-2-methylphenyl)pyrazolo[1,5-a]pyridine-3-carboxamide;
N-(3-fluoro-5-(5-((1R,2S)-2-fluorocyclopropyl)-1,2,4-oxadiazol-3-yl)-2-methylphenyl)pyrazolo[1,5-a]pyridine-3-carboxamide;
methyl 3-(3-(3-fluoro-4-methyl-5-(pyrazolo[1,5-a]pyridine-3-carboxamido)phenyl)-1,2,4-oxadiazol-5-yl)azetidine-1-carboxylate;
N-(5-(5-(3,3-difluorocyclobutyl)-1,2,4-oxadiazol-3-yl)-3-fluoro-2-methylphenyl)-6-morpholinoimidazo[1,2-a]pyridine-3-carboxamide;
N-(3-fluoro-5-(5-((1R,2S)-2-fluorocyclopropyl)-1,2,4-oxadiazol-3-yl)-2-methylphenyl)-6-morpholinoimidazo[1,2-a]pyridine-3-carboxamide;
methyl 3-(3-(3-fluoro-4-methyl-5-(6-morpholinoimidazo[1,2-a]pyridine-3-carboxamido)phenyl)-1,2,4-oxadiazol-5-yl)azetidine-1-carboxylate;
N-(5-(5-(3,3-difluorocyclobutyl)-1,2,4-oxadiazol-3-yl)-3-fluoro-2-methylphenyl)-7-(piperazin-1-yl)imidazo[1,2-a]pyridine-3-carboxamide;
N-(5-(5-(3,3-difluorocyclobutyl)-1,2,4-oxadiazol-3-yl)-3-fluoro-2-methylphenyl)-7-(4-methylpiperazin-1-yl)imidazo[1,2-a]pyridine-3-carboxamide;
methyl 3-(3-(3-(6-chloroimidazo[1,2-a]pyridine-3-carboxamido)-5-fluoro-4-methylphenyl)-1,2,4-oxadiazol-5-yl)azetidine-1-carboxylate;
N-(5-(5-(3,3-difluorocyclobutyl)-1,2,4-oxadiazol-3-yl)-3-fluoro-2-methylphenyl)-7-morpholinoimidazo[1,2-a]pyridine-3-carboxamide;
N-(3-fluoro-5-(5-((1R,2S)-2-fluorocyclopropyl)-1,2,4-oxadiazol-3-yl)-2-methylphenyl)-7-morpholinoimidazo[1,2-a]pyridine-3-carboxamide;
methyl 3-(3-(3-fluoro-4-methyl-5-(7-morpholinoimidazo[1,2-a]pyridine-3-carboxamido)phenyl)-1,2,4-oxadiazol-5-yl)azetidine-1-carboxylate;
methyl 3-(3-(3-(7-chloroimidazo[1,2-a]pyridine-3-carboxamido)-5-fluoro-4-methylphenyl)-1,2,4-oxadiazol-5-yl)azetidine-1-carboxylate;
methyl 3-(3-(3-fluoro-4-methyl-5-(7-methylimidazo[1,2-a]pyridine-3-carboxamido)phenyl)-1,2,4-oxadiazol-5-yl)azetidine-1-carboxylate;
7-(4-acetylpiperazin-1-yl)-N-(5-(5-(3,3-difluorocyclobutyl)-1,2,4-oxadiazol-3-yl)-3-fluoro-2-methylphenyl)imidazo[1,2-a]pyridine-3-carboxamide;
methyl 3-(3-(3-fluoro-5-(6-fluoroimidazo[1,2-a]pyridine-3-carboxamido)-4-methylphenyl)-1,2,4-oxadiazol-5-yl)azetidine-1-carboxylate;
methyl 3-(3-(3-fluoro-4-methyl-5-(6-(piperazin-1-yl)imidazo[1,2-a]pyridine-3-carboxamido)phenyl)-1,2,4-oxadiazol-5-yl)azetidine-1-carboxylate;
methyl 3-(3-(3-fluoro-4-methyl-5-(6-methylimidazo[1,2-a]pyridine-3-carboxamido)phenyl)-1,2,4-oxadiazol-5-yl)azetidine-1-carboxylate;
N-(3-fluoro-5-(5-((1R,2S)-2-fluorocyclopropyl)-1,2,4-oxadiazol-3-yl)-2-methylphenyl)-7-(piperazin-1-yl)imidazo[1,2-a]pyridine-3-carboxamide;
ethyl 3-(3-(3-fluoro-5-(imidazo[1,2-a]pyridine-3-carboxamido)-4-methylphenyl)-1,2,4-oxadiazol-5-yl)azetidine-1-carboxylate;
methyl 3-(3-(3-fluoro-5-(7-fluoroimidazo[1,2-a]pyridine-3-carboxamido)-4-methylphenyl)-1,2,4-oxadiazol-5-yl)azetidine-1-carboxylate;
N-(5-(5-(3,3-difluorocyclobutyl)-1,2,4-oxadiazol-3-yl)-3-fluoro-2-methylphenyl)-6-(piperazin-1-yl)imidazo[1,2-a]pyridine-3-carboxamide;
N-(3-fluoro-5-(5-((1R,2S)-2-fluorocyclopropyl)-1,2,4-oxadiazol-3-yl)-2-methylphenyl)-6-(piperazin-1-yl)imidazo[1,2-a]pyridine-3-carboxamide;
methyl 3-(3-(3-fluoro-5-(6-methoxyimidazo[1,2-a]pyridine-3-carboxamido)-4-methylphenyl)-1,2,4-oxadiazol-5-yl)azetidine-1-carboxylate;
methyl 3-(3-(3-fluoro-4-methyl-5-(6-(4-methylpiperazin-1-yl)imidazo[1,2-a]pyridine-3-carboxamido)phenyl)-1,2,4-oxadiazol-5-yl)azetidine-1-carboxylate;
methyl 3-(3-(3-fluoro-5-(7-methoxyimidazo[1,2-a]pyridine-3-carboxamido)-4-methylphenyl)-1,2,4-oxadiazol-5-yl)azetidine-1-carboxylate;
methyl 3-(3-(3-fluoro-4-methyl-5-(7-(piperazin-1-yl)imidazo[1,2-a]pyridine-3-carboxamido)phenyl)-1,2,4-oxadiazol-5-yl)azetidine-1-carboxylate;
cyclopropyl 3-(3-(3-fluoro-5-(imidazo[1,2-a]pyridine-3-carboxamido)-4-methylphenyl)-1,2,4-oxadiazol-5-yl)azetidine-1-carboxylate;
N-(3-fluoro-2-methyl-5-(5-(1-(methylcarbamoyl)azetidin-3-yl)-1,2,4-oxadiazol-3-yl)phenyl)imidazo[1,2-a]pyridine-3-carboxamide;
N-(5-(5-(3,3-difluorocyclobutyl)-1,2,4-oxadiazol-3-yl)-3-fluoro-2-methylphenyl)-6-(4-methylpiperazin-1-yl)imidazo[1,2-a]pyridine-3-carboxamide;
N-(3-fluoro-5-(5-((1R,2S)-2-fluorocyclopropyl)-1,2,4-oxadiazol-3-yl)-2-methylphenyl)-6-(4-methylpiperazin-1-yl)imidazo[1,2-a]pyridine-3-carboxamide; methyl 3-(3-(3-(7-cyanoimidazo[1,2-a]pyridine-3-carboxamido)-5-fluoro-4-methylphenyl)-1,2,4-oxadiazol-5-yl)azetidine-1-carboxylate;
methyl 3-(3-(3-(6-(4-acetylpiperazin-1-yl)imidazo[1,2-a]pyridine-3-carboxamido)-5-fluoro-4-methylphenyl)-1,2,4-oxadiazol-5-yl)azetidine-1-carboxylate;
methyl 3-(5-(3-fluoro-5-(imidazo[1,2-a]pyridine-3-carboxamido)-4-methylphenyl)-1,2,4-oxadiazol-3-yl)azetidine-1-carboxylate;
6-(4-acetylpiperazin-1-yl)-N-(5-(5-(3,3-difluorocyclobutyl)-1,2,4-oxadiazol-3-yl)-3-fluoro-2-methylphenyl)imidazo[1,2-a]pyridine-3-carboxamide;
6-(4-acetylpiperazin-1-yl)-N-(3-fluoro-5-(5-((1R,2S)-2-fluorocyclopropyl)-1,2,4-oxadiazol-3-yl)-2-methylphenyl)imidazo[1,2-a]pyridine-3-carboxamide;
methyl 3-(3-(3-(6-carbamoylimidazo[1,2-a]pyridine-3-carboxamido)-5-fluoro-4-methylphenyl)-1,2,4-oxadiazol-5-yl)azetidine-1-carboxylate;
2-aminoethyl 3-(3-(3-fluoro-5-(imidazo[1,2-a]pyridine-3-carboxamido)-4-methylphenyl)-1,2,4-oxadiazol-5-yl)azetidine-1-carboxylate;
2-methoxyethyl 3-(3-(3-fluoro-5-(imidazo[1,2-a]pyridine-3-carboxamido)-4-methylphenyl)-1,2,4-oxadiazol-5-yl)azetidine-1-carboxylate;
methyl 3-(3-(3-(7-carbamoylimidazo[1,2-a]pyridine-3-carboxamido)-5-fluoro-4-methylphenyl)-1,2,4-oxadiazol-5-yl)azetidine-1-carboxylate;
isopropyl 3-(3-(3-fluoro-5-(imidazo[1,2-a]pyridine-3-carboxamido)-4-methylphenyl)-1,2,4-oxadiazol-5-yl)azetidine-1-carboxylate;
N-(5-(5-(1-(ethylcarbamoyl)azetidin-3-yl)-1,2,4-oxadiazol-3-yl)-3-fluoro-2-methylphenyl)imidazo[1,2-a]pyridine-3-carboxamide;
N-(3-fluoro-5-(5-(1-(isopropylcarbamoyl)azetidin-3-yl)-1,2,4-oxadiazol-3-yl)-2-methylphenyl)imidazo[1,2-a]pyridine-3-carboxamide;
N-(3-fluoro-5-(5-(1-((2-hydroxyethyl)carbamoyl)azetidin-3-yl)-1,2,4-oxadiazol-3-yl)-2-methylphenyl)imidazo[1,2-a]pyridine-3-carboxamide;
N-(5-(5-(1-(cyclopropylcarbamoyl)azetidin-3-yl)-1,2,4-oxadiazol-3-yl)-3-fluoro-2-methylphenyl)imidazo[1,2-a]pyridine-3-carboxamide;
N-(3-fluoro-5-(5-(1-((2-methoxyethyl)carbamoyl)azetidin-3-yl)-1,2,4-oxadiazol-3-yl)-2-methylphenyl)imidazo[1,2-a]pyridine-3-carboxamide;
N-(5-(5-(1-((2-aminoethyl)carbamoyl)azetidin-3-yl)-1,2,4-oxadiazol-3-yl)-3-fluoro-2-methylphenyl)imidazo[1,2-a]pyridine-3-carboxamide;
N-(3-fluoro-2-methyl-5-(5-(1-(morpholine-4-carbonyl)azetidin-3-yl)-1,2,4-oxadiazol-3-yl)phenyl)imidazo[1,2-a]pyridine-3-carboxamide;
N-(3-fluoro-2-methyl-5-(5-(1-(piperazine-1-carbonyl)azetidin-3-yl)-1,2,4-oxadiazol-3-yl)phenyl)imidazo[1,2-a]pyridine-3-carboxamide;
N-(3-fluoro-2-methyl-5-(5-(1-(4-methylpiperazine-1-carbonyl)azetidin-3-yl)-1,2,4-oxadiazol-3-yl)phenyl)imidazo[1,2-a]pyridine-3-carboxamide;
methyl 3-(3-(3-(6-cyanoimidazo[1,2-a]pyridine-3-carboxamido)-5-fluoro-4-methylphenyl)-1,2,4-oxadiazol-5-yl)azetidine-1-carboxylate;
methyl 3-(3-(3-fluoro-4-methyl-5-(6-(trifluoromethyl)imidazo[1,2-a]pyridine-3-carboxamido)phenyl)-1,2,4-oxadiazol-5-yl)azetidine-1-carboxylate;
methyl 3-(3-(3-fluoro-5-(6-isopropylimidazo[1,2-a]pyridine-3-carboxamido)-4-methylphenyl)-1,2,4-oxadiazol-5-yl)azetidine-1-carboxylate;
methyl 3-(3-(3-(6-cyclopropylimidazo[1,2-a]pyridine-3-carboxamido)-5-fluoro-4-methylphenyl)-1,2,4-oxadiazol-5-yl)azetidine-1-carboxylate;
methyl 3-(3-(3-fluoro-5-(7-isopropylimidazo[1,2-a]pyridine-3-carboxamido)-4-methylphenyl)-1,2,4-oxadiazol-5-yl)azetidine-1-carboxylate;
methyl 3-(3-(3-(7-cyclopropylimidazo[1,2-a]pyridine-3-carboxamido)-5-fluoro-4-methylphenyl)-1,2,4-oxadiazol-5-yl)azetidine-1-carboxylate;
methyl 3-(3-(3-fluoro-4-methyl-5-(7-(4-methylpiperazin-1-yl)imidazo[1,2-a]pyridine-3-carboxamido)phenyl)-1,2,4-oxadiazol-5-yl)azetidine-1-carboxylate;
methyl 4-(3-(3-fluoro-5-(imidazo[1,2-a]pyridine-3-carboxamido)-4-methylphenyl)-1,2,4-oxadiazol-5-yl)piperazine-1-carboxylate;
methyl 4-(3-(3-fluoro-5-(imidazo[1,2-a]pyridine-3-carboxamido)-4-methylphenyl)-1,2,4-oxadiazol-5-yl)-2-methylpiperazine-1-carboxylate;
methyl 4-(3-(3-fluoro-5-(imidazo[1,2-a]pyridine-3-carboxamido)-4-methylphenyl)-1,2,4-oxadiazol-5-yl)-2-isopropylpiperazine-1-carboxylate;
methyl 4-(3-(3-fluoro-5-(imidazo[1,2-a]pyridine-3-carboxamido)-4-methylphenyl)-1,2,4-oxadiazol-5-yl)-3-methylpiperazine-1-carboxylate;
methyl 4-(3-(3-fluoro-5-(imidazo[1,2-a]pyridine-3-carboxamido)-4-methylphenyl)-1,2,4-oxadiazol-5-yl)-3-isopropylpiperazine-1-carboxylate;
N-(5-(5-(4-acetylpiperazin-1-yl)-1,2,4-oxadiazol-3-yl)-3-fluoro-2-methylphenyl)imidazo[1,2-a]pyridine-3-carboxamide;
N-(5-(5-(4-carbamoylpiperazin-1-yl)-1,2,4-oxadiazol-3-yl)-3-fluoro-2-methylphenyl)imidazo[1,2-a]pyridine-3-carboxamide;
N-(3-fluoro-2-methyl-5-(5-(4-methylpiperazin-1-yl)-1,2,4-oxadiazol-3-yl)phenyl)imidazo[1,2-a]pyridine-3-carboxamide;
N-(3-fluoro-2-methyl-5-(5-(piperazin-1-yl)-1,2,4-oxadiazol-3-yl)phenyl)imidazo[1,2-a]pyridine-3-carboxamide;
N-(3-fluoro-2-methyl-5-(5-morpholino-1,2,4-oxadiazol-3-yl)phenyl)imidazo[1,2-a]pyridine-3-carboxamide;
6-fluoro-N-(3-fluoro-5-(5-((1R,2S)-2-fluorocyclopropyl)-1,2,4-oxadiazol-3-yl)-2-methylphenyl)imidazo[1,2-a]pyridine-3-carboxamide;
6-chloro-N-(3-fluoro-5-(5-((1R,2S)-2-fluorocyclopropyl)-1,2,4-oxadiazol-3-yl)-2-methylphenyl)imidazo[1,2-a]pyridine-3-carboxamide;
6-cyano-N-(3-fluoro-5-(5-((1R,2S)-2-fluorocyclopropyl)-1,2,4-oxadiazol-3-yl)-2-methylphenyl)imidazo[1,2-a]pyridine-3-carboxamide;
N3-(3-fluoro-5-(5-((1R,2S)-2-fluorocyclopropyl)-1,2,4-oxadiazol-3-yl)-2-methylphenyl)imidazo[1,2-a]pyridine-3,6-dicarboxamide;
N-(3-fluoro-5-(5-((1R,2S)-2-fluorocyclopropyl)-1,2,4-oxadiazol-3-yl)-2-methylphenyl)-6-(trifluoromethyl)imidazo[1,2-a]pyridine-3-carboxamide;
N-(3-fluoro-5-(5-((1R,2S)-2-fluorocyclopropyl)-1,2,4-oxadiazol-3-yl)-2-methylphenyl)-6-methylimidazo[1,2-a]pyridine-3-carboxamide;
N-(3-fluoro-5-(5-((1R,2S)-2-fluorocyclopropyl)-1,2,4-oxadiazol-3-yl)-2-methylphenyl)-6-isopropylimidazo[1,2-a]pyridine-3-carboxamide;
6-cyclopropyl-N-(3-fluoro-5-(5-((1R,2S)-2-fluorocyclopropyl)-1,2,4-oxadiazol-3-yl)-2-methylphenyl)imidazo[1,2-a]pyridine-3-carboxamide;
7-fluoro-N-(3-fluoro-5-(5-((1R,2S)-2-fluorocyclopropyl)-1,2,4-oxadiazol-3-yl)-2-methylphenyl)imidazo[1,2-a]pyridine-3-carboxamide;
7-chloro-N-(3-fluoro-5-(5-((1R,2S)-2-fluorocyclopropyl)-1,2,4-oxadiazol-3-yl)-2-methylphenyl)imidazo[1,2-a]pyridine-3-carboxamide;
N-(3-fluoro-5-(5-((1R,2S)-2-fluorocyclopropyl)-1,2,4-oxadiazol-3-yl)-2-methylphenyl)-7-(trifluoromethyl)imidazo[1,2-a]pyridine-3-carboxamide;
7-cyano-N-(3-fluoro-5-(5-((1R,2S)-2-fluorocyclopropyl)-1,2,4-oxadiazol-3-yl)-2-methylphenyl)imidazo[1,2-a]pyridine-3-carboxamide;
N3-(3-fluoro-5-(5-((1R,2S)-2-fluorocyclopropyl)-1,2,4-oxadiazol-3-yl)-2-methylphenyl)imidazo[1,2-a]pyridine-3,7-dicarboxamide;
N-(3-fluoro-5-(5-((1R,2S)-2-fluorocyclopropyl)-1,2,4-oxadiazol-3-yl)-2-methylphenyl)-7-methylimidazo[1,2-a]pyridine-3-carboxamide;
N-(3-fluoro-5-(5-((1R,2S)-2-fluorocyclopropyl)-1,2,4-oxadiazol-3-yl)-2-methylphenyl)-7-isopropylimidazo[1,2-a]pyridine-3-carboxamide;
7-cyclopropyl-N-(3-fluoro-5-(5-((1R,2S)-2-fluorocyclopropyl)-1,2,4-oxadiazol-3-yl)-2-methylphenyl)imidazo[1,2-a]pyridine-3-carboxamide;
N-(3-fluoro-5-(5-((1R,2S)-2-fluorocyclopropyl)-1,2,4-oxadiazol-3-yl)-2-methylphenyl)-7-(4-methylpiperazin-1-yl)imidazo[1,2-a]pyridine-3-carboxamide;
methyl 3-(3-(3-fluoro-4-methyl-5-(7-(trifluoromethyl)imidazo[1,2-a]pyridine-3-carboxamido)phenyl)-1,2,4-oxadiazol-5-yl)azetidine-1-carboxylate;
N-(3-fluoro-5-(5-((1S,2R)-2-fluorocyclopropyl)-1,2,4-oxadiazol-3-yl)-2-methylphenyl)imidazo[1,2-a]pyridine-3-carboxamide;
N-(3-fluoro-5-(5-((1R,2R)-2-fluorocyclopropyl)-1,2,4-oxadiazol-3-yl)-2-methylphenyl)imidazo[1,2-a]pyridine-3-carboxamide;
N-(3-fluoro-5-(5-((1S,2S)-2-fluorocyclopropyl)-1,2,4-oxadiazol-3-yl)-2-methylphenyl)imidazo[1,2-a]pyridine-3-carboxamide;
methyl 3-(3-(3-fluoro-5-(6-fluoropyrazolo[1,5-a]pyridine-3-carboxamido)-4-methylphenyl)-1,2,4-oxadiazol-5-yl)azetidine-1-carboxylate;
methyl 3-(3-(3-(6-chloropyrazolo[1,5-a]pyridine-3-carboxamido)-5-fluoro-4-methylphenyl)-1,2,4-oxadiazol-5-yl)azetidine-1-carboxylate;
methyl 3-(3-(3-(6-cyanopyrazolo[1,5-a]pyridine-3-carboxamido)-5-fluoro-4-methylphenyl)-1,2,4-oxadiazol-5-yl)azetidine-1-carboxylate;
methyl 3-(3-(3-(6-carbamoylpyrazolo[1,5-a]pyridine-3-carboxamido)-5-fluoro-4-methylphenyl)-1,2,4-oxadiazol-5-yl)azetidine-1-carboxylate;
methyl 3-(3-(3-fluoro-4-methyl-5-(6-(trifluoromethyl)pyrazolo[1,5-a]pyridine-3-carboxamido)phenyl)-1,2,4-oxadiazol-5-yl)azetidine-1-carboxylate;
methyl 3-(3-(3-fluoro-5-(6-methoxypyrazolo[1,5-a]pyridine-3-carboxamido)-4-methylphenyl)-1,2,4-oxadiazol-5-yl)azetidine-1-carboxylate;
methyl 3-(3-(3-fluoro-5-(6-isopropylpyrazolo[1,5-a]pyridine-3-carboxamido)-4-methylphenyl)-1,2,4-oxadiazol-5-yl)azetidine-1-carboxylate;
methyl 3-(3-(3-(6-cyclopropylpyrazolo[1,5-a]pyridine-3-carboxamido)-5-fluoro-4-methylphenyl)-1,2,4-oxadiazol-5-yl)azetidine-1-carboxylate;
methyl 3-(3-(3-fluoro-4-methyl-5-(6-methylpyrazolo[1,5-a]pyridine-3-carboxamido)phenyl)-1,2,4-oxadiazol-5-yl)azetidine-1-carboxylate;
methyl 3-(3-(3-fluoro-4-methyl-5-(5-methylpyrazolo[1,5-a]pyridine-3-carboxamido)phenyl)-1,2,4-oxadiazol-5-yl)azetidine-1-carboxylate;
methyl 3-(3-(3-(5-cyclopropylpyrazolo[1,5-a]pyridine-3-carboxamido)-5-fluoro-4-methylphenyl)-1,2,4-oxadiazol-5-yl)azetidine-1-carboxylate;
methyl 3-(3-(3-fluoro-5-(5-isopropylpyrazolo[1,5-a]pyridine-3-carboxamido)-4-methylphenyl)-1,2,4-oxadiazol-5-yl)azetidine-1-carboxylate;
methyl 3-(3-(3-fluoro-5-(5-fluoropyrazolo[1,5-a]pyridine-3-carboxamido)-4-methylphenyl)-1,2,4-oxadiazol-5-yl)azetidine-1-carboxylate;
methyl 3-(3-(3-(5-chloropyrazolo[1,5-a]pyridine-3-carboxamido)-5-fluoro-4-methylphenyl)-1,2,4-oxadiazol-5-yl)azetidine-1-carboxylate;
methyl 3-(3-(3-(5-cyanopyrazolo[1,5-a]pyridine-3-carboxamido)-5-fluoro-4-methylphenyl)-1,2,4-oxadiazol-5-yl)azetidine-1-carboxylate;
methyl 3-(3-(3-(5-carbamoylpyrazolo[1,5-a]pyridine-3-carboxamido)-5-fluoro-4-methylphenyl)-1,2,4-oxadiazol-5-yl)azetidine-1-carboxylate;
methyl 3-(3-(3-fluoro-4-methyl-5-(5-(trifluoromethyl)pyrazolo[1,5-a]pyridine-3-carboxamido)phenyl)-1,2,4-oxadiazol-5-yl)azetidine-1-carboxylate;
methyl 3-(3-(3-fluoro-4-methyl-5-(5-morpholinopyrazolo[1,5-a]pyridine-3-carboxamido)phenyl)-1,2,4-oxadiazol-5-yl)azetidine-1-carboxylate;
methyl 3-(3-(3-fluoro-4-methyl-5-(5-(piperazin-1-yl)pyrazolo[1,5-a]pyridine-3-carboxamido)phenyl)-1,2,4-oxadiazol-5-yl)azetidine-1-carboxylate;
methyl 3-(3-(3-fluoro-4-methyl-5-(5-(4-methylpiperazin-1-yl)pyrazolo[1,5-a]pyridine-3-carboxamido)phenyl)-1,2,4-oxadiazol-5-yl)azetidine-1-carboxylate;
methyl 3-(3-(3-fluoro-4-methyl-5-(6-morpholinopyrazolo[1,5-a]pyridine-3-carboxamido)phenyl)-1,2,4-oxadiazol-5-yl)azetidine-1-carboxylate;
methyl 3-(3-(3-fluoro-4-methyl-5-(6-(piperazin-1-yl)pyrazolo[1,5-a]pyridine-3-carboxamido)phenyl)-1,2,4-oxadiazol-5-yl)azetidine-1-carboxylate;
methyl 3-(3-(3-fluoro-4-methyl-5-(6-(4-methylpiperazin-1-yl)pyrazolo[1,5-a]pyridine-3-carboxamido)phenyl)-1,2,4-oxadiazol-5-yl)azetidine-1-carboxylate;
N-(5-(3-(3,3-difluorocyclobutyl)-1,2,4-oxadiazol-5-yl)-3-fluoro-2-methylphenyl)imidazo[1,2-a]pyridine-3-carboxamide;
N-(3-fluoro-5-(3-((1R,2S)-2-fluorocyclopropyl)-1,2,4-oxadiazol-5-yl)-2-methylphenyl)imidazo[1,2-a]pyridine-3-carboxamide;
isopropyl 3-(5-(3-fluoro-5-(imidazo[1,2-a]pyridine-3-carboxamido)-4-methylphenyl)-1,2,4-oxadiazol-3-yl)azetidine-1-carboxylate;
cyclopropyl 3-(5-(3-fluoro-5-(imidazo[1,2-a]pyridine-3-carboxamido)-4-methylphenyl)-1,2,4-oxadiazol-3-yl)azetidine-1-carboxylate;
N-(3-fluoro-2-methyl-5-(3-(1-(methylcarbamoyl)azetidin-3-yl)-1,2,4-oxadiazol-5-yl)phenyl)imidazo[1,2-a]pyridine-3-carboxamide;
N-(3-fluoro-2-methyl-5-(3-(1-(morpholine-4-carbonyl)azetidin-3-yl)-1,2,4-oxadiazol-5-yl)phenyl)imidazo[1,2-a]pyridine-3-carboxamide;
N-(3-fluoro-2-methyl-5-(3-(1-(piperazine-1-carbonyl)azetidin-3-yl)-1,2,4-oxadiazol-5-yl)phenyl)imidazo[1,2-a]pyridine-3-carboxamide;
methyl 3-(5-(3-fluoro-5-(6-fluoroimidazo[1,2-a]pyridine-3-carboxamido)-4-methylphenyl)-1,2,4-oxadiazol-3-yl)azetidine-1-carboxylate;
methyl 3-(5-(3-(6-chloroimidazo[1,2-a]pyridine-3-carboxamido)-5-fluoro-4-methylphenyl)-1,2,4-oxadiazol-3-yl)azetidine-1-carboxylate;
methyl 3-(5-(3-(6-cyanoimidazo[1,2-a]pyridine-3-carboxamido)-5-fluoro-4-methylphenyl)-1,2,4-oxadiazol-3-yl)azetidine-1-carboxylate;
methyl 3-(5-(3-(6-carbamoylimidazo[1,2-a]pyridine-3-carboxamido)-5-fluoro-4-methylphenyl)-1,2,4-oxadiazol-3-yl)azetidine-1-carboxylate;
methyl 3-(5-(3-fluoro-4-methyl-5-(6-(trifluoromethyl)imidazo[1,2-a]pyridine-3-carboxamido)phenyl)-1,2,4-oxadiazol-3-yl)azetidine-1-carboxylate;
methyl 3-(5-(3-fluoro-4-methyl-5-(6-methylimidazo[1,2-a]pyridine-3-carboxamido)phenyl)-1,2,4-oxadiazol-3-yl)azetidine-1-carboxylate;
methyl 3-(5-(3-fluoro-5-(6-isopropylimidazo[1,2-a]pyridine-3-carboxamido)-4-methylphenyl)-1,2,4-oxadiazol-3-yl)azetidine-1-carboxylate;
methyl 3-(5-(3-(6-cyclopropylimidazo[1,2-a]pyridine-3-carboxamido)-5-fluoro-4-methylphenyl)-1,2,4-oxadiazol-3-yl)azetidine-1-carboxylate;
methyl 3-(5-(3-fluoro-4-methyl-5-(6-morpholinoimidazo[1,2-a]pyridine-3-carboxamido)phenyl)-1,2,4-oxadiazol-3-yl)azetidine-1-carboxylate;
methyl 3-(5-(3-fluoro-4-methyl-5-(6-(piperazin-1-yl)imidazo[1,2-a]pyridine-3-carboxamido)phenyl)-1,2,4-oxadiazol-3-yl)azetidine-1-carboxylate;
methyl 3-(5-(3-fluoro-4-methyl-5-(6-(4-methylpiperazin-1-yl)imidazo[1,2-a]pyridine-3-carboxamido)phenyl)-1,2,4-oxadiazol-3-yl)azetidine-1-carboxylate;
methyl 3-(5-(3-fluoro-5-(7-fluoroimidazo[1,2-a]pyridine-3-carboxamido)-4-methylphenyl)-1,2,4-oxadiazol-3-yl)azetidine-1-carboxylate;
methyl 3-(5-(3-fluoro-4-methyl-5-(7-methylimidazo[1,2-a]pyridine-3-carboxamido)phenyl)-1,2,4-oxadiazol-3-yl)azetidine-1-carboxylate;
methyl 3-(5-(3-(7-chloroimidazo[1,2-a]pyridine-3-carboxamido)-5-fluoro-4-methylphenyl)-1,2,4-oxadiazol-3-yl)azetidine-1-carboxylate;
methyl 3-(5-(3-(7-cyanoimidazo[1,2-a]pyridine-3-carboxamido)-5-fluoro-4-methylphenyl)-1,2,4-oxadiazol-3-yl)azetidine-1-carboxylate;
methyl 3-(5-(3-(7-carbamoylimidazo[1,2-a]pyridine-3-carboxamido)-5-fluoro-4-methylphenyl)-1,2,4-oxadiazol-3-yl)azetidine-1-carboxylate;
methyl 3-(5-(3-fluoro-4-methyl-5-(7-(trifluoromethyl)imidazo[1,2-a]pyridine-3-carboxamido)phenyl)-1,2,4-oxadiazol-3-yl)azetidine-1-carboxylate;
methyl 3-(5-(3-fluoro-4-methyl-5-(7-morpholinoimidazo[1,2-a]pyridine-3-carboxamido)phenyl)-1,2,4-oxadiazol-3-yl)azetidine-1-carboxylate;
methyl 3-(5-(3-fluoro-4-methyl-5-(7-(piperazin-1-yl)imidazo[1,2-a]pyridine-3-carboxamido)phenyl)-1,2,4-oxadiazol-3-yl)azetidine-1-carboxylate;
methyl 3-(5-(3-fluoro-4-methyl-5-(7-(4-methylpiperazin-1-yl)imidazo[1,2-a]pyridine-3-carboxamido)phenyl)-1,2,4-oxadiazol-3-yl)azetidine-1-carboxylate;
N-(3-fluoro-2-methyl-5-(3-morpholino-1,2,4-oxadiazol-5-yl)phenyl)imidazo[1,2-a]pyridine-3-carboxamide;
N-(3-fluoro-2-methyl-5-(3-(piperazin-1-yl)-1,2,4-oxadiazol-5-yl)phenyl)imidazo[1,2-a]pyridine-3-carboxamide;
methyl 4-(5-(3-fluoro-5-(imidazo[1,2-a]pyridine-3-carboxamido)-4-methylphenyl)-1,2,4-oxadiazol-3-yl)piperazine-1-carboxylate;
methyl 4-(5-(3-fluoro-5-(imidazo[1,2-a]pyridine-3-carboxamido)-4-methylphenyl)-1,2,4-oxadiazol-3-yl)-2-methylpiperazine-1-carboxylate;
methyl 4-(5-(3-fluoro-5-(imidazo[1,2-a]pyridine-3-carboxamido)-4-methylphenyl)-1,2,4-oxadiazol-3-yl)-2-isopropylpiperazine-1-carboxylate;
methyl 4-(5-(3-fluoro-5-(imidazo[1,2-a]pyridine-3-carboxamido)-4-methylphenyl)-1,2,4-oxadiazol-3-yl)-3-methylpiperazine-1-carboxylate;
methyl 4-(5-(3-fluoro-5-(imidazo[1,2-a]pyridine-3-carboxamido)-4-methylphenyl)-1,2,4-oxadiazol-3-yl)-3-isopropylpiperazine-1-carboxylate;
methyl 3-(5-(3-fluoro-5-(imidazo[1,2-a]pyridine-3-carboxamido)-4-methylphenyl)-1,3,4-oxadiazol-2-yl)azetidine-1-carboxylate;
methyl 3-(4-(3-fluoro-5-(imidazo[1,2-a]pyridine-3-carboxamido)-4-methylphenyl)oxazol-2-yl)azetidine-1-carboxylate;
methyl 3-(5-(3-fluoro-5-(imidazo[1,2-a]pyridine-3-carboxamido)-4-methylphenyl)oxazol-2-yl)azetidine-1-carboxylate;
methyl 3-(2-(3-fluoro-5-(imidazo[1,2-a]pyridine-3-carboxamido)-4-methylphenyl)oxazol-4-yl)azetidine-1-carboxylate;
methyl 3-(5-(3-fluoro-5-(imidazo[1,2-a]pyridine-3-carboxamido)-4-methylphenyl)-4H-1,2,4-triazol-3-yl)azetidine-1-carboxylate;
methyl (R)-3-(3-(3-fluoro-5-(imidazo[1,2-a]pyridine-3-carboxamido)-4-methylphenyl)-1,2,4-oxadiazol-5-yl)piperidine-1-carboxylate;
N-(3-fluoro-5-(5-((1S,2R)-2-fluorocyclopropyl)-1,2,4-oxadiazol-3-yl)-2-methylphenyl)-7-morpholinoimidazo[1,2-a]pyridine-3-carboxamide;
methyl 3-(2-(3-fluoro-5-(imidazo[1,2-a]pyridine-3-carboxamido)-4-methylphenyl)oxazol-5-yl)azetidine-1-carboxylate;
methyl 3-(3-(2,5-difluoro-3-(imidazo[1,2-a]pyridine-3-carboxamido)-4-methylphenyl)-1,2,4-oxadiazol-5-yl)azetidine-1-carboxylate;
methyl (R)-3-(3-(2,5-difluoro-3-(imidazo[1,2-a]pyridine-3-carboxamido)-4-methylphenyl)-1,2,4-oxadiazol-5-yl)pyrrolidine-1-carboxylate;
methyl (R)-3-(3-(2,5-difluoro-3-(imidazo[1,2-a]pyridine-3-carboxamido)-4-methylphenyl)-1,2,4-oxadiazol-5-yl)piperidine-1-carboxylate;
methyl (S)-3-(3-(2,5-difluoro-3-(imidazo[1,2-a]pyridine-3-carboxamido)-4-methylphenyl)-1,2,4-oxadiazol-5-yl)pyrrolidine-1-carboxylate;
methyl (S)-3-(3-(2,5-difluoro-3-(imidazo[1,2-a]pyridine-3-carboxamido)-4-methylphenyl)-1,2,4-oxadiazol-5-yl)piperidine-1-carboxylate;
N-(3-(5-(3,3-difluorocyclobutyl)-1,2,4-oxadiazol-3-yl)-2,5-difluoro-6-methylphenyl)imidazo[1,2-a]pyridine-3-carboxamide;
N-(3-(5-(3,3-difluorocyclobutyl)-1,2,4-oxadiazol-3-yl)-2,5-difluoro-6-methylphenyl)-7-morpholinoimidazo[1,2-a]pyridine-3-carboxamide;
N-(3-(5-(3,3-difluorocyclobutyl)-1,2,4-oxadiazol-3-yl)-2,5-difluoro-6-methylphenyl)-7-(piperazin-1-yl)imidazo[1,2-a]pyridine-3-carboxamide;
N-(2,5-difluoro-3-(5-((1R,2S)-2-fluorocyclopropyl)-1,2,4-oxadiazol-3-yl)-6-methylphenyl)imidazo[1,2-a]pyridine-3-carboxamide;
N-(2,5-difluoro-3-(5-((1S,2R)-2-fluorocyclopropyl)-1,2,4-oxadiazol-3-yl)-6-methylphenyl)imidazo[1,2-a]pyridine-3-carboxamide;
N-(2,5-difluoro-3-(5-((1R,2S)-2-fluorocyclopropyl)-1,2,4-oxadiazol-3-yl)-6-methylphenyl)-7-morpholinoimidazo[1,2-a]pyridine-3-carboxamide;
N-(2,5-difluoro-3-(5-((1S,2R)-2-fluorocyclopropyl)-1,2,4-oxadiazol-3-yl)-6-methylphenyl)-7-morpholinoimidazo[1,2-a]pyridine-3-carboxamide;
N-(2,5-difluoro-3-(5-((1S,2R)-2-fluorocyclopropyl)-1,2,4-oxadiazol-3-yl)-6-methylphenyl)-7-methylimidazo[1,2-a]pyridine-3-carboxamide;
N-(2,5-difluoro-3-(5-((1S,2R)-2-fluorocyclopropyl)-1,2,4-oxadiazol-3-yl)-6-methylphenyl)-7-methoxyimidazo[1,2-a]pyridine-3-carboxamide;
methyl 3-(3-(2,5-difluoro-4-methyl-3-(7-methylimidazo[1,2-a]pyridine-3-carboxamido)phenyl)-1,2,4-oxadiazol-5-yl)azetidine-1-carboxylate;
methyl 3-(3-(2,5-difluoro-3-(7-methoxyimidazo[1,2-a]pyridine-3-carboxamido)-4-methylphenyl)-1,2,4-oxadiazol-5-yl)azetidine-1-carboxylate;
methyl 3-(3-(2,5-difluoro-4-methyl-3-(7-morpholinoimidazo[1,2-a]pyridine-3-carboxamido)phenyl)-1,2,4-oxadiazol-5-yl)azetidine-1-carboxylate;
methyl 3-(3-(2,5-difluoro-4-methyl-3-(7-(piperazin-1-yl)imidazo[1,2-a]pyridine-3-carboxamido)phenyl)-1,2,4-oxadiazol-5-yl)azetidine-1-carboxylate;
methyl 3-(3-(2,5-difluoro-4-methyl-3-(7-(4-methylpiperazin-1-yl)imidazo[1,2-a]pyridine-3-carboxamido)phenyl)-1,2,4-oxadiazol-5-yl)azetidine-1-carboxylate;
methyl 3-(3-(2,5-difluoro-4-methyl-3-(6-methylimidazo[1,2-a]pyridine-3-carboxamido)phenyl)-1,2,4-oxadiazol-5-yl)azetidine-1-carboxylate;
methyl 3-(3-(2,5-difluoro-3-(6-methoxyimidazo[1,2-a]pyridine-3-carboxamido)-4-methylphenyl)-1,2,4-oxadiazol-5-yl)azetidine-1-carboxylate;
methyl 3-(3-(2,5-difluoro-4-methyl-3-(6-morpholinoimidazo[1,2-a]pyridine-3-carboxamido)phenyl)-1,2,4-oxadiazol-5-yl)azetidine-1-carboxylate;
methyl 3-(3-(2,5-difluoro-4-methyl-3-(6-(piperazin-1-yl)imidazo[1,2-a]pyridine-3-carboxamido)phenyl)-1,2,4-oxadiazol-5-yl)azetidine-1-carboxylate;
methyl 3-(3-(2,5-difluoro-4-methyl-3-(6-(4-methylpiperazin-1-yl)imidazo[1,2-a]pyridine-3-carboxamido)phenyl)-1,2,4-oxadiazol-5-yl)azetidine-1-carboxylate;
N-(3-(5-(3,3-difluorocyclobutyl)-1,2,4-oxadiazol-3-yl)-2,5-difluoro-6-methylphenyl)pyrazolo[1,5-a]pyridine-3-carboxamide;
N-(2,5-difluoro-3-(5-((1R,2S)-2-fluorocyclopropyl)-1,2,4-oxadiazol-3-yl)-6-methylphenyl)pyrazolo[1,5-a]pyridine-3-carboxamide;
methyl 3-(3-(2,5-difluoro-4-methyl-3-(pyrazolo[1,5-a]pyridine-3-carboxamido)phenyl)-1,2,4-oxadiazol-5-yl)azetidine-1-carboxylate;
N-(3-(3-(3,3-difluorocyclobutyl)-1,2,4-oxadiazol-5-yl)-2,5-difluoro-6-methylphenyl)imidazo[1,2-a]pyridine-3-carboxamide;
N-(2,5-difluoro-3-(3-((1R,2S)-2-fluorocyclopropyl)-1,2,4-oxadiazol-5-yl)-6-methylphenyl)imidazo[1,2-a]pyridine-3-carboxamide;
methyl 3-(5-(2,5-difluoro-3-(imidazo[1,2-a]pyridine-3-carboxamido)-4-methylphenyl)-1,2,4-oxadiazol-3-yl)azetidine-1-carboxylate;
methyl 3-(5-(2,5-difluoro-4-methyl-3-(7-methylimidazo[1,2-a]pyridine-3-carboxamido)phenyl)-1,2,4-oxadiazol-3-yl)azetidine-1-carboxylate;
methyl 3-(5-(2,5-difluoro-3-(7-methoxyimidazo[1,2-a]pyridine-3-carboxamido)-4-methylphenyl)-1,2,4-oxadiazol-3-yl)azetidine-1-carboxylate;
methyl 3-(5-(2,5-difluoro-4-methyl-3-(7-morpholinoimidazo[1,2-a]pyridine-3-carboxamido)phenyl)-1,2,4-oxadiazol-3-yl)azetidine-1-carboxylate; and
methyl 3-(5-(2,5-difluoro-4-methyl-3-(7-(piperazin-1-yl)imidazo[1,2-a]pyridine-3-carboxamido)phenyl)-1,2,4-oxadiazol-3-yl)azetidine-1-carboxylate.

### Preparation of Compounds

The compounds used in the synthetic chemistry reactions described herein are made according to organic synthesis techniques known to those skilled in this art, starting from commercially available chemicals and/or from compounds described in the chemical literature. “Commercially available chemicals” are obtained from standard commercial sources including Acros Organics (Pittsburgh, PA), Aldrich Chemical (Milwaukee, WI, including Sigma Chemical and Fluka), Apin Chemicals Ltd. (Milton Park, UK), Avocado Research (Lancashire, U.K.), BDH Inc. (Toronto, Canada), Bionet (Cornwall, U.K.), Chemservice Inc. (West Chester, PA), Crescent Chemical Co. (Hauppauge, NY), Eastman Organic Chemicals, Eastman Kodak Company (Rochester, NY), Fisher Scientific Co. (Pittsburgh, PA), Fisons Chemicals (Leicestershire, UK), Frontier Scientific (Logan, UT), ICN Biomedicals, Inc. (Costa Mesa, CA), Key Organics (Cornwall, U.K.), Lancaster Synthesis (Windham, NH), Maybridge Chemical Co. Ltd. (Cornwall, U.K.), Parish Chemical Co. (Orem, UT), Pfaltz & Bauer, Inc. (Waterbury, CN), Polyorganix (Houston, TX), Pierce Chemical Co. (Rockford, IL), Riedel de Haen AG (Hanover, Germany), Spectrum Quality Product, Inc. (New Brunswick, NJ), TCI America (Portland, OR), Trans World Chemicals, Inc. (Rockville, MD), and Wako Chemicals USA, Inc. (Richmond, VA).

Suitable reference books and treatise that detail the synthesis of reactants useful in the preparation of compounds described herein, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc.

Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Additional suitable reference books and treatise that detail the synthesis of reactants useful in the preparation of compounds described herein, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3-527-29074-5; Hoffman, R. V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modern Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Solomons, T. W. G. “Organic Chemistry” 7th Edition (2000) John Wiley & Sons, ISBN: 0-471-19095-0; Stowell, J. C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes.

Specific and analogous reactants are optionally identified through the indices of known chemicals prepared by the Chemical Abstract Service of the American Chemical Society, which are available in most public and university libraries, as well as through on-line databases (contact the American Chemical Society, Washington, D.C. for more details). Chemicals that are known but not commercially available in catalogs are optionally prepared by custom chemical synthesis houses, where many of the standard chemical supply houses (e.g., those listed above) provide custom synthesis services. A reference useful for the preparation and selection of pharmaceutical salts of the compounds described herein is P. H. Stahl & C. G.

Wermuth “Handbook of Pharmaceutical Salts”, Verlag Helvetica Chimica Acta, Zurich, 2002.

### Pharmaceutical Compositions

In certain embodiments, the c-KIT kinase inhibitory compound described herein is administered as a pure chemical. In other embodiments, the c-KIT kinase inhibitory compound described herein is combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected on the basis of a chosen route of administration and standard pharmaceutical practice as described, for example, in Remington: The Science and Practice of Pharmacy (Gennaro, 21^{st} Ed. Mack Pub. Co., Easton, PA (2005)).

Provided herein is a pharmaceutical composition comprising at least one c-KIT kinase inhibitory compound as described herein, or a stereoisomer, pharmaceutically acceptable salt, hydrate, or solvate thereof, together with one or more pharmaceutically acceptable carriers. The carrier(s) (or excipient(s)) is acceptable or suitable if the carrier is compatible with the other ingredients of the composition and not deleterious to the recipient (i.e., the subject or the patient) of the composition.

In some embodiments, the compounds described herein, or a pharmaceutically acceptable salt or solvate thereof, are formulated into pharmaceutical compositions. In specific embodiments, pharmaceutical compositions are formulated in a conventional manner using one or more physiologically acceptable carriers comprising excipients and auxiliaries which facilitate processing of the active compounds into preparations which can be used pharmaceutically.

Proper formulation is dependent upon the route of administration chosen. Any pharmaceutically acceptable techniques, carriers, and excipients are used as suitable to formulate the pharmaceutical compositions described herein: Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins1999).

Provided herein are pharmaceutical compositions comprising a compound of Formula (I), or a pharmaceutically acceptable salt or solvate thereof, and a pharmaceutically acceptable diluent(s), excipient(s), or carrier(s). In certain embodiments, the compounds described herein, or a pharmaceutically acceptable salt or solvate thereof, are administered as pharmaceutical compositions in which compounds of Formula (I), or a pharmaceutically acceptable salt or solvate thereof, are mixed with other active ingredients, as in combination therapy. In specific embodiments, the pharmaceutical compositions include one or more compounds of Formula (I), or a pharmaceutically acceptable salt or solvate thereof.

A pharmaceutical composition, as used herein, refers to a mixture of a compound of Formula (I), or a pharmaceutically acceptable salt or solvate thereof, with other chemical components, such as carriers, stabilizers, diluents, dispersing agents, suspending agents, thickening agents, and/or excipients. In certain embodiments, the pharmaceutical composition facilitates administration of the compound to an organism. In some embodiments, practicing the methods of treatment or use provided herein, therapeutically effective amounts of compounds of Formula (I), or a pharmaceutically acceptable salt or solvate thereof, provided herein are administered in a pharmaceutical composition to a mammal having a disease or condition to be treated. In specific embodiments, the mammal is a human. In certain embodiments, therapeutically effective amounts vary depending on the severity of the disease, the age and relative health of the subject, the potency of the compound used and other factors. The compounds described herein are used singly or in combination with one or more therapeutic agents as components of mixtures.

In one embodiment, one or more compounds of Formula (I), or a pharmaceutically acceptable salt or solvate thereof, is formulated in an aqueous solution. In specific embodiments, the aqueous solution is selected from, by way of example only, a physiologically compatible buffer, such as Hank's solution, Ringer's solution, or physiological saline buffer. In other embodiments, one or more compound of Formula (I), or a pharmaceutically acceptable salt or solvate thereof, is formulated for transmucosal administration. In specific embodiments, transmucosal formulations include penetrants that are appropriate to the barrier to be permeated. In still other embodiments wherein the compounds described herein, or a pharmaceutically acceptable salt or solvate thereof, are formulated for other parenteral injections, appropriate formulations include aqueous or nonaqueous solutions. In specific embodiments, such solutions include physiologically compatible buffers and/or excipients.

In another embodiment, compounds described herein, or a pharmaceutically acceptable salt or solvate thereof, are formulated for oral administration. Compounds described herein, or a pharmaceutically acceptable salt or solvate thereof, including compounds of Formula (I), are formulated by combining the active compounds with, e.g., pharmaceutically acceptable carriers or excipients. In various embodiments, the compounds described herein, or a pharmaceutically acceptable salt or solvate thereof, are formulated in oral dosage forms that include, by way of example only, tablets, powders, pills, dragees, capsules, liquids, gels, syrups, elixirs, slurries, suspensions, and the like.

In certain embodiments, pharmaceutical preparations for oral use are obtained by mixing one or more solid excipient with one or more of the compounds described herein, or a pharmaceutically acceptable salt or solvate thereof, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as: for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. In specific embodiments, disintegrating agents are optionally added. Disintegrating agents include, by way of example only, cross-linked croscarmellose sodium, polyvinylpyrrolidone, agar, or alginic acid or a salt thereof such as sodium alginate.

In one embodiment, dosage forms, such as dragee cores and tablets, are provided with one or more suitable coating. In specific embodiments, concentrated sugar solutions are used for coating the dosage form. The sugar solutions, optionally contain additional components, such as by way of example only, gum arabic, talc, polyvinylpyrrolidone, carbopol gel, polyethylene glycol, and/or titanium dioxide, lacquer solutions, and suitable organic solvents or solvent mixtures. Dyestuffs and/or pigments are also optionally added to the coatings for identification purposes. Additionally, the dyestuffs and/or pigments are optionally utilized to characterize different combinations of active compound doses.

In certain embodiments, therapeutically effective amounts of at least one of the compounds described herein, or a pharmaceutically acceptable salt or solvate thereof, are formulated into other oral dosage forms. Oral dosage forms include push-fit capsules made of gelatin, as well as soft, sealed capsules made of gelatin and a plasticizer, such as glycerol or sorbitol. In specific embodiments, push-fit capsules contain the active ingredients in admixture with one or more filler. Fillers include, by way of example only, lactose, binders such as starches, and/or lubricants such as talc or magnesium stearate and, optionally, stabilizers. In other embodiments, soft capsules, contain one or more active compound that is dissolved or suspended in a suitable liquid. Suitable liquids include, by way of example only, one or more fatty oil, liquid paraffin, or liquid polyethylene glycol. In addition, stabilizers are optionally added.

In other embodiments, therapeutically effective amounts of at least one of the compounds described herein, or a pharmaceutically acceptable salt or solvate thereof, are formulated for buccal or sublingual administration. Formulations suitable for buccal or sublingual administration include, by way of example only, tablets, lozenges, or gels. In still other embodiments, the compounds described herein, or a pharmaceutically acceptable salt or solvate thereof, are formulated for parental injection, including formulations suitable for bolus injection or continuous infusion. In specific embodiments, formulations for injection are presented in unit dosage form (e.g., in ampoules) or in multi-dose containers. Preservatives are, optionally, added to the injection formulations. In still other embodiments, the pharmaceutical composition of a compound of Formula (I), or a pharmaceutically acceptable salt or solvate thereof, is formulated in a form suitable for parenteral injection as sterile suspension, solution or emulsion in oily or aqueous vehicles. Parenteral injection formulations optionally contain formulatory agents such as suspending, stabilizing and/or dispersing agents. In specific embodiments, pharmaceutical formulations for parenteral administration include aqueous solutions of the active compounds in water-soluble form. In additional embodiments, suspensions of the active compounds are prepared as appropriate oily injection suspensions. Suitable lipophilic solvents or vehicles for use in the pharmaceutical compositions described herein include, by way of example only, fatty oils such as sesame oil, or synthetic fatty acid esters, such as ethyl oleate or triglycerides, or liposomes. In certain specific embodiments, aqueous injection suspensions contain substances which increase the viscosity of the suspension, such as sodium carboxymethyl cellulose, sorbitol, or dextran. Optionally, the suspension contains suitable stabilizers or agents which increase the solubility of the compounds to allow for the preparation of highly concentrated solutions. Alternatively, in other embodiments, the active ingredient is in powder form for constitution with a suitable vehicle, e.g., sterile pyrogen-free water, before use.

In still other embodiments, the compounds of Formula (I), or a pharmaceutically acceptable salt or solvate thereof, are administered topically. The compounds described herein are formulated into a variety of topically administrable compositions, such as solutions, suspensions, lotions, gels, pastes, medicated sticks, balms, creams, or ointments. Such pharmaceutical compositions optionally contain solubilizers, stabilizers, tonicity enhancing agents, buffers, and preservatives.

In yet other embodiments, the compounds of Formula (I), or a pharmaceutically acceptable salt or solvate thereof, are formulated for transdermal administration. In specific embodiments, transdermal formulations employ transdermal delivery devices and transdermal delivery patches and can be lipophilic emulsions or buffered, aqueous solutions, dissolved and/or dispersed in a polymer or an adhesive. In various embodiments, such patches are constructed for continuous, pulsatile, or on demand delivery of pharmaceutical agents. In additional embodiments, the transdermal delivery of the compounds of Formula (I), or a pharmaceutically acceptable salt or solvate thereof, is accomplished by means of iontophoretic patches and the like. In certain embodiments, transdermal patches provide controlled delivery of the compounds of Formula (I), or a pharmaceutically acceptable salt or solvate thereof. In specific embodiments, the rate of absorption is slowed by using rate-controlling membranes or by trapping the compound within a polymer matrix or gel. In alternative embodiments, absorption enhancers are used to increase absorption. Absorption enhancers or carriers include absorbable pharmaceutically acceptable solvents that assist passage through the skin. For example, in one embodiment, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin.

In other embodiments, the compounds of Formula (I), or a pharmaceutically acceptable salt or solvate thereof, are formulated for administration by inhalation. Various forms suitable for administration by inhalation include, but are not limited to, aerosols, mists, or powders. Pharmaceutical compositions of Formula (I), or a pharmaceutically acceptable salt or solvate thereof, are conveniently delivered in the form of an aerosol spray presentation from pressurized packs or a nebuliser, with the use of a suitable propellant (e.g., dichlorodifluoromethane, trichlorofluoromethane, dichlorotetrafluoroethane, carbon dioxide or other suitable gas). In specific embodiments, the dosage unit of a pressurized aerosol is determined by providing a valve to deliver a metered amount. In certain embodiments, capsules, and cartridges of, such as, by way of example only, gelatin for use in an inhaler or insufflator are formulated containing a powder mix of the compound and a suitable powder base such as lactose or starch.

In still other embodiments, the compounds of Formula (I), or a pharmaceutically acceptable salt or solvate thereof, are formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone, PEG, and the like. In suppository forms of the compositions, a low-melting wax such as, but not limited to, a mixture of fatty acid glycerides, optionally in combination with cocoa butter is first melted.

In certain embodiments, pharmaceutical compositions are formulated in any conventional manner using one or more physiologically acceptable carriers comprising excipients and auxiliaries which facilitate processing of the active compounds into preparations which can be used pharmaceutically. Proper formulation is dependent upon the route of administration chosen. Any pharmaceutically acceptable techniques, carriers, and excipients are optionally used as suitable. Pharmaceutical compositions comprising a compound of Formula (I), or a pharmaceutically acceptable salt or solvate thereof, are manufactured in a conventional manner, such as, by way of example only, by means of conventional mixing, dissolving, granulating, dragee-making, levigating, emulsifying, encapsulating, entrapping or compression processes.

Pharmaceutical compositions include at least one pharmaceutically acceptable carrier, diluent, or excipient and at least one compound of Formula (I), or a pharmaceutically acceptable salt or solvate thereof, described herein as an active ingredient. The active ingredient is in free-acid or free-base form, or in a pharmaceutically acceptable salt form. In addition, the methods and pharmaceutical compositions described herein include the use of N-oxides, crystalline forms (also known as polymorphs), as well as active metabolites of these compounds having the same type of activity. All tautomers of the compounds described herein are included within the scope of the compounds presented herein. Additionally, the compounds described herein encompass unsolvated as well as solvated forms with pharmaceutically acceptable solvents such as water, ethanol, and the like. The solvated forms of the compounds presented herein are also considered to be disclosed herein. In addition, the pharmaceutical compositions optionally include other medicinal or pharmaceutical agents, carriers, adjuvants, such as preserving, stabilizing, wetting or emulsifying agents, solution promoters, salts for regulating the osmotic pressure, buffers, and/or other therapeutically valuable substances.

Methods for the preparation of compositions comprising the compounds described herein include formulating the compounds with one or more inert, pharmaceutically acceptable excipients or carriers to form a solid, semi-solid or liquid. Solid compositions include, but are not limited to, powders, tablets, dispersible granules, capsules, cachets, and suppositories. Liquid compositions include solutions in which a compound is dissolved, emulsions comprising a compound, or a solution containing liposomes, micelles, or nanoparticles comprising a compound as disclosed herein. Semi-solid compositions include, but are not limited to, gels, suspensions, and creams. The form of the pharmaceutical compositions described herein include liquid solutions or suspensions, solid forms suitable for solution or suspension in a liquid prior to use, or as emulsions. These compositions also optionally contain minor amounts of nontoxic, auxiliary substances, such as wetting or emulsifying agents, pH buffering agents, and so forth.

In some embodiments, a pharmaceutical composition comprising at least one compound of Formula (I), or a pharmaceutically acceptable salt or solvate thereof, illustratively takes the form of a liquid where the agents are present in solution, in suspension or both. Typically when the composition is administered as a solution or suspension a first portion of the agent is present in solution and a second portion of the agent is present in particulate form, in suspension in a liquid matrix. In some embodiments, a liquid composition includes a gel formulation. In other embodiments, the liquid composition is aqueous.

In certain embodiments, useful aqueous suspension contain one or more polymers as suspending agents. Useful polymers include water-soluble polymers such as cellulosic polymers, e.g., hydroxypropyl methylcellulose, and water-insoluble polymers such as cross-linked carboxyl-containing polymers. Certain pharmaceutical compositions described herein comprise a mucoadhesive polymer, selected for example from carboxymethylcellulose, carbomer (acrylic acid polymer), poly(methylmethacrylate), polyacrylamide, polycarbophil, acrylic acid/butyl acrylate copolymer, sodium alginate and dextran.

Useful pharmaceutical compositions also, optionally, include solubilizing agents to aid in the solubility of a compound of Formula (I), or a pharmaceutically acceptable salt or solvate thereof. The term “solubilizing agent” generally includes agents that result in formation of a micellar solution or a true solution of the agent. Certain acceptable nonionic surfactants, for example polysorbate 80, are useful as solubilizing agents, as can ophthalmically acceptable glycols, polyglycols, e.g., polyethylene glycol 400, and glycol ethers.

Furthermore, useful pharmaceutical compositions optionally include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

Additionally, useful compositions also, optionally, include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

Other useful pharmaceutical compositions optionally include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

Still other useful compositions include one or more surfactants to enhance physical stability or for other purposes. Suitable nonionic surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40.

Still other useful compositions include one or more antioxidants to enhance chemical stability where required. Suitable antioxidants include, by way of example only, ascorbic acid and sodium metabisulfite.

In certain embodiments, aqueous suspension compositions are packaged in single-dose non-reclosable containers. Alternatively, multiple-dose reclosable containers are used, in which case it is typical to include a preservative in the composition.

In alternative embodiments, other delivery systems for hydrophobic pharmaceutical compounds are employed. Liposomes and emulsions are examples of delivery vehicles or carriers useful herein. In certain embodiments, organic solvents such as N-methylpyrrolidone are also employed. In additional embodiments, the compounds described herein are delivered using a sustained-release system, such as semipermeable matrices of solid hydrophobic polymers containing the therapeutic agent. Various sustained-release materials are useful herein. In some embodiments, sustained-release capsules release the compounds for a few weeks up to over 100 days. Depending on the chemical nature and the biological stability of the therapeutic reagent, additional strategies for protein stabilization are employed.

In certain embodiments, the formulations described herein comprise one or more antioxidants, metal chelating agents, thiol containing compounds and/or other general stabilizing agents. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (e) about 0.01% to about 2% w/v ascorbic acid, (f) 0.003% to about 0.02% w/v polysorbate 80, (g) 0.001% to about 0.05% w/v. polysorbate 20, (h) arginine, (i) heparin, (j) dextran sulfate, (k) cyclodextrins, (l) pentosan polysulfate and other heparinoids, (m) divalent cations such as magnesium and zinc; or (n) combinations thereof.

### Routes of Administration

Suitable routes of administration include, but are not limited to, oral, intravenous, rectal, aerosol, parenteral, ophthalmic, pulmonary, transmucosal, transdermal, vaginal, otic, nasal, and topical administration. In addition, by way of example only, parenteral delivery includes intramuscular, subcutaneous, intravenous, intramedullary injections, as well as intrathecal, direct intraventricular, intraperitoneal, intralymphatic, and intranasal injections.

In certain embodiments, a compound as described herein is administered in a local rather than systemic manner, for example, via injection of the compound directly into an organ, often in a depot preparation or sustained release formulation. In specific embodiments, long acting formulations are administered by implantation (for example subcutaneously or intramuscularly) or by intramuscular injection. Furthermore, in other embodiments, the drug is delivered in a targeted drug delivery system, for example, in a liposome coated with organ-specific antibody. In such embodiments, the liposomes are targeted to and taken up selectively by the organ. In yet other embodiments, the compound as described herein is provided in the form of a rapid release formulation, in the form of an extended release formulation, or in the form of an intermediate release formulation. In yet other embodiments, the compound described herein is administered topically.

One embodiment provides a pharmaceutical composition comprising a pharmaceutically acceptable excipient and a compound of Formula (I), or a pharmaceutically acceptable salt or solvate thereof. In some embodiments, the compound of Formula (I) is selected from the group consisting of:
N-(5-(5-(3,3-difluorocyclobutyl)-1,2,4-oxadiazol-3-yl)-3-fluoro-2-methylphenyl)imidazo[1,2-a]pyridine-3-carboxamide;
N-(3-fluoro-5-(5-((1R,2S)-2-fluorocyclopropyl)-1,2,4-oxadiazol-3-yl)-2-methylphenyl)imidazo[1,2-a]pyridine-3-carboxamide;
methyl 3-(3-(3-fluoro-5-(imidazo[1,2-a]pyridine-3-carboxamido)-4-methylphenyl)-1,2,4-oxadiazol-5-yl)azetidine-1-carboxylate;
N-(5-(5-(3,3-difluorocyclobutyl)-1,2,4-oxadiazol-3-yl)-3-fluoro-2-methylphenyl)pyrazolo[1,5-a]pyridine-3-carboxamide;
N-(3-fluoro-5-(5-((1R,2S)-2-fluorocyclopropyl)-1,2,4-oxadiazol-3-yl)-2-methylphenyl)pyrazolo[1,5-a]pyridine-3-carboxamide;
methyl 3-(3-(3-fluoro-4-methyl-5-(pyrazolo[1,5-a]pyridine-3-carboxamido)phenyl)-1,2,4-oxadiazol-5-yl)azetidine-1-carboxylate;
N-(5-(5-(3,3-difluorocyclobutyl)-1,2,4-oxadiazol-3-yl)-3-fluoro-2-methylphenyl)-6-morpholinoimidazo[1,2-a]pyridine-3-carboxamide;
N-(3-fluoro-5-(5-((1R,2S)-2-fluorocyclopropyl)-1,2,4-oxadiazol-3-yl)-2-methylphenyl)-6-morpholinoimidazo[1,2-a]pyridine-3-carboxamide;
methyl 3-(3-(3-fluoro-4-methyl-5-(6-morpholinoimidazo[1,2-a]pyridine-3-carboxamido)phenyl)-1,2,4-oxadiazol-5-yl)azetidine-1-carboxylate;
N-(5-(5-(3,3-difluorocyclobutyl)-1,2,4-oxadiazol-3-yl)-3-fluoro-2-methylphenyl)-7-(piperazin-1-yl)imidazo[1,2-a]pyridine-3-carboxamide;
N-(5-(5-(3,3-difluorocyclobutyl)-1,2,4-oxadiazol-3-yl)-3-fluoro-2-methylphenyl)-7-(4-methylpiperazin-1-yl)imidazo[1,2-a]pyridine-3-carboxamide;
methyl 3-(3-(3-(6-chloroimidazo[1,2-a]pyridine-3-carboxamido)-5-fluoro-4-methylphenyl)-1,2,4-oxadiazol-5-yl)azetidine-1-carboxylate;
N-(5-(5-(3,3-difluorocyclobutyl)-1,2,4-oxadiazol-3-yl)-3-fluoro-2-methylphenyl)-7-morpholinoimidazo[1,2-a]pyridine-3-carboxamide;
N-(3-fluoro-5-(5-((1R,2S)-2-fluorocyclopropyl)-1,2,4-oxadiazol-3-yl)-2-methylphenyl)-7-morpholinoimidazo[1,2-a]pyridine-3-carboxamide;
methyl 3-(3-(3-fluoro-4-methyl-5-(7-morpholinoimidazo[1,2-a]pyridine-3-carboxamido)phenyl)-1,2,4-oxadiazol-5-yl)azetidine-1-carboxylate;
methyl 3-(3-(3-(7-chloroimidazo[1,2-a]pyridine-3-carboxamido)-5-fluoro-4-methylphenyl)-1,2,4-oxadiazol-5-yl)azetidine-1-carboxylate;
methyl 3-(3-(3-fluoro-4-methyl-5-(7-methylimidazo[1,2-a]pyridine-3-carboxamido)phenyl)-1,2,4-oxadiazol-5-yl)azetidine-1-carboxylate;
7-(4-acetylpiperazin-1-yl)-N-(5-(5-(3,3-difluorocyclobutyl)-1,2,4-oxadiazol-3-yl)-3-fluoro-2-methylphenyl)imidazo[1,2-a]pyridine-3-carboxamide;
methyl 3-(3-(3-fluoro-5-(6-fluoroimidazo[1,2-a]pyridine-3-carboxamido)-4-methylphenyl)-1,2,4-oxadiazol-5-yl)azetidine-1-carboxylate;
methyl 3-(3-(3-fluoro-4-methyl-5-(6-(piperazin-1-yl)imidazo[1,2-a]pyridine-3-carboxamido)phenyl)-1,2,4-oxadiazol-5-yl)azetidine-1-carboxylate;
methyl 3-(3-(3-fluoro-4-methyl-5-(6-methylimidazo[1,2-a]pyridine-3-carboxamido)phenyl)-1,2,4-oxadiazol-5-yl)azetidine-1-carboxylate;
N-(3-fluoro-5-(5-((1R,2S)-2-fluorocyclopropyl)-1,2,4-oxadiazol-3-yl)-2-methylphenyl)-7-(piperazin-1-yl)imidazo[1,2-a]pyridine-3-carboxamide;
ethyl 3-(3-(3-fluoro-5-(imidazo[1,2-a]pyridine-3-carboxamido)-4-methylphenyl)-1,2,4-oxadiazol-5-yl)azetidine-1-carboxylate;
methyl 3-(3-(3-fluoro-5-(7-fluoroimidazo[1,2-a]pyridine-3-carboxamido)-4-methylphenyl)-1,2,4-oxadiazol-5-yl)azetidine-1-carboxylate;
N-(5-(5-(3,3-difluorocyclobutyl)-1,2,4-oxadiazol-3-yl)-3-fluoro-2-methylphenyl)-6-(piperazin-1-yl)imidazo[1,2-a]pyridine-3-carboxamide;
N-(3-fluoro-5-(5-((1R,2S)-2-fluorocyclopropyl)-1,2,4-oxadiazol-3-yl)-2-methylphenyl)-6-(piperazin-1-yl)imidazo[1,2-a]pyridine-3-carboxamide;
methyl 3-(3-(3-fluoro-5-(6-methoxyimidazo[1,2-a]pyridine-3-carboxamido)-4-methylphenyl)-1,2,4-oxadiazol-5-yl)azetidine-1-carboxylate;
methyl 3-(3-(3-fluoro-4-methyl-5-(6-(4-methylpiperazin-1-yl)imidazo[1,2-a]pyridine-3-carboxamido)phenyl)-1,2,4-oxadiazol-5-yl)azetidine-1-carboxylate;
methyl 3-(3-(3-fluoro-5-(7-methoxyimidazo[1,2-a]pyridine-3-carboxamido)-4-methylphenyl)-1,2,4-oxadiazol-5-yl)azetidine-1-carboxylate;
methyl 3-(3-(3-fluoro-4-methyl-5-(7-(piperazin-1-yl)imidazo[1,2-a]pyridine-3-carboxamido)phenyl)-1,2,4-oxadiazol-5-yl)azetidine-1-carboxylate;
cyclopropyl 3-(3-(3-fluoro-5-(imidazo[1,2-a]pyridine-3-carboxamido)-4-methylphenyl)-1,2,4-oxadiazol-5-yl)azetidine-1-carboxylate;
N-(3-fluoro-2-methyl-5-(5-(1-(methylcarbamoyl)azetidin-3-yl)-1,2,4-oxadiazol-3-yl)phenyl)imidazo[1,2-a]pyridine-3-carboxamide;
N-(5-(5-(3,3-difluorocyclobutyl)-1,2,4-oxadiazol-3-yl)-3-fluoro-2-methylphenyl)-6-(4-methylpiperazin-1-yl)imidazo[1,2-a]pyridine-3-carboxamide;
N-(3-fluoro-5-(5-((1R,2S)-2-fluorocyclopropyl)-1,2,4-oxadiazol-3-yl)-2-methylphenyl)-6-(4-methylpiperazin-1-yl)imidazo[1,2-a]pyridine-3-carboxamide;
methyl 3-(3-(3-(7-cyanoimidazo[1,2-a]pyridine-3-carboxamido)-5-fluoro-4-methylphenyl)-1,2,4-oxadiazol-5-yl)azetidine-1-carboxylate;
methyl 3-(3-(3-(6-(4-acetylpiperazin-1-yl)imidazo[1,2-a]pyridine-3-carboxamido)-5-fluoro-4-methylphenyl)-1,2,4-oxadiazol-5-yl)azetidine-1-carboxylate;
methyl 3-(5-(3-fluoro-5-(imidazo[1,2-a]pyridine-3-carboxamido)-4-methylphenyl)-1,2,4-oxadiazol-3-yl)azetidine-1-carboxylate;
6-(4-acetylpiperazin-1-yl)-N-(5-(5-(3,3-difluorocyclobutyl)-1,2,4-oxadiazol-3-yl)-3-fluoro-2-methylphenyl)imidazo[1,2-a]pyridine-3-carboxamide;
6-(4-acetylpiperazin-1-yl)-N-(3-fluoro-5-(5-((1R,2S)-2-fluorocyclopropyl)-1,2,4-oxadiazol-3-yl)-2-methylphenyl)imidazo[1,2-a]pyridine-3-carboxamide;
methyl 3-(3-(3-(6-carbamoylimidazo[1,2-a]pyridine-3-carboxamido)-5-fluoro-4-methylphenyl)-1,2,4-oxadiazol-5-yl)azetidine-1-carboxylate;
2-aminoethyl 3-(3-(3-fluoro-5-(imidazo[1,2-a]pyridine-3-carboxamido)-4-methylphenyl)-1,2,4-oxadiazol-5-yl)azetidine-1-carboxylate;
2-methoxyethyl 3-(3-(3-fluoro-5-(imidazo[1,2-a]pyridine-3-carboxamido)-4-methylphenyl)-1,2,4-oxadiazol-5-yl)azetidine-1-carboxylate;
methyl 3-(3-(3-(7-carbamoylimidazo[1,2-a]pyridine-3-carboxamido)-5-fluoro-4-methylphenyl)-1,2,4-oxadiazol-5-yl)azetidine-1-carboxylate;
isopropyl 3-(3-(3-fluoro-5-(imidazo[1,2-a]pyridine-3-carboxamido)-4-methylphenyl)-1,2,4-oxadiazol-5-yl)azetidine-1-carboxylate;
N-(5-(5-(1-(ethylcarbamoyl)azetidin-3-yl)-1,2,4-oxadiazol-3-yl)-3-fluoro-2-methylphenyl)imidazo[1,2-a]pyridine-3-carboxamide;
N-(3-fluoro-5-(5-(1-(isopropylcarbamoyl)azetidin-3-yl)-1,2,4-oxadiazol-3-yl)-2-methylphenyl)imidazo[1,2-a]pyridine-3-carboxamide;
N-(3-fluoro-5-(5-(1-((2-hydroxyethyl)carbamoyl)azetidin-3-yl)-1,2,4-oxadiazol-3-yl)-2-methylphenyl)imidazo[1,2-a]pyridine-3-carboxamide;
N-(5-(5-(1-(cyclopropylcarbamoyl)azetidin-3-yl)-1,2,4-oxadiazol-3-yl)-3-fluoro-2-methylphenyl)imidazo[1,2-a]pyridine-3-carboxamide;
N-(3-fluoro-5-(5-(1-((2-methoxyethyl)carbamoyl)azetidin-3-yl)-1,2,4-oxadiazol-3-yl)-2-methylphenyl)imidazo[1,2-a]pyridine-3-carboxamide;
N-(5-(5-(1-((2-aminoethyl)carbamoyl)azetidin-3-yl)-1,2,4-oxadiazol-3-yl)-3-fluoro-2-methylphenyl)imidazo[1,2-a]pyridine-3-carboxamide;
N-(3-fluoro-2-methyl-5-(5-(1-(morpholine-4-carbonyl)azetidin-3-yl)-1,2,4-oxadiazol-3-yl)phenyl)imidazo[1,2-a]pyridine-3-carboxamide;
N-(3-fluoro-2-methyl-5-(5-(1-(piperazine-1-carbonyl)azetidin-3-yl)-1,2,4-oxadiazol-3-yl)phenyl)imidazo[1,2-a]pyridine-3-carboxamide;
N-(3-fluoro-2-methyl-5-(5-(1-(4-methylpiperazine-1-carbonyl)azetidin-3-yl)-1,2,4-oxadiazol-3-yl)phenyl)imidazo[1,2-a]pyridine-3-carboxamide;
methyl 3-(3-(3-(6-cyanoimidazo[1,2-a]pyridine-3-carboxamido)-5-fluoro-4-methylphenyl)-1,2,4-oxadiazol-5-yl)azetidine-1-carboxylate;
methyl 3-(3-(3-fluoro-4-methyl-5-(6-(trifluoromethyl)imidazo[1,2-a]pyridine-3-carboxamido)phenyl)-1,2,4-oxadiazol-5-yl)azetidine-1-carboxylate;
methyl 3-(3-(3-fluoro-5-(6-isopropylimidazo[1,2-a]pyridine-3-carboxamido)-4-methylphenyl)-1,2,4-oxadiazol-5-yl)azetidine-1-carboxylate;
methyl 3-(3-(3-(6-cyclopropylimidazo[1,2-a]pyridine-3-carboxamido)-5-fluoro-4-methylphenyl)-1,2,4-oxadiazol-5-yl)azetidine-1-carboxylate;
methyl 3-(3-(3-fluoro-5-(7-isopropylimidazo[1,2-a]pyridine-3-carboxamido)-4-methylphenyl)-1,2,4-oxadiazol-5-yl)azetidine-1-carboxylate;
methyl 3-(3-(3-(7-cyclopropylimidazo[1,2-a]pyridine-3-carboxamido)-5-fluoro-4-methylphenyl)-1,2,4-oxadiazol-5-yl)azetidine-1-carboxylate;
methyl 3-(3-(3-fluoro-4-methyl-5-(7-(4-methylpiperazin-1-yl)imidazo[1,2-a]pyridine-3-carboxamido)phenyl)-1,2,4-oxadiazol-5-yl)azetidine-1-carboxylate;
methyl 4-(3-(3-fluoro-5-(imidazo[1,2-a]pyridine-3-carboxamido)-4-methylphenyl)-1,2,4-oxadiazol-5-yl)piperazine-1-carboxylate;
methyl 4-(3-(3-fluoro-5-(imidazo[1,2-a]pyridine-3-carboxamido)-4-methylphenyl)-1,2,4-oxadiazol-5-yl)-2-methylpiperazine-1-carboxylate;
methyl 4-(3-(3-fluoro-5-(imidazo[1,2-a]pyridine-3-carboxamido)-4-methylphenyl)-1,2,4-oxadiazol-5-yl)-2-isopropylpiperazine-1-carboxylate;
methyl 4-(3-(3-fluoro-5-(imidazo[1,2-a]pyridine-3-carboxamido)-4-methylphenyl)-1,2,4-oxadiazol-5-yl)-3-methylpiperazine-1-carboxylate;
methyl 4-(3-(3-fluoro-5-(imidazo[1,2-a]pyridine-3-carboxamido)-4-methylphenyl)-1,2,4-oxadiazol-5-yl)-3-isopropylpiperazine-1-carboxylate;
N-(5-(5-(4-acetylpiperazin-1-yl)-1,2,4-oxadiazol-3-yl)-3-fluoro-2-methylphenyl)imidazo[1,2-a]pyridine-3-carboxamide;
N-(5-(5-(4-carbamoylpiperazin-1-yl)-1,2,4-oxadiazol-3-yl)-3-fluoro-2-methylphenyl)imidazo[1,2-a]pyridine-3-carboxamide;
N-(3-fluoro-2-methyl-5-(5-(4-methylpiperazin-1-yl)-1,2,4-oxadiazol-3-yl)phenyl)imidazo[1,2-a]pyridine-3-carboxamide;
N-(3-fluoro-2-methyl-5-(5-(piperazin-1-yl)-1,2,4-oxadiazol-3-yl)phenyl)imidazo[1,2-a]pyridine-3-carboxamide;
N-(3-fluoro-2-methyl-5-(5-morpholino-1,2,4-oxadiazol-3-yl)phenyl)imidazo[1,2-a]pyridine-3-carboxamide;
6-fluoro-N-(3-fluoro-5-(5-((1R,2S)-2-fluorocyclopropyl)-1,2,4-oxadiazol-3-yl)-2-methylphenyl)imidazo[1,2-a]pyridine-3-carboxamide;
6-chloro-N-(3-fluoro-5-(5-((1R,2S)-2-fluorocyclopropyl)-1,2,4-oxadiazol-3-yl)-2-methylphenyl)imidazo[1,2-a]pyridine-3-carboxamide;
6-cyano-N-(3-fluoro-5-(5-((1R,2S)-2-fluorocyclopropyl)-1,2,4-oxadiazol-3-yl)-2-methylphenyl)imidazo[1,2-a]pyridine-3-carboxamide;
N3-(3-fluoro-5-(5-((1R,2S)-2-fluorocyclopropyl)-1,2,4-oxadiazol-3-yl)-2-methylphenyl)imidazo[1,2-a]pyridine-3,6-dicarboxamide;
N-(3-fluoro-5-(5-((1R,2S)-2-fluorocyclopropyl)-1,2,4-oxadiazol-3-yl)-2-methylphenyl)-6-(trifluoromethyl)imidazo[1,2-a]pyridine-3-carboxamide;
N-(3-fluoro-5-(5-((1R,2S)-2-fluorocyclopropyl)-1,2,4-oxadiazol-3-yl)-2-methylphenyl)-6-methylimidazo[1,2-a]pyridine-3-carboxamide;
N-(3-fluoro-5-(5-((1R,2S)-2-fluorocyclopropyl)-1,2,4-oxadiazol-3-yl)-2-methylphenyl)-6-isopropylimidazo[1,2-a]pyridine-3-carboxamide;
6-cyclopropyl-N-(3-fluoro-5-(5-((1R,2S)-2-fluorocyclopropyl)-1,2,4-oxadiazol-3-yl)-2-methylphenyl)imidazo[1,2-a]pyridine-3-carboxamide;
7-fluoro-N-(3-fluoro-5-(5-((1R,2S)-2-fluorocyclopropyl)-1,2,4-oxadiazol-3-yl)-2-methylphenyl)imidazo[1,2-a]pyridine-3-carboxamide;
7-chloro-N-(3-fluoro-5-(5-((1R,2S)-2-fluorocyclopropyl)-1,2,4-oxadiazol-3-yl)-2-methylphenyl)imidazo[1,2-a]pyridine-3-carboxamide;
N-(3-fluoro-5-(5-((1R,2S)-2-fluorocyclopropyl)-1,2,4-oxadiazol-3-yl)-2-methylphenyl)-7-(trifluoromethyl)imidazo[1,2-a]pyridine-3-carboxamide;
7-cyano-N-(3-fluoro-5-(5-((1R,2S)-2-fluorocyclopropyl)-1,2,4-oxadiazol-3-yl)-2-methylphenyl)imidazo[1,2-a]pyridine-3-carboxamide;
N3-(3-fluoro-5-(5-((1R,2S)-2-fluorocyclopropyl)-1,2,4-oxadiazol-3-yl)-2-methylphenyl)imidazo[1,2-a]pyridine-3,7-dicarboxamide;
N-(3-fluoro-5-(5-((1R,2S)-2-fluorocyclopropyl)-1,2,4-oxadiazol-3-yl)-2-methylphenyl)-7-methylimidazo[1,2-a]pyridine-3-carboxamide;
N-(3-fluoro-5-(5-((1R,2S)-2-fluorocyclopropyl)-1,2,4-oxadiazol-3-yl)-2-methylphenyl)-7-isopropylimidazo[1,2-a]pyridine-3-carboxamide;
7-cyclopropyl-N-(3-fluoro-5-(5-((1R,2S)-2-fluorocyclopropyl)-1,2,4-oxadiazol-3-yl)-2-methylphenyl)imidazo[1,2-a]pyridine-3-carboxamide;
N-(3-fluoro-5-(5-((1R,2S)-2-fluorocyclopropyl)-1,2,4-oxadiazol-3-yl)-2-methylphenyl)-7-(4-methylpiperazin-1-yl)imidazo[1,2-a]pyridine-3-carboxamide;
methyl 3-(3-(3-fluoro-4-methyl-5-(7-(trifluoromethyl)imidazo[1,2-a]pyridine-3-carboxamido)phenyl)-1,2,4-oxadiazol-5-yl)azetidine-1-carboxylate;
N-(3-fluoro-5-(5-((1S,2R)-2-fluorocyclopropyl)-1,2,4-oxadiazol-3-yl)-2-methylphenyl)imidazo[1,2-a]pyridine-3-carboxamide;
N-(3-fluoro-5-(5-((1R,2R)-2-fluorocyclopropyl)-1,2,4-oxadiazol-3-yl)-2-methylphenyl)imidazo[1,2-a]pyridine-3-carboxamide;
N-(3-fluoro-5-(5-((1S,2S)-2-fluorocyclopropyl)-1,2,4-oxadiazol-3-yl)-2-methylphenyl)imidazo[1,2-a]pyridine-3-carboxamide;
methyl 3-(3-(3-fluoro-5-(6-fluoropyrazolo[1,5-a]pyridine-3-carboxamido)-4-methylphenyl)-1,2,4-oxadiazol-5-yl)azetidine-1-carboxylate;
methyl 3-(3-(3-(6-chloropyrazolo[1,5-a]pyridine-3-carboxamido)-5-fluoro-4-methylphenyl)-1,2,4-oxadiazol-5-yl)azetidine-1-carboxylate;
methyl 3-(3-(3-(6-cyanopyrazolo[1,5-a]pyridine-3-carboxamido)-5-fluoro-4-methylphenyl)-1,2,4-oxadiazol-5-yl)azetidine-1-carboxylate;
methyl 3-(3-(3-(6-carbamoylpyrazolo[1,5-a]pyridine-3-carboxamido)-5-fluoro-4-methylphenyl)-1,2,4-oxadiazol-5-yl)azetidine-1-carboxylate;
methyl 3-(3-(3-fluoro-4-methyl-5-(6-(trifluoromethyl)pyrazolo[1,5-a]pyridine-3-carboxamido)phenyl)-1,2,4-oxadiazol-5-yl)azetidine-1-carboxylate;
methyl 3-(3-(3-fluoro-5-(6-methoxypyrazolo[1,5-a]pyridine-3-carboxamido)-4-methylphenyl)-1,2,4-oxadiazol-5-yl)azetidine-1-carboxylate;
methyl 3-(3-(3-fluoro-5-(6-isopropylpyrazolo[1,5-a]pyridine-3-carboxamido)-4-methylphenyl)-1,2,4-oxadiazol-5-yl)azetidine-1-carboxylate;
methyl 3-(3-(3-(6-cyclopropylpyrazolo[1,5-a]pyridine-3-carboxamido)-5-fluoro-4-methylphenyl)-1,2,4-oxadiazol-5-yl)azetidine-1-carboxylate;
methyl 3-(3-(3-fluoro-4-methyl-5-(6-methylpyrazolo[1,5-a]pyridine-3-carboxamido)phenyl)-1,2,4-oxadiazol-5-yl)azetidine-1-carboxylate;
methyl 3-(3-(3-fluoro-4-methyl-5-(5-methylpyrazolo[1,5-a]pyridine-3-carboxamido)phenyl)-1,2,4-oxadiazol-5-yl)azetidine-1-carboxylate;
methyl 3-(3-(3-(5-cyclopropylpyrazolo[1,5-a]pyridine-3-carboxamido)-5-fluoro-4-methylphenyl)-1,2,4-oxadiazol-5-yl)azetidine-1-carboxylate;
methyl 3-(3-(3-fluoro-5-(5-isopropylpyrazolo[1,5-a]pyridine-3-carboxamido)-4-methylphenyl)-1,2,4-oxadiazol-5-yl)azetidine-1-carboxylate;
methyl 3-(3-(3-fluoro-5-(5-fluoropyrazolo[1,5-a]pyridine-3-carboxamido)-4-methylphenyl)-1,2,4-oxadiazol-5-yl)azetidine-1-carboxylate;
methyl 3-(3-(3-(5-chloropyrazolo[1,5-a]pyridine-3-carboxamido)-5-fluoro-4-methylphenyl)-1,2,4-oxadiazol-5-yl)azetidine-1-carboxylate;
methyl 3-(3-(3-(5-cyanopyrazolo[1,5-a]pyridine-3-carboxamido)-5-fluoro-4-methylphenyl)-1,2,4-oxadiazol-5-yl)azetidine-1-carboxylate;
methyl 3-(3-(3-(5-carbamoylpyrazolo[1,5-a]pyridine-3-carboxamido)-5-fluoro-4-methylphenyl)-1,2,4-oxadiazol-5-yl)azetidine-1-carboxylate;
methyl 3-(3-(3-fluoro-4-methyl-5-(5-(trifluoromethyl)pyrazolo[1,5-a]pyridine-3-carboxamido)phenyl)-1,2,4-oxadiazol-5-yl)azetidine-1-carboxylate;
methyl 3-(3-(3-fluoro-4-methyl-5-(5-morpholinopyrazolo[1,5-a]pyridine-3-carboxamido)phenyl)-1,2,4-oxadiazol-5-yl)azetidine-1-carboxylate;
methyl 3-(3-(3-fluoro-4-methyl-5-(5-(piperazin-1-yl)pyrazolo[1,5-a]pyridine-3-carboxamido)phenyl)-1,2,4-oxadiazol-5-yl)azetidine-1-carboxylate;
methyl 3-(3-(3-fluoro-4-methyl-5-(5-(4-methylpiperazin-1-yl)pyrazolo[1,5-a]pyridine-3-carboxamido)phenyl)-1,2,4-oxadiazol-5-yl)azetidine-1-carboxylate;
methyl 3-(3-(3-fluoro-4-methyl-5-(6-morpholinopyrazolo[1,5-a]pyridine-3-carboxamido)phenyl)-1,2,4-oxadiazol-5-yl)azetidine-1-carboxylate;
methyl 3-(3-(3-fluoro-4-methyl-5-(6-(piperazin-1-yl)pyrazolo[1,5-a]pyridine-3-carboxamido)phenyl)-1,2,4-oxadiazol-5-yl)azetidine-1-carboxylate;
methyl 3-(3-(3-fluoro-4-methyl-5-(6-(4-methylpiperazin-1-yl)pyrazolo[1,5-a]pyridine-3-carboxamido)phenyl)-1,2,4-oxadiazol-5-yl)azetidine-1-carboxylate;
N-(5-(3-(3,3-difluorocyclobutyl)-1,2,4-oxadiazol-5-yl)-3-fluoro-2-methylphenyl)imidazo[1,2-a]pyridine-3-carboxamide;
N-(3-fluoro-5-(3-((1R,2S)-2-fluorocyclopropyl)-1,2,4-oxadiazol-5-yl)-2-methylphenyl)imidazo[1,2-a]pyridine-3-carboxamide;
isopropyl 3-(5-(3-fluoro-5-(imidazo[1,2-a]pyridine-3-carboxamido)-4-methylphenyl)-1,2,4-oxadiazol-3-yl)azetidine-1-carboxylate;
cyclopropyl 3-(5-(3-fluoro-5-(imidazo[1,2-a]pyridine-3-carboxamido)-4-methylphenyl)-1,2,4-oxadiazol-3-yl)azetidine-1-carboxylate;
N-(3-fluoro-2-methyl-5-(3-(1-(methylcarbamoyl)azetidin-3-yl)-1,2,4-oxadiazol-5-yl)phenyl)imidazo[1,2-a]pyridine-3-carboxamide;
N-(3-fluoro-2-methyl-5-(3-(1-(morpholine-4-carbonyl)azetidin-3-yl)-1,2,4-oxadiazol-5-yl)phenyl)imidazo[1,2-a]pyridine-3-carboxamide;
N-(3-fluoro-2-methyl-5-(3-(1-(piperazine-1-carbonyl)azetidin-3-yl)-1,2,4-oxadiazol-5-yl)phenyl)imidazo[1,2-a]pyridine-3-carboxamide;
methyl 3-(5-(3-fluoro-5-(6-fluoroimidazo[1,2-a]pyridine-3-carboxamido)-4-methylphenyl)-1,2,4-oxadiazol-3-yl)azetidine-1-carboxylate;
methyl 3-(5-(3-(6-chloroimidazo[1,2-a]pyridine-3-carboxamido)-5-fluoro-4-methylphenyl)-1,2,4-oxadiazol-3-yl)azetidine-1-carboxylate;
methyl 3-(5-(3-(6-cyanoimidazo[1,2-a]pyridine-3-carboxamido)-5-fluoro-4-methylphenyl)-1,2,4-oxadiazol-3-yl)azetidine-1-carboxylate;
methyl 3-(5-(3-(6-carbamoylimidazo[1,2-a]pyridine-3-carboxamido)-5-fluoro-4-methylphenyl)-1,2,4-oxadiazol-3-yl)azetidine-1-carboxylate;
methyl 3-(5-(3-fluoro-4-methyl-5-(6-(trifluoromethyl)imidazo[1,2-a]pyridine-3-carboxamido)phenyl)-1,2,4-oxadiazol-3-yl)azetidine-1-carboxylate;
methyl 3-(5-(3-fluoro-4-methyl-5-(6-methylimidazo[1,2-a]pyridine-3-carboxamido)phenyl)-1,2,4-oxadiazol-3-yl)azetidine-1-carboxylate;
methyl 3-(5-(3-fluoro-5-(6-isopropylimidazo[1,2-a]pyridine-3-carboxamido)-4-methylphenyl)-1,2,4-oxadiazol-3-yl)azetidine-1-carboxylate;
methyl 3-(5-(3-(6-cyclopropylimidazo[1,2-a]pyridine-3-carboxamido)-5-fluoro-4-methylphenyl)-1,2,4-oxadiazol-3-yl)azetidine-1-carboxylate;
methyl 3-(5-(3-fluoro-4-methyl-5-(6-morpholinoimidazo[1,2-a]pyridine-3-carboxamido)phenyl)-1,2,4-oxadiazol-3-yl)azetidine-1-carboxylate;
methyl 3-(5-(3-fluoro-4-methyl-5-(6-(piperazin-1-yl)imidazo[1,2-a]pyridine-3-carboxamido)phenyl)-1,2,4-oxadiazol-3-yl)azetidine-1-carboxylate;
methyl 3-(5-(3-fluoro-4-methyl-5-(6-(4-methylpiperazin-1-yl)imidazo[1,2-a]pyridine-3-carboxamido)phenyl)-1,2,4-oxadiazol-3-yl)azetidine-1-carboxylate;
methyl 3-(5-(3-fluoro-5-(7-fluoroimidazo[1,2-a]pyridine-3-carboxamido)-4-methylphenyl)-1,2,4-oxadiazol-3-yl)azetidine-1-carboxylate;
methyl 3-(5-(3-fluoro-4-methyl-5-(7-methylimidazo[1,2-a]pyridine-3-carboxamido)phenyl)-1,2,4-oxadiazol-3-yl)azetidine-1-carboxylate;
methyl 3-(5-(3-(7-chloroimidazo[1,2-a]pyridine-3-carboxamido)-5-fluoro-4-methylphenyl)-1,2,4-oxadiazol-3-yl)azetidine-1-carboxylate;
methyl 3-(5-(3-(7-cyanoimidazo[1,2-a]pyridine-3-carboxamido)-5-fluoro-4-methylphenyl)-1,2,4-oxadiazol-3-yl)azetidine-1-carboxylate;
methyl 3-(5-(3-(7-carbamoylimidazo[1,2-a]pyridine-3-carboxamido)-5-fluoro-4-methylphenyl)-1,2,4-oxadiazol-3-yl)azetidine-1-carboxylate;
methyl 3-(5-(3-fluoro-4-methyl-5-(7-(trifluoromethyl)imidazo[1,2-a]pyridine-3-carboxamido)phenyl)-1,2,4-oxadiazol-3-yl)azetidine-1-carboxylate;
methyl 3-(5-(3-fluoro-4-methyl-5-(7-morpholinoimidazo[1,2-a]pyridine-3-carboxamido)phenyl)-1,2,4-oxadiazol-3-yl)azetidine-1-carboxylate;
methyl 3-(5-(3-fluoro-4-methyl-5-(7-(piperazin-1-yl)imidazo[1,2-a]pyridine-3-carboxamido)phenyl)-1,2,4-oxadiazol-3-yl)azetidine-1-carboxylate;
methyl 3-(5-(3-fluoro-4-methyl-5-(7-(4-methylpiperazin-1-yl)imidazo[1,2-a]pyridine-3-carboxamido)phenyl)-1,2,4-oxadiazol-3-yl)azetidine-1-carboxylate;
N-(3-fluoro-2-methyl-5-(3-morpholino-1,2,4-oxadiazol-5-yl)phenyl)imidazo[1,2-a]pyridine-3-carboxamide;
N-(3-fluoro-2-methyl-5-(3-(piperazin-1-yl)-1,2,4-oxadiazol-5-yl)phenyl)imidazo[1,2-a]pyridine-3-carboxamide;
methyl 4-(5-(3-fluoro-5-(imidazo[1,2-a]pyridine-3-carboxamido)-4-methylphenyl)-1,2,4-oxadiazol-3-yl)piperazine-1-carboxylate;
methyl 4-(5-(3-fluoro-5-(imidazo[1,2-a]pyridine-3-carboxamido)-4-methylphenyl)-1,2,4-oxadiazol-3-yl)-2-methylpiperazine-1-carboxylate;
methyl 4-(5-(3-fluoro-5-(imidazo[1,2-a]pyridine-3-carboxamido)-4-methylphenyl)-1,2,4-oxadiazol-3-yl)-2-isopropylpiperazine-1-carboxylate;
methyl 4-(5-(3-fluoro-5-(imidazo[1,2-a]pyridine-3-carboxamido)-4-methylphenyl)-1,2,4-oxadiazol-3-yl)-3-methylpiperazine-1-carboxylate;
methyl 4-(5-(3-fluoro-5-(imidazo[1,2-a]pyridine-3-carboxamido)-4-methylphenyl)-1,2,4-oxadiazol-3-yl)-3-isopropylpiperazine-1-carboxylate;
methyl 3-(5-(3-fluoro-5-(imidazo[1,2-a]pyridine-3-carboxamido)-4-methylphenyl)-1,3,4-oxadiazol-2-yl)azetidine-1-carboxylate;
methyl 3-(4-(3-fluoro-5-(imidazo[1,2-a]pyridine-3-carboxamido)-4-methylphenyl)oxazol-2-yl)azetidine-1-carboxylate;
methyl 3-(5-(3-fluoro-5-(imidazo[1,2-a]pyridine-3-carboxamido)-4-methylphenyl)oxazol-2-yl)azetidine-1-carboxylate;
methyl 3-(2-(3-fluoro-5-(imidazo[1,2-a]pyridine-3-carboxamido)-4-methylphenyl)oxazol-4-yl)azetidine-1-carboxylate;
methyl 3-(5-(3-fluoro-5-(imidazo[1,2-a]pyridine-3-carboxamido)-4-methylphenyl)-4H-1,2,4-triazol-3-yl)azetidine-1-carboxylate;
methyl (R)-3-(3-(3-fluoro-5-(imidazo[1,2-a]pyridine-3-carboxamido)-4-methylphenyl)-1,2,4-oxadiazol-5-yl)piperidine-1-carboxylate;
N-(3-fluoro-5-(5-((1S,2R)-2-fluorocyclopropyl)-1,2,4-oxadiazol-3-yl)-2-methylphenyl)-7-morpholinoimidazo[1,2-a]pyridine-3-carboxamide;
methyl 3-(2-(3-fluoro-5-(imidazo[1,2-a]pyridine-3-carboxamido)-4-methylphenyl)oxazol-5-yl)azetidine-1-carboxylate;
methyl 3-(3-(2,5-difluoro-3-(imidazo[1,2-a]pyridine-3-carboxamido)-4-methylphenyl)-1,2,4-oxadiazol-5-yl)azetidine-1-carboxylate;
methyl (R)-3-(3-(2,5-difluoro-3-(imidazo[1,2-a]pyridine-3-carboxamido)-4-methylphenyl)-1,2,4-oxadiazol-5-yl)pyrrolidine-1-carboxylate;
methyl (R)-3-(3-(2,5-difluoro-3-(imidazo[1,2-a]pyridine-3-carboxamido)-4-methylphenyl)-1,2,4-oxadiazol-5-yl)piperidine-1-carboxylate;
methyl (S)-3-(3-(2,5-difluoro-3-(imidazo[1,2-a]pyridine-3-carboxamido)-4-methylphenyl)-1,2,4-oxadiazol-5-yl)pyrrolidine-1-carboxylate;
methyl (S)-3-(3-(2,5-difluoro-3-(imidazo[1,2-a]pyridine-3-carboxamido)-4-methylphenyl)-1,2,4-oxadiazol-5-yl)piperidine-1-carboxylate;
N-(3-(5-(3,3-difluorocyclobutyl)-1,2,4-oxadiazol-3-yl)-2,5-difluoro-6-methylphenyl)imidazo[1,2-a]pyridine-3-carboxamide;
N-(3-(5-(3,3-difluorocyclobutyl)-1,2,4-oxadiazol-3-yl)-2,5-difluoro-6-methylphenyl)-7-morpholinoimidazo[1,2-a]pyridine-3-carboxamide;
N-(3-(5-(3,3-difluorocyclobutyl)-1,2,4-oxadiazol-3-yl)-2,5-difluoro-6-methylphenyl)-7-(piperazin-1-yl)imidazo[1,2-a]pyridine-3-carboxamide;
N-(2,5-difluoro-3-(5-((1R,2S)-2-fluorocyclopropyl)-1,2,4-oxadiazol-3-yl)-6-methylphenyl)imidazo[1,2-a]pyridine-3-carboxamide;
N-(2,5-difluoro-3-(5-((1S,2R)-2-fluorocyclopropyl)-1,2,4-oxadiazol-3-yl)-6-methylphenyl)imidazo[1,2-a]pyridine-3-carboxamide;
N-(2,5-difluoro-3-(5-((1R,2S)-2-fluorocyclopropyl)-1,2,4-oxadiazol-3-yl)-6-methylphenyl)-7-morpholinoimidazo[1,2-a]pyridine-3-carboxamide;
N-(2,5-difluoro-3-(5-((1S,2R)-2-fluorocyclopropyl)-1,2,4-oxadiazol-3-yl)-6-methylphenyl)-7-morpholinoimidazo[1,2-a]pyridine-3-carboxamide;
N-(2,5-difluoro-3-(5-((1S,2R)-2-fluorocyclopropyl)-1,2,4-oxadiazol-3-yl)-6-methylphenyl)-7-methylimidazo[1,2-a]pyridine-3-carboxamide;
N-(2,5-difluoro-3-(5-((1S,2R)-2-fluorocyclopropyl)-1,2,4-oxadiazol-3-yl)-6-methylphenyl)-7-methoxyimidazo[1,2-a]pyridine-3-carboxamide;
methyl 3-(3-(2,5-difluoro-4-methyl-3-(7-methylimidazo[1,2-a]pyridine-3-carboxamido)phenyl)-1,2,4-oxadiazol-5-yl)azetidine-1-carboxylate;
methyl 3-(3-(2,5-difluoro-3-(7-methoxyimidazo[1,2-a]pyridine-3-carboxamido)-4-methylphenyl)-1,2,4-oxadiazol-5-yl)azetidine-1-carboxylate;
methyl 3-(3-(2,5-difluoro-4-methyl-3-(7-morpholinoimidazo[1,2-a]pyridine-3-carboxamido)phenyl)-1,2,4-oxadiazol-5-yl)azetidine-1-carboxylate;
methyl 3-(3-(2,5-difluoro-4-methyl-3-(7-(piperazin-1-yl)imidazo[1,2-a]pyridine-3-carboxamido)phenyl)-1,2,4-oxadiazol-5-yl)azetidine-1-carboxylate;
methyl 3-(3-(2,5-difluoro-4-methyl-3-(7-(4-methylpiperazin-1-yl)imidazo[1,2-a]pyridine-3-carboxamido)phenyl)-1,2,4-oxadiazol-5-yl)azetidine-1-carboxylate;
methyl 3-(3-(2,5-difluoro-4-methyl-3-(6-methylimidazo[1,2-a]pyridine-3-carboxamido)phenyl)-1,2,4-oxadiazol-5-yl)azetidine-1-carboxylate;
methyl 3-(3-(2,5-difluoro-3-(6-methoxyimidazo[1,2-a]pyridine-3-carboxamido)-4-methylphenyl)-1,2,4-oxadiazol-5-yl)azetidine-1-carboxylate;
methyl 3-(3-(2,5-difluoro-4-methyl-3-(6-morpholinoimidazo[1,2-a]pyridine-3-carboxamido)phenyl)-1,2,4-oxadiazol-5-yl)azetidine-1-carboxylate;
methyl 3-(3-(2,5-difluoro-4-methyl-3-(6-(piperazin-1-yl)imidazo[1,2-a]pyridine-3-carboxamido)phenyl)-1,2,4-oxadiazol-5-yl)azetidine-1-carboxylate;
methyl 3-(3-(2,5-difluoro-4-methyl-3-(6-(4-methylpiperazin-1-yl)imidazo[1,2-a]pyridine-3-carboxamido)phenyl)-1,2,4-oxadiazol-5-yl)azetidine-1-carboxylate;
N-(3-(5-(3,3-difluorocyclobutyl)-1,2,4-oxadiazol-3-yl)-2,5-difluoro-6-methylphenyl)pyrazolo[1,5-a]pyridine-3-carboxamide;
N-(2,5-difluoro-3-(5-((1R,2S)-2-fluorocyclopropyl)-1,2,4-oxadiazol-3-yl)-6-methylphenyl)pyrazolo[1,5-a]pyridine-3-carboxamide;
methyl 3-(3-(2,5-difluoro-4-methyl-3-(pyrazolo[1,5-a]pyridine-3-carboxamido)phenyl)-1,2,4-oxadiazol-5-yl)azetidine-1-carboxylate;
N-(3-(3-(3,3-difluorocyclobutyl)-1,2,4-oxadiazol-5-yl)-2,5-difluoro-6-methylphenyl)imidazo[1,2-a]pyridine-3-carboxamide;
N-(2,5-difluoro-3-(3-((1R,2S)-2-fluorocyclopropyl)-1,2,4-oxadiazol-5-yl)-6-methylphenyl)imidazo[1,2-a]pyridine-3-carboxamide;
methyl 3-(5-(2,5-difluoro-3-(imidazo[1,2-a]pyridine-3-carboxamido)-4-methylphenyl)-1,2,4-oxadiazol-3-yl)azetidine-1-carboxylate;
methyl 3-(5-(2,5-difluoro-4-methyl-3-(7-methylimidazo[1,2-a]pyridine-3-carboxamido)phenyl)-1,2,4-oxadiazol-3-yl)azetidine-1-carboxylate;
methyl 3-(5-(2,5-difluoro-3-(7-methoxyimidazo[1,2-a]pyridine-3-carboxamido)-4-methylphenyl)-1,2,4-oxadiazol-3-yl)azetidine-1-carboxylate;
methyl 3-(5-(2,5-difluoro-4-methyl-3-(7-morpholinoimidazo[1,2-a]pyridine-3-carboxamido)phenyl)-1,2,4-oxadiazol-3-yl)azetidine-1-carboxylate; and
methyl 3-(5-(2,5-difluoro-4-methyl-3-(7-(piperazin-1-yl)imidazo[1,2-a]pyridine-3-carboxamido)phenyl)-1,2,4-oxadiazol-3-yl)azetidine-1-carboxylate; or a pharmaceutically acceptable salt or solvate thereof.

One embodiment provides a method of preparing a pharmaceutical composition comprising mixing a compound of Formula (I), or a pharmaceutically acceptable salt or solvate thereof, and a pharmaceutically acceptable carrier.

In certain embodiments, the c-KIT kinase inhibitory compound as described by Formula (I), or a pharmaceutically acceptable salt or solvate thereof, is substantially pure, in that it contains less than about 5%, or less than about 2%, or less than about 1%, or less than about 0.5%, or less than about 0.1%, of other organic small molecules, such as unreacted intermediates or synthesis by-products that are created, for example, in one or more of the steps of a synthesis method.

Suitable oral dosage forms include, for example, tablets, pills, sachets, or capsules of hard or soft gelatin, methylcellulose or of another suitable material easily dissolved in the digestive tract. In some embodiments, suitable nontoxic solid carriers are used which include, for example, pharmaceutical grades of mannitol, lactose, starch, magnesium stearate, sodium saccharin, talcum, cellulose, glucose, sucrose, magnesium carbonate, and the like. (See, e.g., Remington: The Science and Practice of Pharmacy (Gennaro, 21^{st} Ed. Mack Pub. Co., Easton, PA (2005)).

In some embodiments, the c-KIT kinase inhibitory compound as described by Formula (I), or a pharmaceutically acceptable salt or solvate thereof, is formulated for administration by injection. In some instances, the injection formulation is an aqueous formulation. In some instances, the injection formulation is a non-aqueous formulation. In some instances, the injection formulation is an oil-based formulation, such as sesame oil, or the like.

The dose of the composition comprising at least one c-KIT kinase inhibitory compound as described herein differs depending upon the subject or patient's (e.g., human) condition. In some embodiments, such factors include general health status, age, and other factors.

Pharmaceutical compositions are administered in a manner appropriate to the disease to be treated (or prevented). An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the composition(s) in an amount sufficient to provide therapeutic and/or prophylactic benefit (e.g., an improved clinical outcome, such as more frequent complete or partial remissions, or longer disease-free and/or overall survival, or a lessening of symptom severity. Optimal doses are generally determined using experimental models and/or clinical trials. The optimal dose depends upon the body mass, weight, or blood volume of the patient.

Oral doses typically range from about 1.0 mg to about 1000 mg, one to four times, or more, per day.

### Methods of Treatment

One embodiment provides a compound of Formula (I), or a pharmaceutically acceptable salt or solvate thereof, for use in a method of treatment of the human or animal body.

One embodiment provides a compound of Formula (I), or a pharmaceutically acceptable salt or solvate thereof, for use in a method of treatment of cancer, inflammatory, allergic, or autoimmune disease.

One embodiment provides the use of a compound of Formula (I), or a pharmaceutically acceptable salt or solvate thereof, in the manufacture of a medicament for the treatment of cancer, inflammatory, allergic, or autoimmune disease.

One embodiment provides a compound of Formula (I), or a pharmaceutically acceptable salt or solvate thereof, for use in a method of treatment of c-KIT-mediated disease.

One embodiment provides the use of a compound of Formula (I), or a pharmaceutically acceptable salt or solvate thereof, in the manufacture of a medicament for the treatment of c-KIT-mediated disease.

One embodiment provides a method of treating cancer, inflammatory, allergic, and autoimmune disease in a patient in need thereof, comprising administering to the patient a compound of Formula (I), or a pharmaceutically acceptable salt or solvate thereof.

One embodiment provides a method of treating cancer, inflammatory, allergic, or autoimmune disease in a patient in need thereof, comprising administering to the patient a pharmaceutical composition comprising a compound of Formula (I), or a pharmaceutically acceptable salt or solvate thereof, and a pharmaceutically acceptable excipient.

One embodiment provides a compound of Formula (I), or a pharmaceutically acceptable salt or solvate thereof, for use in a method of treatment of a disease selected from the group consisting of mastocytosis, asthma, chronic urticaria, rheumatoid arthritis, psoriasis, atopic dermatitis, neurofibromatosis, multiple sclerosis, and idiopathic pulmonary fibrosis.

One embodiment provides a pharmaceutical composition comprising a compound of Formula (I), or a pharmaceutically acceptable salt or solvate thereof, and a pharmaceutically acceptable excipient for use in a method of treatment of a disease selected from the group consisting of mastocytosis, asthma, chronic urticaria, rheumatoid arthritis, psoriasis, atopic dermatitis, neurofibromatosis, multiple sclerosis, and idiopathic pulmonary fibrosis.

One embodiment provides a use of a compound of Formula (I), or a pharmaceutically acceptable salt or solvate thereof, in the manufacture of a medicament for the treatment of a disease selected from the group consisting of mastocytosis, asthma, chronic urticaria, rheumatoid arthritis, psoriasis, atopic dermatitis, neurofibromatosis, multiple sclerosis, and idiopathic pulmonary fibrosis.

One embodiment provides a compound of Formula (I), or a pharmaceutically acceptable salt or solvate thereof, for use in a method of treating cancer wherein the cancer is selected from the group consisting of leukemia, acute myeloid leukemia, chronic myeloid leukemia, acute lymphoblastic leukemia, lymphoma, germ cell tumor, hematopoietic cancers, gastrointestinal stromal tumor, uveal melanoma, colorectal carcinoma, breast carcinoma, small-cell lung carcinoma, neuroblastoma, gynecological tumor, malignant mesothelioma, papillary renal cell carcinoma, papillary renal carcinoma, mastocytosis, mast cell leukemia, thymic carcinoma, colorectal carcinoma, small-cell lung carcinoma, and neuroblastoma.

One embodiment provides the use of a compound of Formula (I), or a pharmaceutically acceptable salt or solvate thereof, in the manufacture of a medicament for the treatment of cancer, wherein the cancer is selected from the group consisting of leukemia, acute myeloid leukemia, chronic myeloid leukemia, acute lymphoblastic leukemia, lymphoma, germ cell tumor, hematopoietic cancers, gastrointestinal stromal tumor, uveal melanoma, colorectal carcinoma, breast carcinoma, small-cell lung carcinoma, neuroblastoma, gynecological tumor, malignant mesothelioma, papillary renal cell carcinoma, papillary renal carcinoma, mastocytosis, mast cell leukemia, thymic carcinoma, colorectal carcinoma, small-cell lung carcinoma, and neuroblastoma.

One embodiment provides a method of treating cancer in a patient in need thereof, comprising administering to the patient a compound of Formula (I), or a pharmaceutically acceptable salt or solvate thereof. One embodiment provides a method of treating cancer in a patient in need thereof, comprising administering to the patient a pharmaceutical composition comprising a compound of Formula (I), or pharmaceutically acceptable salt or solvate thereof, and a pharmaceutically acceptable excipient. Another embodiment provides the method of treating cancer wherein the cancer is selected from the group consisting of leukemia, acute myeloid leukemia, chronic myeloid leukemia, acute lymphoblastic leukemia, lymphoma, germ cell tumor, hematopoietic cancers, gastrointestinal stromal tumor, uveal melanoma, colorectal carcinoma, breast carcinoma, small-cell lung carcinoma, neuroblastoma, gynecological tumor, malignant mesothelioma, papillary renal cell carcinoma, papillary renal carcinoma, mastocytosis, mast cell leukemia, thymic carcinoma, colorectal carcinoma, small-cell lung carcinoma, and neuroblastoma. Another embodiment provides the method of treating cancer wherein the cancer is gastrointestinal stromal tumor. Another embodiment provides the method of treating cancer wherein the cancer is acute myeloid leukemia. Another embodiment provides the method of treating cancer wherein the cancer is melanoma.

Provided herein is the method wherein the pharmaceutical composition is administered orally. Provided herein is the method wherein the pharmaceutical composition is administered by injection. Provided herein is the method wherein the pharmaceutical composition is administered by inhalation.

One embodiment provides a method of inhibiting a c-KIT kinase comprising contacting the c-KIT kinase with a compound of Formula (I). Another embodiment provides the method of inhibiting a c-KIT kinase, wherein the c-KIT kinase is contacted in an in vivo setting. Another embodiment provides the method of inhibiting a c-KIT kinase, wherein the c-KIT kinase is contacted in an in vitro setting.

In some embodiments the compound of Formula (I), or a pharmaceutically acceptable salt or solvate thereof, is selected from the group consisting of:
N-(5-(5-(3,3-difluorocyclobutyl)-1,2,4-oxadiazol-3-yl)-3-fluoro-2-methylphenyl)imidazo[1,2-a]pyridine-3-carboxamide;
N-(3-fluoro-5-(5-((1R,2S)-2-fluorocyclopropyl)-1,2,4-oxadiazol-3-yl)-2-methylphenyl)imidazo[1,2-a]pyridine-3-carboxamide;
methyl 3-(3-(3-fluoro-5-(imidazo[1,2-a]pyridine-3-carboxamido)-4-methylphenyl)-1,2,4-oxadiazol-5-yl)azetidine-1-carboxylate;
N-(5-(5-(3,3-difluorocyclobutyl)-1,2,4-oxadiazol-3-yl)-3-fluoro-2-methylphenyl)pyrazolo[1,5-a]pyridine-3-carboxamide;
N-(3-fluoro-5-(5-((1R,2S)-2-fluorocyclopropyl)-1,2,4-oxadiazol-3-yl)-2-methylphenyl)pyrazolo[1,5-a]pyridine-3-carboxamide;
methyl 3-(3-(3-fluoro-4-methyl-5-(pyrazolo[1,5-a]pyridine-3-carboxamido)phenyl)-1,2,4-oxadiazol-5-yl)azetidine-1-carboxylate;
N-(5-(5-(3,3-difluorocyclobutyl)-1,2,4-oxadiazol-3-yl)-3-fluoro-2-methylphenyl)-6-morpholinoimidazo[1,2-a]pyridine-3-carboxamide;
N-(3-fluoro-5-(5-((1R,2S)-2-fluorocyclopropyl)-1,2,4-oxadiazol-3-yl)-2-methylphenyl)-6-morpholinoimidazo[1,2-a]pyridine-3-carboxamide;
methyl 3-(3-(3-fluoro-4-methyl-5-(6-morpholinoimidazo[1,2-a]pyridine-3-carboxamido)phenyl)-1,2,4-oxadiazol-5-yl)azetidine-1-carboxylate;
N-(5-(5-(3,3-difluorocyclobutyl)-1,2,4-oxadiazol-3-yl)-3-fluoro-2-methylphenyl)-7-(piperazin-1-yl)imidazo[1,2-a]pyridine-3-carboxamide;
N-(5-(5-(3,3-difluorocyclobutyl)-1,2,4-oxadiazol-3-yl)-3-fluoro-2-methylphenyl)-7-(4-methylpiperazin-1-yl)imidazo[1,2-a]pyridine-3-carboxamide;
methyl 3-(3-(3-(6-chloroimidazo[1,2-a]pyridine-3-carboxamido)-5-fluoro-4-methylphenyl)-1,2,4-oxadiazol-5-yl)azetidine-1-carboxylate;
N-(5-(5-(3,3-difluorocyclobutyl)-1,2,4-oxadiazol-3-yl)-3-fluoro-2-methylphenyl)-7-morpholinoimidazo[1,2-a]pyridine-3-carboxamide;
N-(3-fluoro-5-(5-((1R,2S)-2-fluorocyclopropyl)-1,2,4-oxadiazol-3-yl)-2-methylphenyl)-7-morpholinoimidazo[1,2-a]pyridine-3-carboxamide;
methyl 3-(3-(3-fluoro-4-methyl-5-(7-morpholinoimidazo[1,2-a]pyridine-3-carboxamido)phenyl)-1,2,4-oxadiazol-5-yl)azetidine-1-carboxylate;
methyl 3-(3-(3-(7-chloroimidazo[1,2-a]pyridine-3-carboxamido)-5-fluoro-4-methylphenyl)-1,2,4-oxadiazol-5-yl)azetidine-1-carboxylate;
methyl 3-(3-(3-fluoro-4-methyl-5-(7-methylimidazo[1,2-a]pyridine-3-carboxamido)phenyl)-1,2,4-oxadiazol-5-yl)azetidine-1-carboxylate;
7-(4-acetylpiperazin-1-yl)-N-(5-(5-(3,3-difluorocyclobutyl)-1,2,4-oxadiazol-3-yl)-3-fluoro-2-methylphenyl)imidazo[1,2-a]pyridine-3-carboxamide;
methyl 3-(3-(3-fluoro-5-(6-fluoroimidazo[1,2-a]pyridine-3-carboxamido)-4-methylphenyl)-1,2,4-oxadiazol-5-yl)azetidine-1-carboxylate;
methyl 3-(3-(3-fluoro-4-methyl-5-(6-(piperazin-1-yl)imidazo[1,2-a]pyridine-3-carboxamido)phenyl)-1,2,4-oxadiazol-5-yl)azetidine-1-carboxylate;
methyl 3-(3-(3-fluoro-4-methyl-5-(6-methylimidazo[1,2-a]pyridine-3-carboxamido)phenyl)-1,2,4-oxadiazol-5-yl)azetidine-1-carboxylate;
N-(3-fluoro-5-(5-((1R,2S)-2-fluorocyclopropyl)-1,2,4-oxadiazol-3-yl)-2-methylphenyl)-7-(piperazin-1-yl)imidazo[1,2-a]pyridine-3-carboxamide;
ethyl 3-(3-(3-fluoro-5-(imidazo[1,2-a]pyridine-3-carboxamido)-4-methylphenyl)-1,2,4-oxadiazol-5-yl)azetidine-1-carboxylate;
methyl 3-(3-(3-fluoro-5-(7-fluoroimidazo[1,2-a]pyridine-3-carboxamido)-4-methylphenyl)-1,2,4-oxadiazol-5-yl)azetidine-1-carboxylate;
N-(5-(5-(3,3-difluorocyclobutyl)-1,2,4-oxadiazol-3-yl)-3-fluoro-2-methylphenyl)-6-(piperazin-1-yl)imidazo[1,2-a]pyridine-3-carboxamide;
N-(3-fluoro-5-(5-((1R,2S)-2-fluorocyclopropyl)-1,2,4-oxadiazol-3-yl)-2-methylphenyl)-6-(piperazin-1-yl)imidazo[1,2-a]pyridine-3-carboxamide;
methyl 3-(3-(3-fluoro-5-(6-methoxyimidazo[1,2-a]pyridine-3-carboxamido)-4-methylphenyl)-1,2,4-oxadiazol-5-yl)azetidine-1-carboxylate;
methyl 3-(3-(3-fluoro-4-methyl-5-(6-(4-methylpiperazin-1-yl)imidazo[1,2-a]pyridine-3-carboxamido)phenyl)-1,2,4-oxadiazol-5-yl)azetidine-1-carboxylate;
methyl 3-(3-(3-fluoro-5-(7-methoxyimidazo[1,2-a]pyridine-3-carboxamido)-4-methylphenyl)-1,2,4-oxadiazol-5-yl)azetidine-1-carboxylate;
methyl 3-(3-(3-fluoro-4-methyl-5-(7-(piperazin-1-yl)imidazo[1,2-a]pyridine-3-carboxamido)phenyl)-1,2,4-oxadiazol-5-yl)azetidine-1-carboxylate;
cyclopropyl 3-(3-(3-fluoro-5-(imidazo[1,2-a]pyridine-3-carboxamido)-4-methylphenyl)-1,2,4-oxadiazol-5-yl)azetidine-1-carboxylate;
N-(3-fluoro-2-methyl-5-(5-(1-(methylcarbamoyl)azetidin-3-yl)-1,2,4-oxadiazol-3-yl)phenyl)imidazo[1,2-a]pyridine-3-carboxamide;
N-(5-(5-(3,3-difluorocyclobutyl)-1,2,4-oxadiazol-3-yl)-3-fluoro-2-methylphenyl)-6-(4-methylpiperazin-1-yl)imidazo[1,2-a]pyridine-3-carboxamide;
N-(3-fluoro-5-(5-((1R,2S)-2-fluorocyclopropyl)-1,2,4-oxadiazol-3-yl)-2-methylphenyl)-6-(4-methylpiperazin-1-yl)imidazo[1,2-a]pyridine-3-carboxamide;
methyl 3-(3-(3-(7-cyanoimidazo[1,2-a]pyridine-3-carboxamido)-5-fluoro-4-methylphenyl)-1,2,4-oxadiazol-5-yl)azetidine-1-carboxylate;
methyl 3-(3-(3-(6-(4-acetylpiperazin-1-yl)imidazo[1,2-a]pyridine-3-carboxamido)-5-fluoro-4-methylphenyl)-1,2,4-oxadiazol-5-yl)azetidine-1-carboxylate;
methyl 3-(5-(3-fluoro-5-(imidazo[1,2-a]pyridine-3-carboxamido)-4-methylphenyl)-1,2,4-oxadiazol-3-yl)azetidine-1-carboxylate;
6-(4-acetylpiperazin-1-yl)-N-(5-(5-(3,3-difluorocyclobutyl)-1,2,4-oxadiazol-3-yl)-3-fluoro-2-methylphenyl)imidazo[1,2-a]pyridine-3-carboxamide;
6-(4-acetylpiperazin-1-yl)-N-(3-fluoro-5-(5-((1R,2S)-2-fluorocyclopropyl)-1,2,4-oxadiazol-3-yl)-2-methylphenyl)imidazo[1,2-a]pyridine-3-carboxamide;
methyl 3-(3-(3-(6-carbamoylimidazo[1,2-a]pyridine-3-carboxamido)-5-fluoro-4-methylphenyl)-1,2,4-oxadiazol-5-yl)azetidine-1-carboxylate;
2-aminoethyl 3-(3-(3-fluoro-5-(imidazo[1,2-a]pyridine-3-carboxamido)-4-methylphenyl)-1,2,4-oxadiazol-5-yl)azetidine-1-carboxylate;
2-methoxyethyl 3-(3-(3-fluoro-5-(imidazo[1,2-a]pyridine-3-carboxamido)-4-methylphenyl)-1,2,4-oxadiazol-5-yl)azetidine-1-carboxylate;
methyl 3-(3-(3-(7-carbamoylimidazo[1,2-a]pyridine-3-carboxamido)-5-fluoro-4-methylphenyl)-1,2,4-oxadiazol-5-yl)azetidine-1-carboxylate;
isopropyl 3-(3-(3-fluoro-5-(imidazo[1,2-a]pyridine-3-carboxamido)-4-methylphenyl)-1,2,4-oxadiazol-5-yl)azetidine-1-carboxylate;
N-(5-(5-(1-(ethylcarbamoyl)azetidin-3-yl)-1,2,4-oxadiazol-3-yl)-3-fluoro-2-methylphenyl)imidazo[1,2-a]pyridine-3-carboxamide;
N-(3-fluoro-5-(5-(1-(isopropylcarbamoyl)azetidin-3-yl)-1,2,4-oxadiazol-3-yl)-2-methylphenyl)imidazo[1,2-a]pyridine-3-carboxamide;
N-(3-fluoro-5-(5-(1-((2-hydroxyethyl)carbamoyl)azetidin-3-yl)-1,2,4-oxadiazol-3-yl)-2-methylphenyl)imidazo[1,2-a]pyridine-3-carboxamide;
N-(5-(5-(1-(cyclopropylcarbamoyl)azetidin-3-yl)-1,2,4-oxadiazol-3-yl)-3-fluoro-2-methylphenyl)imidazo[1,2-a]pyridine-3-carboxamide;
N-(3-fluoro-5-(5-(1-((2-methoxyethyl)carbamoyl)azetidin-3-yl)-1,2,4-oxadiazol-3-yl)-2-methylphenyl)imidazo[1,2-a]pyridine-3-carboxamide;
N-(5-(5-(1-((2-aminoethyl)carbamoyl)azetidin-3-yl)-1,2,4-oxadiazol-3-yl)-3-fluoro-2-methylphenyl)imidazo[1,2-a]pyridine-3-carboxamide;
N-(3-fluoro-2-methyl-5-(5-(1-(morpholine-4-carbonyl)azetidin-3-yl)-1,2,4-oxadiazol-3-yl)phenyl)imidazo[1,2-a]pyridine-3-carboxamide;
N-(3-fluoro-2-methyl-5-(5-(1-(piperazine-1-carbonyl)azetidin-3-yl)-1,2,4-oxadiazol-3-yl)phenyl)imidazo[1,2-a]pyridine-3-carboxamide;
N-(3-fluoro-2-methyl-5-(5-(1-(4-methylpiperazine-1-carbonyl)azetidin-3-yl)-1,2,4-oxadiazol-3-yl)phenyl)imidazo[1,2-a]pyridine-3-carboxamide;
methyl 3-(3-(3-(6-cyanoimidazo[1,2-a]pyridine-3-carboxamido)-5-fluoro-4-methylphenyl)-1,2,4-oxadiazol-5-yl)azetidine-1-carboxylate;
methyl 3-(3-(3-fluoro-4-methyl-5-(6-(trifluoromethyl)imidazo[1,2-a]pyridine-3-carboxamido)phenyl)-1,2,4-oxadiazol-5-yl)azetidine-1-carboxylate;
methyl 3-(3-(3-fluoro-5-(6-isopropylimidazo[1,2-a]pyridine-3-carboxamido)-4-methylphenyl)-1,2,4-oxadiazol-5-yl)azetidine-1-carboxylate;
methyl 3-(3-(3-(6-cyclopropylimidazo[1,2-a]pyridine-3-carboxamido)-5-fluoro-4-methylphenyl)-1,2,4-oxadiazol-5-yl)azetidine-1-carboxylate;
methyl 3-(3-(3-fluoro-5-(7-isopropylimidazo[1,2-a]pyridine-3-carboxamido)-4-methylphenyl)-1,2,4-oxadiazol-5-yl)azetidine-1-carboxylate;
methyl 3-(3-(3-(7-cyclopropylimidazo[1,2-a]pyridine-3-carboxamido)-5-fluoro-4-methylphenyl)-1,2,4-oxadiazol-5-yl)azetidine-1-carboxylate;
methyl 3-(3-(3-fluoro-4-methyl-5-(7-(4-methylpiperazin-1-yl)imidazo[1,2-a]pyridine-3-carboxamido)phenyl)-1,2,4-oxadiazol-5-yl)azetidine-1-carboxylate;
methyl 4-(3-(3-fluoro-5-(imidazo[1,2-a]pyridine-3-carboxamido)-4-methylphenyl)-1,2,4-oxadiazol-5-yl)piperazine-1-carboxylate;
methyl 4-(3-(3-fluoro-5-(imidazo[1,2-a]pyridine-3-carboxamido)-4-methylphenyl)-1,2,4-oxadiazol-5-yl)-2-methylpiperazine-1-carboxylate;
methyl 4-(3-(3-fluoro-5-(imidazo[1,2-a]pyridine-3-carboxamido)-4-methylphenyl)-1,2,4-oxadiazol-5-yl)-2-isopropylpiperazine-1-carboxylate;
methyl 4-(3-(3-fluoro-5-(imidazo[1,2-a]pyridine-3-carboxamido)-4-methylphenyl)-1,2,4-oxadiazol-5-yl)-3-methylpiperazine-1-carboxylate;
methyl 4-(3-(3-fluoro-5-(imidazo[1,2-a]pyridine-3-carboxamido)-4-methylphenyl)-1,2,4-oxadiazol-5-yl)-3-isopropylpiperazine-1-carboxylate;
N-(5-(5-(4-acetylpiperazin-1-yl)-1,2,4-oxadiazol-3-yl)-3-fluoro-2-methylphenyl)imidazo[1,2-a]pyridine-3-carboxamide;
N-(5-(5-(4-carbamoylpiperazin-1-yl)-1,2,4-oxadiazol-3-yl)-3-fluoro-2-methylphenyl)imidazo[1,2-a]pyridine-3-carboxamide;
N-(3-fluoro-2-methyl-5-(5-(4-methylpiperazin-1-yl)-1,2,4-oxadiazol-3-yl)phenyl)imidazo[1,2-a]pyridine-3-carboxamide;
N-(3-fluoro-2-methyl-5-(5-(piperazin-1-yl)-1,2,4-oxadiazol-3-yl)phenyl)imidazo[1,2-a]pyridine-3-carboxamide;
N-(3-fluoro-2-methyl-5-(5-morpholino-1,2,4-oxadiazol-3-yl)phenyl)imidazo[1,2-a]pyridine-3-carboxamide;
6-fluoro-N-(3-fluoro-5-(5-((1R,2S)-2-fluorocyclopropyl)-1,2,4-oxadiazol-3-yl)-2-methylphenyl)imidazo[1,2-a]pyridine-3-carboxamide;
6-chloro-N-(3-fluoro-5-(5-((1R,2S)-2-fluorocyclopropyl)-1,2,4-oxadiazol-3-yl)-2-methylphenyl)imidazo[1,2-a]pyridine-3-carboxamide;
6-cyano-N-(3-fluoro-5-(5-((1R,2S)-2-fluorocyclopropyl)-1,2,4-oxadiazol-3-yl)-2-methylphenyl)imidazo[1,2-a]pyridine-3-carboxamide;
N3-(3-fluoro-5-(5-((1R,2S)-2-fluorocyclopropyl)-1,2,4-oxadiazol-3-yl)-2-methylphenyl)imidazo[1,2-a]pyridine-3,6-dicarboxamide;
N-(3-fluoro-5-(5-((1R,2S)-2-fluorocyclopropyl)-1,2,4-oxadiazol-3-yl)-2-methylphenyl)-6-(trifluoromethyl)imidazo[1,2-a]pyridine-3-carboxamide;
N-(3-fluoro-5-(5-((1R,2S)-2-fluorocyclopropyl)-1,2,4-oxadiazol-3-yl)-2-methylphenyl)-6-methylimidazo[1,2-a]pyridine-3-carboxamide;
N-(3-fluoro-5-(5-((1R,2S)-2-fluorocyclopropyl)-1,2,4-oxadiazol-3-yl)-2-methylphenyl)-6-isopropylimidazo[1,2-a]pyridine-3-carboxamide;
6-cyclopropyl-N-(3-fluoro-5-(5-((1R,2S)-2-fluorocyclopropyl)-1,2,4-oxadiazol-3-yl)-2-methylphenyl)imidazo[1,2-a]pyridine-3-carboxamide;
7-fluoro-N-(3-fluoro-5-(5-((1R,2S)-2-fluorocyclopropyl)-1,2,4-oxadiazol-3-yl)-2-methylphenyl)imidazo[1,2-a]pyridine-3-carboxamide;
7-chloro-N-(3-fluoro-5-(5-((1R,2S)-2-fluorocyclopropyl)-1,2,4-oxadiazol-3-yl)-2-methylphenyl)imidazo[1,2-a]pyridine-3-carboxamide;
N-(3-fluoro-5-(5-((1R,2S)-2-fluorocyclopropyl)-1,2,4-oxadiazol-3-yl)-2-methylphenyl)-7-(trifluoromethyl)imidazo[1,2-a]pyridine-3-carboxamide;
7-cyano-N-(3-fluoro-5-(5-((1R,2S)-2-fluorocyclopropyl)-1,2,4-oxadiazol-3-yl)-2-methylphenyl)imidazo[1,2-a]pyridine-3-carboxamide;
N3-(3-fluoro-5-(5-((1R,2S)-2-fluorocyclopropyl)-1,2,4-oxadiazol-3-yl)-2-methylphenyl)imidazo[1,2-a]pyridine-3,7-dicarboxamide;
N-(3-fluoro-5-(5-((1R,2S)-2-fluorocyclopropyl)-1,2,4-oxadiazol-3-yl)-2-methylphenyl)-7-methylimidazo[1,2-a]pyridine-3-carboxamide;
N-(3-fluoro-5-(5-((1R,2S)-2-fluorocyclopropyl)-1,2,4-oxadiazol-3-yl)-2-methylphenyl)-7-isopropylimidazo[1,2-a]pyridine-3-carboxamide;
7-cyclopropyl-N-(3-fluoro-5-(5-((1R,2S)-2-fluorocyclopropyl)-1,2,4-oxadiazol-3-yl)-2-methylphenyl)imidazo[1,2-a]pyridine-3-carboxamide;
N-(3-fluoro-5-(5-((1R,2S)-2-fluorocyclopropyl)-1,2,4-oxadiazol-3-yl)-2-methylphenyl)-7-(4-methylpiperazin-1-yl)imidazo[1,2-a]pyridine-3-carboxamide;
methyl 3-(3-(3-fluoro-4-methyl-5-(7-(trifluoromethyl)imidazo[1,2-a]pyridine-3-carboxamido)phenyl)-1,2,4-oxadiazol-5-yl)azetidine-1-carboxylate;
N-(3-fluoro-5-(5-((1S,2R)-2-fluorocyclopropyl)-1,2,4-oxadiazol-3-yl)-2-methylphenyl)imidazo[1,2-a]pyridine-3-carboxamide;
N-(3-fluoro-5-(5-((1R,2R)-2-fluorocyclopropyl)-1,2,4-oxadiazol-3-yl)-2-methylphenyl)imidazo[1,2-a]pyridine-3-carboxamide;
N-(3-fluoro-5-(5-((1S,2S)-2-fluorocyclopropyl)-1,2,4-oxadiazol-3-yl)-2-methylphenyl)imidazo[1,2-a]pyridine-3-carboxamide;
methyl 3-(3-(3-fluoro-5-(6-fluoropyrazolo[1,5-a]pyridine-3-carboxamido)-4-methylphenyl)-1,2,4-oxadiazol-5-yl)azetidine-1-carboxylate;
methyl 3-(3-(3-(6-chloropyrazolo[1,5-a]pyridine-3-carboxamido)-5-fluoro-4-methylphenyl)-1,2,4-oxadiazol-5-yl)azetidine-1-carboxylate;
methyl 3-(3-(3-(6-cyanopyrazolo[1,5-a]pyridine-3-carboxamido)-5-fluoro-4-methylphenyl)-1,2,4-oxadiazol-5-yl)azetidine-1-carboxylate;
methyl 3-(3-(3-(6-carbamoylpyrazolo[1,5-a]pyridine-3-carboxamido)-5-fluoro-4-methylphenyl)-1,2,4-oxadiazol-5-yl)azetidine-1-carboxylate;
methyl 3-(3-(3-fluoro-4-methyl-5-(6-(trifluoromethyl)pyrazolo[1,5-a]pyridine-3-carboxamido)phenyl)-1,2,4-oxadiazol-5-yl)azetidine-1-carboxylate;
methyl 3-(3-(3-fluoro-5-(6-methoxypyrazolo[1,5-a]pyridine-3-carboxamido)-4-methylphenyl)-1,2,4-oxadiazol-5-yl)azetidine-1-carboxylate;
methyl 3-(3-(3-fluoro-5-(6-isopropylpyrazolo[1,5-a]pyridine-3-carboxamido)-4-methylphenyl)-1,2,4-oxadiazol-5-yl)azetidine-1-carboxylate;
methyl 3-(3-(3-(6-cyclopropylpyrazolo[1,5-a]pyridine-3-carboxamido)-5-fluoro-4-methylphenyl)-1,2,4-oxadiazol-5-yl)azetidine-1-carboxylate;
methyl 3-(3-(3-fluoro-4-methyl-5-(6-methylpyrazolo[1,5-a]pyridine-3-carboxamido)phenyl)-1,2,4-oxadiazol-5-yl)azetidine-1-carboxylate;
methyl 3-(3-(3-fluoro-4-methyl-5-(5-methylpyrazolo[1,5-a]pyridine-3-carboxamido)phenyl)-1,2,4-oxadiazol-5-yl)azetidine-1-carboxylate;
methyl 3-(3-(3-(5-cyclopropylpyrazolo[1,5-a]pyridine-3-carboxamido)-5-fluoro-4-methylphenyl)-1,2,4-oxadiazol-5-yl)azetidine-1-carboxylate;
methyl 3-(3-(3-fluoro-5-(5-isopropylpyrazolo[1,5-a]pyridine-3-carboxamido)-4-methylphenyl)-1,2,4-oxadiazol-5-yl)azetidine-1-carboxylate;
methyl 3-(3-(3-fluoro-5-(5-fluoropyrazolo[1,5-a]pyridine-3-carboxamido)-4-methylphenyl)-1,2,4-oxadiazol-5-yl)azetidine-1-carboxylate;
methyl 3-(3-(3-(5-chloropyrazolo[1,5-a]pyridine-3-carboxamido)-5-fluoro-4-methylphenyl)-1,2,4-oxadiazol-5-yl)azetidine-1-carboxylate;
methyl 3-(3-(3-(5-cyanopyrazolo[1,5-a]pyridine-3-carboxamido)-5-fluoro-4-methylphenyl)-1,2,4-oxadiazol-5-yl)azetidine-1-carboxylate;
methyl 3-(3-(3-(5-carbamoylpyrazolo[1,5-a]pyridine-3-carboxamido)-5-fluoro-4-methylphenyl)-1,2,4-oxadiazol-5-yl)azetidine-1-carboxylate;
methyl 3-(3-(3-fluoro-4-methyl-5-(5-(trifluoromethyl)pyrazolo[1,5-a]pyridine-3-carboxamido)phenyl)-1,2,4-oxadiazol-5-yl)azetidine-1-carboxylate;
methyl 3-(3-(3-fluoro-4-methyl-5-(5-morpholinopyrazolo[1,5-a]pyridine-3-carboxamido)phenyl)-1,2,4-oxadiazol-5-yl)azetidine-1-carboxylate;
methyl 3-(3-(3-fluoro-4-methyl-5-(5-(piperazin-1-yl)pyrazolo[1,5-a]pyridine-3-carboxamido)phenyl)-1,2,4-oxadiazol-5-yl)azetidine-1-carboxylate;
methyl 3-(3-(3-fluoro-4-methyl-5-(5-(4-methylpiperazin-1-yl)pyrazolo[1,5-a]pyridine-3-carboxamido)phenyl)-1,2,4-oxadiazol-5-yl)azetidine-1-carboxylate;
methyl 3-(3-(3-fluoro-4-methyl-5-(6-morpholinopyrazolo[1,5-a]pyridine-3-carboxamido)phenyl)-1,2,4-oxadiazol-5-yl)azetidine-1-carboxylate;
methyl 3-(3-(3-fluoro-4-methyl-5-(6-(piperazin-1-yl)pyrazolo[1,5-a]pyridine-3-carboxamido)phenyl)-1,2,4-oxadiazol-5-yl)azetidine-1-carboxylate;
methyl 3-(3-(3-fluoro-4-methyl-5-(6-(4-methylpiperazin-1-yl)pyrazolo[1,5-a]pyridine-3-carboxamido)phenyl)-1,2,4-oxadiazol-5-yl)azetidine-1-carboxylate;
N-(5-(3-(3,3-difluorocyclobutyl)-1,2,4-oxadiazol-5-yl)-3-fluoro-2-methylphenyl)imidazo[1,2-a]pyridine-3-carboxamide;
N-(3-fluoro-5-(3-((1R,2S)-2-fluorocyclopropyl)-1,2,4-oxadiazol-5-yl)-2-methylphenyl)imidazo[1,2-a]pyridine-3-carboxamide;
isopropyl 3-(5-(3-fluoro-5-(imidazo[1,2-a]pyridine-3-carboxamido)-4-methylphenyl)-1,2,4-oxadiazol-3-yl)azetidine-1-carboxylate;
cyclopropyl 3-(5-(3-fluoro-5-(imidazo[1,2-a]pyridine-3-carboxamido)-4-methylphenyl)-1,2,4-oxadiazol-3-yl)azetidine-1-carboxylate;
N-(3-fluoro-2-methyl-5-(3-(1-(methylcarbamoyl)azetidin-3-yl)-1,2,4-oxadiazol-5-yl)phenyl)imidazo[1,2-a]pyridine-3-carboxamide;
N-(3-fluoro-2-methyl-5-(3-(1-(morpholine-4-carbonyl)azetidin-3-yl)-1,2,4-oxadiazol-5-yl)phenyl)imidazo[1,2-a]pyridine-3-carboxamide;
N-(3-fluoro-2-methyl-5-(3-(1-(piperazine-1-carbonyl)azetidin-3-yl)-1,2,4-oxadiazol-5-yl)phenyl)imidazo[1,2-a]pyridine-3-carboxamide;
methyl 3-(5-(3-fluoro-5-(6-fluoroimidazo[1,2-a]pyridine-3-carboxamido)-4-methylphenyl)-1,2,4-oxadiazol-3-yl)azetidine-1-carboxylate;
methyl 3-(5-(3-(6-chloroimidazo[1,2-a]pyridine-3-carboxamido)-5-fluoro-4-methylphenyl)-1,2,4-oxadiazol-3-yl)azetidine-1-carboxylate;
methyl 3-(5-(3-(6-cyanoimidazo[1,2-a]pyridine-3-carboxamido)-5-fluoro-4-methylphenyl)-1,2,4-oxadiazol-3-yl)azetidine-1-carboxylate;
methyl 3-(5-(3-(6-carbamoylimidazo[1,2-a]pyridine-3-carboxamido)-5-fluoro-4-methylphenyl)-1,2,4-oxadiazol-3-yl)azetidine-1-carboxylate;
methyl 3-(5-(3-fluoro-4-methyl-5-(6-(trifluoromethyl)imidazo[1,2-a]pyridine-3-carboxamido)phenyl)-1,2,4-oxadiazol-3-yl)azetidine-1-carboxylate;
methyl 3-(5-(3-fluoro-4-methyl-5-(6-methylimidazo[1,2-a]pyridine-3-carboxamido)phenyl)-1,2,4-oxadiazol-3-yl)azetidine-1-carboxylate;
methyl 3-(5-(3-fluoro-5-(6-isopropylimidazo[1,2-a]pyridine-3-carboxamido)-4-methylphenyl)-1,2,4-oxadiazol-3-yl)azetidine-1-carboxylate;
methyl 3-(5-(3-(6-cyclopropylimidazo[1,2-a]pyridine-3-carboxamido)-5-fluoro-4-methylphenyl)-1,2,4-oxadiazol-3-yl)azetidine-1-carboxylate;
methyl 3-(5-(3-fluoro-4-methyl-5-(6-morpholinoimidazo[1,2-a]pyridine-3-carboxamido)phenyl)-1,2,4-oxadiazol-3-yl)azetidine-1-carboxylate;
methyl 3-(5-(3-fluoro-4-methyl-5-(6-(piperazin-1-yl)imidazo[1,2-a]pyridine-3-carboxamido)phenyl)-1,2,4-oxadiazol-3-yl)azetidine-1-carboxylate;
methyl 3-(5-(3-fluoro-4-methyl-5-(6-(4-methylpiperazin-1-yl)imidazo[1,2-a]pyridine-3-carboxamido)phenyl)-1,2,4-oxadiazol-3-yl)azetidine-1-carboxylate;
methyl 3-(5-(3-fluoro-5-(7-fluoroimidazo[1,2-a]pyridine-3-carboxamido)-4-methylphenyl)-1,2,4-oxadiazol-3-yl)azetidine-1-carboxylate;
methyl 3-(5-(3-fluoro-4-methyl-5-(7-methylimidazo[1,2-a]pyridine-3-carboxamido)phenyl)-1,2,4-oxadiazol-3-yl)azetidine-1-carboxylate;
methyl 3-(5-(3-(7-chloroimidazo[1,2-a]pyridine-3-carboxamido)-5-fluoro-4-methylphenyl)-1,2,4-oxadiazol-3-yl)azetidine-1-carboxylate;
methyl 3-(5-(3-(7-cyanoimidazo[1,2-a]pyridine-3-carboxamido)-5-fluoro-4-methylphenyl)-1,2,4-oxadiazol-3-yl)azetidine-1-carboxylate;
methyl 3-(5-(3-(7-carbamoylimidazo[1,2-a]pyridine-3-carboxamido)-5-fluoro-4-methylphenyl)-1,2,4-oxadiazol-3-yl)azetidine-1-carboxylate;
methyl 3-(5-(3-fluoro-4-methyl-5-(7-(trifluoromethyl)imidazo[1,2-a]pyridine-3-carboxamido)phenyl)-1,2,4-oxadiazol-3-yl)azetidine-1-carboxylate;
methyl 3-(5-(3-fluoro-4-methyl-5-(7-morpholinoimidazo[1,2-a]pyridine-3-carboxamido)phenyl)-1,2,4-oxadiazol-3-yl)azetidine-1-carboxylate;
methyl 3-(5-(3-fluoro-4-methyl-5-(7-(piperazin-1-yl)imidazo[1,2-a]pyridine-3-carboxamido)phenyl)-1,2,4-oxadiazol-3-yl)azetidine-1-carboxylate;
methyl 3-(5-(3-fluoro-4-methyl-5-(7-(4-methylpiperazin-1-yl)imidazo[1,2-a]pyridine-3-carboxamido)phenyl)-1,2,4-oxadiazol-3-yl)azetidine-1-carboxylate;
N-(3-fluoro-2-methyl-5-(3-morpholino-1,2,4-oxadiazol-5-yl)phenyl)imidazo[1,2-a]pyridine-3-carboxamide;
N-(3-fluoro-2-methyl-5-(3-(piperazin-1-yl)-1,2,4-oxadiazol-5-yl)phenyl)imidazo[1,2-a]pyridine-3-carboxamide;
methyl 4-(5-(3-fluoro-5-(imidazo[1,2-a]pyridine-3-carboxamido)-4-methylphenyl)-1,2,4-oxadiazol-3-yl)piperazine-1-carboxylate;
methyl 4-(5-(3-fluoro-5-(imidazo[1,2-a]pyridine-3-carboxamido)-4-methylphenyl)-1,2,4-oxadiazol-3-yl)-2-methylpiperazine-1-carboxylate;
methyl 4-(5-(3-fluoro-5-(imidazo[1,2-a]pyridine-3-carboxamido)-4-methylphenyl)-1,2,4-oxadiazol-3-yl)-2-isopropylpiperazine-1-carboxylate;
methyl 4-(5-(3-fluoro-5-(imidazo[1,2-a]pyridine-3-carboxamido)-4-methylphenyl)-1,2,4-oxadiazol-3-yl)-3-methylpiperazine-1-carboxylate;
methyl 4-(5-(3-fluoro-5-(imidazo[1,2-a]pyridine-3-carboxamido)-4-methylphenyl)-1,2,4-oxadiazol-3-yl)-3-isopropylpiperazine-1-carboxylate;
methyl 3-(5-(3-fluoro-5-(imidazo[1,2-a]pyridine-3-carboxamido)-4-methylphenyl)-1,3,4-oxadiazol-2-yl)azetidine-1-carboxylate;
methyl 3-(4-(3-fluoro-5-(imidazo[1,2-a]pyridine-3-carboxamido)-4-methylphenyl)oxazol-2-yl)azetidine-1-carboxylate;
methyl 3-(5-(3-fluoro-5-(imidazo[1,2-a]pyridine-3-carboxamido)-4-methylphenyl)oxazol-2-yl)azetidine-1-carboxylate;
methyl 3-(2-(3-fluoro-5-(imidazo[1,2-a]pyridine-3-carboxamido)-4-methylphenyl)oxazol-4-yl)azetidine-1-carboxylate;
methyl 3-(5-(3-fluoro-5-(imidazo[1,2-a]pyridine-3-carboxamido)-4-methylphenyl)-4H-1,2,4-triazol-3-yl)azetidine-1-carboxylate;
methyl (R)-3-(3-(3-fluoro-5-(imidazo[1,2-a]pyridine-3-carboxamido)-4-methylphenyl)-1,2,4-oxadiazol-5-yl)piperidine-1-carboxylate;
N-(3-fluoro-5-(5-((1S,2R)-2-fluorocyclopropyl)-1,2,4-oxadiazol-3-yl)-2-methylphenyl)-7-morpholinoimidazo[1,2-a]pyridine-3-carboxamide;
methyl 3-(2-(3-fluoro-5-(imidazo[1,2-a]pyridine-3-carboxamido)-4-methylphenyl)oxazol-5-yl)azetidine-1-carboxylate;
methyl 3-(3-(2,5-difluoro-3-(imidazo[1,2-a]pyridine-3-carboxamido)-4-methylphenyl)-1,2,4-oxadiazol-5-yl)azetidine-1-carboxylate;
methyl (R)-3-(3-(2,5-difluoro-3-(imidazo[1,2-a]pyridine-3-carboxamido)-4-methylphenyl)-1,2,4-oxadiazol-5-yl)pyrrolidine-1-carboxylate;
methyl (R)-3-(3-(2,5-difluoro-3-(imidazo[1,2-a]pyridine-3-carboxamido)-4-methylphenyl)-1,2,4-oxadiazol-5-yl)piperidine-1-carboxylate;
methyl (S)-3-(3-(2,5-difluoro-3-(imidazo[1,2-a]pyridine-3-carboxamido)-4-methylphenyl)-1,2,4-oxadiazol-5-yl)pyrrolidine-1-carboxylate;
methyl (S)-3-(3-(2,5-difluoro-3-(imidazo[1,2-a]pyridine-3-carboxamido)-4-methylphenyl)-1,2,4-oxadiazol-5-yl)piperidine-1-carboxylate;
N-(3-(5-(3,3-difluorocyclobutyl)-1,2,4-oxadiazol-3-yl)-2,5-difluoro-6-methylphenyl)imidazo[1,2-a]pyridine-3-carboxamide;
N-(3-(5-(3,3-difluorocyclobutyl)-1,2,4-oxadiazol-3-yl)-2,5-difluoro-6-methylphenyl)-7-morpholinoimidazo[1,2-a]pyridine-3-carboxamide;
N-(3-(5-(3,3-difluorocyclobutyl)-1,2,4-oxadiazol-3-yl)-2,5-difluoro-6-methylphenyl)-7-(piperazin-1-yl)imidazo[1,2-a]pyridine-3-carboxamide;
N-(2,5-difluoro-3-(5-((1R,2S)-2-fluorocyclopropyl)-1,2,4-oxadiazol-3-yl)-6-methylphenyl)imidazo[1,2-a]pyridine-3-carboxamide;
N-(2,5-difluoro-3-(5-((1S,2R)-2-fluorocyclopropyl)-1,2,4-oxadiazol-3-yl)-6-methylphenyl)imidazo[1,2-a]pyridine-3-carboxamide;
N-(2,5-difluoro-3-(5-((1R,2S)-2-fluorocyclopropyl)-1,2,4-oxadiazol-3-yl)-6-methylphenyl)-7-morpholinoimidazo[1,2-a]pyridine-3-carboxamide;
N-(2,5-difluoro-3-(5-((1S,2R)-2-fluorocyclopropyl)-1,2,4-oxadiazol-3-yl)-6-methylphenyl)-7-morpholinoimidazo[1,2-a]pyridine-3-carboxamide;
N-(2,5-difluoro-3-(5-((1S,2R)-2-fluorocyclopropyl)-1,2,4-oxadiazol-3-yl)-6-methylphenyl)-7-methylimidazo[1,2-a]pyridine-3-carboxamide;
N-(2,5-difluoro-3-(5-((1S,2R)-2-fluorocyclopropyl)-1,2,4-oxadiazol-3-yl)-6-methylphenyl)-7-methoxyimidazo[1,2-a]pyridine-3-carboxamide;
methyl 3-(3-(2,5-difluoro-4-methyl-3-(7-methylimidazo[1,2-a]pyridine-3-carboxamido)phenyl)-1,2,4-oxadiazol-5-yl)azetidine-1-carboxylate;
methyl 3-(3-(2,5-difluoro-3-(7-methoxyimidazo[1,2-a]pyridine-3-carboxamido)-4-methylphenyl)-1,2,4-oxadiazol-5-yl)azetidine-1-carboxylate;
methyl 3-(3-(2,5-difluoro-4-methyl-3-(7-morpholinoimidazo[1,2-a]pyridine-3-carboxamido)phenyl)-1,2,4-oxadiazol-5-yl)azetidine-1-carboxylate;
methyl 3-(3-(2,5-difluoro-4-methyl-3-(7-(piperazin-1-yl)imidazo[1,2-a]pyridine-3-carboxamido)phenyl)-1,2,4-oxadiazol-5-yl)azetidine-1-carboxylate;
methyl 3-(3-(2,5-difluoro-4-methyl-3-(7-(4-methylpiperazin-1-yl)imidazo[1,2-a]pyridine-3-carboxamido)phenyl)-1,2,4-oxadiazol-5-yl)azetidine-1-carboxylate;
methyl 3-(3-(2,5-difluoro-4-methyl-3-(6-methylimidazo[1,2-a]pyridine-3-carboxamido)phenyl)-1,2,4-oxadiazol-5-yl)azetidine-1-carboxylate;
methyl 3-(3-(2,5-difluoro-3-(6-methoxyimidazo[1,2-a]pyridine-3-carboxamido)-4-methylphenyl)-1,2,4-oxadiazol-5-yl)azetidine-1-carboxylate;
methyl 3-(3-(2,5-difluoro-4-methyl-3-(6-morpholinoimidazo[1,2-a]pyridine-3-carboxamido)phenyl)-1,2,4-oxadiazol-5-yl)azetidine-1-carboxylate;
methyl 3-(3-(2,5-difluoro-4-methyl-3-(6-(piperazin-1-yl)imidazo[1,2-a]pyridine-3-carboxamido)phenyl)-1,2,4-oxadiazol-5-yl)azetidine-1-carboxylate;
methyl 3-(3-(2,5-difluoro-4-methyl-3-(6-(4-methylpiperazin-1-yl)imidazo[1,2-a]pyridine-3-carboxamido)phenyl)-1,2,4-oxadiazol-5-yl)azetidine-1-carboxylate;
N-(3-(5-(3,3-difluorocyclobutyl)-1,2,4-oxadiazol-3-yl)-2,5-difluoro-6-methylphenyl)pyrazolo[1,5-a]pyridine-3-carboxamide;
N-(2,5-difluoro-3-(5-((1R,2S)-2-fluorocyclopropyl)-1,2,4-oxadiazol-3-yl)-6-methylphenyl)pyrazolo[1,5-a]pyridine-3-carboxamide;
methyl 3-(3-(2,5-difluoro-4-methyl-3-(pyrazolo[1,5-a]pyridine-3-carboxamido)phenyl)-1,2,4-oxadiazol-5-yl)azetidine-1-carboxylate;
N-(3-(3-(3,3-difluorocyclobutyl)-1,2,4-oxadiazol-5-yl)-2,5-difluoro-6-methylphenyl)imidazo[1,2-a]pyridine-3-carboxamide;
N-(2,5-difluoro-3-(3-((1R,2S)-2-fluorocyclopropyl)-1,2,4-oxadiazol-5-yl)-6-methylphenyl)imidazo[1,2-a]pyridine-3-carboxamide;
methyl 3-(5-(2,5-difluoro-3-(imidazo[1,2-a]pyridine-3-carboxamido)-4-methylphenyl)-1,2,4-oxadiazol-3-yl)azetidine-1-carboxylate;
methyl 3-(5-(2,5-difluoro-4-methyl-3-(7-methylimidazo[1,2-a]pyridine-3-carboxamido)phenyl)-1,2,4-oxadiazol-3-yl)azetidine-1-carboxylate;
methyl 3-(5-(2,5-difluoro-3-(7-methoxyimidazo[1,2-a]pyridine-3-carboxamido)-4-methylphenyl)-1,2,4-oxadiazol-3-yl)azetidine-1-carboxylate;
methyl 3-(5-(2,5-difluoro-4-methyl-3-(7-morpholinoimidazo[1,2-a]pyridine-3-carboxamido)phenyl)-1,2,4-oxadiazol-3-yl)azetidine-1-carboxylate; and
methyl 3-(5-(2,5-difluoro-4-methyl-3-(7-(piperazin-1-yl)imidazo[1,2-a]pyridine-3-carboxamido)phenyl)-1,2,4-oxadiazol-3-yl)azetidine-1-carboxylate.

Other embodiments and uses will be apparent to one skilled in the art in light of the present disclosures. The following examples are provided merely as illustrative of various embodiments and shall not be construed to limit the invention in any way.

### EXAMPLES

### I. Chemical Synthesis

In some embodiments, the c-KIT kinase inhibitory compounds disclosed herein are synthesized according to the following examples. As used below, and throughout the description of the invention, the following abbreviations, unless otherwise indicated, shall be understood to have the following meanings:
° C. degrees Celsius
δ_{H} chemical shift in parts per million downfield from tetramethylsilane
DCM dichloromethane (CH₂Cl₂)
DMF dimethylformamide
DMSO dimethylsulfoxide
EA ethyl acetate
ESI electrospray ionization
Et ethyl
g gram(s)
h hour(s)
HPLC high performance liquid chromatography
Hz hertz
J coupling constant (in NMR spectrometry)
LCMS liquid chromatography mass spectrometry
μ micro
m multiplet (spectral); meter(s); milli
M molar
M⁺ parent molecular ion
Me methyl
MHz megahertz
min minute(s)
mol mole(s); molecular (as in mol wt)
mL milliliter
MS mass spectrometry
nm nanometer(s)
NMR nuclear magnetic resonance
pH potential of hydrogen; a measure of the acidity or basicity of an aqueous solution
PE petroleum ether
RT room temperature
s singlet (spectral)
t triplet (spectral)
T temperature
TFA trifluoroacetic acid
THE tetrahydrofuran

### Example 1: Preparation of N-(5-(5-(3,3-difluorocyclobutyl)-1,2,4-oxadiazol-3-yl)-3-fluoro-2-methylphenyl)imidazo[1,2-a]pyridine-3-carboxamide

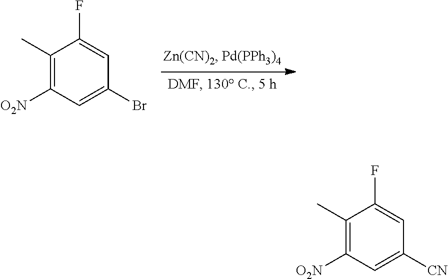

To a solution of 5-bromo-1-fluoro-2-methyl-3-nitrobenzene (50 g, 213.7 mmol, 1.0 eq) in DMF (1000 mL) were added Zn(CN)₂ (37.5 g, 320.5 mmol, 1.5 eq) and Pd(PPh₃)₄ (12.4 g, 10.6 mmol, 0.05 eq). The mixture was stirred at 130° C. for 5 h, then the reaction mixture was diluted with water (2000 mL) and extracted with EtOAc (500 mL×3). The combined organic layers were washed with brine (300 mL×2), dried with anhydrous sodium sulfate, filtered, and concentrated under vacuum. The residue was purified by column chromatography on silica gel (PE/EA=100/1, v/v) to afford 3-fluoro-4-methyl-5-nitrobenzonitrile as a yellow solid (25.1 g, 65.7%).

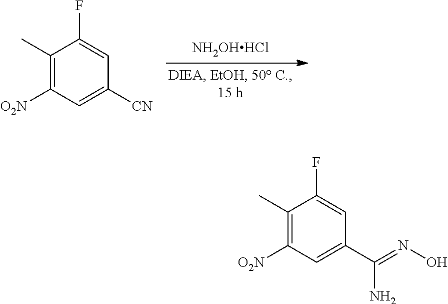

A mixture of 3-fluoro-4-methyl-5-nitrobenzonitrile (25.1 g, 139.4 mmol, 1.0 eq), NH₂OH HCl (14.5 g, 209.2 mmol, 1.5 eq) and DIEA (48.6 mL, 278.9 mmol, 2.0 eq) in EtOH (300 mL) was heated at 50° C. for 15 h. The solvent was concentrated under vacuum and the crude product was washed with water, the solid was collected by vacuum filtration and dried under vacuum to afford 3-fluoro-N′-hydroxy-4-methyl-5-nitrobenzimidamide as a white solid (23.2 g, 79.3%). LRMS (M+H⁺) m/z calculated 214.1, found 214.2. ¹H NMR (DMSO-d₆, 400 MHz) δ 10.03 (s, 1H), 8.15 (s, 1H), 7.85 (dd, 1H), 6.09 (s, 2H), 2.39 (d, 3H).

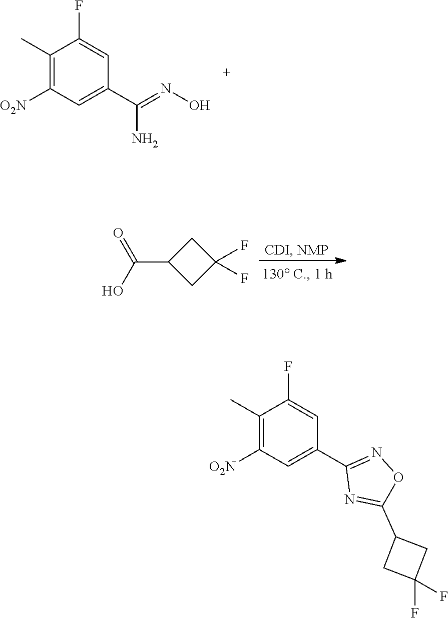 [SMILES: Cc1c(F)cc(/C(N)=N/O)cc1[N+](=O)[O-] ; O=C(O)C1CC(F)(F)C1 ; Cc1c(F)cc(-c2noc(C3CC(F)(F)C3)n2)cc1[N+](=O)[O-]]

To a solution of 3,3-difluorocyclobutane-1-carboxylic acid (4.0 g, 29.6 mmol, 1.0 eq) in anhydrous NMP (80 mL) was added CDI (7.2 g, 44.4 mmol, 1.5 eq). The reaction was stirred for 15 minutes and 3-fluoro-N′-hydroxy-4-methyl-5-nitrobenzimidamide (4.2 g, 19.7 mmol, 0.7 eq) was added and the reaction was stirred for 25 minutes. The reaction was stirred at 130° C. for 1 h. The crude product was diluted with EtOAc (200 mL). The organic layer was washed with water (200 mL) and brine (200 mL). The mixture was concentrated under vacuum and and purified by column chromatography on silica gel (PE/EA=5/1, v/v) to afford 5-(3,3-difluorocyclobutyl)-3-(3-fluoro-4-methyl-5-nitrophenyl)-1,2,4-oxadiazole as a yellow solid (5.5 g, 88.7%).

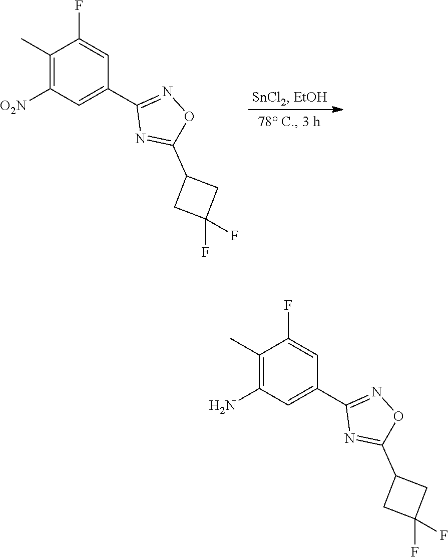 [SMILES: Cc1c(F)cc(-c2noc(C3CC(F)(F)C3)n2)cc1[N+](=O)[O-] ; Cc1c(N)cc(-c2noc(C3CC(F)(F)C3)n2)cc1F]

To a suspension of 5-(3,3-difluorocyclobutyl)-3-(3-fluoro-4-methyl-5-nitrophenyl)-1,2,4-oxadiazole (5.5 g, 17.6 mmol, 1.0 eq) in EtOH (90 mL) was added SnCl₂ (10.0 g, 52.7 mmol, 3.0 eq). The reaction mixture was heated at 78° C. for 3 h. The reaction was cooled to room temperature and the solvent was partially concentrated. The pH was adjusted to slightly basic with saturated solution of sodium bicarbonate (90 mL). The resulting white suspension was filtered and washed with water (90 mL) and EtOAc (90 mL). The aqueous layer was extracted with EtOAc (150 mL×2), and the combined organic layers were washed with brine (150 mL) and dried over anhydrous sodium sulfate. The mixture was concentrated and and purified by column chromatography on silica gel (PE/EA=5/1, v/v) to afford 5-(5-(3,3-difluorocyclobutyl)-1,2,4-oxadiazol-3-yl)-3-fluoro-2-methylaniline as a white solid (2.3 g. 46.9%). LRMS (M+H⁺) m/z calculated 284.1, found 284.2. ¹H NMR (DMSO-d₆, 400 MHz) δ 7.18 (d, 1H), 6.88 (dd, 1H), 5.582 (s, 2H), 3.84-3.85 (m, 1H), 3.03-3.20 (m, 4H), 2.01 (d, 3H).

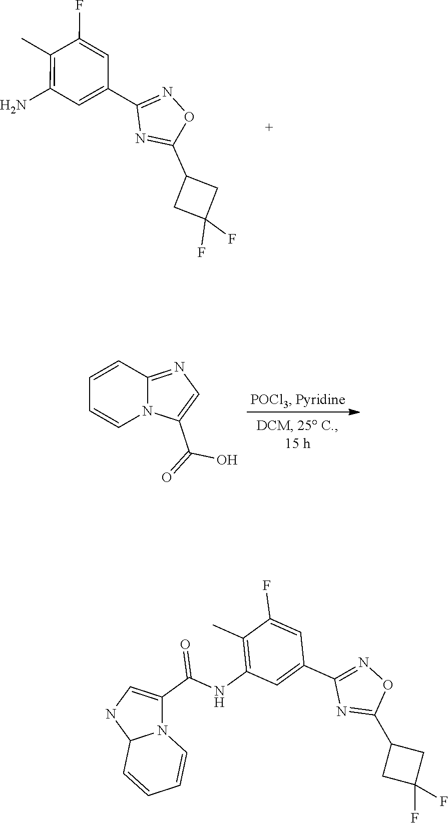 [SMILES: Cc1c(N)cc(-c2noc(C3CC(F)(F)C3)n2)cc1F ; O=C(O)c1cnc2ccccn12 ; Cc1c(F)cc(-c2noc(C3CC(F)(F)C3)n2)cc1NC(=O)C1=CNC2C=CC=CN12]

To a solution of 5-(5-(3,3-difluorocyclobutyl)-1,2,4-oxadiazol-3-yl)-3-fluoro-2-methylaniline (300 mg, 1.1 mmol, 1.0 eq), imidazo[1,2-a]pyridine-3-carboxylic acid (196.1 mg, 1.2 mmol, 1.1 eq) and pyridine (0.4 mL, 5.3 mmol, 5.0 eq) in DCM (30 mL) was added POCl₃ (0.3 mL, 3.2 mmol, 3.0 eq) on ice. The reaction mixture was stirred at 25° C. for 15 h. The reaction mixture was diluted with water (50 mL). The aqueous layer was extracted with DCM (50 mL×2), and the combined organic layers were washed with brine (50 mL) and dried over anhydrous sodium sulfate. The mixture was concentrated under vacuum and purified by reverse-phase HPLC (MeCN/H₂O=7/3, v/v) to afford N-(5-(5-(3,3-difluorocyclobutyl)-1,2,4-oxadiazol-3-yl)-3-fluoro-2-methylphenyl)imidazo[1,2-a]pyridine-3-carboxamide as a white solid (72.7 mg. 15.9%). LRMS (M+H⁺) m/z calculated 428.1, found 428.0. ¹H NMR (DMSO-d₆, 400 MHz) δ 10.22 (brs, 1H), 9.45 (d, 1H), 8.60 (s, 1H), 7.97 (s, 1H), 7.79 (d, 1H), 7.65 (d, 1H), 7.51-7.56 (m, 1H), 7.19 (t, 1H), 3.87-3.97 (m, 1H), 3.02-3.26 (m, 4H), 2.25 (s, 3H).

Example 2: Preparation of N-(3-fluoro-5-(5-((1R,2S)-2-fluorocyclopropyl)-1,2,4-oxadiazol-3-yl)-2-methylphenyl)imidazo[1,2-a]pyridine-3-carboxamide and N-(3-fluoro-5-(5-((1S,2R)-2-fluorocyclopropyl)-1,2,4-oxadiazol-3-yl)-2-methylphenyl)imidazo[1,2-a]pyridine-3-carboxamide (racemic, trans)

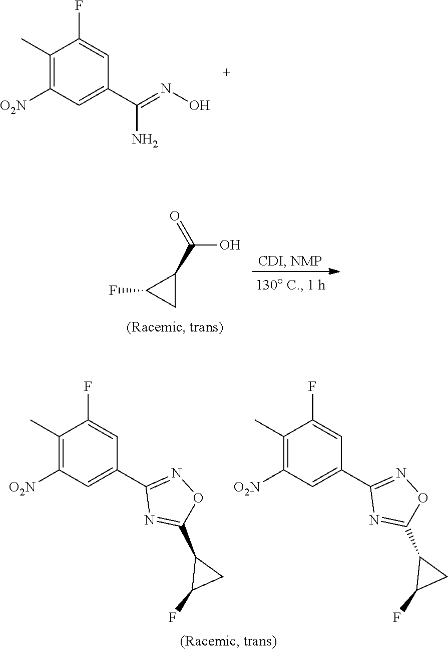 [SMILES: Cc1c(F)cc(/C(N)=N/O)cc1[N+](=O)[O-] ; O=C(O)[C@H]1C[C@@H]1F ; Cc1c(F)cc(-c2noc([C@H]3C[C@H]3F)n2)cc1[N+](=O)[O-] ; Cc1c(F)cc(-c2noc([C@@H]3C[C@H]3F)n2)cc1[N+](=O)[O-]]

To a solution of (1R,2S)-2-fluorocyclopropane-1-carboxylic acid and (1S,2R)-2-fluorocyclopropane-1-carboxylic acid (racemic, trans, 3.1 g, 29.6 mmol, 1.0 eq) in anhydrous NMP (80 mL) was added CDI (7.2 g, 44.4 mmol, 1.5 eq). The reaction was stirred for 15 minutes then 3-fluoro-N′-hydroxy-4-methyl-5-nitrobenzimidamide (4.2 g, 19.7 mmol, 0.7 eq) was added and the reaction was stirred for 25 minutes. Next, the reaction was stirred at 130° C. for 1 h. The crude product was diluted with EtOAc (200 mL). The organic was washed with water (200 mL) and brine (200 mL). The mixture was concentrated under vacuum and purified by column chromatography on silica gel (PE/EA=5/1, v/v) to afford 3-(3-fluoro-4-methyl-5-nitrophenyl)-5-((1R,2S)-2-fluorocyclopropyl)-1,2,4-oxadiazole and 3-(3-fluoro-4-methyl-5-nitrophenyl)-5-((1S,2R)-2-fluorocyclopropyl)-1,2,4-oxadiazole (racemic, trans) as a yellow oil (5.3 g, 96.3%).

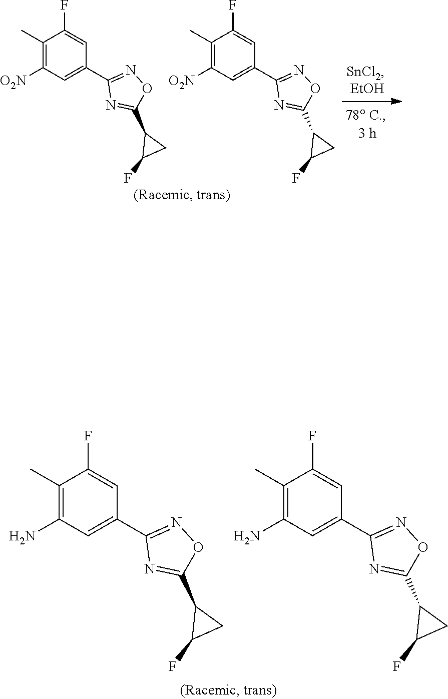 [SMILES: Cc1c(F)cc(-c2noc([C@H]3C[C@H]3F)n2)cc1[N+](=O)[O-] ; Cc1c(F)cc(-c2noc([C@@H]3C[C@H]3F)n2)cc1[N+](=O)[O-] ; Cc1c(N)cc(-c2noc([C@H]3C[C@H]3F)n2)cc1F ; Cc1c(N)cc(-c2noc([C@@H]3C[C@H]3F)n2)cc1F]

To a suspension of 3-(3-fluoro-4-methyl-5-nitrophenyl)-5-((1R,2S)-2-fluorocyclopropyl)-1,2,4-oxadiazole and 3-(3-fluoro-4-methyl-5-nitrophenyl)-5-((1S,2R)-2-fluorocyclopropyl)-1,2,4-oxadiazole (racemic, trans, 5.3 g, 18.9 mmol, 1.0 eq) in EtOH (90 mL) was added SnCl₂ (10.7 g, 56.5 mmol, 3.0 eq). The reaction mixture was heated at 78° C. for 3 h. The reaction was cooled to room temperature and the solvent was partially concentrated under vacuum. The pH was adjusted to slightly basic pH with a saturated solution of sodium bicarbonate. The resulting white suspension was filtered and washed with water and EtOAc. The aqueous layer was extracted with EtOAc (150 mL×2) and washed with water (150 mL), and the combined organic layers were washed with brine (150 mL) and dried over anhydrous sodium sulfate. The mixture was concentrated under vacuum and purified by column chromatography on silica gel (PE/EA=5/1, v/v) to afford 3-fluoro-5-(5-((1R,2S)-2-fluorocyclopropyl)-1,2,4-oxadiazol-3-yl)-2-methylaniline and 3-fluoro-5-(5-((1S,2R)-2-fluorocyclopropyl)-1,2,4-oxadiazol-3-yl)-2-methylaniline (racemic, trans) as a white solid (1.8 g. 38.3%). LRMS (M+H⁺) m/z calculated 252.1, found 252.2. ¹H NMR (DMSO-d₆, 400 MHz) δ 7.12 (d, 1H), 6.82 (dd, 1H), 5.56 (brs, 2H), 5.32-5.35 (m, 0.5H), 5.16-5.19 (m, 0.5H), 2.97-3.06 (m, 1H), 2.00 (d, 3H), 1.87-1.98 (m, 1H), 1.51-1.60 (m, 1H).

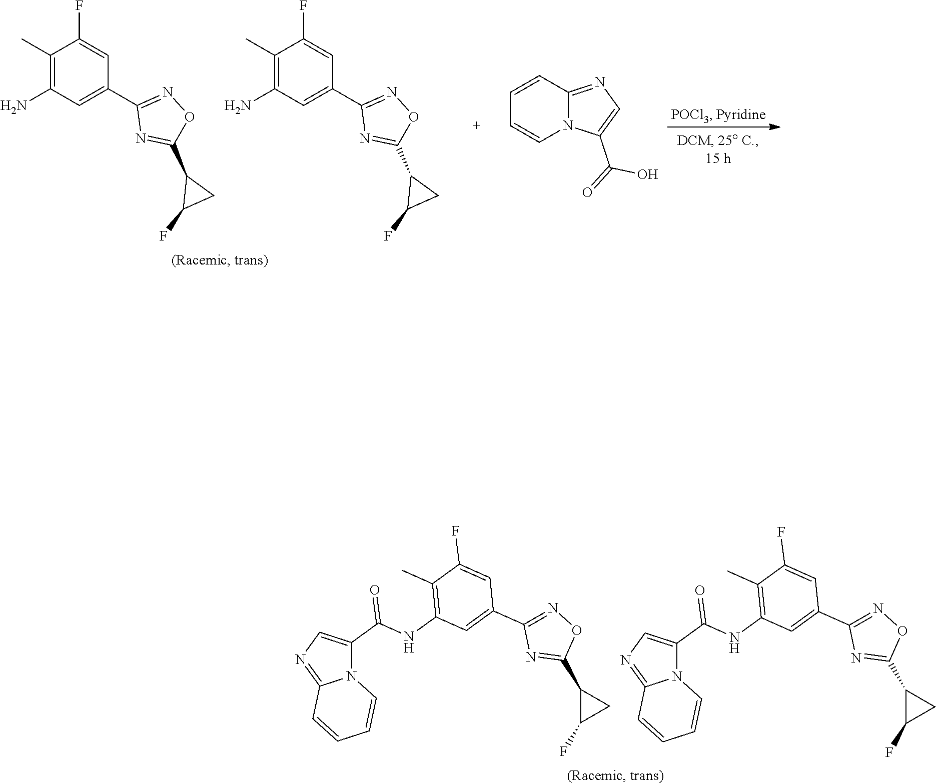

To a solution of 3-fluoro-5-(5-((1R,2S)-2-fluorocyclopropyl)-1,2,4-oxadiazol-3-yl)-2-methylaniline and 3-fluoro-5-(5-((1S,2R)-2-fluorocyclopropyl)-1,2,4-oxadiazol-3-yl)-2-methylaniline (racemic, trans, 300 mg, 1.2 mmol, 1.0 eq), imidazo[1,2-a]pyridine-3-carboxylic acid (213.8 mg, 1.3 mmol, 1.1 eq) and pyridine (0.5 mL, 6.0 mmol, 5.0 eq) in DCM (30 mL) was added POCl₃ (0.3 mL, 3.6 mmol, 3.0 eq) in an ice bath. The reaction mixture was stirred at 25° C. for 15 h. The reaction mixture was diluted with water (50 mL). The aqueous layer was extracted with DCM (50 mL×2), and the combined organic layers were washed with brine (50 mL) and dried over anhydrous sodium sulfate. The mixture was concentrated under vacuum and the resulting residue was purified by reverse-phase HPLC (MeCN/H₂O=7/3, v/v) to afford N-(3-fluoro-5-(5-((1R,2S)-2-fluorocyclopropyl)-1,2,4-oxadiazol-3-yl)-2-methylphenyl)imidazo[1,2-a]pyridine-3-carboxamide and N-(3-fluoro-5-(5-((1S,2SR)-2-fluorocyclopropyl)-1,2,4-oxadiazol-3-yl)-2-methylphenyl)imidazo[1,2-a]pyridine-3-carboxamide (racemate) as a white solid (102.4 mg, 21.2%). LRMS (M+H⁺) m/z calculated 396.1, found 396.0. ¹H NMR (DMSO-d₆, 400 MHz) δ 10.22 (brs, 1H), 9.44 (d, 1H), 8.60 (s, 1H), 7.91 (s, 1H), 7.79 (d, 1H), 7.61 (d, 1H), 7.53 (t, 1H), 7.19 (t, 1H), 5.20-5.38 (m, 1H), 3.03-3.12 (m, 1H), 2.25 (s, 3H), 1.90-2.01 (m, 1H), 1.55-1.64 (m, 1H).

### Example 3: Preparation of Methyl 3-(3-(3-fluoro-5-(imidazo[1,2-a]pyridine-3-carboxamido)-4-methylphenyl)-1,2,4-oxadiazol-5-yl)azetidine-1-carboxylate

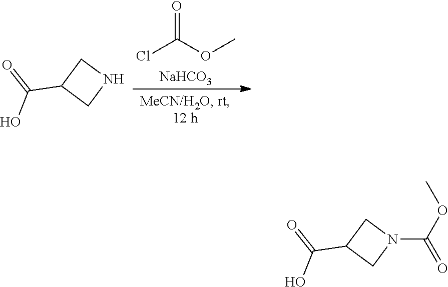

Azetidine-3-carboxylic acid (25 g, 247.5 mmol, 1.0 eq) and NaHCO₃ (31.2 g, 371.3 mmol, 1.5 eq) was dissolved in MeCN (300 mL) and water (30 mL). Methyl carbonochloridate (23.3 mmL, 297.0 mmol, 1.2 eq) was dissolved in MeCN (20 mL) and added dropwise to the reaction mixture in an ice bath. The reaction mixture was stirred for 12 h at room temperature and evaporated in vacuum. To the residue was added water (100 mL) and the aqueous phase was extracted with EtOAc (2×200 mL). The aqueous phase was acidified with conc. HCl (12 M) until pH=2 and extracted with DCM (200 mL×3). The combined organic layers were dried over anhydrous sodium sulfate and concentrated under vacuum to afford 1-(methoxycarbonyl)azetidine-3-carboxylic acid as a yellow solid (20 g, 51.3%). LRMS (M+H⁺) m/z calculated 160.1, found 160.2. ¹H-NMR (CD₃OD) δ 4.07-4.18 (m, 4H), 3.65 (s, 3H), 3.39-3.50 (m, 1H).

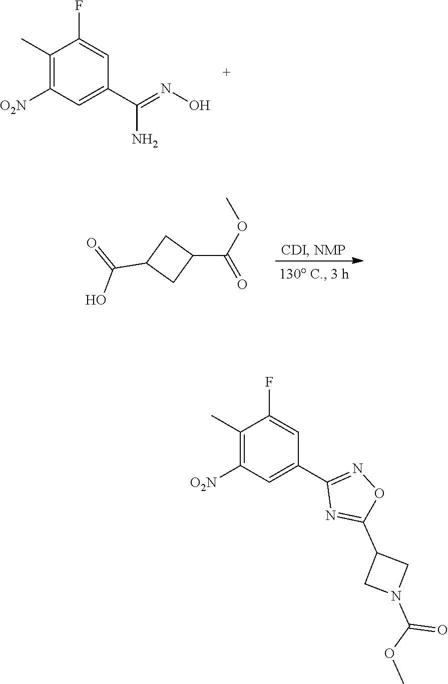 [SMILES: Cc1c(F)cc(/C(N)=N/O)cc1[N+](=O)[O-] ; COC(=O)C1CC(C(=O)O)C1 ; COC(=O)N1CC(c2nc(-c3cc(F)c(C)c([N+](=O)[O-])c3)no2)C1]

To a solution of 1-(methoxycarbonyl)azetidine-3-carboxylic acid (5.6 g, 35.2 mmol, 1.0 eq) in anhydrous NMP (80 mL) was added CDI (8.6 g, 52.8 mmol, 1.5 eq). The reaction was stirred for 15 minutes. 3-Fluoro-N′-hydroxy-4-methyl-5-nitrobenzimidamide (5.0 g, 23.5 mmol, 0.7 eq) was added and the reaction was stirred for 25 minutes. The reaction was stirred at 130° C. for 1 h. The reaction mixture was diluted with EtOAc (200 mL). The organic layer was washed with water (200 mL) and brine (200 mL). The mixture was concentrated, and the resulting residue was purified by column chromatography on silica gel (PE/EA=5/1, v/v) to afford methyl 3-(3-(3-fluoro-4-methyl-5-nitrophenyl)-1,2,4-oxadiazol-5-yl)azetidine-1-carboxylate as a yellow solid (5.5 g, 69.6%). LRMS (M+H⁺) m/z calculated 337.1, found 337.1.

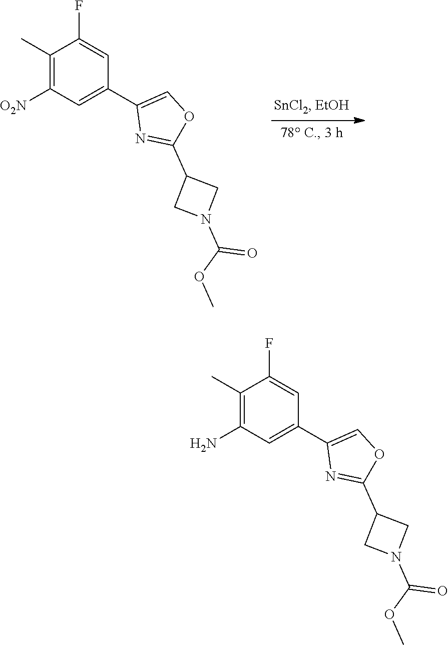 [SMILES: COC(=O)N1CC(c2nc(-c3cc(F)c(C)c([N+](=O)[O-])c3)co2)C1 ; COC(=O)N1CC(c2nc(-c3cc(N)c(C)c(F)c3)co2)C1]

To a suspension of methyl 3-(3-(3-fluoro-4-methyl-5-nitrophenyl)-1,2,4-oxadiazol-5-yl)azetidine-1-carboxylate (5.5 g, 16.4 mmol, 1.0 eq) in EtOH (60 mL) was added SnCl₂ (9.3 g, 49.1 mmol, 3.0 eq). The reaction mixture was stirred at 78° C. for 3 h. The reaction was cooled to room temperature and the solvent was partially concentrated under vacuum. The pH was adjusted to slightly basic with saturated solution of sodium bicarbonate. The resulting white suspension was filtered and washed with water (60 mL) and EtOAc (60 mL). The aqueous layer was extracted with EtOAc (150 mL×2), and the combined organic layers were washed with brine (150 mL) and dried over anhydrous sodium sulfate. The mixture was concentrated under vacuum and and purified by column chromatography on silica gel (PE/EA=5/1, v/v) to afford 5-(5-(3,3-difluorocyclobutyl)-1,2,4-oxadiazol-3-yl)-3-fluoro-2-methylaniline as a white solid (2.5 g. 50.0%). LRMS (M+H⁺) m/z calculated 307.1, found 307.1. ¹H NMR (DMSO-d₆, 400 MHz) δ 7.195 (s, 1H), 6.87-6.89 (m, 1H), 5.58 (brs, 2H), 4.10-4.36 (m, 5H), 3.60 (s, 3H), 2.01 (d, 3H).

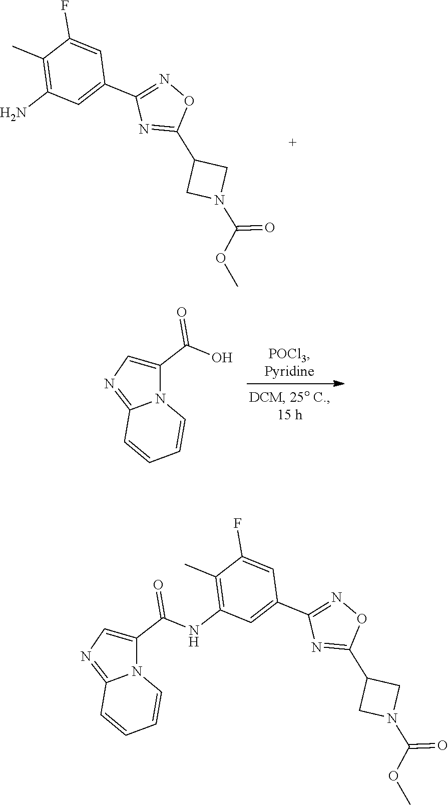 [SMILES: COC(=O)N1CC(c2nc(-c3cc(N)c(C)c(F)c3)no2)C1 ; O=C(O)c1cnc2ccccn12 ; COC(=O)N1CC(c2nc(-c3cc(F)c(C)c(NC(=O)c4cnc5ccccn45)c3)no2)C1]

To a solution of methyl 3-(3-(3-amino-5-fluoro-4-methylphenyl)-1,2,4-oxadiazol-5-yl)azetidine-1-carboxylate (1.5 g, 4.9 mmol, 1.0 eq), imidazo[1,2-a]pyridine-3-carboxylic acid (873.5 mg, 5.4 mmol, 1.1 eq) and pyridine (1.9 mL, 24.5 mmol, 5.0 eq) in DCM (80 mL) was added POCl₃ (1.4 mL, 14.7 mmol, 3.0 eq) in an ice bath. The reaction mixture was stirred at 25° C. for 15 h. The reaction mixture was diluted with water (50 mL). The aqueous layer was extracted with DCM (50 mL×2), and the combined organic layers were washed with brine (50 mL) and dried over anhydrous sodium sulfate. The mixture was concentrated and the resulting residue was purified by reverse-phase HPLC(MeCN/H₂O=7/3, v/v) to afford methyl 3-(3-(3-fluoro-5-(imidazo[1,2-a]pyridine-3-carboxamido)-4-methylphenyl)-1,2,4-oxadiazol-5-yl)azetidine-1-carboxylate as a white solid (249.1 mg. 11.3%). LRMS (M+H⁺) m/z calculated 451.1, found 451.1. ¹H NMR (DMSO-d₆, 400 MHz) δ 10.22 (s, 1H), 9.45 (d, 1H), 8.61 (s, 1H), 7.99 (s, 1H), 7.79 (d, 1H), 7.67 (d, 1H), 7.54 (t, 1H), 7.19 (m, 1H), 4.18-4.38 (m, 5H), 3.60 (s, 3H), 2.26 (s, 3H).

### Example 4: Preparation of N-(5-(5-(3,3-difluorocyclobutyl)-1,2,4-oxadiazol-3-yl)-3-fluoro-2-methylphenyl)pyrazolo[1,5-a]pyridine-3-carboxamide

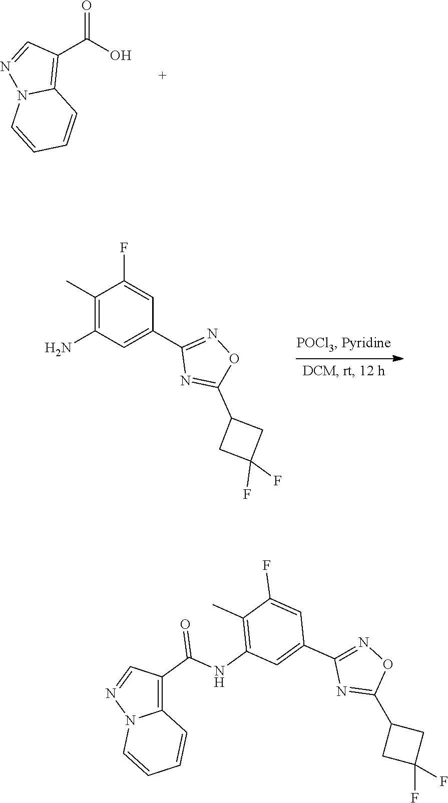 [SMILES: O=C(O)c1cnn2ccccc12 ; Cc1c(N)cc(-c2noc(C3CC(F)(F)C3)n2)cc1F ; Cc1c(F)cc(-c2noc(C3CC(F)(F)C3)n2)cc1NC(=O)c1cnn2ccccc12]

To a solution of 5-(5-(3,3-difluorocyclobutyl)-1,2,4-oxadiazol-3-yl)-3-fluoro-2-methylaniline (349.4 mg, 1.2 mmol, 1.0 eq), pyrazolo[1,5-a]pyridine-3-carboxylic acid (200 mg, 1.2 mmol, 1.0 eq) and pyridine (0.5 mL, 6.2 mmol, 5.0 eq) in DCM (50 mL) was added POCl₃ (0.3 mL, 3.7 mmol, 3.0 eq) on ice. The reaction mixture was stirred at 25° C. for 15 h. The reaction mixture was diluted with water (50 mL). The aqueous layer was extracted with DCM (80 mL×2) and washed with water (50 mL), and the combined organic layers were washed with brine (50 mL) and dried over anhydrous sodium sulfate. The mixture was concentrated under vacuum and the resulting residue was purified by Prep-HPLC to afford N-(5-(5-(3,3-difluorocyclobutyl)-1,2,4-oxadiazol-3-yl)-3-fluoro-2-methylphenyl)pyrazolo[1,5-a]pyridine-3-carboxamide (59.3 mg, 11.0%). LRMS (M+H⁺) m/z calculated 428.1, found 428.1. ¹H NMR (DMSO-d₆, 400 MHz) δ 9.95 (s, 1H), 8.86 (d, 1H), 8.79 (s, 1H), 8.23 (d, 1H), 7.99 (s, 1H), 7.63 (d, 1H), 7.55 (t, 1H), 7.14 (t, 1H), 3.87-3.91 (m, 1H), 3.04-3.32 (m, 4H), 2.25 (s, 3H).

Example 5: Preparation of N-(3-fluoro-5-(5-((1R,2S)-2-fluorocyclopropyl)-1,2,4-oxadiazol-3-yl)-2-methylphenyl)pyrazolo[1,5-a]pyridine-3-carboxamide and N-(3-fluoro-5-(5-((1S,2R)-2-fluorocyclopropyl)-1,2,4-oxadiazol-3-yl)-2-methylphenyl)pyrazolo[1,5-a]pyridine-3-carboxamide (racemic, trans)

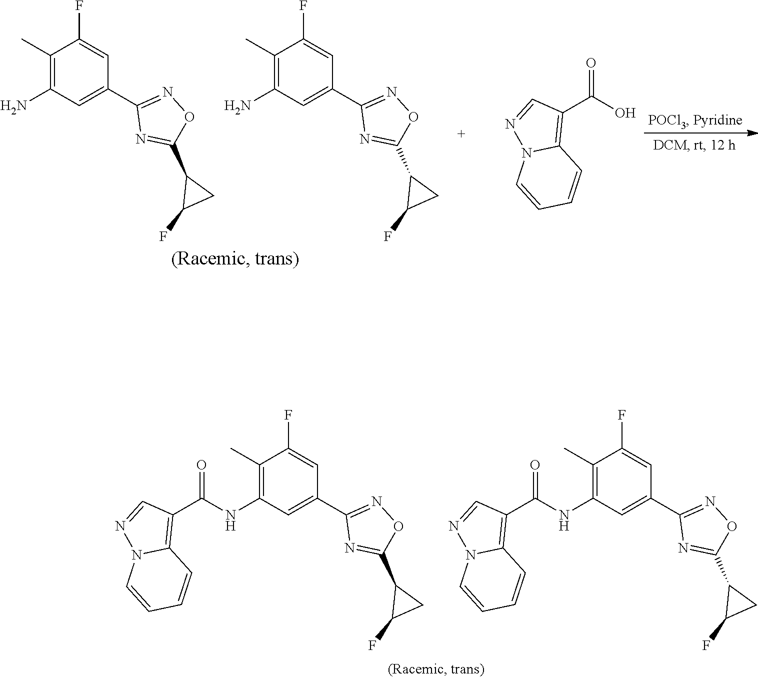

To a solution of 3-fluoro-5-(5-((1R,2S)-2-fluorocyclopropyl)-1,2,4-oxadiazol-3-yl)-2-methylaniline and 3-fluoro-5-(5-((1S,2R)-2-fluorocyclopropyl)-1,2,4-oxadiazol-3-yl)-2-methylaniline (racemic, trans, 309.9 mg, 1.2 mmol, 1.0 eq), pyrazolo[1,5-a]pyridine-3-carboxylic acid (200.0 mg, 1.2 mmol, 1.1 eq) and pyridine (0.5 mL, 6.2 mmol, 5.0 eq) in DCM (30 mL) was added POCl₃ (0.3 mL, 3.7 mmol, 3.0 eq) on ice. The reaction mixture was stirred at 25° C. for 15 h. The reaction mixture was diluted with water (50 mL). The aqueous layer was extracted with DCM (50 mL×2) and washed with water (150 mL), and the combined organic layers were washed with brine (50 mL) and dried over anhydrous sodium sulfate. The mixture was concentrated under vacuum, and the resulting residue was purified by Prep-HPLC to afford N-(3-fluoro-5-(5-((1R,2S)-2-fluorocyclopropyl)-1,2,4-oxadiazol-3-yl)-2-methylphenyl)pyrazolo[1,5-a]pyridine-3-carboxamide and N-(3-fluoro-5-(5-((1S,2R)-2-fluorocyclopropyl)-1,2,4-oxadiazol-3-yl)-2-methylphenyl)pyrazolo[1,5-a]pyridine-3-carboxamide (racemic, trans) as a white solid (91.0 mg, 18.7%). LRMS (M+H⁺) m/z calculated 396.1, found 396.0. ¹H NMR (DMSO-d₆, 400 MHz) δ 9.94 (s, 1H), 8.86 (d, 1H), 8.78 (s, 1H), 8.22 (d, 1H), 7.92 (s, 1H), 7.59 (d, 1H), 7.53 (d, 1H), 7.14 (t, 1H), 5.20-5.40 (m, 1H), 3.03-3.11 (m, 1H), 2.24 (s, 3H), 1.90-2.01 (m, 1H), 1.55-1.64 (m, 1H).

### Example 6: Preparation of Methyl 3-(3-(3-fluoro-4-methyl-5-(pyrazolo[1,5-a]pyridine-3-carboxamido)phenyl)-1,2,4-oxadiazol-5-yl)azetidine-1-carboxylate

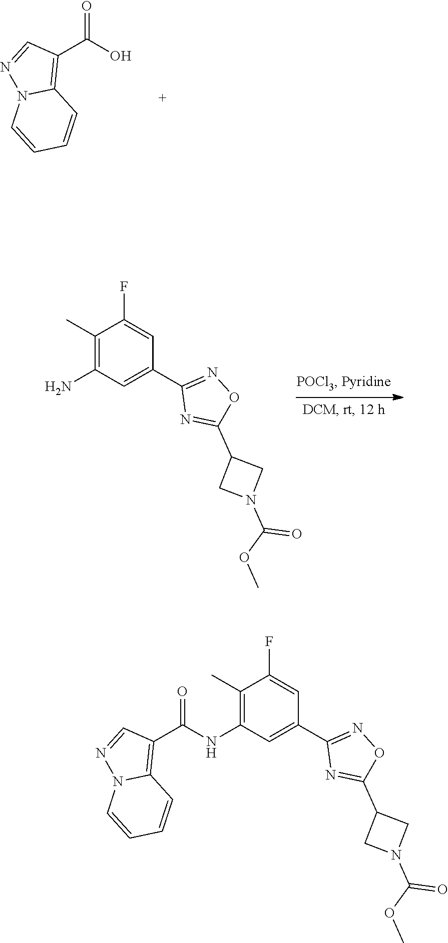 [SMILES: O=C(O)c1cnn2ccccc12 ; COC(=O)N1CC(c2nc(-c3cc(N)c(C)c(F)c3)no2)C1 ; COC(=O)N1CC(c2nc(-c3cc(F)c(C)c(NC(=O)c4cnn5ccccc45)c3)no2)C1]

To a solution of methyl 3-(3-(3-amino-5-fluoro-4-methylphenyl)-1,2,4-oxadiazol-5-yl)azetidine-1-carboxylate (188.9 mg, 0.62 mmol, 1.0 eq), pyrazolo[1,5-a]pyridine-3-carboxylic acid (100.0 mg, 0.62 mmol, 1.0 eq) and pyridine (0.3 mL, 3.1 mmol, 5.0 eq) in DCM (50 mL) was added POCl₃ (0.2 mL, 1.9 mmol, 3.0 eq) on ice. The reaction mixture was stirred at 25° C. for 15 h. The reaction mixture was diluted with water (50 mL). The aqueous layer was extracted with DCM (50 mL×2), and the combined organic layers were washed with brine (50 mL) and dried over anhydrous sodium sulfate. The mixture was concentrated under vacuum, and the resulting residue was purified by Prep-HPLC to afford methyl 3-(3-(3-fluoro-4-methyl-5-(pyrazolo[1,5-a]pyridine-3-carboxamido)phenyl)-1,2,4-oxadiazol-5-yl)azetidine-1-carboxylate as a white solid (13.3 mg, 4.7%). LRMS (M+H⁺) m/z calculated 451.1, found 451.0. ¹H NMR (DMSO-d₆, 400 MHz) δ 9.89 (s, 1H), 8.86 (d, 1H), 8.79 (s, 1H), 8.24 (d, 1H), 8.01 (s, 1H), 7.63 (d, 1H), 7.54 (t, 1H), 7.14 (t, 1H), 4.20-4.38 (m, 5H), 3.60 (s, 3H), 2.25 (d, 3H).

### Example 7: Preparation of N-(5-(5-(3,3-difluorocyclobutyl)-1,2,4-oxadiazol-3-yl)-3-fluoro-2-methylphenyl)-6-morpholinoimidazo[1,2-a]pyridine-3-carboxamide

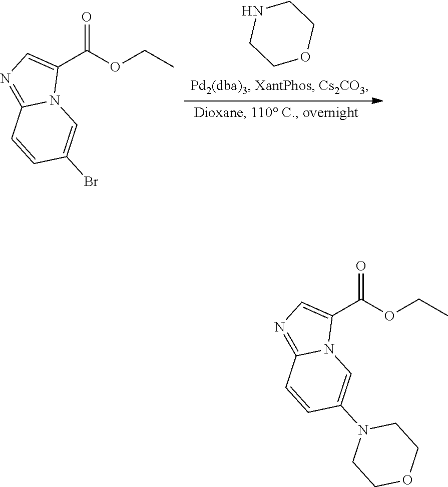 [SMILES: CCOC(=O)c1cnc2ccc(Br)cn12 ; C1COCCN1 ; CCOC(=O)c1cnc2ccc(N3CCOCC3)cn12]

A mixture of ethyl 6-bromoimidazo[1,2-a]pyridine-3-carboxylate (3 g, 11.2 mmol, 1.0 eq), morpholine (2.9 mL, 33.6 mmol, 3.0 eq), Cs₂CO₃ (7.3 g, 22.4 mmol, 2.0 eq), XPhos (648.1 mg, 1.1 mmol, 0.1 eq) and Pd₂(dba)₃ (512.7 mg, 0.6 mmol, 0.05 eq) in Dioxane (100 mL) was stirred at 110° C. overnight, and then the reaction mixture was concentrated under vacuum. The resulting residue was purified by column chromatography on silica gel (PE/EA=5/1, v/v) to afford ethyl 6-morpholinoimidazo[1,2-a]pyridine-3-carboxylate as a yellow solid (2.6 g, 83.8%). LRMS (M+H⁺) m/z calculated 276.1, found 276.0.

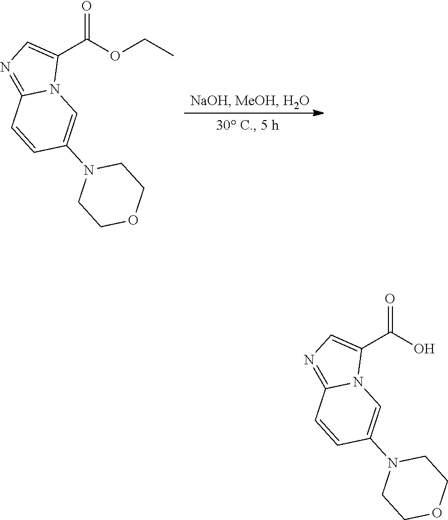

A solution of ethyl 6-morpholinoimidazo[1,2-a]pyridine-3-carboxylate (1 g, 3.6 mmol, 1.0 eq) and NaOH (218.2 mg, 5.5 mmol, 1.5 eq) in MeOH/H₂O (30 mL/5 mL) was stirred at 30° C. for 5 h, and the solvent was partially concentrated under vacuum. The pH was adjusted to pH 5 with conc. HCl, then the solid was collected by vacuum filtration and dried under vacuum to afford 6-morpholinoimidazo[1,2-a]pyridine-3-carboxylic acid as a white solid (780 mg, 86.6%). LRMS (M+H⁺) m/z calculated 248.1, found 248.0.

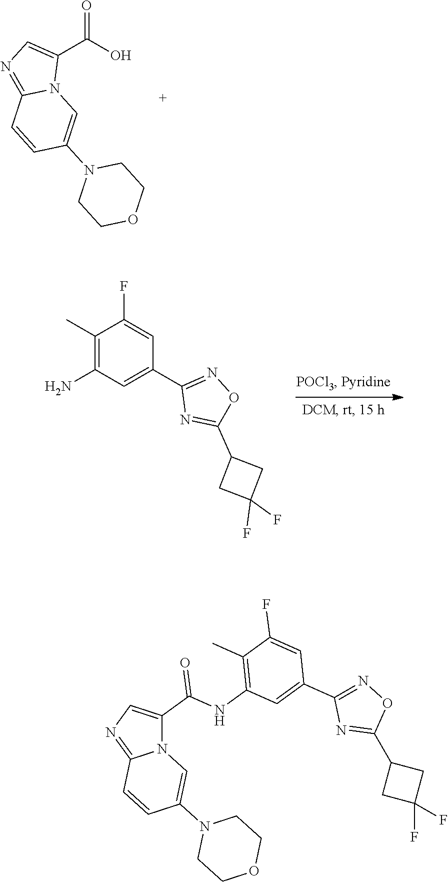 [SMILES: O=C(O)c1cnc2ccc(N3CCOCC3)cn12 ; Cc1c(N)cc(-c2noc(C3CC(F)(F)C3)n2)cc1F ; Cc1c(F)cc(-c2noc(C3CC(F)(F)C3)n2)cc1NC(=O)c1cnc2ccc(N3CCOCC3)cn12]

To a solution of 6-morpholinoimidazo[1,2-a]pyridine-3-carboxylic acid (100.0 mg, 0.40 mmol, 1.0 eq), 5-(5-(3,3-difluorocyclobutyl)-1,2,4-oxadiazol-3-yl)-3-fluoro-2-methylaniline (114.6 mg, 0.40 mmol, 1.0 eq) and pyridine (0.2 mL, 2.0 mmol, 5.0 eq) in DCM (50 mL) was added POCl₃ (0.1 mL, 1.2 mmol, 3.0 eq) on ice. The reaction mixture was stirred at 25° C. for 15 h. The reaction mixture was diluted with water (50 mL). The aqueous layer was extracted with DCM (50 mL×2), and the combined organic layers were washed with brine (50 mL) and dried over anhydrous sodium sulfate. The mixture was concentrated, and the resulting residue was purified by Prep-HPLC to afford N-(5-(5-(3,3-difluorocyclobutyl)-1,2,4-oxadiazol-3-yl)-3-fluoro-2-methylphenyl)-6-morpholinoimidazo[1,2-a]pyridine-3-carboxamide as a white solid (6.5 mg, 3.1%). LRMS (M+H⁺) m/z calculated 513.2, found 513.1. ¹H NMR (DMSO-d₆, 400 MHz) δ 8.94 (s, 1H), 8.51 (s, 1H), 7.95 (s, 1H), 7.64.7.71 (m, 2H), 7.55 (d, 1H), 3.86-3.92 (m, 1H), 3.74-3.77 (m, 4H), 3.16-3.23 (m, 2H), 3.04-3.23 (m, 6H), 2.25 (s, 3H).

Example 8: Preparation of N-(3-fluoro-5-(5-((1R,2S)-2-fluorocyclopropyl)-1,2,4-oxadiazol-3-yl)-2-methylphenyl)-6-morpholinoimidazo[1,2-a]pyridine-3-carboxamide and N-(3-fluoro-5-(5-((1S,2R)-2-fluorocyclopropyl)-1,2,4-oxadiazol-3-yl)-2-methylphenyl)-6-morpholinoimidazo[1,2-a]pyridine-3-carboxamide (racemic, trans)

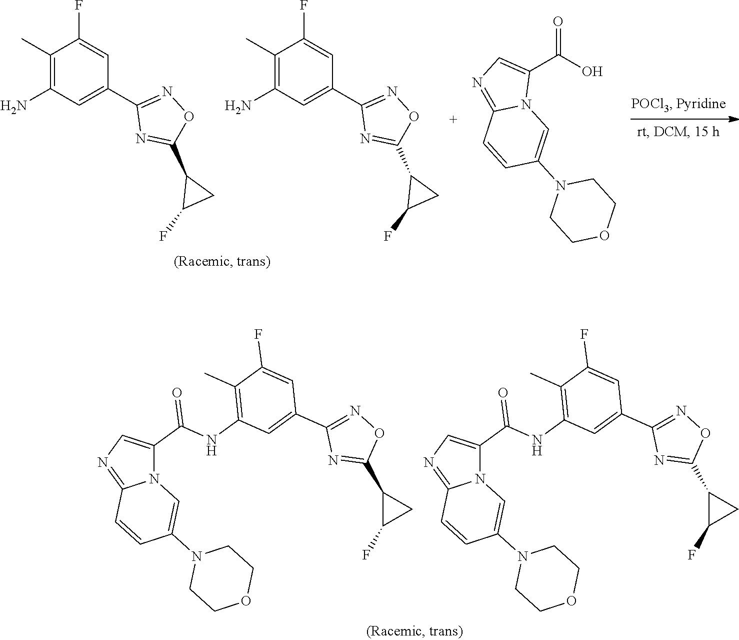

To a solution of 3-fluoro-5-(5-((1R,2S)-2-fluorocyclopropyl)-1,2,4-oxadiazol-3-yl)-2-methylaniline and 3-fluoro-5-(5-((1S,2R)-2-fluorocyclopropyl)-1,2,4-oxadiazol-3-yl)-2-methylaniline (racemic, trans, 101.6 mg, 0.40 mmol, 1.0 eq), 6-morpholinoimidazo[1,2-a]pyridine-3-carboxylic acid (100.0 mg, 0.40 mmol, 1.0 eq) and pyridine (0.2 mL, 2.0 mmol, 5.0 eq) in DCM (30 mL) was added POCl₃ (0.1 mL, 1.2 mmol, 3.0 eq) on ice. The reaction mixture was stirred at 25° C. for 15 h. The reaction mixture was diluted with water (50 mL). The aqueous layer was extracted with DCM (50 mL×2), and the combined organic layers were washed with brine (50 mL) and dried over anhydrous sodium sulfate. The mixture was concentrated under vacuum, and the resulting residue was purified by Prep-HPLC to afford N-(3-fluoro-5-(5-((1R,2S)-2-fluorocyclopropyl)-1,2,4-oxadiazol-3-yl)-2-methylphenyl)-6-morpholinoimidazo[1,2-a]pyridine-3-carboxamide and N-(3-fluoro-5-(5-((1S,2R)-2-fluorocyclopropyl)-1,2,4-oxadiazol-3-yl)-2-methylphenyl)-6-morpholinoimidazo[1,2-a]pyridine-3-carboxamide (racemic, trans) as a white solid (11.7 mg, 6.1%). LRMS (M+H⁺) m/z calculated 481.2, found 481.1. ¹H NMR (DMSO-d₆, 400 MHz) δ 8.94 (d, 1H), 8.50 (s, 1H), 7.90 (s, 1H), 7.68 (d, 1H), 7.60 (d, 1H), 7.55 (d, 1H), 5.35-5.23 (m, 1H), 3.74-3.79 (m, 4H), 3.03-3.09 (m, 5H), 2.24 (s, 3H), 1.90-2.02 (m, 1H), 1.55-1.64 (m, 1H).

### Example 9: Preparation of Methyl 3-(3-(3-fluoro-4-methyl-5-(6-morpholinoimidazo[1,2-a]pyridine-3-carboxamido)phenyl)-1,2,4-oxadiazol-5-yl)azetidine-1-carboxylate

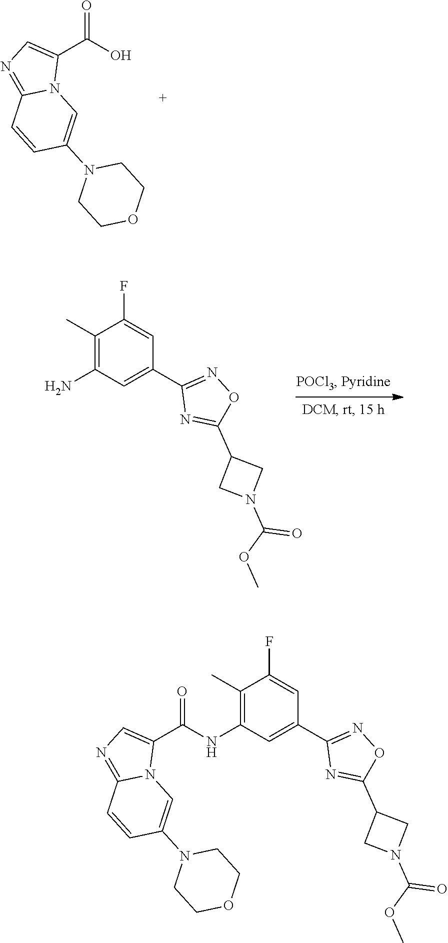 [SMILES: O=C(O)c1cnc2ccc(N3CCOCC3)cn12 ; COC(=O)N1CC(c2nc(-c3cc(N)c(C)c(F)c3)no2)C1 ; COC(=O)N1CC(c2nc(-c3cc(F)c(C)c(NC(=O)c4cnc5ccc(N6CCOCC6)cn45)c3)no2)C1]

To a solution of 6-morpholinoimidazo[1,2-a]pyridine-3-carboxylic acid (150 mg, 0.61 mmol, 1.0 eq), methyl 3-(3-(3-amino-5-fluoro-4-methylphenyl)-1,2,4-oxadiazol-5-yl)azetidine-1-carboxylate (185.8 mg, 0.61 mmol, 1.0 eq) and pyridine (0.3 mL, 3.0 mmol, 5.0 eq) in DCM (50 mL) was added POCl₃ (0.2 mL, 1.8 mmol, 3.0 eq) on ice. The reaction mixture was stirred at 25° C. for 15 h. The reaction mixture was diluted with water (50 mL). The aqueous layer was extracted with DCM (50 mL×2) and washed with water (100 mL), and the combined organic layers were washed with brine (50 mL) and dried over anhydrous sodium sulfate. The mixture was concentrated under vacuum, and the resulting residue was purified by Prep-HPLC to afford methyl 3-(3-(3-fluoro-4-methyl-5-(6-morpholinoimidazo[1,2-a]pyridine-3-carboxamido)phenyl)-1,2,4-oxadiazol-5-yl)azetidine-1-carboxylate (47.9 mg, 14.7%). LRMS (M+H⁺) m/z calculated 536.2, found 536.1. ¹H NMR (DMSO-d₆, 400 MHz) δ 10.13 (s, 1H), 8.94 (d, 1H), 8.51 (s, 1H), 7.97 (s, 1H), 7.65-7.70 (m, 2H), 7.55 (d, 1H), 4.20-4.38 (m, 5H), 3.75-3.79 (m, 4H), 3.60 (s, 3H), 3.06-3.09 (m, 4H), 2.25 (d, 3H).

### Example 10: Preparation of N-(5-(5-(3,3-difluorocyclobutyl)-1,2,4-oxadiazol-3-yl)-3-fluoro-2-methylphenyl)-7-(piperazin-1-yl)imidazo[1,2-a]pyridine-3-carboxamide

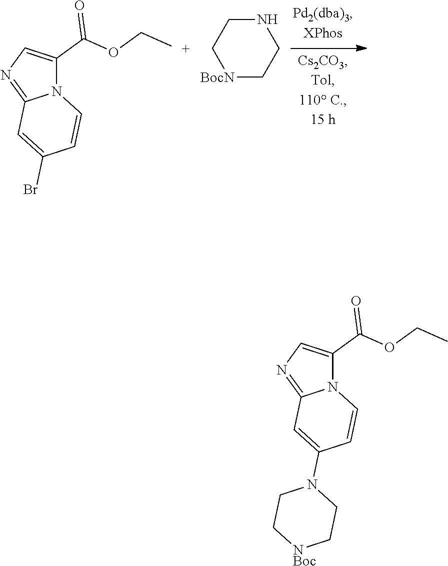

A mixture of ethyl 7-bromoimidazo[1,2-a]pyridine-3-carboxylate (1 g, 3.7 mmol, 1.0 eq), tert-butyl piperazine-1-carboxylate (2.1 g, 11.2 mmol, 3.0 eq), Cs₂CO₃ (2.4 g, 7.5 mmol, 2.0 eq), XPhos (216 mg, 0.37 mmol, 0.1 eq) and Pd₂(dba)₃ (170.9 mg, 0.2 mmol, 0.05 eq) in toluene (100 mL) was stirred at 110° C. overnight, then the reaction mixture was concentrated under vacuum. The resulting residue was purified by column chromatography on silica gel (PE/EA=5/1, v/v) to afford ethyl 7-(4-(tert-butoxycarbonyl)piperazin-1-yl)imidazo[1,2-a]pyridine-3-carboxylate as a yellow solid (1.3 g, 92.8%). LRMS (M+H⁺) m/z calculated 375.2, found 375.2.

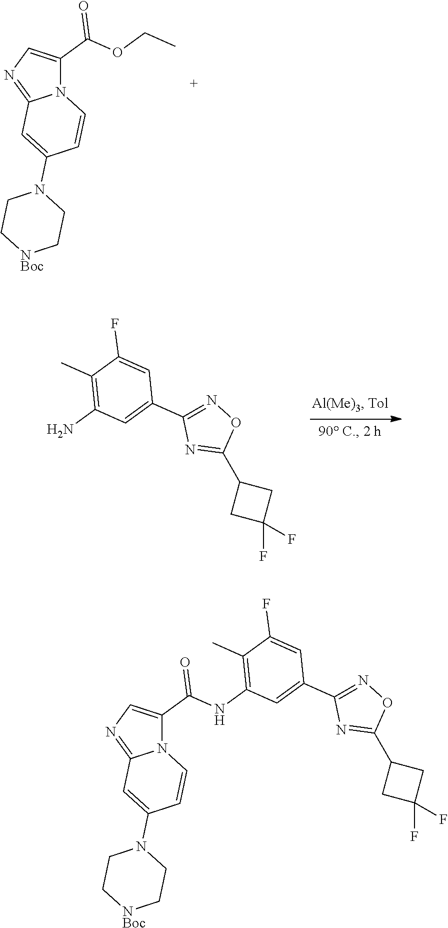 [SMILES: CCOC(=O)c1cnc2cc(N3CCN(C(=O)OC(C)(C)C)CC3)ccn12 ; Cc1c(N)cc(-c2noc(C3CC(F)(F)C3)n2)cc1F ; Cc1c(F)cc(-c2noc(C3CC(F)(F)C3)n2)cc1NC(=O)c1cnc2cc(N3CCN(C(=O)OC(C)(C)C)CC3)ccn12]

To a solution of ethyl 7-(4-(tert-butoxycarbonyl)piperazin-1-yl)imidazo[1,2-a]pyridine-3-carboxylate (600 mg, 1.6 mmol, 1.0 eq) and 5-(5-(3,3-difluorocyclobutyl)-1,2,4-oxadiazol-3-yl)-3-fluoro-2-methylaniline (454.0 mg, 1.6 mmol, 1.0 eq) in toluene (50 mL) was added Al(Me)₃ (2 M in toluene, 4.8 mL, 4.8 mmol, 3.0 eq) on ice. The reaction mixture was stirred at 90° C. for 2 h. The reaction mixture was concentrated under vacuum. The residue was purified by column chromatography on silica gel (PE/EA=2/1, v/v) to afford tert-butyl 4-(3-((5-(5-(3,3-difluorocyclobutyl)-1,2,4-oxadiazol-3-yl)-3-fluoro-2-methylphenyl)carbamoyl)imidazo[1,2-a]pyridin-7-yl)piperazine-)-carboxylate as a yellow solid (450 mg, 45.9). LRMS (M+H⁺) m/z calculated 612.2, found 612.1.

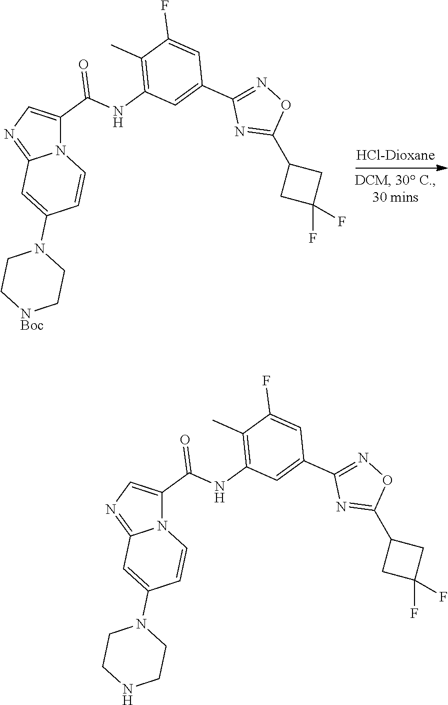 [SMILES: Cc1c(F)cc(-c2noc(C3CC(F)(F)C3)n2)cc1NC(=O)c1cnc2cc(N3CCN(C(=O)OC(C)(C)C)CC3)ccn12 ; Cc1c(F)cc(-c2noc(C3CC(F)(F)C3)n2)cc1NC(=O)c1cnc2cc(N3CCNCC3)ccn12]

To a solution of tert-butyl 4-(3-((5-(5-(3,3-difluorocyclobutyl)-1,2,4-oxadiazol-3-yl)-3-fluoro-2-methylphenyl)carbamoyl)imidazo[1,2-a]pyridin-7-yl)piperazine-1-carboxylate (100 mg, 0.16 mmol, 1.0 eq) in DCM (10 mL) was added 4 N HCl/Dioxane (5 mL). The reaction was stirred at 30° C. for 30 minutes. The mixture was concentrated, and the resulting residue was purified by prep-HPLC to afford N-(5-(5-(3,3-difluorocyclobutyl)-1,2,4-oxadiazol-3-yl)-3-fluoro-2-methylphenyl)-7-(piperazin-1-yl)imidazo[1,2-a]pyridine-3-carboxamide as a white solid (35.4 mg, 42.10%). LRMS (M+H⁺) m/z calculated 512.2, found 512.1. ¹H NMR (CDCl₃, 400 MHz) δ 9.25 (d, 1H), 8.42 (s, 1H), 8.06 (s, 1H), 7.62 (d, 1H), 7.48 (d, 1H), 6.87 (d, 1H), 6.78 (d, 1H), 3.63-3.68 (m, 1H), 3.32-3.34 (m, 4H), 3.03-3.17 (m, 8H), 2.30 (s, 3H).

### Example 11: Preparation of N-(5-(5-(3,3-difluorocyclobutyl)-1,2,4-oxadiazol-3-yl)-3-fluoro-2-methylphenyl)-7-(4-methylpiperazin-1-yl)imidazo[1,2-a]pyridine-3-carboxamide

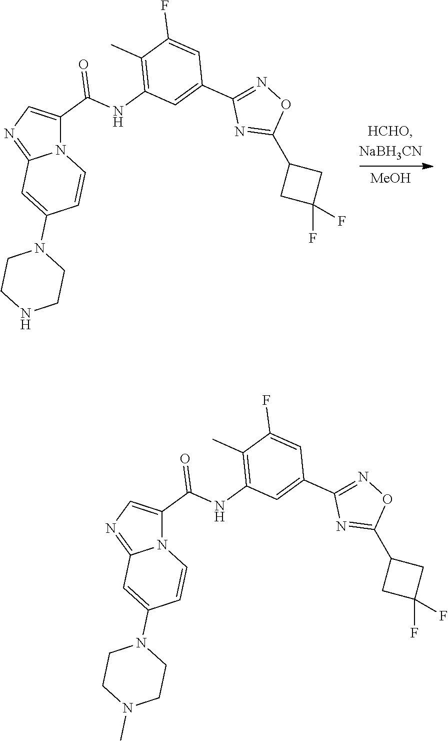 [SMILES: "MeOH" ; Cc1c(F)cc(-c2noc(C3CC(F)(F)C3)n2)cc1NC(=O)c1cnc2cc(N3CCNCC3)ccn12 ; Cc1c(F)cc(-c2noc(C3CC(F)(F)C3)n2)cc1NC(=O)c1cnc2cc(N3CCN(C)CC3)ccn12]

To a solution of N-(5-(5-(3,3-difluorocyclobutyl)-1,2,4-oxadiazol-3-yl)-3-fluoro-2-methylphenyl)-7-(piperazin-1-yl)imidazo[1,2-a]pyridine-3-carboxamide (140 mg, 0.27 mmol, 1.0 eq) in MeOH (15 mL) was added formaldehyde (37 wt % solution in water, 0.2 mL, 2.7 mmol, 10.0 eq) and NaBH₃CN. The solution was stirred at room temperature for 2 h, and then partitioned between DCM (20 mL) and saturated NaHCO₃ solution (20 mL). The aqueous layer was extracted with DCM (2×20 mL). The organic fraction was concentrated and purified by prep-HPLC to afford N-(5-(5-(3,3-difluorocyclobutyl)-1,2,4-oxadiazol-3-yl)-3-fluoro-2-methylphenyl)-7-(4-methylpiperazin-1-yl)imidazo[1,2-a]pyridine-3-carboxamide as a white solid (9.4 mg, 6.5%). LRMS (M+H⁺) m/z calculated 526.2, found 526.1. ¹H NMR (CDCl₃, 400 MHz) δ 9.30 (d, 1H), 8.33-8.39 (m, 2H), 7.63 (d, 1H), 6.91 (s, 1H), 6.79 (d, 1H), 3.63-3.68 (m, 1H), 3.41-3.48 (m, 4H), 3.08-3.16 (m, 4H), 2.70-2.73 (m, 4H), 2.45 (s, 3H), 2.34 (s, 1H).

### Example 12: Preparation of Methyl 3-(3-(3-(6-chloroimidazo[1,2-a]pyridine-3-carboxamido)-5-fluoro-4-methylphenyl)-1,2,4-oxadiazol-5-yl)azetidine-1-carboxylate

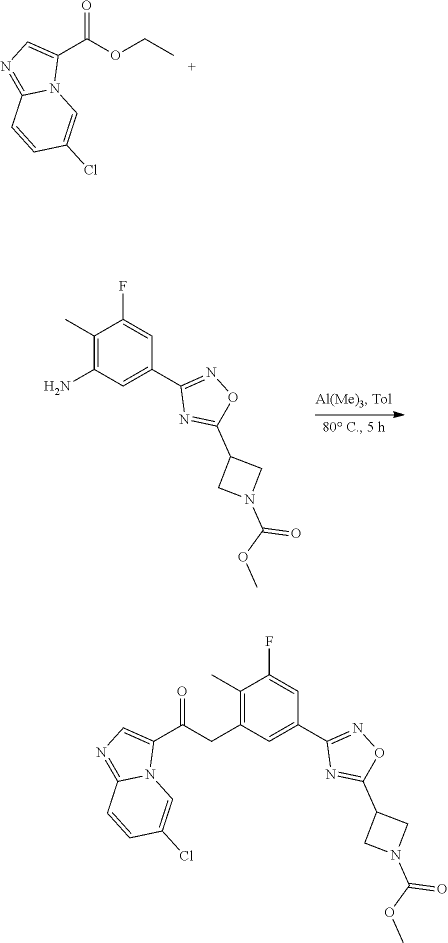 [SMILES: CCOC(=O)c1cnc2ccc(Cl)cn12 ; COC(=O)N1CC(c2nc(-c3cc(N)c(C)c(F)c3)no2)C1 ; COC(=O)N1CC(c2nc(-c3cc(F)c(C)c(CC(=O)c4cnc5ccc(Cl)cn45)c3)no2)C1]

To a solution of ethyl 6-chloroimidazo[1,2-a]pyridine-3-carboxylate (250 mg, 1.1 mmol, 1.0 eq) and methyl 3-(3-(3-amino-5-fluoro-4-methylphenyl)-1,2,4-oxadiazol-5-yl)azetidine-1-carboxylate (341.5 mg, 1.1 mmol, 1.0 eq) in toluene (50 mL) was added Al(Me)₃ (2 M in toluene, 1.6 mL, 3.3 mmol, 3.0 eq) on ice. The reaction mixture was stirred at 80° C. for 5 h. The reaction mixture was concentrated under vacuum. The resulting residue was partitioned between DCM (100 mL) and saturated NaHCO₃ solution (100 mL), filtered and the aqueous layer was extracted with DCM (2×100 mL). The combined organic layers were concentrated, and the resulting residue was purified by prep-HPLC to afford methyl 3-(3-(3-(6-chloroimidazo[1,2-a]pyridine-3-carboxamido)-5-fluoro-4-methylphenyl)-1,2,4-oxadiazol-5-yl)azetidine-1-carboxylate as a white solid (61.0 mg, 11.2%). LRMS (M+H⁺) m/z calculated 485.1, found 485.0. ¹H NMR (DMSO-d₆, 400 MHz) δ 10.34 (brs, 1H), 9.53 (d, 1H), 8.64 (s, 1H), 7.99 (s, 1H), 7.86 (d, 1H), 7.68 (d, 1H), 7.62 (d, 1H), 4.21-4.41 (m, 5H), 3.61 (s, 3H), 2.25 (s, 3H).

### Example 13: Preparation of N-(5-(5-(3,3-difluorocyclobutyl)-1,2,4-oxadiazol-3-yl)-3-fluoro-2-methylphenyl)-7-morpholinoimidazo[1,2-a]pyridine-3-carboxamide

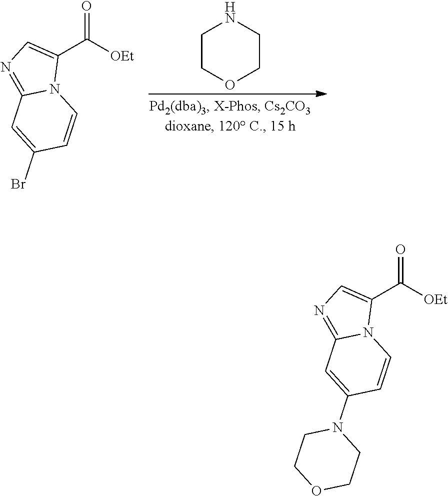 [SMILES: CCOC(=O)c1cnc2cc(Br)ccn12 ; C1COCCN1 ; CCOC(=O)c1cnc2cc(N3CCOCC3)ccn12]

To a solution of ethyl 7-bromoimidazo[1,2-a]pyridine-3-carboxylate (1.00 g, 3.71 mmol, 1.0 eq) in dioxane (40 mL) were added morpholine(1.61 g, 18.5 mmol, 5 eq), Pd₂(dba)₃ (169 mg, 0.185 mmol, 0.05 eq), X-Phos (267 mg, 0.556 mmol. 0.15 eq) and Cs₂CO₃ (2.42 g, 7.42 mmol, 2 eq). The reaction mixture was stirred at 120° C. for 15 h, diluted with water (100 mL), and extracted with EtOAc (80 mL×3). The combined organic layers were washed with brine (100 mL×2), dried with anhydrous sodium sulfate, filtered, and concentrated. The resulting residue was purified by column chromatography on silica gel (EA) to afford ethyl 7-morpholinoimidazo[1,2-a]pyridine-3-carboxylate as a yellow solid (950 mg, 92.5%). LCMS (M+H⁺) m/z calculated 276.1, found 276.1.

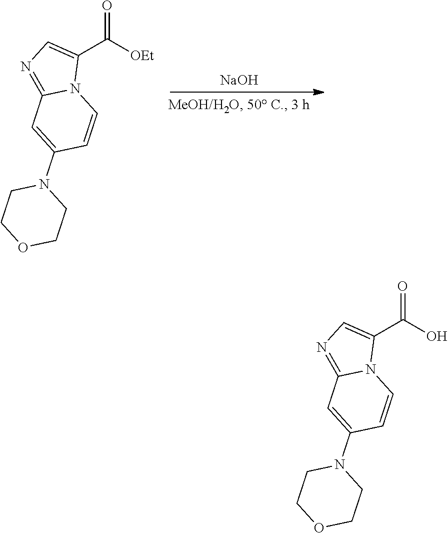 [SMILES: CCOC(=O)c1cnc2cc(N3CCOCC3)ccn12 ; "NaOH" ; O=C(O)c1cnc2cc(N3CCOCC3)ccn12]

A solution of ethyl 7-morpholinoimidazo[1,2-a]pyridine-3-carboxylate (800 mg, 3.6 mmol, 1.0 eq) and NaOH (218.2 mg, 5.5 mmol, 3 eq) in MeOH/H₂O (15 mL/5 mL) was stirred at 50° C. for 3 h, and the solvent was partially removed under vacuum. The pH was adjusted to 5 with diluted HCl. The precipitate was collected by filtration and dried to afford 7-morpholinoimidazo[1,2-a]pyridine-3-carboxylic acid as a white solid (390 mg, 54.3%). LCMS (M+H⁺) m/z calculated 248.1, found 248.1.

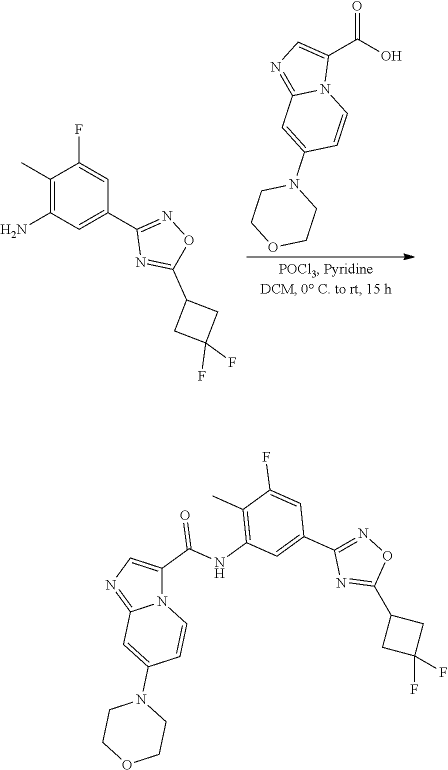 [SMILES: Cc1c(N)cc(-c2noc(C3CC(F)(F)C3)n2)cc1F ; O=C(O)c1cnc2cc(N3CCOCC3)ccn12 ; Cc1c(F)cc(-c2noc(C3CC(F)(F)C3)n2)cc1NC(=O)c1cnc2cc(N3CCOCC3)ccn12]

To a solution of 7-morpholinoimidazo[1,2-a]pyridine-3-carboxylic acid (100.0 mg, 0.40 mmol, 1.0 eq), 5-(5-(3,3-difluorocyclobutyl)-1,2,4-oxadiazol-3-yl)-3-fluoro-2-methylaniline (114.6 mg, 0.40 mmol, 1.0 eq) and pyridine (0.2 mL, 2.0 mmol, 5.0 eq) in DCM (50 mL) was added POCl₃ (0.1 mL, 1.2 mmol, 3.0 eq) at 0° C. The reaction mixture was stirred at rt for 15 h. The reaction mixture was diluted with water (50 mL) and extracted with DCM (50 mL×2). The combined organic layers were washed with brine (50 mL), dried over anhydrous sodium sulfate, filtered, and concentrated. The resulting residue was purified by Prep-HPLC to afford N-(5-(5-(3,3-difluorocyclobutyl)-1,2,4-oxadiazol-3-yl)-3-fluoro-2-methylphenyl)-7-morpholinoimidazo[1,2-a]pyridine-3-carboxamide as a white solid (69.5 mg, 33.6%). LRMS (M+H⁺) m/z calculated 513.2, found 513.1. ¹H NMR (DMSO-d₆, 400 MHz) δ 9.98 (s, 1H), 9.17 (d, 1H), 8.42 (s, 1H), 7.97 (s, 1H), 7.62 (d, 1H), 7.09 (dd, 1H), 6.91 (s, 1H), 3.86-3.92 (m, 1H), 3.74-3.77 (m, 4H), 3.16-3.23 (m, 4H), 3.04-3.23 (m, 4H), 2.25 (s, 3H).

### Example 14: Preparation of N-(3-fluoro-5-(5-((1R,2S)-2-fluorocyclopropyl)-1,2,4-oxadiazol-3-yl)-2-methylphenyl)-7-morpholinoimidazo[1,2-a]pyridine-3-carboxamide

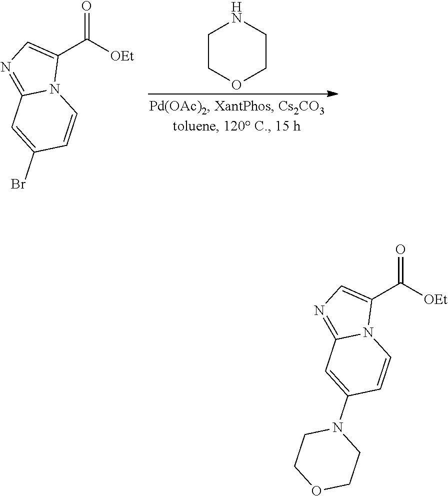 [SMILES: CCOC(=O)c1cnc2cc(Br)ccn12 ; C1COCCN1 ; CCOC(=O)c1cnc2cc(N3CCOCC3)ccn12]

The mixture of ethyl 7-bromoimidazo[1,2-a]pyridine-3-carboxylate (2.5 g, 9.3 mmol, 1.0 eq), morpholine (1.6 g, 18.6 mmol, 2.0 eq), palladium (II) acetate (154.0 mg, 0.93 mmol, 0.1 eq), Xantphos (538.5 mg, 0.93 mmol, 0.1 eq) and Cs₂CO₃ (6.0 g, 18.6 mmol, 2.0 eq) in toluene (50 mL) was stirred at 120° C. for 15 h under N₂. The reaction was quenched by adding H₂O (100 mL) and extracted with EtOAc (50 mL×3). The combined organic layers were washed with brine (100 mL), dried over anhydrous sodium sulfate, filtered, and concentrated. The resulting residue was purified by column chromatography on silica gel (PE/EtOAc=2/1, v/v) to afford ethyl 7-morpholinoimidazo[1,2-a]pyridine-3-carboxylate as a pale yellow solid (2.3 g, 89.8%). LCMS (M+H⁺) m/z calculated 276.1, found 276.1.

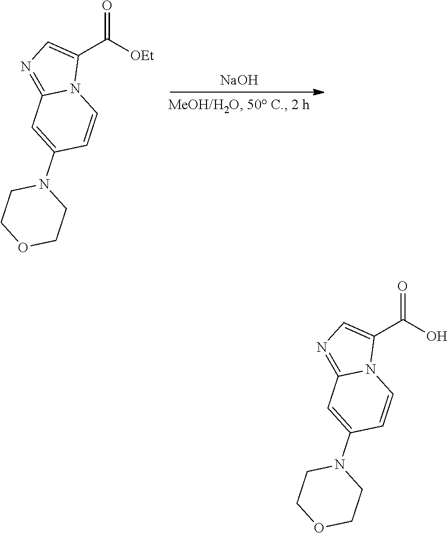 [SMILES: CCOC(=O)c1cnc2cc(N3CCOCC3)ccn12 ; "NaOH" ; O=C(O)c1cnc2cc(N3CCOCC3)ccn12]

The mixture of ethyl 7-morpholinoimidazo[1,2-a]pyridine-3-carboxylate (2.3 g, 8.4 mmol, 1.0 eq) and NaOH (1.0 g, 25.2 mmol, 3.0 eq) in MeOH (30.0 mL) and H₂O (10 mL) was stirred at 50° C. for 2 h. The mixture was concentrated and diluted by H₂O, and acidified to pH 2.0 by adding 1N HCl. The precipitate was filtered and dried to give 7-morpholinoimidazo[1,2-a]pyridine-3-carboxylic acid as a pale yellow solid (1.1 g, 55.0%). LCMS (M+H⁺) m/z calculated 248.1, found 248.1.

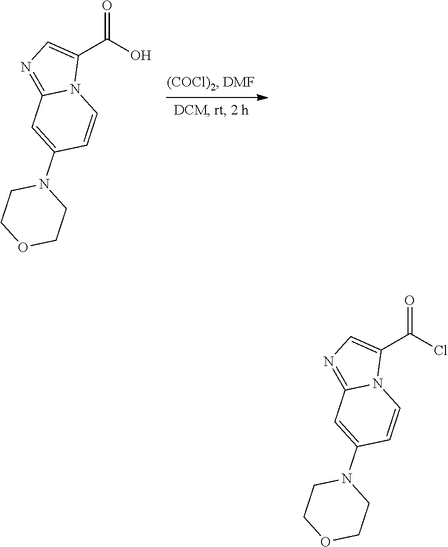 [SMILES: O=C(O)c1cnc2cc(N3CCOCC3)ccn12 ; O=C(Cl)c1cnc2cc(N3CCOCC3)ccn12]

To the solution of 7-morpholinoimidazo[1,2-a]pyridine-3-carboxylic acid (1.1 g, 4.5 mmol, 1.0 eq) in DCM (20.0 mL) were added 4 drops of DMF and oxalyl chloride (2.9 g, 22.5 mmol, 5.0 eq). The reaction mixture was stirred at rt for 2 h, and concentrated to afford crude 7-morpholinoimidazo[1,2-a]pyridine-3-carbonyl chloride as pale yellow solid (1.2 g, quant.), which was used in the next step without further purification.

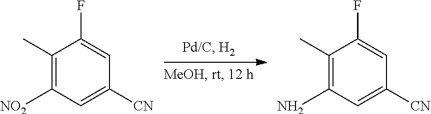

The mixture of 3-fluoro-4-methyl-5-nitrobenzonitrile (2.5 g, 13.9 mmol, 1.0 eq) and 250 mg Pd/C in MeOH (30.0 mL) was stirred under a stream of H₂ at rt for 12 h. The mixture was filtered through celite and concentrated. The resulting residue was purified by column chromatography on silica gel (PE/EtOAc=2/1, v/v) to afford 3-amino-5-fluoro-4-methylbenzonitrile as a yellow solid (1.2 g, 57.1%). LCMS (M+H⁺) m/z calculated 151.1, found 151.1.

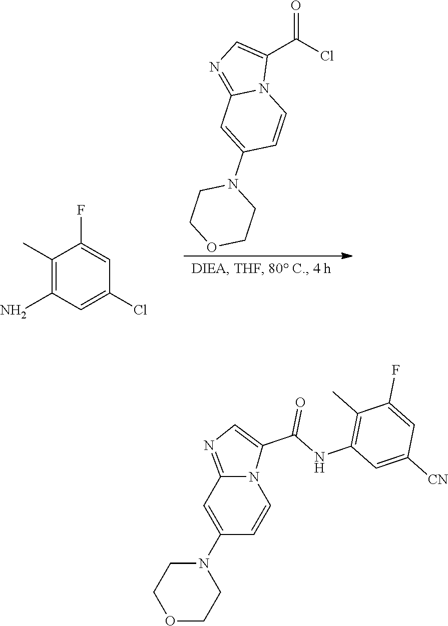

To a solution of 3-amino-5-fluoro-4-methylbenzonitrile (600.0 mg, 4.0 mmol, 1.0 eq) and DIEA (1.5 g, 12.0 mmol, 3.0 eq) in THF (10.0 mL) was added morpholinoimidazo[1,2-a]pyridine-3-carbonyl chloride (2.1 g, 8.0 mmol, 2.0 eq) at 0° C. The resulting solution was stirred at 80° C. for 4 h. The mixture was concentrated and purified by column chromatography on silica gel (PE/EtOAc=1/1, v/v) to afford N-(5-cyano-3-fluoro-2-methylphenyl)-7-morpholinoimidazo[1,2-a]pyridine-3-carboxamide as a yellow solid (200.0 mg, 13.3%). LCMS (M+H⁺) m/z calculated 380.1, found 380.1.

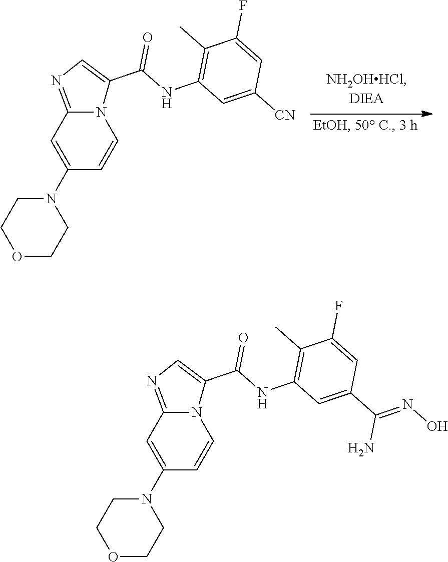 [SMILES: Cc1c(F)cc(C#N)cc1NC(=O)c1cnc2cc(N3CCOCC3)ccn12 ; Cc1c(F)cc(/C(N)=N/O)cc1NC(=O)c1cnc2cc(N3CCOCC3)ccn12]

The mixture of N-(5-cyano-3-fluoro-2-methylphenyl)-7-morpholinoimidazo[1,2-a]pyridine-3-carboxamide (200.0 mg, 0.53 mmol, 1.0 eq), hydroxylamine hydrochloride (75.9 mg, 1.1 mmol, 2.0 eq) and N,N-Diisopropylethylamine (341.9 mg, 2.7 mmol, 5.0 eq) in EtOH (10.0 mL) was stirred at 50° C. for 3 h. The reaction was quenched by H₂O (50 mL) and extracted with EtOAc (50 mL×3). The combined organic layers were washed with brine (50 mL), dried over anhydrous sodium sulfate, filtered, and concentrated. The resulting residue was purified by column chromatography on silica gel (PE/EtOAc=1/1, v/v) to afford (Z)-N-(3-fluoro-5-(N′-hydroxycarbamimidoyl)-2-methylphenyl)-7-morpholinoimidazo[1,2-a]pyridine-3-carboxamide as a pale yellow solid (250.0 mg, quant.). LCMS (M+H⁺) m/z calculated 413.2, found 413.2.

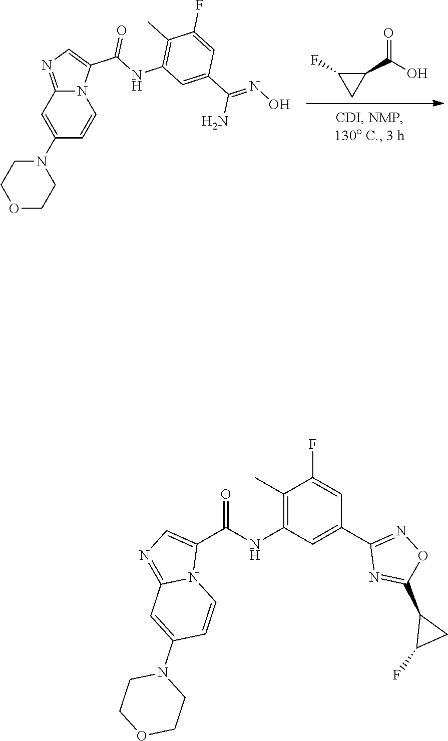 [SMILES: Cc1c(F)cc(/C(N)=N/O)cc1NC(=O)c1cnc2cc(N3CCOCC3)ccn12 ; O=C(O)[C@H]1C[C@@H]1F ; Cc1c(F)cc(-c2noc([C@H]3C[C@@H]3F)n2)cc1NC(=O)c1cnc2cc(N3CCOCC3)ccn12]

To the mixture of (1R,2S)-2-fluorocyclopropane-1-carboxylic acid (47.3 mg, 0.45 mmol, 1.5 eq) in NMP (5.0 mL) was added 1,1′-Carbonyldiimidazole (72.9 mg, 0.45 mmol, 1.5 eq). The mixture was stirred at 40° C. for 2.0 h. (Z)-N-(3-fluoro-5-(N′-hydroxycarbamimidoyl)-2-methylphenyl)imidazo[1,2-a]pyridine-3-carboxamide (125.0 mg, 0.3 mmol, 1.0 eq) was then added and stirring continued at 130° C. for 3 h. The reaction was quenched by H₂O (50.0 mL) and extracted with EtOAc (50 mL×3). The combined organic layers were washed with brine (100 mL), dried over anhydrous sodium sulfate, filtered, and concentrated. The resulting residue was purified by prep-HPLC to afford N-(3-fluoro-5-(5-((1R,2S)-2-fluorocyclopropyl)-1,2,4-oxadiazol-3-yl)-2-methylphenyl)-7-morpholinoimidazo[1,2-a]pyridine-3-carboxamide as a white solid (29.5 mg, 20.0%). LCMS (M+H⁺) m/z calculated 481.2, found 481.2. ¹H NMR (DMSO-d₆, 400 MHz) δ 9.96 (s, 1H), 9.14-9.16 (d, 1H), 8.40 (s, 1H), 7.90 (s, 1H), 7.56-7.59 (m, 1H), 7.08-7.10 (m, 1H), 6.90-6.91 (d, 1H), 5.20-5.21 (t, 1H), 3.74-3.77 (t, 4H), 3.25-3.31 (t, 4H), 3.06-3.08 (m, 1H), 2.23-2.24 (d, 3H), 1.92-1.98 (m, 1H), 1.56-1.61 (m, 1H).

### Example 15: Preparation of Methyl 3-(3-(3-fluoro-4-methyl-5-(7-morpholinoimidazo[1,2-a]pyridine-3-carboxamido)phenyl)-1,2,4-oxadiazol-5-yl)azetidine-1-carboxylate

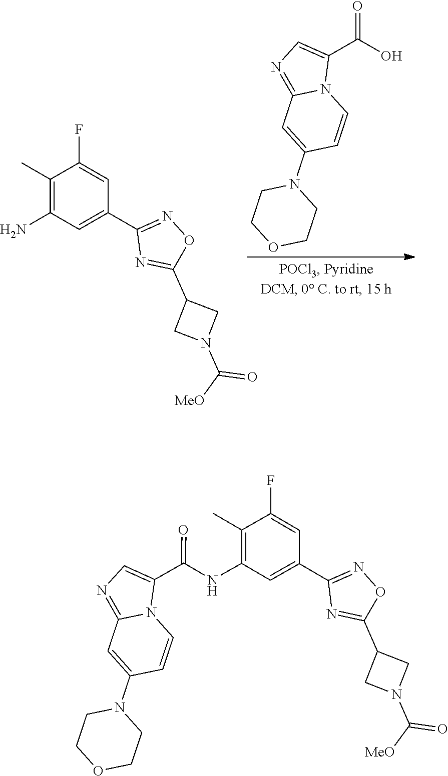 [SMILES: COC(=O)N1CC(c2nc(-c3cc(N)c(C)c(F)c3)no2)C1 ; O=C(O)c1cnc2cc(N3CCOCC3)ccn12 ; COC(=O)N1CC(c2nc(-c3cc(F)c(C)c(NC(=O)c4cnc5cc(N6CCOCC6)ccn45)c3)no2)C1]

To a solution of 7-morpholinoimidazo[1,2-a]pyridine-3-carboxylic acid (120.0 mg, 0.486 mmol, 1.0 eq), methyl 3-(3-(3-amino-5-fluoro-4-methylphenyl)-1,2,4-oxadiazol-5-yl)azetidine-1-carboxylate (148.7 mg, 0.486 mmol, 1.0 eq) and pyridine (0.2 mL, 2.43 mmol, 5.0 eq) in DCM (30 mL) was added POCl₃ (0.1 mL, 1.46 mmol, 3.0 eq) at 0° C. The reaction mixture was stirred at rt for 15 h. Upon reaction completion, the reaction was diluted with water (50 mL) and extracted with DCM (50 mL×2). The combined organic layers were washed with brine (50 mL), dried over anhydrous sodium sulfate, filtered, and concentrated. The resulting residue was purified by Prep-HPLC to afford methyl 3-(3-(3-fluoro-4-methyl-5-(7-morpholinoimidazo[1,2-a]pyridine-3-carboxamido)phenyl)-1,2,4-oxadiazol-5-yl)azetidine-1-carboxylate (59.4 mg, 22.9%). LCMS (M+H⁺) m/z calculated 536.2, found 536.1. ¹H NMR (DMSO-d₆, 400 MHz) δ 9.97 (s, 1H), 9.17 (d, 1H), 8.42 (s, 1H), 7.99 (s, 1H), 7.64 (d, 1H), 7.09 (dd, 1H), 6.91 (d, 1H), 4.45-4.33 (m, 2H), 4.33-4.24 (m, 1H), 4.24-4.13 (m, 2H), 3.81-3.70 (m, 4H), 3.60 (s, 3H), 3.26-3.32 (m, 4H), 2.25 (d, 3H).

### Example 16: Preparation of Methyl 3-(3-(3-(7-chloroimidazo[1,2-a]pyridine-3-carboxamido)-5-fluoro-4-methylphenyl)-1,2,4-oxadiazol-5-yl)azetidine-1-carboxylate

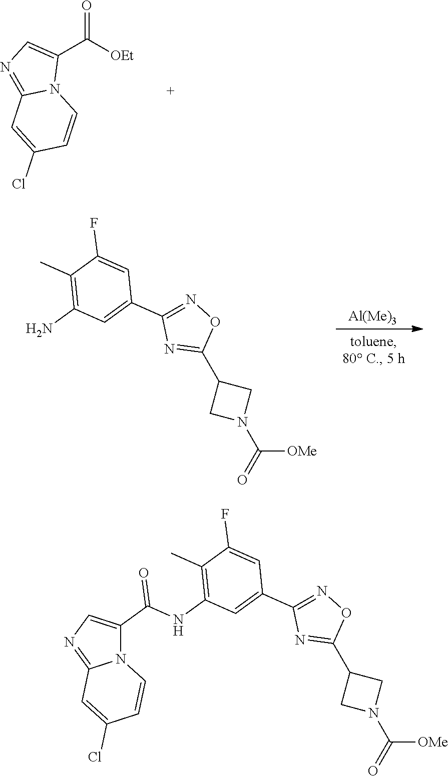

To a stirring solution of ethyl ethyl 7-chloroimidazo[1,2-a]pyridine-3-carboxylate (250 mg, 1.1 mmol, 1.0 eq) and methyl 3-(3-(3-amino-5-fluoro-4-methylphenyl)-1,2,4-oxadiazol-5-yl)azetidine-1-carboxylate (341.5 mg, 1.1 mmol, 1.0 eq) in toluene (50 mL) was added Al(Me)₃ (2 M in toluene, 1.6 mL, 3.3 mmol, 3.0 eq) at 0° C. The reaction mixture was stirred at 80° C. for 5 h. The reaction mixture was concentrated under vacuum. The resulting residue was partitioned between DCM (100 mL) and saturated NaHCO₃ solution (100 mL). The aqueous layer was extracted with DCM (100 mL×2). The combined organic layers were concentrated, and the resulting residue was purified by prep-HPLC to afford methyl 3-(3-(3-(7-chloroimidazo[1,2-a]pyridine-3-carboxamido)-5-fluoro-4-methylphenyl)-1,2,4-oxadiazol-5-yl)azetidine-1-carboxylate as a white solid (58.2 mg, 10.7%). LCMS (M+H⁺) m/z calculated 485.1, found 485.0. ¹H NMR (DMSO-d₆, 400 MHz) δ 10.29 (s, 1H), 9.42 (d, 1H), 8.61 (s, 1H), 7.98-8.00 (m, 2H), 7.66 (dd, 1H), 7.27 (dd, 1H), 4.19-4.38 (m, 5H), 3.60 (s, 3H), 2.25 (d, 3H).

### Example 17: Preparation of Methyl 3-(3-(3-fluoro-4-methyl-5-(7-methylimidazo[1,2-a]pyridine-3-carboxamido)phenyl)-1,2,4-oxadiazol-5-yl)azetidine-1-carboxylate

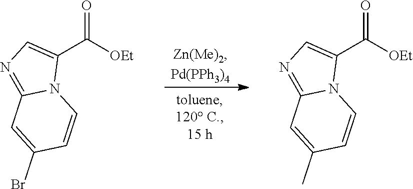

To a stirring solution of ethyl 7-bromoimidazo[1,2-a]pyridine-3-carboxylate (500 mg, 1.9 mmol, 1.0 eq) and Pd(PPh₃)₄ (214.9 mg, 0.19 mmol, 0.1 eq) in toluene (30 mL) was added Zn(Me)₂ (1 M in toluene, 5.6 mL, 5.6 mmol, 3.0 eq) at 0° C. The reaction was stirred at 120° C. for 15 h. The reaction was concentrated and purified by column chromatography on silica gel (PE/EA=2/1, v/v) to afford ethyl 7-methylimidazo[1,2-a]pyridine-3-carboxylate as a yellow solid (190 mg, 50.0%). LCMS (M+H⁺) m/z calculated 205.1, found 205.0.

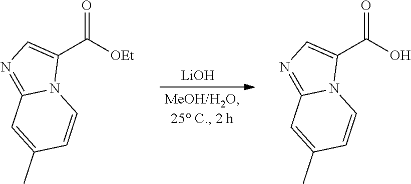

A solution of ethyl 7-methylimidazo[1,2-a]pyridine-3-carboxylate (170 mg, 0.83 mmol, 1.0 eq) and LiOH (40.0 mg, 1.7 mmol, 2.0 eq) in MeOH/H₂O (30 mL/5 mL) was stirred at 25° C. for 2 h. The solvent was partially removed under vacuum. The pH was adjusted to 5 with conc. HCl. The white solid was collected by filtration and dried under vacuum to afford 7-methylimidazo[1,2-a]pyridine-3-carboxylic acid as a white solid (120.0 mg, 81.6%). LCMS (M+H⁺) m/z calculated 177.1, found 177.0.

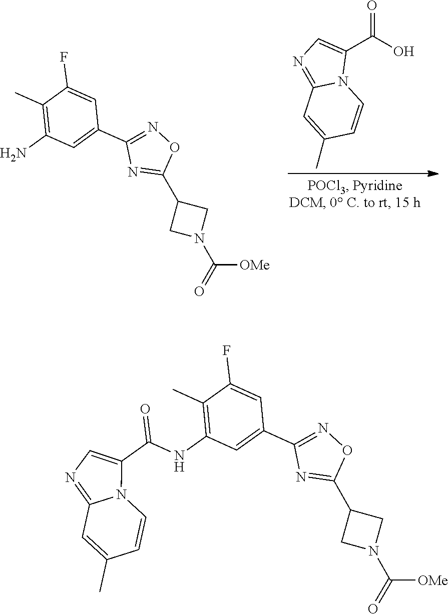 [SMILES: COC(=O)N1CC(c2nc(-c3cc(N)c(C)c(F)c3)no2)C1 ; Cc1ccn2c(C(=O)O)cnc2c1 ; COC(=O)N1CC(c2nc(-c3cc(F)c(C)c(NC(=O)c4cnc5cc(C)ccn45)c3)no2)C1]

To a solution of 7-methylimidazo[1,2-a]pyridine-3-carboxylic acid (120.0 mg, 0.68 mmol, 1.0 eq), methyl 3-(3-(3-amino-5-fluoro-4-methylphenyl)-1,2,4-oxadiazol-5-yl)azetidine-1-carboxylate (208.6 mg, 0.68 mmol, 1.0 eq) and pyridine (0.3 mL, 3.4 mmol, 5.0 eq) in DCM (30 mL) was added POCl₃ (0.2 mL, 2.0 mmol, 3.0 eq) at 0° C. The reaction mixture was stirred at 25° C. for 15 h. The reaction was diluted with water (50 mL) and extracted with DCM (50 mL×2).The combined organic layers were washed with brine (50 mL), dried over anhydrous sodium sulfate, filtered, and concentrated under vacuum. The resulting residue was purified by Prep-HPLC to afford methyl 3-(3-(3-fluoro-4-methyl-5-(7-methylimidazo[1,2-a]pyridine-3-carboxamido)phenyl)-1,2,4-oxadiazol-5-yl)azetidine-1-carboxylate (16.0 mg, 5.1%). LCMS (M+H⁺) m/z calculated 465.2, found 465.1. ¹H NMR (DMSO-d₆, 400 MHz) δ 10.14 (s, 1H), 9.31 (d, 1H), 8.53 (s, 1H), 7.99 (s, 1H), 7.66 (d, 1H), 7.58 (s, 1H), 7.04 (d, 1H), 4.18-4.38 (m, 5H), 3.60 (s, 3H), 2.46 (s, 3H), 2.25 (s, 3H).

### Example 18: Preparation of 7-(4-acetylpiperazin-1-yl)-N-(5-(5-(3,3-difluorocyclobutyl)-1,2,4-oxadiazol-3-yl)-3-fluoro-2-methylphenyl)imidazo[1,2-a]pyridine-3-carboxamide

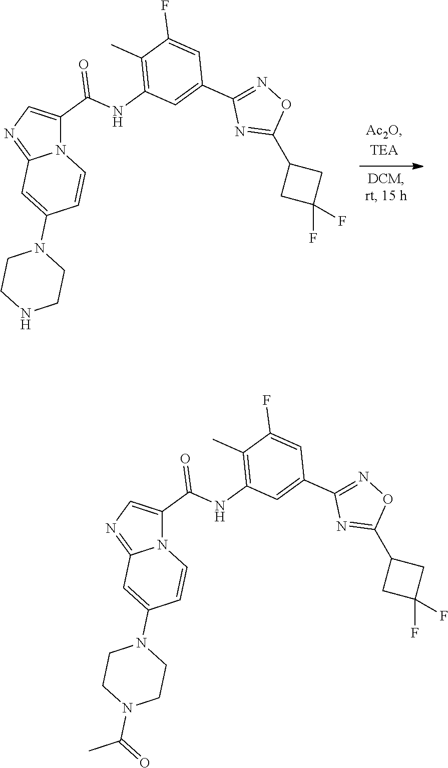 [SMILES: Cc1c(F)cc(-c2noc(C3CC(F)(F)C3)n2)cc1NC(=O)c1cnc2cc(N3CCNCC3)ccn12 ; "DCM,rt, 15 h" ; CC(=O)N1CCN(c2ccn3c(C(=O)Nc4cc(-c5noc(C6CC(F)(F)C6)n5)cc(F)c4C)cnc3c2)CC1]

To a solution of N-(5-(5-(3,3-difluorocyclobutyl)-1,2,4-oxadiazol-3-yl)-3-fluoro-2-methylphenyl)-7-(piperazin-1-yl)imidazo[1,2-a]pyridine-3-carboxamide (140 mg, 0.27 mmol, 1.0 eq) in DCM (15 mL) were added acetic anhydride (149.7 mg, 1.4 mmol, 5.0 eq) and TEA (277.7 mg, 2.7 mmol, 10.0 eq). The solution was stirred at rt for 15 h, and then partitioned between DCM (20 mL) and saturated NaHCO₃ solution (20 mL). The aqueous layer was extracted with DCM (20 mL×2) and concentrated. The resulting residue was purified by prep-HPLC to afford 7-(4-acetylpiperazin-1-yl)-N-(5-(5-(3,3-difluorocyclobutyl)-1,2,4-oxadiazol-3-yl)-3-fluoro-2-methylphenyl)imidazo[1,2-a]pyridine-3-carboxamide as a white solid (65.3 mg, 43.0%). LCMS (M+H⁺) m/z calculated 554.2, found 554.1. ¹H NMR (DMSO-d₆, 400 MHz) δ 10.49 (s, 1H), 9.22 (d, 1H), 8.67 (s, 1H), 7.95 (s, 1H), 7.69 (d, 1H), 7.37 (dd, 1H), 6.99 (dd, 1H), 3.86-3.92 (m, 1H), 3.64 (s, 6H), 3.55-3.58 (m, 2H), 3.05-3.24 (m, 4H), 2.25 (s, 3H), 2.07 (s, 3H).

### Example 19: Preparation of Methyl 3-(3-(3-fluoro-5-(6-fluoroimidazo[1,2-a]pyridine-3-carboxamido)-4-methylphenyl)-1,2,4-oxadiazol-5-yl)azetidine-1-carboxylate

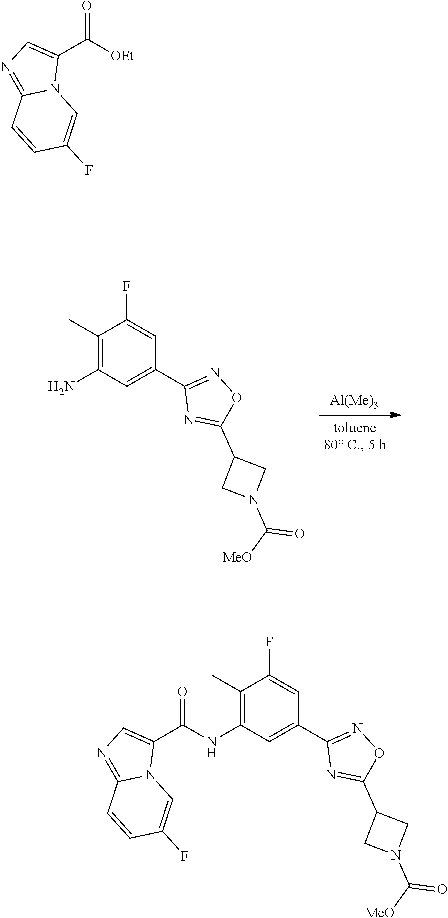 [SMILES: CCOC(=O)c1cnc2ccc(F)cn12 ; COC(=O)N1CC(c2nc(-c3cc(N)c(C)c(F)c3)no2)C1 ; COC(=O)N1CC(c2nc(-c3cc(F)c(C)c(NC(=O)c4cnc5ccc(F)cn45)c3)no2)C1 ; "Al(Me)3"]

To a stirring solution of ethyl 6-fluoroimidazo[1,2-a]pyridine-3-carboxylate (300 mg, 1.4 mmol, 1.0 eq) and methyl 3-(3-(3-amino-5-fluoro-4-methylphenyl)-1,2,4-oxadiazol-5-yl)azetidine-1-carboxylate (441.3 mg, 1.4 mmol, 1.0 eq) in toluene (50 mL) was added Al(Me)₃ (2 M in toluene, 2.2 mL, 4.3 mmol, 3.0 eq) at 0° C. The reaction mixture was stirred at 80° C. for 5 h. The reaction mixture was concentrated and partitioned between DCM (100 mL) and saturated NaHCO₃ solution (100 mL), and filtered. The aqueous layer was extracted with DCM (100 mL×2). The combined organic phases were concentrated and the resulting residue was purified by prep-HPLC to afford methyl 3-(3-(3-fluoro-5-(6-fluoroimidazo[1,2-a]pyridine-3-carboxamido)-4-methylphenyl)-1,2,4-oxadiazol-5-yl)azetidine-1-carboxylate as a white solid (203.9 mg, 30.0%). LCMS (M+H⁺) m/z calculated 469.1, found 469.0. ¹H NMR (DMSO-d₆, 400 MHz) δ 10.35 (s, 1H), 9.48 (d, 1H), 8.69 (s, 1H), 7.98 (s, 1H), 7.92 (dd, 1H), 7.67-7.74 (m, 2H), 4.20-4.39 (m, 5H), 3.61 (s, 3H), 2.26 (s, 3H).

### Example 20: Preparation of Methyl 3-(3-(3-fluoro-4-methyl-5-(6-(piperazin-1-yl)imidazo[1,2-a]pyridine-3-carboxamido)phenyl)-1,2,4-oxadiazol-5-yl)azetidine-1-carboxylate

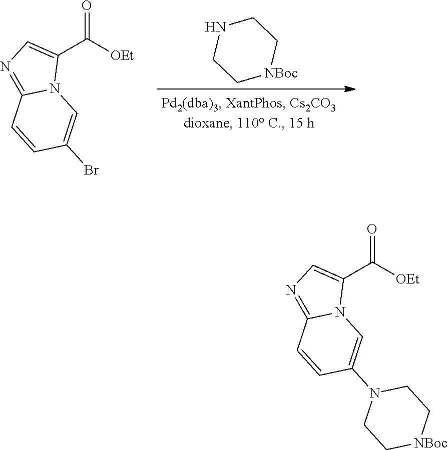 [SMILES: CCOC(=O)c1cnc2ccc(Br)cn12 ; CC(C)(C)OC(=O)N1CCNCC1 ; CCOC(=O)c1cnc2ccc(N3CCN(C(=O)OC(C)(C)C)CC3)cn12]

A mixture of ethyl 6-bromoimidazo[1,2-a]pyridine-3-carboxylate (5 g, 18.6 mmol, 1.0 eq), tert-butyl piperazine-1-carboxylate (10.4 g, 55.8 mmol, 3.0 eq), Cs₂CO₃ (12.1 g, 37.2 mmol, 2.0 eq), XantPhos (1.1 g, 1.9 mmol, 0.1 eq) and Pd₂(dba)₃ (851.3 mg, 0.9 mmol, 0.05 eq) in dioxane (100 mL) was stirred at 110° C. overnight. The reaction mixture was diluted with water (100 mL) and extracted with EtOAc (100 mL×3). The combined organic layers were washed with brine (100 mL×2), dried with anhydrous sodium sulfate, filtered, and concentrated. The resulting residue was purified by column chromatography on silica gel (PE/EA=5/1, v/v) to afford ethyl 6-(4-(tert-butoxycarbonyl)piperazin-1-yl)imidazo[1,2-a]pyridine-3-carboxylate as a yellow solid (6.5 g, 92.8%). LCMS (M+H⁺) m/z calculated 375.2, found 375.1.

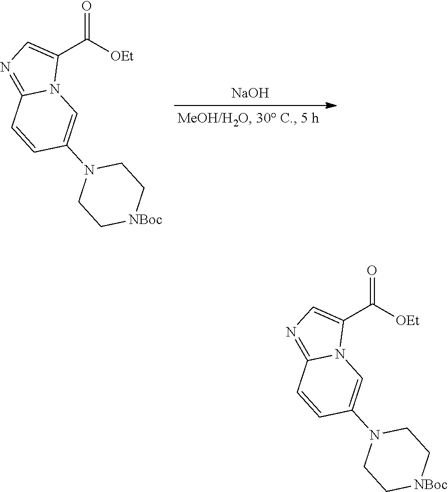 [SMILES: CCOC(=O)c1cnc2ccc(N3CCN(C(=O)OC(C)(C)C)CC3)cn12 ; "NaOH" ; CCOC(=O)c1cnc2ccc(N3CCN(C(=O)OC(C)(C)C)CC3)cn12]

A solution of ethyl 6-(4-(tert-butoxycarbonyl)piperazin-1-yl)imidazo[1,2-a]pyridine-3-carboxylate (5 g, 13.3 mmol, 1.0 eq) and NaOH (802.1 mg, 20.1 mmol, 1.5 eq) in MeOH/H₂O (30 mL/5 mL) was stirred at 30° C. for 5 h, and the solvent was partially removed under vacuum. The pH was adjusted to 6 with conc. HCl. The solid was collected by filtration and dried to afford 6-(4-(tert-butoxycarbonyl)piperazin-1-yl)imidazo[1,2-a]pyridine-3-carboxylic acid as a white solid (4.0 g, 87.0%). LCMS (M+H⁺) m/z calculated 347.2, found 347.1.

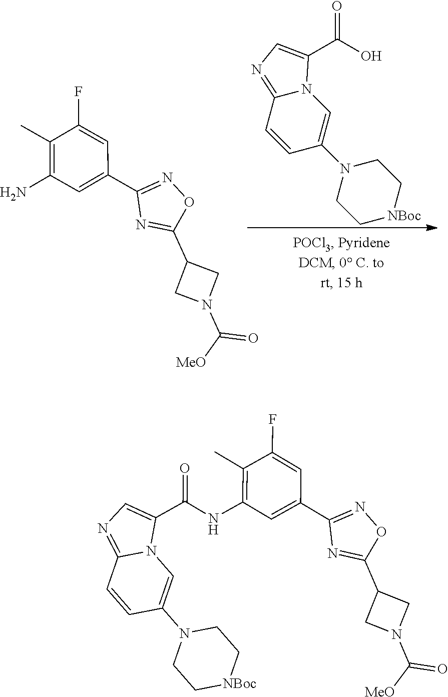 [SMILES: COC(=O)N1CC(c2nc(-c3cc(N)c(C)c(F)c3)no2)C1 ; CC(C)(C)OC(=O)N1CCN(c2ccc3ncc(C(=O)O)n3c2)CC1 ; COC(=O)N1CC(c2nc(-c3cc(F)c(C)c(NC(=O)c4cnc5ccc(N6CCN(C(=O)OC(C)(C)C)CC6)cn45)c3)no2)C1]

To a solution of 6-(4-(tert-butoxycarbonyl)piperazin-1-yl)imidazo[1,2-a]pyridine-3-carboxylic acid (1 g, 2.9 mmol, 1.0 eq), methyl 3-(3-(3-amino-5-fluoro-4-methylphenyl)-1,2,4-oxadiazol-5-yl)azetidine-1-carboxylate (884.4 mg, 2.9 mmol, 1.0 eq) and pyridine (1.2 mL, 24.5 mmol, 5.0 eq) in DCM (50 mL) was added POCl₃ (0.8 mL, 1.2 mmol, 3.0 eq) at 0° C. The reaction mixture was stirred at rt for 15 h, and diluted with water (50 mL). The aqueous layer was extracted with DCM (50 mL×2). The combined organic layers were washed with brine (50 mL), dried over anhydrous sodium sulfate, filtered, and concentrated to afford tert-butyl 4-(3-((3-fluoro-5-(5-(1-(methoxycarbonyl)azetidin-3-yl)-1,2,4-oxadiazol-3-yl)-2-methylphenyl)carbamoyl)imidazo[1,2-a]pyridin-6-yl)piperazine-1-carboxylate as a yellow solid (1.2 g, 66.7%). LCMS (M+H⁺) m/z calculated 635.3, found 635.2.

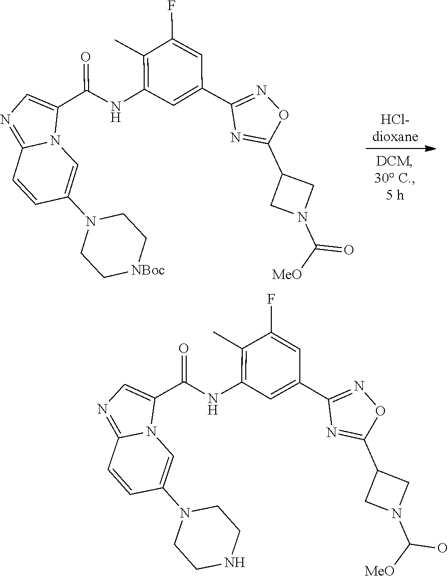

To a stirring solution of tert-butyl 4-(3-((3-fluoro-5-(5-(1-(methoxycarbonyl)azetidin-3-yl)-1,2,4-oxadiazol-3-yl)-2-methylphenyl)carbamoyl)imidazo[1,2-a]pyridin-6-yl)piperazine-1-carboxylate (100 mg, 0.16 mmol, 1.0 eq) in DCM (10 mL) was added 4 N HCl/dioxane (5 mL). The reaction was stirred at 30° C. for 5 h. The mixture was concentrated and purified by prep-HPLC to afford methyl 3-(3-(3-fluoro-4-methyl-5-(6-(piperazin-1-yl)imidazo[1,2-a]pyridine-3-carboxamido)phenyl)-1,2,4-oxadiazol-5-yl)azetidine-1-carboxylate as a white solid (24.9 mg, 29.8%). LCMS (M+H⁺) m/z calculated 535.2, found 535.1. ¹H NMR (DMSO-d₆, 400 MHz) δ 10.14 (s, 1H), 8.93 (s, 1H), 8.51 (s, 1H), 7.96 (s, 1H), 7.65-7.69 (m, 2H), 7.54 (d, 1H), 4.20-4.38 (m, 5H), 3.59 (s, 3H), 2.98-3.09 (m, 8H), 2.25 (s, 3H).

### Example 21: Preparation of Methyl 3-(3-(3-fluoro-4-methyl-5-(6-methylimidazo[1,2-a]pyridine-3-carboxamido)phenyl)-1,2,4-oxadiazol-5-yl)azetidine-1-carboxylate

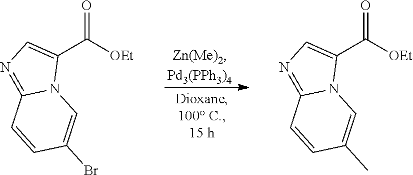

To a stirring solution of ethyl ethyl 6-bromoimidazo[1,2-a]pyridine-3-carboxylate (200 mg, 0.74 mmol, 1.0 eq) and Pd(PPh₃)₄ (85.9 mg, 0.074 mmol, 0.1 eq) in dioxane (30 mL) was added Zn(Me)₂ (1 M in toluene, 2.2 mL, 2.2 mmol, 3.0 eq) at 0° C. The reaction mixture was stirred at 100° C. for 15 h. The reaction mixture was concentrated and purified by column chromatography on silica gel (PE/EA=2/1, v/v) to afford ethyl 6-methylimidazo[1,2-a]pyridine-3-carboxylate as a yellow solid (190 mg, quant.). LCMS (M+H⁺) m/z calculated 205.1, found 205.0.

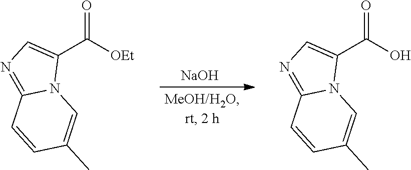

A solution of ethyl 6-methylimidazo[1,2-a]pyridine-3-carboxylate (190 mg, 0.93 mmol, 1.0 eq) and NaOH (74.5 mg, 1.9 mmol, 2.0 eq) in MeOH/H₂O (30 mL/5 mL) was stirred at room temperature for 2 h, and the solvent was partially removed under vacuum. The pH was adjusted to 5 with conc. HCl. The precipitate was collected by filtration and dried to afford 6-methylimidazo[1,2-a]pyridine-3-carboxylic acid as a white solid (150.0 mg, 91.4%). LCMS (M+H⁺) m/z calculated 177.1, found 177.0.

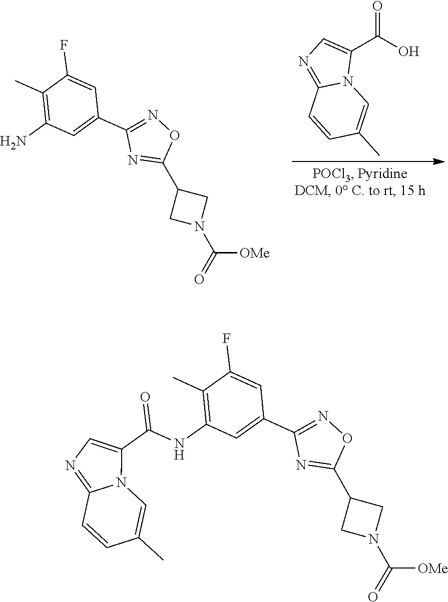 [SMILES: COC(=O)N1CC(c2nc(-c3cc(N)c(C)c(F)c3)no2)C1 ; Cc1ccc2ncc(C(=O)O)n2c1 ; COC(=O)N1CC(c2nc(-c3cc(F)c(C)c(NC(=O)c4cnc5ccc(C)cn45)c3)no2)C1]

To a solution of 6-methylimidazo[1,2-a]pyridine-3-carboxylic acid (150.0 mg, 0.85 mmol, 1.0 eq), methyl 3-(3-(3-amino-5-fluoro-4-methylphenyl)-1,2,4-oxadiazol-5-yl)azetidine-1-carboxylate (260.8 mg, 0.85 mmol, 1.0 eq) and pyridine (0.4 mL, 4.3 mmol, 5.0 eq) in DCM (30 mL) was added POCl₃ (0.24 mL, 2.0 mmol, 3.0 eq) at 0° C. The reaction mixture was stirred at rt for 15 h. The reaction mixture was diluted with water (50 mL) and extracted with DCM (50 mL×2). The combined organic layers were washed with brine (50 mL), dried over anhydrous sodium sulfate, filtered, and concentrated. The resulting residue was purified by Prep-HPLC to afford methyl 3-(3-(3-fluoro-4-methyl-5-(6-methylimidazo[1,2-a]pyridine-3-carboxamido)phenyl)-1,2,4-oxadiazol-5-yl)azetidine-1-carboxylate (9.7 mg, 2.5%). LCMS (M+H⁺) m/z calculated 465.2, found 465.1. ¹H NMR (DMSO-d₆, 400 MHz) δ 10.17 (s, 1H), 9.28 (d, 1H), 8.55 (s, 1H), 8.00 (s, 1H), 7.65-7.72 (m, 2H), 7.39 (dd, 1H), 4.20-4.38 (m, 5H), 3.60 (s, 3H), 2.46 (s, 3H), 2.25 (s, 3H).

Example 22: Preparation of N-(3-fluoro-5-(5-((1R,2S)-2-fluorocyclopropyl)-1,2,4-oxadiazol-3-yl)-2-methylphenyl)-7-(piperazin-1-yl)imidazo[1,2-a]pyridine-3-carboxamide and N-(3-fluoro-5-(5-((1S,2R)-2-fluorocyclopropyl)-1,2,4-oxadiazol-3-yl)-2-methylphenyl)-7-(piperazin-1-yl)imidazo[1,2-a]pyridine-3-carboxamide(racemic, trans)

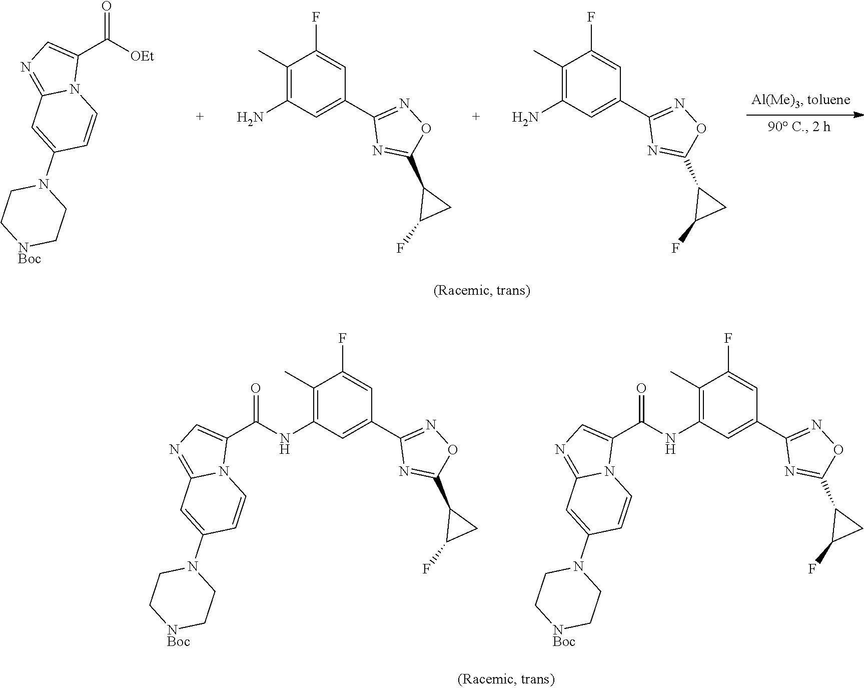

To a stirring solution of ethyl 7-(4-(tert-butoxycarbonyl)piperazin-1-yl)imidazo[1,2-a]pyridine-3-carboxylate (500 mg, 1.3 mmol, 1.0 eq), 3-fluoro-5-(5-((1R,2S)-2-fluorocyclopropyl)-1,2,4-oxadiazol-3-yl)-2-methylaniline and 3-fluoro-5-(5-((1S,2R)-2-fluorocyclopropyl)-1,2,4-oxadiazol-3-yl)-2-methylaniline (racemic, trans, 335.6 mg, 1.3 mmol, 1.0 eq) in toluene (50 mL) was added Al(Me)₃ (2 M in toluene, 2.0 mL, 4.0 mmol, 3.0 eq) at 0° C. The reaction mixture was stirred at 90° C. for 2 h. The reaction mixture was concentrated and partitioned between DCM (100 mL) and saturated NaHCO₃ solution (100 mL). The aqueous layer was extracted with DCM (100 mL×2), and concentrated. The resulting residue was purified by column chromatography on silica gel (DCM/MeOH=50/1, v/v) to afford tert-butyl 4-(3-((3-fluoro-5-(5-((1R,2S)-2-fluorocyclopropyl)-1,2,4-oxadiazol-3-yl)-2-methylphenyl)carbamoyl)imidazo[1,2-a]pyridin-7-yl)piperazine-1-carboxylate and tert-butyl 4-(3-((3-fluoro-5-(5-((1S,2R)-2-fluorocyclopropyl)-1,2,4-oxadiazol-3-yl)-2-methylphenyl)carbamoyl)imidazo[1,2-a]pyridin-7-yl)piperazine-1-carboxylate (racemic, trans) as a white solid (300.0 mg, 38.7%). LCMS (M+H⁺) m/z calculated 580.2, found 580.1.

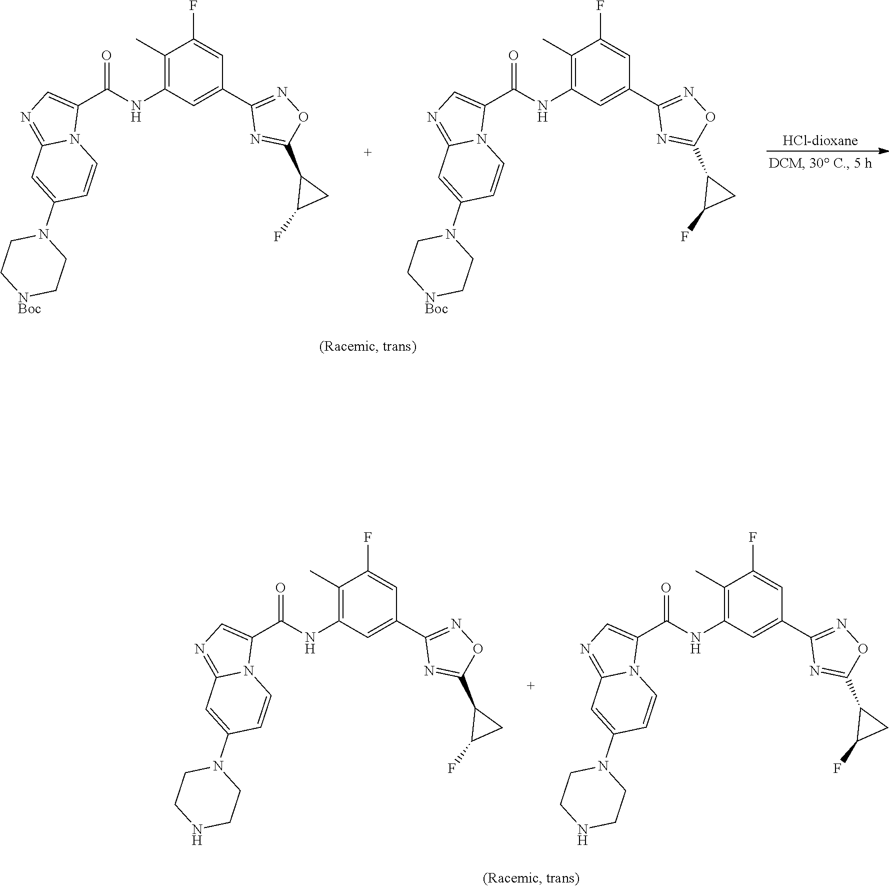 [SMILES: Cc1c(F)cc(-c2noc([C@H]3C[C@@H]3F)n2)cc1NC(=O)c1cnc2cc(N3CCN(C(=O)OC(C)(C)C)CC3)ccn12 ; Cc1c(F)cc(-c2noc([C@@H]3C[C@H]3F)n2)cc1NC(=O)c1cnc2cc(N3CCN(C(=O)OC(C)(C)C)CC3)ccn12 ; Cc1c(F)cc(-c2noc([C@H]3C[C@@H]3F)n2)cc1NC(=O)c1cnc2cc(N3CCNCC3)ccn12 ; Cc1c(F)cc(-c2noc([C@@H]3C[C@H]3F)n2)cc1NC(=O)c1cnc2cc(N3CCNCC3)ccn12]

To a stirring solution of tert-butyl 4-(3-((3-fluoro-5-(5-((1R,2S)-2-fluorocyclopropyl)-1,2,4-oxadiazol-3-yl)-2-methylphenyl)carbamoyl)imidazo[1,2-a]pyridin-7-yl)piperazine-1-carboxylate and tert-butyl 4-(3-((3-fluoro-5-(5-((1S,2R)-2-fluorocyclopropyl)-1,2,4-oxadiazol-3-yl)-2-methylphenyl)carbamoyl)imidazo[1,2-a]pyridin-7-yl)piperazine-1-carboxylate (racemic, trans, 300 mg, 0.52 mmol, 1.0 eq) in DCM (10 mL) was added 4 N HCl/dioxane (5 mL). The reaction was stirred at 30° C. for 5 h. The mixture was concentrated and purified by prep-HPLC to afford N-(3-fluoro-5-(5-((1R,2S)-2-fluorocyclopropyl)-1,2,4-oxadiazol-3-yl)-2-methylphenyl)-7-(piperazin-1-yl)imidazo[1,2-a]pyridine-3-carboxamide and N-(3-fluoro-5-(5-((1S,2R)-2-fluorocyclopropyl)-1,2,4-oxadiazol-3-yl)-2-methylphenyl)-7-(piperazin-1-yl)imidazo[1,2-a]pyridine-3-carboxamide(racemic, trans) as a white solid (189.5 mg, 76.4%). LCMS (M+H⁺) m/z calculated 480.2, found 480.1. ¹H NMR (DMSO-d₆, 400 MHz) δ 9.95 (s, 1H), 9.13 (d, 1H), 8.39 (s, 1H), 7.90 (s, 1H), 7.57 (d, 1H), 7.09 (dd, 1H), 6.87 (d, 1H), 5.20-5.38 (m, 1H), 3.29 (m, 8H), 2.66-2.68 (m, 1H), 2.23 (s, 3H), 1.90-2.00 (m, 1H), 1.57-1.62 (m, 1H).

### Example 23: Preparation of ethyl 3-(3-(3-fluoro-5-(imidazo[1,2-a]pyridine-3-carboxamido)-4-methylphenyl)-1,2,4-oxadiazol-5-yl)azetidine-1-carboxylate

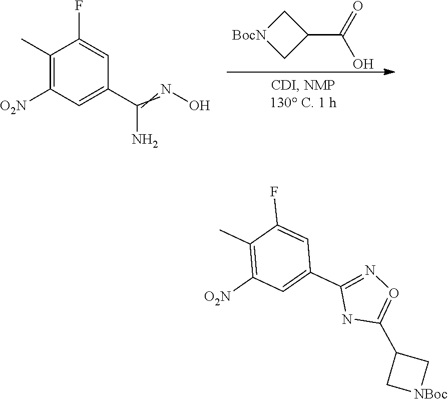

To a solution of 3,3-difluorocyclobutane-1-carboxylic acid (55.0 g, 258.2 mmol, 1.0 eq) in anhydrous NMP (180 mL) was added CDI (62.7 g, 287.3 mmol, 1.5 eq). The reaction was stirred for 15 minutes. And 1-(tert-butoxycarbonyl)azetidine-3-carboxylic acid (51.9 g, 258.2 mmol, 1.0 eq) was added and the reaction was stirred for 25 minutes. The reaction was stirred at 130° C. for 1 h. The crude product was diluted with EtOAc (300 mL). The organic layer was washed with water (200 mL) and brine (200 mL). The mixture was concentrated and purified by column chromatography on silica gel (PE/EA=5/1, v/v) to afford tert-butyl 3-(3-(3-fluoro-4-methyl-5-nitrophenyl)-1,2,4-oxadiazol-5-yl)azetidine-1-carboxylate as a yellow solid (100 g, ca 100%).

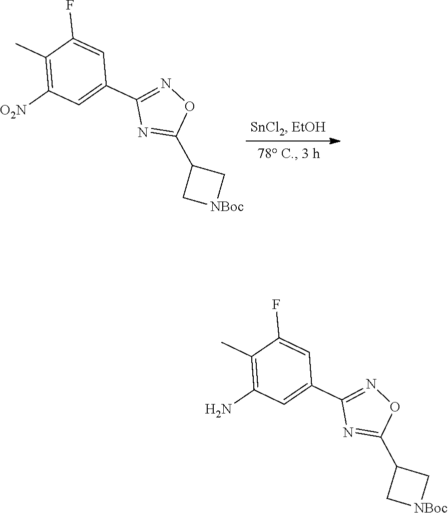 [SMILES: Cc1c(F)cc(-c2noc(C3CN(C(=O)OC(C)(C)C)C3)n2)cc1[N+](=O)[O-] ; Cc1c(N)cc(-c2noc(C3CN(C(=O)OC(C)(C)C)C3)n2)cc1F]

To a stirring suspension of tert-butyl 3-(3-(3-fluoro-4-methyl-5-nitrophenyl)-1,2,4-oxadiazol-5-yl)azetidine-1-carboxylate (100.0 g, 264.6 mmol, 1.0 eq) in EtOH (190 mL) was added SnCl₂ (150.5 g, 793.7 mmol, 3.0 eq). The reaction mixture was heated at 78° C. for 3 h. The reaction was cooled to rt and the solvent was partially concentrated. The pH was adjusted to slightly basic with saturated solution of sodium bicarbonate (190 mL). The resulting white suspension was filtered and washed with water (190 mL) and EtOAc (190 mL). The aqueous layer was extracted with EtOAc (250 mL×2). The combined organic layers were washed with brine (250 mL), dried over anhydrous sodium sulfate, filtered, and concentrated. The resulting residue was purified by column chromatography on silica gel (PE/EA=5/1, v/v) to afford tert-butyl 3-(3-(3-amino-5-fluoro-4-methylphenyl)-1,2,4-oxadiazol-5-yl)azetidine-1-carboxylate as a white solid (45.0 g. 48.9%). LCMS (M+H⁺) m/z calculated 349.2, found 349.1.

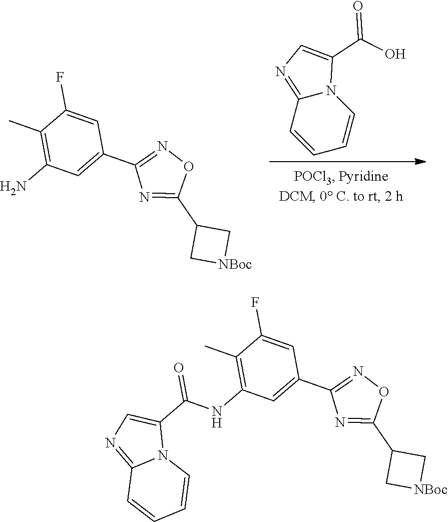 [SMILES: Cc1c(N)cc(-c2noc(C3CN(C(=O)OC(C)(C)C)C3)n2)cc1F ; O=C(O)c1cnc2ccccn12 ; Cc1c(F)cc(-c2noc(C3CN(C(=O)OC(C)(C)C)C3)n2)cc1NC(=O)c1cnc2ccccn12]

To a stirring solution of tert-butyl 3-(3-(3-amino-5-fluoro-4-methylphenyl)-1,2,4-oxadiazol-5-yl)azetidine-1-carboxylate (45 g, 129.3 mmol, 1.0 eq), imidazo[1,2-a]pyridine-3-carboxylic acid (20.9 g, 129.3 mmol, 1.0 eq) and pyridine (52.1 mL, 646.6 mmol, 5.0 eq) in DCM (30 mL) was added POCl₃ (36.0 mL, 387.9 mmol, 3.0 eq) at 0° C. The reaction mixture was stirred at rt for 2 h. The reaction mixture was diluted with water (150 mL) and extracted with DCM (250 mL×2). The combined organic layers were washed with brine (150 mL), dried over anhydrous sodium sulfate, filtered, and concentrated. The resulting residue was purified by column chromatography on silica gel (DCM/MeOH=50/1, v/v) to afford tert-butyl 3-(3-(3-fluoro-5-(imidazo[1,2-a]pyridine-3-carboxamido)-4-methylphenyl)-1,2,4-oxadiazol-5-yl)azetidine-1-carboxylate as a white solid (16.0 mg. 25.1%). LCMS (M+H⁺) m/z calculated 493.2, found 493.1.

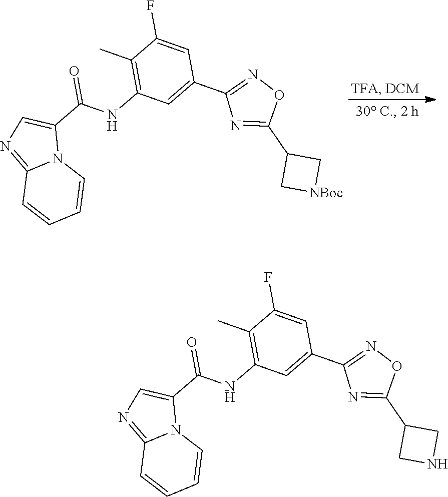 [SMILES: Cc1c(F)cc(-c2noc(C3CN(C(=O)OC(C)(C)C)C3)n2)cc1NC(=O)c1cnc2ccccn12 ; Cc1c(F)cc(-c2noc(C3CNC3)n2)cc1NC(=O)c1cnc2ccccn12]

To a stirring solution of tert-butyl 3-(3-(3-fluoro-5-(imidazo[1,2-a]pyridine-3-carboxamido)-4-methylphenyl)-1,2,4-oxadiazol-5-yl)azetidine-1-carboxylate (16 g, 32.5 mmol, 1.0 eq) in DCM (50 mL) was added TFA (30 mL). The reaction was stirred at 30° C. for 2 h. The mixture was concentrated to afford N-(5-(5-(azetidin-3-yl)-1,2,4-oxadiazol-3-yl)-3-fluoro-2-methylphenyl)imidazo[1,2-a]pyridine-3-carboxamide as a yellow oil (25.0 g, quant.). LCMS (M+H⁺) m/z calculated 392.2, found 392.1.

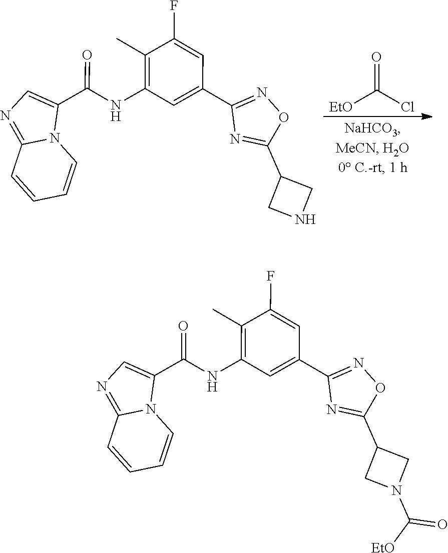 [SMILES: Cc1c(F)cc(-c2noc(C3CNC3)n2)cc1NC(=O)c1cnc2ccccn12 ; CCOC(=O)Cl ; CCOC(=O)N1CC(c2nc(-c3cc(F)c(C)c(NC(=O)c4cnc5ccccn45)c3)no2)C1]

N-(5-(5-(azetidin-3-yl)-1,2,4-oxadiazol-3-yl)-3-fluoro-2-methylphenyl)imidazo[1,2-a]pyridine-3-carboxamide (500 mg, 1.3 mmol, 1.0 eq) and NaHCO₃ (535.7 mg, 6.4 mmol, 5.0 eq) was dissolved in MeCN (50 mL) and water (5 mL). Ethyl carbonochloridate (165.3 mg, 1.5 mmol, 1.2 eq) was dissolved in MeCN (20 mL) and then added dropwise to the reaction mixture at 0° C. The reaction mixture was stirred for 1 h at rt. The reaction mixture was concentrated. The resulting residue was diluted with water (10 mL) and extracted with EtOAc (2×20 mL). The combined organic layers were dried over anhydrous sodium sulfate, filtered, and concentrated. The resulting residue was purified by Prep-HPLC to afford ethyl 3-(3-(3-fluoro-5-(imidazo[1,2-a]pyridine-3-carboxamido)-4-methylphenyl)-1,2,4-oxadiazol-5-yl)azetidine-1-carboxylate (65.3 mg, 11.0%). LCMS (M+H⁺) m/z calculated 465.2, found 465.1. ¹H NMR (CDCl₃, 400 MHz) δ 9.53 (d, 1H), 8.42 (s, 1H), 8.23 (s, 1H), 7.76 (d, 1H), 7.62-7.69 (m, 2H), 7.43-7.48 (m, 1H), 7.05-7.08 (m, 1H), 4.36-4.47 (m, 4H), 4.08-4.18 (m, 3H), 2.34 (d, 3H), 1.27 (t, 3H).

### Example 24: Preparation of Methyl 3-(3-(3-fluoro-5-(7-fluoroimidazo[1,2-a]pyridine-3-carboxamido)-4-methylphenyl)-1,2,4-oxadiazol-5-yl)azetidine-1-carboxylate

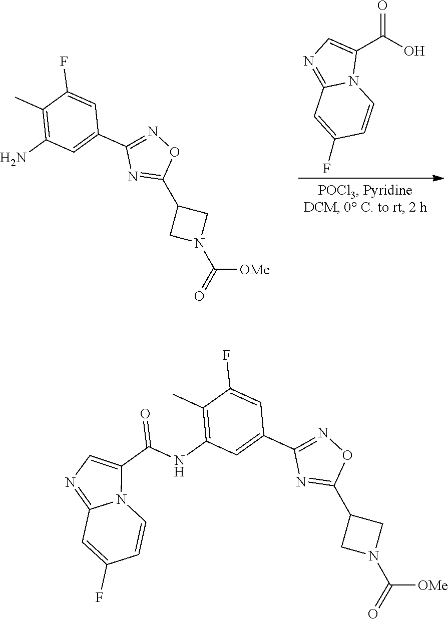 [SMILES: COC(=O)N1CC(c2nc(-c3cc(N)c(C)c(F)c3)no2)C1 ; O=C(O)c1cnc2cc(F)ccn12 ; COC(=O)N1CC(c2nc(-c3cc(F)c(C)c(NC(=O)c4cnc5cc(F)ccn45)c3)no2)C1]

To a solution of 7-fluoroimidazo[1,2-a]pyridine-3-carboxylic acid (100.0 mg, 0.56 mmol, 1.0 eq), methyl 3-(3-(3-amino-5-fluoro-4-methylphenyl)-1,2,4-oxadiazol-5-yl)azetidine-1-carboxylate (170.0 mg, 0.56 mmol, 1.0 eq) and pyridine (0.20 mL, 2.8 mmol, 5.0 eq) in DCM (30 mL) was added POCl₃ (0.15 mL, 1.7 mmol, 3.0 eq) at 0° C. The reaction mixture was stirred at rt for 2 h. The reaction mixture was diluted with water (50 mL) and extracted with DCM (50 mL×2). The combined organic layers were washed with brine (50 mL), dried over anhydrous sodium sulfate, filtered, and concentrated. The resulting residue was purified by Prep-HPLC to afford methyl 3-(3-(3-fluoro-5-(7-fluoroimidazo[1,2-a]pyridine-3-carboxamido)-4-methylphenyl)-1,2,4-oxadiazol-5-yl)azetidine-1-carboxylate (33.6 mg, 13.1%). LCMS (M+H⁺) m/z calculated 469.1, found 469.0. ¹H NMR (DMSO-d₆, 400 MHz) δ 10.25 (s, 1H), 9.47 (t, 1H), 8.59 (s, 1H), 7.98 (s, 1H), 7.69 (t, 2H), 7.26 (t, 1H), 4.19-4.40 (m, 5H), 3.60 (s, 3H), 2.25 (d, 3H).

### Example 25: Preparation of N-(5-(5-(3,3-difluorocyclobutyl)-1,2,4-oxadiazol-3-yl)-3-fluoro-2-methylphenyl)-6-(piperazin-1-yl)imidazo[1,2-a]pyridine-3-carboxamide

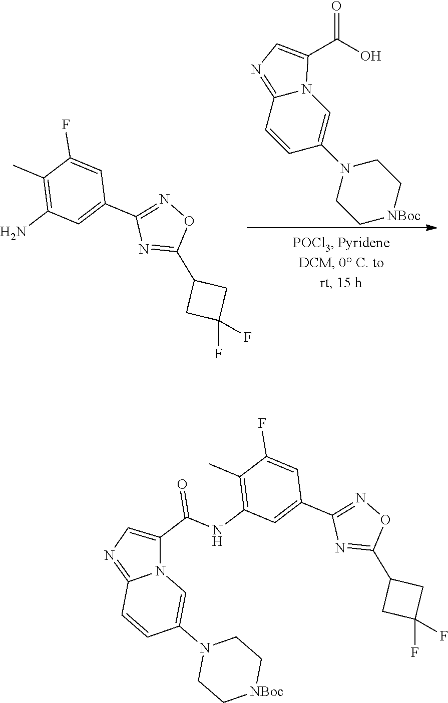 [SMILES: Cc1c(N)cc(-c2noc(C3CC(F)(F)C3)n2)cc1F ; CC(C)(C)OC(=O)N1CCN(c2ccc3ncc(C(=O)O)n3c2)CC1 ; Cc1c(F)cc(-c2noc(C3CC(F)(F)C3)n2)cc1NC(=O)c1cnc2ccc(N3CCN(C(=O)OC(C)(C)C)CC3)cn12]

To a solution of 6-(4-(tert-butoxycarbonyl)piperazin-1-yl)imidazo[1,2-a]pyridine-3-carboxylic acid (500.0 mg, 1.4 mmol, 1.0 eq), 5-(5-(3,3-difluorocyclobutyl)-1,2,4-oxadiazol-3-yl)-3-fluoro-2-methylaniline (409.0 mg, 1.4 mmol, 1.0 eq) and pyridine (0.60 mL, 7.2 mmol, 5.0 eq) in DCM (30 mL) was added POCl₃ (0.40 mL, 4.3 mmol, 3.0 eq) at 0° C. The reaction mixture was stirred at rt for 2 h, diluted with water (50 mL) and extracted with DCM (50 mL×2). The combined organic layers were washed with brine (50 mL), dried over anhydrous sodium sulfate, filtered, and concentrated to afford tert-butyl 4-(3-((5-(5-(3,3-difluorocyclobutyl)-1,2,4-oxadiazol-3-yl)-3-fluoro-2-methylphenyl)carbamoyl)imidazo[1,2-a]pyridin-6-yl)piperazine-1-carboxylate (250.0 mg, 28.3%). LCMS (M+H⁺) m/z calculated 612.2, found 612.1.

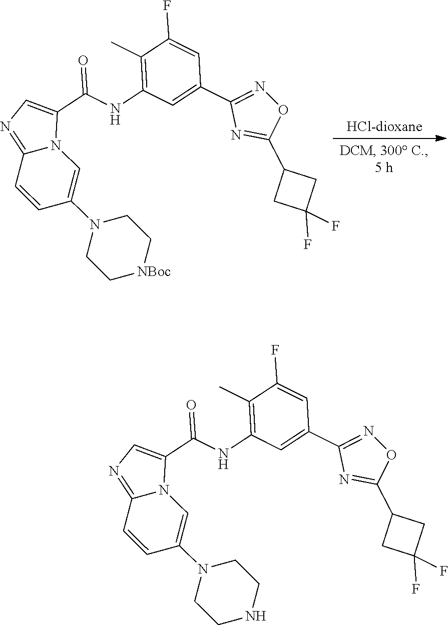 [SMILES: Cc1c(F)cc(-c2noc(C3CC(F)(F)C3)n2)cc1NC(=O)c1cnc2ccc(N3CCN(C(=O)OC(C)(C)C)CC3)cn12 ; Cc1c(F)cc(-c2noc(C3CC(F)(F)C3)n2)cc1NC(=O)c1cnc2ccc(N3CCNCC3)cn12]

To a stirring solution of tert-butyl 4-(3-((5-(5-(3,3-difluorocyclobutyl)-1,2,4-oxadiazol-3-yl)-3-fluoro-2-methylphenyl)carbamoyl)imidazo[1,2-a]pyridin-6-yl)piperazine-1-carboxylate (250 mg, 0.41 mmol, 1.0 eq) in DCM (10 mL) was added 4 N HCl/dioxane (10 mL). The reaction was stirred at 30° C. for 5 h. The mixture was concentrated and purified by prep-HPLC to afford N-(5-(5-(3,3-difluorocyclobutyl)-1,2,4-oxadiazol-3-yl)-3-fluoro-2-methylphenyl)-6-(piperazin-1-yl)imidazo[1,2-a]pyridine-3-carboxamide as a white solid (11.0 mg, 5.3%). LCMS (M+H⁺) m/z calculated 512.2, found 512.1. ¹H NMR (DMSO-d₆, 400 MHz) δ 10.13 (s, 1H), 8.91 (d, 1H), 8.49 (s, 1H), 7.95 (s, 1H), 7.64 (d, 2H), 7.52 (dd, 1H), 3.87-3.92 (m, 1H), 2.97-3.24 (m, 8H), 2.84-2.87 (m, 4H), 2.25 (d, 3H).

Example 26: Preparation of N-(3-fluoro-5-(5-((1R,2S)-2-fluorocyclopropyl)-1,2,4-oxadiazol-3-yl)-2-methylphenyl)-6-(piperazin-1-yl)imidazo[1,2-a]pyridine-3-carboxamide and N-(3-fluoro-5-(5-((1S,2R)-2-fluorocyclopropyl)-1,2,4-oxadiazol-3-yl)-2-methylphenyl)-6-(piperazin-1-yl)imidazo[1,2-a]pyridine-3-carboxamide (racemic, trans)

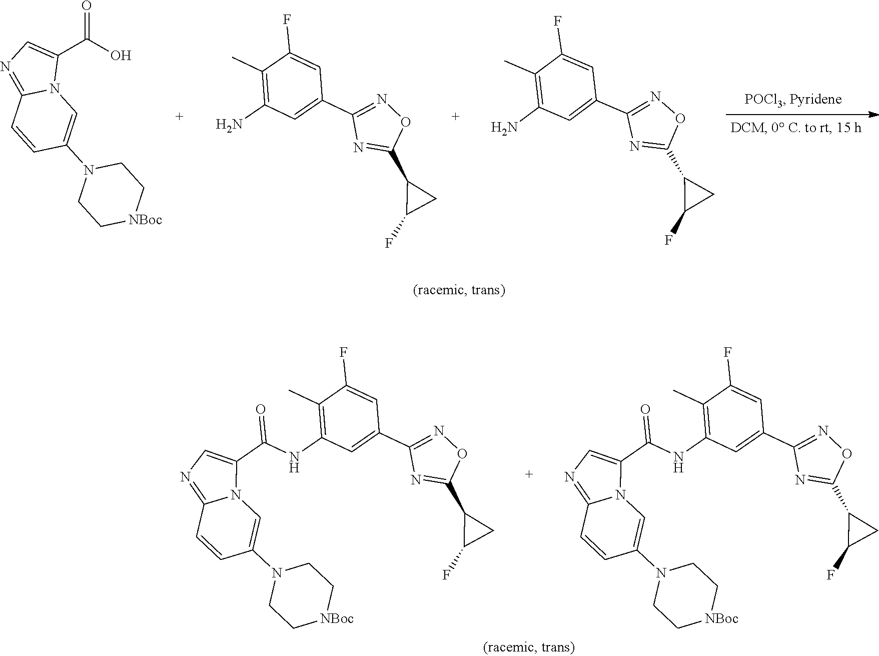

To a solution of 6-(4-(tert-butoxycarbonyl)piperazin-1-yl)imidazo[1,2-a]pyridine-3-carboxylic acid (1 g, 2.9 mmol, 1.0 eq), 3-fluoro-5-(5-((1R,2S)-2-fluorocyclopropyl)-1,2,4-oxadiazol-3-yl)-2-methylaniline and 3-fluoro-5-(5-((1S,2R)-2-fluorocyclopropyl)-1,2,4-oxadiazol-3-yl)-2-methylaniline (racemic, trans, 725.4 mg, 2.9 mmol, 1.0 eq) and pyridine (1.2 mL, 14.4 mmol, 5.0 eq) in DCM (50 mL) was added POCl₃ (0.80 mL, 8.7 mmol, 3.0 eq) at 0° C. The reaction mixture was stirred at 25° C. for 15 h. The reaction mixture was diluted with water (50 mL) and extracted with DCM (50 mL×2). The combined organic layers were washed with brine (50 mL), dried over anhydrous sodium sulfate, filtered, and concentrated to afford tert-butyl 4-(3-((3-fluoro-5-(5-((1R,2S)-2-fluorocyclopropyl)-1,2,4-oxadiazol-3-yl)-2-methylphenyl)carbamoyl)imidazo[1,2-a]pyridin-6-yl)piperazine-1-carboxylate and tert-butyl 4-(3-((3-fluoro-5-(5-((1S,2R)-2-fluorocyclopropyl)-1,2,4-oxadiazol-3-yl)-2-methylphenyl)carbamoyl)imidazo[1,2-a]pyridin-6-yl)piperazine-1-carboxylate (racemic, trans, 460.0 mg, 27.1%). LCMS (M+H⁺) m/z calculated 580.2, found 580.1.

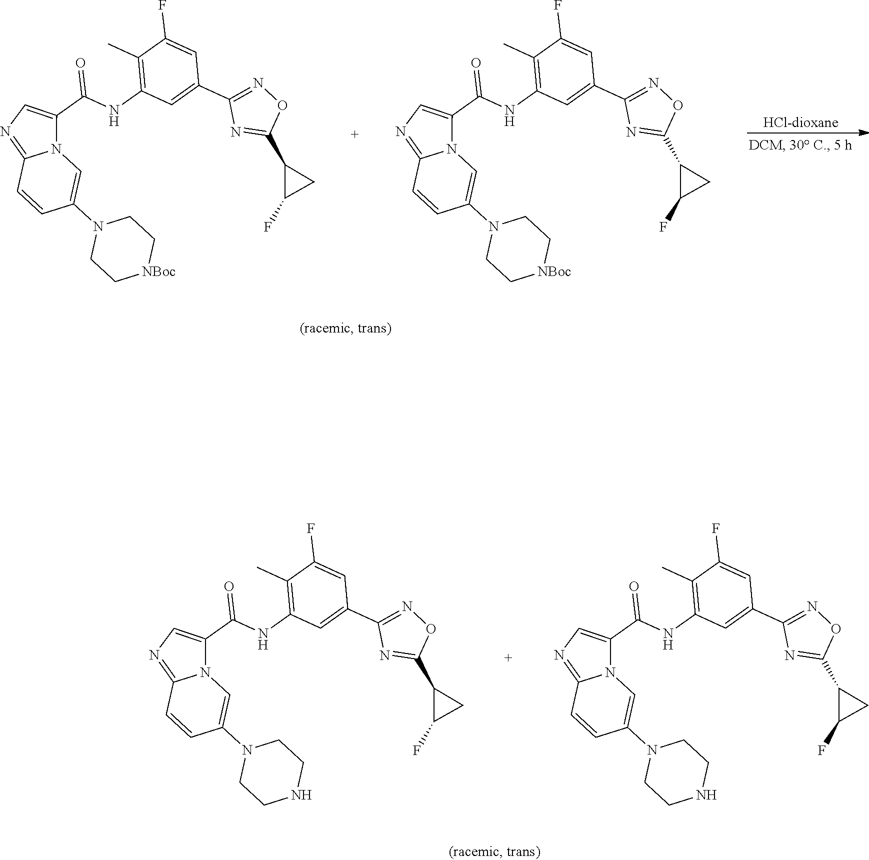 [SMILES: Cc1c(F)cc(-c2noc([C@H]3C[C@@H]3F)n2)cc1NC(=O)c1cnc2ccc(N3CCN(C(=O)OC(C)(C)C)CC3)cn12 ; Cc1c(F)cc(-c2noc([C@@H]3C[C@H]3F)n2)cc1NC(=O)c1cnc2ccc(N3CCN(C(=O)OC(C)(C)C)CC3)cn12 ; Cc1c(F)cc(-c2noc([C@H]3C[C@@H]3F)n2)cc1NC(=O)c1cnc2ccc(N3CCNCC3)cn12 ; Cc1c(F)cc(-c2noc([C@@H]3C[C@H]3F)n2)cc1NC(=O)c1cnc2ccc(N3CCNCC3)cn12]

To a stirring solution of tert-butyl 4-(3-((3-fluoro-5-(5-((1R,2S)-2-fluorocyclopropyl)-1,2,4-oxadiazol-3-yl)-2-methylphenyl)carbamoyl)imidazo[1,2-a]pyridin-6-yl)piperazine-1-carboxylate and tert-butyl 4-(3-((3-fluoro-5-(5-((1S,2R)-2-fluorocyclopropyl)-1,2,4-oxadiazol-3-yl)-2-methylphenyl)carbamoyl)imidazo[1,2-a]pyridin-6-yl)piperazine-1-carboxylate (racemic, trans, 300 mg, 0.79 mmol, 1.0 eq) in DCM (10 mL) was added 4 N HCl in dioxane (5 mL). The reaction was stirred at 30° C. for 5 h. The mixture was concentrated and purified by prep-HPLC to afford N-(3-fluoro-5-(5-((1R,2S)-2-fluorocyclopropyl)-1,2,4-oxadiazol-3-yl)-2-methylphenyl)-6-(piperazin-1-yl)imidazo[1,2-a]pyridine-3-carboxamide and N-(3-fluoro-5-(5-((1S,2R)-2-fluorocyclopropyl)-1,2,4-oxadiazol-3-yl)-2-methylphenyl)-6-(piperazin-1-yl)imidazo[1,2-a]pyridine-3-carboxamide (racemic, trans) as a white solid (20.2 mg, 5.3%). LCMS (M+H⁺) m/z calculated 480.2, found 480.1. ¹H NMR (DMSO-d₆, 400 MHz) δ 10.11 (s, 1H), 8.91 (d, 1H), 8.48 (s, 1H), 7.89 (s, 1H), 7.64 (d, 1H), 7.59 (d, 1H), 7.51 (dd, 1H), 5.20-5.38 (m, 1H), 2.97-3.11 (m, 6H), 2.83-2.87 (m, 4H), 2.24 (s, 3H), 1.90-2.00 (m, 1H), 1.57-1.62 (m, 1H).

### Example 27: Preparation of Methyl 3-(3-(3-fluoro-5-(6-methoxyimidazo[1,2-a]pyridine-3-carboxamido)-4-methylphenyl)-1,2,4-oxadiazol-5-yl)azetidine-1-carboxylate

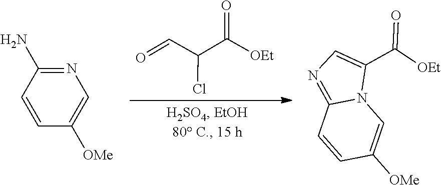 [SMILES: COc1ccc(N)nc1 ; CCOC(=O)C(Cl)C=O ; CCOC(=O)c1cnc2ccc(OC)cn12]

To a solution of ethyl 2-chloro-3-oxopropanoate (2.4 g, 16.1 mmol, 4.0 eq) and 5-methoxypyridin-2-amine (500 mg, 4.0 mmol, 1.0 eq) in EtOH (20 mL) was added conc. H₂SO₄ (0.3 mL). The reaction mixture was stirred at 80° C. for 15 h, and concentrated. The pH was adjusted to slightly basic with saturated solution of sodium bicarbonate. The resulting solution was extracted with EtOAc (80 mL×3). The combined organic phases were washed with brine (70 mL), dried over anhydrous sodium sulfate, filtered, and concentrated. The resulting residue was purified by column chromatography on silica gel (PE/EA=2/1, v/v) to afford ethyl 6-methoxyimidazo[1,2-a]pyridine-3-carboxylate as a yellow solid (669 mg, 75.4%). LCMS (M+H⁺) m/z calculated 221.1, found 221.0.

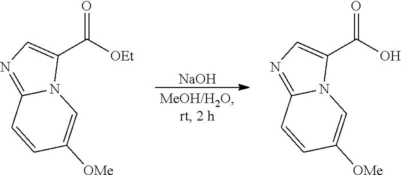 [SMILES: CCOC(=O)c1cnc2ccc(OC)cn12 ; "NaOH" ; COc1ccc2ncc(C(=O)O)n2c1]

A solution of ethyl 6-methoxyimidazo[1,2-a]pyridine-3-carboxylate (669 mg, 3.0 mmol, 1.0 eq) and NaOH (243.3 mg, 6.1 mmol, 2.0 eq) in MeOH/H₂O (30 mL/5 mL) was stirred at rt for 2 h. The solvent was partially removed under vacuum. The pH was adjusted to 5 with conc. HCl. The precipitate was collected by filtration and dried to afford 6-methoxyimidazo[1,2-a]pyridine-3-carboxylic acid as a white solid (386.0 mg, 66.2%). LCMS (M+H⁺) m/z calculated 193.1, found 193.0.

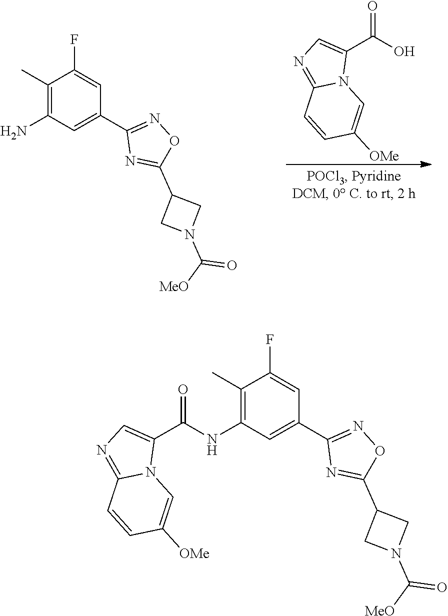 [SMILES: COC(=O)N1CC(c2nc(-c3cc(N)c(C)c(F)c3)no2)C1 ; COc1ccc2ncc(C(=O)O)n2c1 ; COC(=O)N1CC(c2nc(-c3cc(F)c(C)c(NC(=O)c4cnc5ccc(OC)cn45)c3)no2)C1]

To a solution of 6-methoxyimidazo[1,2-a]pyridine-3-carboxylic acid (150.0 mg, 0.78 mmol, 1.0 eq), methyl 3-(3-(3-amino-5-fluoro-4-methylphenyl)-1,2,4-oxadiazol-5-yl)azetidine-1-carboxylate (239.1 mg, 0.78 mmol, 1.0 eq) and pyridine (0.31 mL, 3.9 mmol, 5.0 eq) in DCM (20 mL) was added POCl₃ (0.21 mL, 2.3 mmol, 3.0 eq) at 0° C. The reaction mixture was stirred at rt for 2 h, diluted with water (50 mL) and extracted with DCM (50 mL×2). The combined organic layers were washed with brine (50 mL), dried over anhydrous sodium sulfate, filtered, and concentrated. The resulting residue was purified by Prep-HPLC to afford methyl 3-(3-(3-fluoro-5-(6-methoxyimidazo[1,2-a]pyridine-3-carboxamido)-4-methylphenyl)-1,2,4-oxadiazol-5-yl)azetidine-1-carboxylate (93.7 mg, 25.0%). LCMS (M+H⁺) m/z calculated 481.2, found 481.1. ¹H NMR (DMSO-d₆, 400 MHz) δ 10.19 (s, 1H), 9.10 (d, 1H), 8.55 (s, 1H), 7.98 (s, 1H), 7.72 (d, 1H), 7.67 (d, 1H), 7.32 (dd, 1H), 4.20-4.38 (m, 5H), 3.83 (s, 3H), 3.60 (s, 3H), 2.26 (s, 3H).

### Example 28: Preparation of Methyl 3-(3-(3-fluoro-4-methyl-5-(6-(4-methylpiperazin-1-yl)imidazo[1,2-a]pyridine-3-carboxamido)phenyl)-1,2,4-oxadiazol-5-yl)azetidine-1-carboxylate

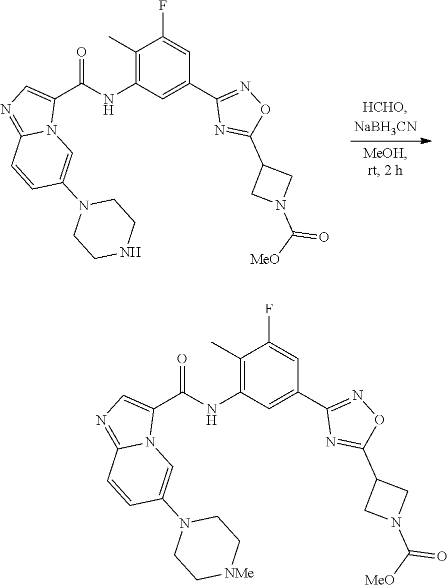 [SMILES: COC(=O)N1CC(c2nc(-c3cc(F)c(C)c(NC(=O)c4cnc5ccc(N6CCNCC6)cn45)c3)no2)C1 ; COC(=O)N1CC(c2nc(-c3cc(F)c(C)c(NC(=O)c4cnc5ccc(N6CCN(C)CC6)cn45)c3)no2)C1]

To a solution of methyl 3-(3-(3-fluoro-4-methyl-5-(6-(piperazin-1-yl)imidazo[1,2-a]pyridine-3-carboxamido)phenyl)-1,2,4-oxadiazol-5-yl)azetidine-1-carboxylate (50 mg, 0.094 mmol, 1.0 eq) in MeOH (15 mL) was added formaldehyde (37 wt % solution in water, 0.1 mL, 0.94 mmol, 10.0 eq) and NaBH₃CN (17.7 mg, 0.28 mmol, 3.0 eq). The solution was stirred at rt for 2 h, and then partitioned between DCM (20 mL) and saturated NaHCO₃ solution (20 mL). The aqueous layer was extracted with DCM (20 mL×2). The combined oraganic phases were concentrated under vacuum. The resulting residue was purified by prep-HPLC to afford methyl 3-(3-(3-fluoro-4-methyl-5-(6-(4-methylpiperazin-1-yl)imidazo[1,2-a]pyridine-3-carboxamido)phenyl)-1,2,4-oxadiazol-5-yl)azetidine-1-carboxylate as a white solid (12.4 mg, 24.3%). LCMS (M+H⁺) m/z calculated 549.2, found 549.1. ¹H NMR (DMSO-d₆, 400 MHz) δ 10.12 (s, 1H), 8.92 (d, 1H), 8.49 (s, 1H), 7.96 (s, 1H), 7.67 (d, 2H), 7.53 (dd, 1H), 4.20-4.38 (m, 5H), 3.59 (s, 3H), 3.07-3.11 (m, 4H), 2.50-2.53 (m, 4H), 2.25 (s, 3H), 2.22 (s, 3H).

### Example 29: Preparation of Methyl 3-(3-(3-fluoro-5-(7-methoxyimidazo[1,2-a]pyridine-3-carboxamido)-4-methylphenyl)-1,2,4-oxadiazol-5-yl)azetidine-1-carboxylate

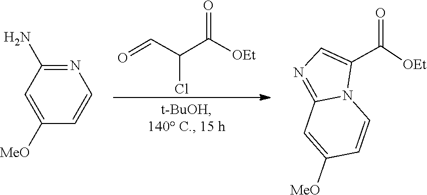 [SMILES: COc1ccnc(N)c1 ; CCOC(=O)C(Cl)C=O ; CCOC(=O)c1cnc2cc(OC)ccn12]

A solution of 4-methoxypyridin-2-amine (2.4 g, 16.1 mmol, 4.0 eq) and 5-methoxypyridin-2-amine (500 mg, 4.0 mmol, 1.0 eq) in t-BuOH (20 mL) was stirred at 140° C. for 15 h and concentrated in vacuum. The resulting residue was purified by column chromatography on silica gel (PE/EA=2/1, v/v) to afford ethyl 7-methoxyimidazo[1,2-a]pyridine-3-carboxylate as a yellow solid (320 mg, 36.1%). LCMS (M+H⁺) m/z calculated 221.1, found 221.0.

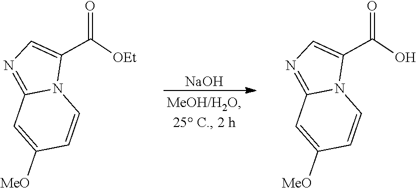 [SMILES: CCOC(=O)c1cnc2cc(OC)ccn12 ; "NaOH" ; COc1ccn2c(C(=O)O)cnc2c1]

A solution of ethyl 7-methoxyimidazo[1,2-a]pyridine-3-carboxylate (320 mg, 1.5 mmol, 1.0 eq) and NaOH (116.3 mg, 2.9 mmol, 2.0 eq) in MeOH/H₂O (30 mL/5 mL) was stirred at 25° C. for 2 h, and the solvent was partially removed under vacuum. The pH was adjusted to 5 with conc. HCl. The precipitate was collected by filtration and dried to afford 7-methoxyimidazo[1,2-a]pyridine-3-carboxylic acid as a white solid (250.0 mg, 89.6%). LCMS (M+H⁺) m/z calculated 193.1, found 193.0.

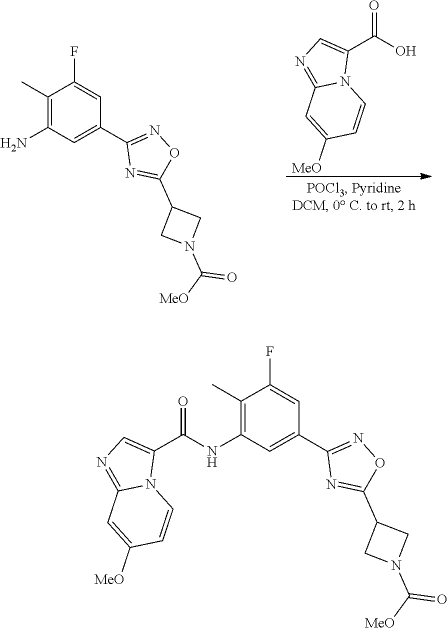 [SMILES: COC(=O)N1CC(c2nc(-c3cc(N)c(C)c(F)c3)no2)C1 ; COc1ccn2c(C(=O)O)cnc2c1 ; COC(=O)N1CC(c2nc(-c3cc(F)c(C)c(NC(=O)c4cnc5cc(OC)ccn45)c3)no2)C1]

To a solution of 7-methoxyimidazo[1,2-a]pyridine-3-carboxylic acid (250.0 mg, 1.3 mmol, 1.0 eq), methyl 3-(3-(3-amino-5-fluoro-4-methylphenyl)-1,2,4-oxadiazol-5-yl)azetidine-1-carboxylate (398.4 mg, 1.3 mmol, 1.0 eq) and pyridine (0.52 mL, 6.5 mmol, 5.0 eq) in DCM (20 mL) was added POCl₃ (0.36 mL, 3.9 mmol, 3.0 eq) at 0° C. The reaction mixture was stirred at 25° C. for 2 h. The reaction mixture was diluted with water (50 mL) and extracted with DCM (50 mL×2). The combined organic layers were washed with brine (50 mL), dried over anhydrous sodium sulfate, filtered, and concentrated. The resulting residue was purified by Prep-HPLC to afford methyl 3-(3-(3-fluoro-5-(7-methoxyimidazo[1,2-a]pyridine-3-carboxamido)-4-methylphenyl)-1,2,4-oxadiazol-5-yl)azetidine-1-carboxylate (189.5 mg, 30.3%). LCMS (M+H⁺) m/z calculated 481.2, found 481.1. ¹H NMR (DMSO-d₆, 400 MHz) δ 10.10 (s, 1H), 9.25 (d, 1H), 8.47 (s, 1H), 7.98 (s, 1H), 7.65 (dd, 1H), 7.17 (d, 1H), 6.87 (dd, 1H), 4.20-4.38 (m, 5H), 3.90 (s, 3H), 3.60 (s, 3H), 2.25 (d, 3H).

### Example 30: Preparation of Methyl 3-(3-(3-fluoro-4-methyl-5-(7-(piperazin-1-yl)imidazo[1,2-a]pyridine-3-carboxamido)phenyl)-1,2,4-oxadiazol-5-yl)azetidine-1-carboxylate

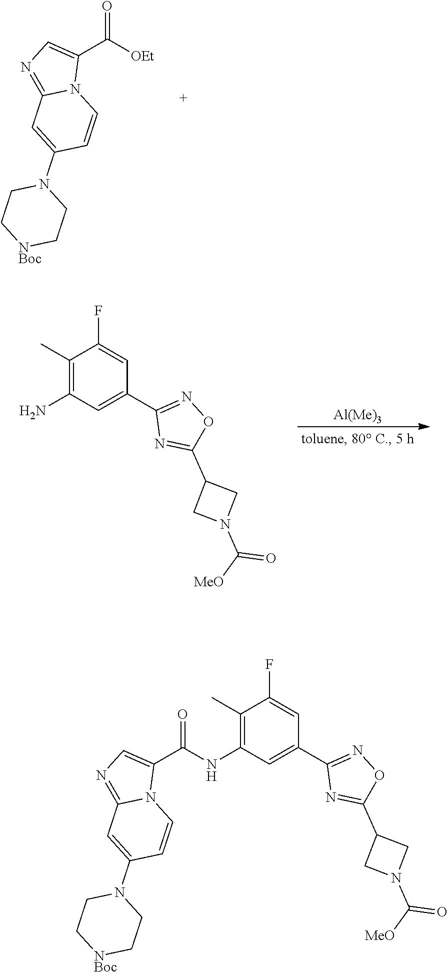 [SMILES: CCOC(=O)c1cnc2cc(N3CCN(C(=O)OC(C)(C)C)CC3)ccn12 ; COC(=O)N1CC(c2nc(-c3cc(N)c(C)c(F)c3)no2)C1 ; COC(=O)N1CC(c2nc(-c3cc(F)c(C)c(NC(=O)c4cnc5cc(N6CCN(C(=O)OC(C)(C)C)CC6)ccn45)c3)no2)C1 ; "Al(Me)3"]

To a stirring solution of ethyl 7-(4-(tert-butoxycarbonyl)piperazin-1-yl)imidazo[1,2-a]pyridine-3-carboxylate (500 mg, 1.3 mmol, 1.0 eq) and methyl 3-(3-(3-amino-5-fluoro-4-methylphenyl)-1,2,4-oxadiazol-5-yl)azetidine-1-carboxylate (409.1 mg, 1.3 mmol, 1.0 eq) in toluene (50 mL) was added Al(Me)₃ (2 M in toluene, 2.0 mL, 4.0 mmol, 3.0 eq) at 0° C. The reaction mixture was stirred at 80° C. for 5 h. The reaction mixture was concentrated under vacuum. The resulting residue was partitioned between DCM (100 mL) and saturated NaHCO₃ solution (100 mL). The aqueous layer was extracted with DCM (100 mL×2). The combined organic layers were concentrated and purified by column chromatography on silica gel (DCM/MeOH=50/1, v/v) to afford tert-butyl 4-(3-((3-fluoro-5-(5-(1-(methoxycarbonyl)azetidin-3-yl)-1,2,4-oxadiazol-3-yl)-2-methylphenyl)carbamoyl)imidazo[1,2-a]pyridin-7-yl)piperazine-1-carboxylate as a yellow solid (200.6 mg, 23.6%). LCMS (M+H⁺) m/z calculated 635.3, found 635.2.

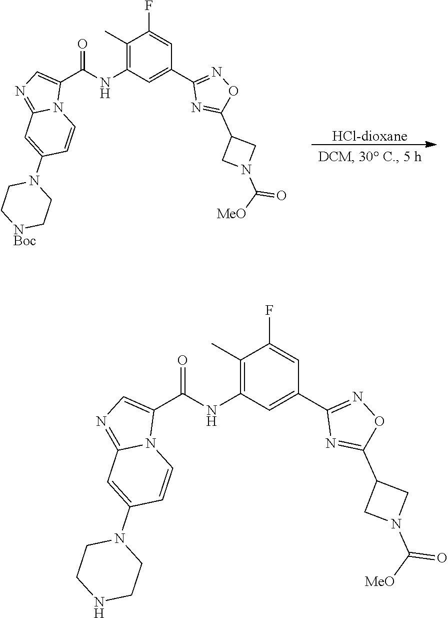 [SMILES: COC(=O)N1CC(c2nc(-c3cc(F)c(C)c(NC(=O)c4cnc5cc(N6CCN(C(=O)OC(C)(C)C)CC6)ccn45)c3)no2)C1 ; "HCl-dioxane" ; COC(=O)N1CC(c2nc(-c3cc(F)c(C)c(NC(=O)c4cnc5cc(N6CCNCC6)ccn45)c3)no2)C1]

To a stirring solution of tert-butyl 4-(3-((3-fluoro-5-(5-(1-(methoxycarbonyl)azetidin-3-yl)-1,2,4-oxadiazol-3-yl)-2-methylphenyl)carbamoyl)imidazo[1,2-a]pyridin-7-yl)piperazine-1-carboxylate (200 mg, 0.32 mmol, 1.0 eq) in DCM (10 mL) was added 4 N HCl in dioxane (5 mL). The reaction was stirred at 30° C. for 5 h. The mixture was concentrated and purified by prep-HPLC to afford methyl 3-(3-(3-fluoro-4-methyl-5-(7-(piperazin-1-yl)imidazo[1,2-a]pyridine-3-carboxamido)phenyl)-1,2,4-oxadiazol-5-yl)azetidine-1-carboxylate as a white solid (8.8 mg, 5.3%). LCMS (M+H⁺) m/z calculated 535.2, found 535.1. ¹H NMR (DMSO-d₆, 400 MHz) δ 9.94 (s, 1H), 9.13 (d, 1H), 8.39 (s, 1H), 7.98 (s, 1H), 7.62 (dd, 1H), 7.06 (dd, 1H), 6.84 (d, 1H), 4.19-4.38 (m, 5H), 3.59 (s, 3H), 3.22-3.31 (m, 4H), 2.82-2.85 (m, 4H), 2.24 (s, 3H).

### Example 31: Preparation of Cyclopropyl 3-(3-(3-fluoro-5-(imidazo[1,2-a]pyridine-3-carboxamido)-4-methylphenyl)-1,2,4-oxadiazol-5-yl)azetidine-1-carboxylate

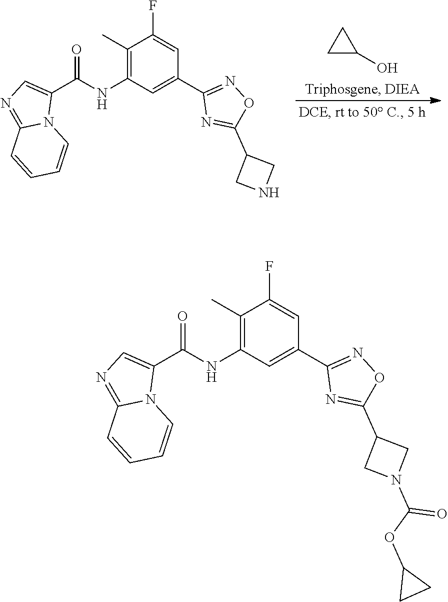

To a solution of cyclopropanol (29.6 mg, 0.51 mmol, 1.0 eq), TEA (515.3 mg, 5.1 mmol, 10.0 eq) in DCE (10 mL) was added triphosgene (151.5 mg, 0.51 mmol, 1.0 eq) at 0° C. The solution was warmed to rt and stirred for 1 h, and N-(5-(5-(azetidin-3-yl)-1,2,4-oxadiazol-3-yl)-3-fluoro-2-methylphenyl)imidazo[1,2-a]pyridine-3-carboxamide (200.0 mg, 0.51 mmol, 1.0 eq) was added. The resulting solution was heated at 50° C. for 1 h. After cooling to rt, the solution was diluted with DCM (50 mL). The resulting solution was washed with brine (30 mL), dried over anhydrous sodium suflate, filtered and concentrated. The resulting residue was purified by prep-HPLC to afford cyclopropyl 3-(3-(3-fluoro-5-(imidazo[1,2-a]pyridine-3-carboxamido)-4-methylphenyl)-1,2,4-oxadiazol-5-yl)azetidine-1-carboxylate (15.2 mg, 6.3%) as a white solid. LCMS (M+H⁺) m/z calculated 477.2, found 477.0. ¹H NMR (DMSO-d₆, 400 MHz) δ 9.52 (d, 1H), 8.49 (s, 1H), 8.02 (s, 1H), 7.69-7.76 (m, 2H), 7.58 (dd, 1H), 7.18 (t, 1H), 4.41-4.46 (m, 2H), 4.22-4.30 (m, 3H), 4.03 (t, 1H), 2.31 (s, 3H), 0.67-0.69 (m, 4H).

### Example 32: Preparation of N-(3-fluoro-2-methyl-5-(5-(1-(methylcarbamoyl)azetidin-3-yl)-1,2,4-oxadiazol-3-yl)phenyl)imidazo[1,2-a]pyridine-3-carboxamide

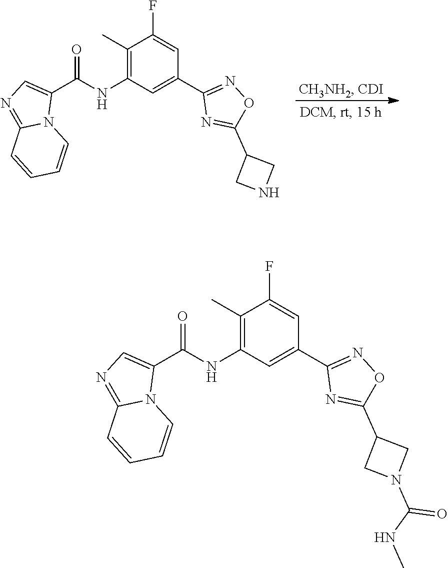

A solution of CDI (330.1 mg, 2.0 mmol, 4.0 eq) and Methylamine (1 M in THF, 2.0 mL, 2.0 mmol, 4.0 eq) in THE (10 ml) was stirred 15 minutes at rt. N-(5-(5-(azetidin-3-yl)-1,2,4-oxadiazol-3-yl)-3-fluoro-2-methylphenyl)imidazo[1,2-a]pyridine-3-carboxamide (200.0 mg, 0.51 mmol, 1.0 eq) was then added and the resulting mixture was stirred overnight at rt. The reaction mixture was diluted with water (15 ml) and extracted with EtOAc (20 mL×3). The combined organic layers were dried over anhydrous sodium sulfate, filtered, and concentrated. The resulting residue was purified by prep-HPLC to afford N-(3-fluoro-2-methyl-5-(5-(1-(methylcarbamoyl)azetidin-3-yl)-1,2,4-oxadiazol-3-yl)phenyl)imidazo[1,2-a]pyridine-3-carboxamide (25.6 mg, 11.2%) as a white solid. LCMS (M+H⁺) m/z calculated 450.2, found 450.0. ¹H NMR (DMSO-d₆, 400 MHz) δ 10.22 (s, 1H), 9.45 (d, 1H), 8.61 (s, 1H), 7.99 (s, 1 H), 7.79 (d, 1H), 7.67 (dd, 1H), 7.54 (dd, 1H), 7.19 (t, 1H), 6.43 (d, 1H), 4.22-4.24 (m, 3H), 4.05-4.07 (m, 2H), 2.55 (s, 3H), 2.25 (d, 3H).

### Example 33: Preparation of N-(5-(5-(3,3-difluorocyclobutyl)-1,2,4-oxadiazol-3-yl)-3-fluoro-2-methylphenyl)-6-(4-methylpiperazin-1-yl)imidazo[1,2-a]pyridine-3-carboxamide

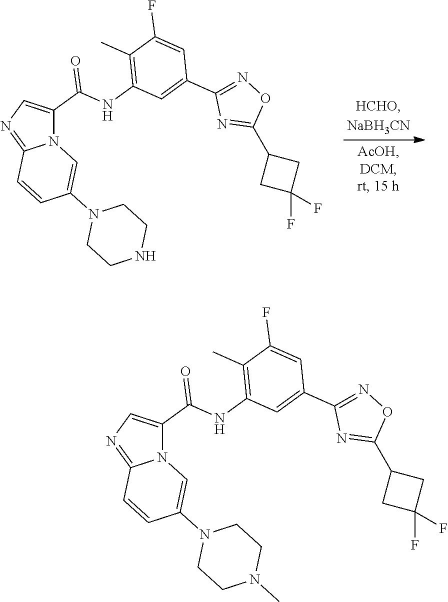 [SMILES: Cc1c(F)cc(-c2noc(C3CC(F)(F)C3)n2)cc1NC(=O)c1cnc2ccc(N3CCNCC3)cn12 ; Cc1c(F)cc(-c2noc(C3CC(F)(F)C3)n2)cc1NC(=O)c1cnc2ccc(N3CCN(C)CC3)cn12]

To a solution of methyl N-(5-(5-(3,3-difluorocyclobutyl)-1,2,4-oxadiazol-3-yl)-3-fluoro-2-methylphenyl)-6-(piperazin-1-yl)imidazo[1,2-a]pyridine-3-carboxamide (40 mg, 0.078 mmol, 1.0 eq) in MeOH (10 mL) was added formaldehyde (37 wt % solution in water, 0.1 mL, 0.78 mmol, 10.0 eq), NaBH₃CN (14.6 mg, 0.23 mmol, 3.0 eq) and AcOH (1 drop). The solution was stirred at rt for 15 h, and then partitioned between DCM (20 mL) and saturated NaHCO₃ solution (20 mL). The aqueous layer was extracted with DCM (20 mL×2), and the combined organic phases were concentrated. The resulting residue was purified by prep-HPLC to afford N-(5-(5-(3,3-difluorocyclobutyl)-1,2,4-oxadiazol-3-yl)-3-fluoro-2-methylphenyl)-6-(4-methylpiperazin-1-yl)imidazo[1,2-a]pyridine-3-carboxamide as a white solid (5.2 mg, 12.7%). LCMS (M+H⁺) m/z calculated 526.2, found 526.1. ¹H NMR (CDCl₃, 400 MHz) δ 9.08 (s, 1H), 8.34 (s, 1H), 8.17 (s, 1H), 7.63-7.67 (m, 3H), 7.32 (d, 1H), 3.64-3.69 (m, 1H), 3.37 (s, 4H), 3.08-3.17 (m, 4H), 2.87 (s, 4H), 2.55 (s, 3H), 2.33 (d, 3H).

Example 34: Preparation of N-(3-fluoro-5-(5-((1R,2S)-2-fluorocyclopropyl)-1,2,4-oxadiazol-3-yl)-2-methylphenyl)-6-(4-methylpiperazin-1-yl)imidazo[1,2-a]pyridine-3-carboxamide and N-(3-fluoro-5-(5-((1S,2R)-2-fluorocyclopropyl)-1,2,4-oxadiazol-3-yl)-2-methylphenyl)-6-(4-methylpiperazin-1-yl)imidazo[1,2-a]pyridine-3-carboxamide (racemic, trans)

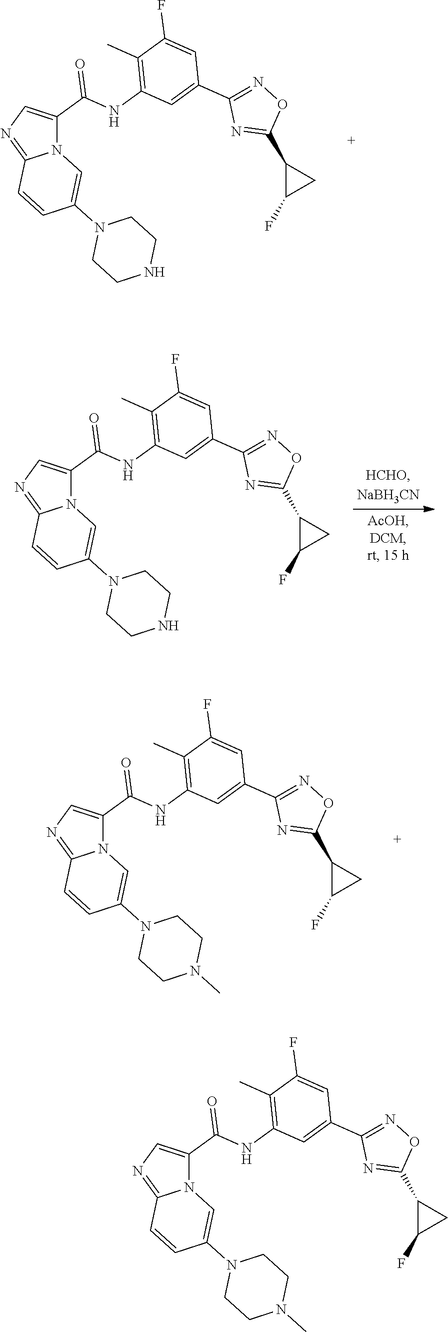 [SMILES: Cc1c(F)cc(-c2noc([C@H]3C[C@@H]3F)n2)cc1NC(=O)c1cnc2ccc(N3CCNCC3)cn12 ; Cc1c(F)cc(-c2noc([C@@H]3C[C@H]3F)n2)cc1NC(=O)c1cnc2ccc(N3CCNCC3)cn12 ; Cc1c(F)cc(-c2noc([C@H]3C[C@@H]3F)n2)cc1NC(=O)c1cnc2ccc(N3CCN(C)CC3)cn12 ; Cc1c(F)cc(-c2noc([C@@H]3C[C@H]3F)n2)cc1NC(=O)c1cnc2ccc(N3CCN(C)CC3)cn12]

To a solution of N-(3-fluoro-5-(5-((1R,2S)-2-fluorocyclopropyl)-1,2,4-oxadiazol-3-yl)-2-methylphenyl)-6-(piperazin-1-yl)imidazo[1,2-a]pyridine-3-carboxamide and N-(3-fluoro-5-(5-((1S,2R)-2-fluorocyclopropyl)-1,2,4-oxadiazol-3-yl)-2-methylphenyl)-6-(piperazin-1-yl)imidazo[1,2-a]pyridine-3-carboxamide (racemic, trans, 100 mg, 0.21 mmol, 1.0 eq) in MeOH (10 mL) were added formaldehyde (37 wt % solution in water, 0.3 mL, 2.1 mmol, 10.0 eq), NaBH₃CN (39.5 mg, 0.63 mmol, 3.0 eq) and AcOH (2 drops). The solution was stirred at rt for 15 h, and then partitioned between DCM (20 mL) and saturated NaHCO₃ solution (20 mL). The aqueous layer was extracted with DCM (20 mL×2). The combined organic phases were concentrated. The resulting residue was purified by prep-HPLC to N-(3-fluoro-5-(5-((1R,2S)-2-fluorocyclopropyl)-1,2,4-oxadiazol-3-yl)-2-methylphenyl)-6-(4-methylpiperazin-1-yl)imidazo[1,2-a]pyridine-3-carboxamide and N-(3-fluoro-5-(5-((1S,2R)-2-fluorocyclopropyl)-1,2,4-oxadiazol-3-yl)-2-methylphenyl)-6-(4-methylpiperazin-1-yl)imidazo[1,2-a]pyridine-3-carboxamide (racemic, trans) as a white solid (11.7 mg, 11.7%). LCMS (M+H⁺) m/z calculated 494.2, found 494.0. ¹H NMR (CDCl₃, 400 MHz) δ 9.06 (d, 1H), 8.29 (s, 1H), 8.14 (s, 1H), 7.59-7.65 (m, 3H), 7.31 (dd, 1H), 4.96-5.15 (m, 1H), 3.31 (s, 4H), 2.68-2.77 (m, 4H), 2.50 (s, 3H), 2.32 (d, 3H), 1.58-1.90 (m, 3H).

### Example 35: Preparation of Methyl 3-(3-(3-(7-cyanoimidazo[1,2-a]pyridine-3-carboxamido)-5-fluoro-4-methylphenyl)-1,2,4-oxadiazol-5-yl)azetidine-1-carboxylate

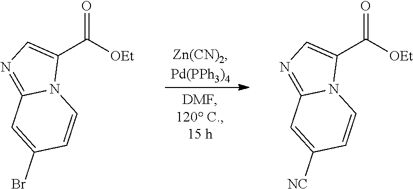 [SMILES: CCOC(=O)c1cnc2cc(Br)ccn12 ; CCOC(=O)c1cnc2cc(C#N)ccn12]

To a solution of ethyl 7-bromoimidazo[1,2-a]pyridine-3-carboxylate (1 g, 3.7 mmol, 1.0 eq) in DMF (50 mL) were added Zn(CN)₂ (654.9 mg, 5.6 mmol. 1.5 eq) and Pd(PPh₃)₄ (431.3 mg,
0.37 mmol, 0.1 eq). The mixture was stirred at 120° C. for 15 h, then the reaction mixture was diluted with water (100 mL) and extracted with EtOAc (100 mL×3). The combined organic layers were washed with brine (100 mL×2), dried with anhydrous sodium sulfate, filtered, and concentrated. The resulting residue was purified by column chromatography on silica gel (PE/EA=3/1, v/v) to afford ethyl 7-cyanoimidazo[1,2-a]pyridine-3-carboxylate as a yellow solid (600.0 g, 74.8%). LCMS (M+H⁺) m/z calculated 216.1, found 216.0.

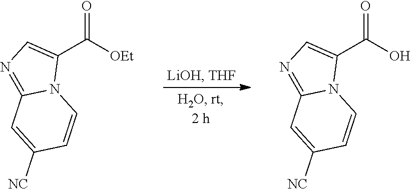 [SMILES: CCOC(=O)c1cnc2cc(C#N)ccn12 ; N#Cc1ccn2c(C(=O)O)cnc2c1]

A solution of ethyl 7-cyanoimidazo[1,2-a]pyridine-3-carboxylate (600.0 mg, 2.8 mmol, 1.0 eq) and LiOH (134.0 mg, 5.6 mmol, 2.0 eq) in THF/H₂O (30 mL/5 mL) was stirred at rt for 2 h, and the solvent was partially removed under vacuum. The pH was adjusted to 5 with conc. HCl. The precipitate was collected by vacuum filtration and dried under vacuum to afford 7-cyanoimidazo[1,2-a]pyridine-3-carboxylic acid as a white solid (500.0 mg, 95.9%). LCMS (M+H⁺) m/z calculated 188.0, found 187.9.

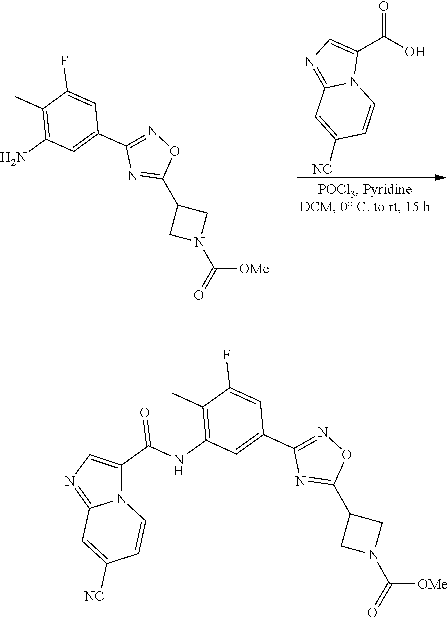 [SMILES: COC(=O)N1CC(c2nc(-c3cc(N)c(C)c(F)c3)no2)C1 ; N#Cc1ccn2c(C(=O)O)cnc2c1 ; COC(=O)N1CC(c2nc(-c3cc(F)c(C)c(NC(=O)c4cnc5cc(C#N)ccn45)c3)no2)C1]

To a solution of 7-cyanoimidazo[1,2-a]pyridine-3-carboxylic acid (500.0 mg, 2.7 mmol, 1.0 eq), methyl 3-(3-(3-amino-5-fluoro-4-methylphenyl)-1,2,4-oxadiazol-5-yl)azetidine-1-carboxylate (818.2 mg, 2.7 mmol, 1.0 eq) and pyridine (1.1 mL, 13.4 mmol, 5.0 eq) in DCM (50.0 mL) was added POCl₃ (0.75 mL, 3.9 mmol, 3.0 eq) at 0° C. The reaction mixture was stirred at rt for 15 h, diluted with water (50 mL) and extracted with DCM (50 mL×2). The combined organic layers were washed with brine (50 mL), dried over anhydrous sodium sulfate, filtered, and concentrated. The resulting residue was purified by Prep-HPLC to afford methyl 3-(3-(3-(7-cyanoimidazo[1,2-a]pyridine-3-carboxamido)-5-fluoro-4-methylphenyl)-1,2,4-oxadiazol-5-yl)azetidine-1-carboxylate (54.0 mg, 4.3%). LCMS (M+H⁺) m/z calculated 476.1, found 476.0. ¹H NMR (DMSO-d₆, 400 MHz) δ 10.45 (s, 1H), 9.52 (d, 1H), 8.78 (s, 1H), 8.58 (s, 1H), 7.99 (s, 1H), 7.68 (dd, 1H), 7.47 (dd, 1H), 4.19-4.38 (m, 5H), 3.60 (s, 3H), 2.25 (d, 3H).

### Example 36: Preparation of Methyl 3-(3-(3-(6-(4-acetylpiperazin-1-yl)imidazo[1,2-a]pyridine-3-carboxamido)-5-fluoro-4-methylphenyl)-1,2,4-oxadiazol-5-yl)azetidine-1-carboxylate

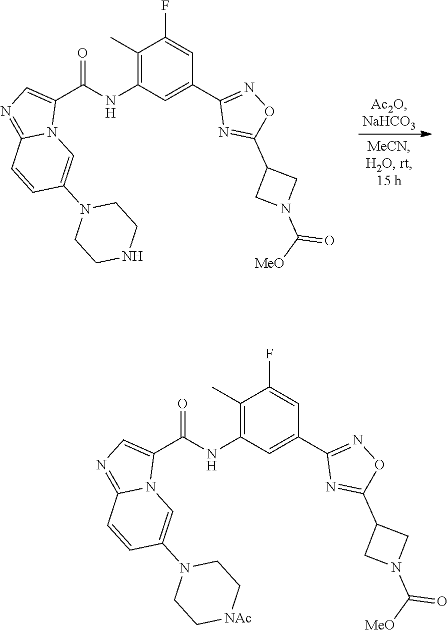

To a solution of methyl 3-(3-(3-fluoro-4-methyl-5-(6-(piperazin-1-yl)imidazo[1,2-a]pyridine-3-carboxamido)phenyl)-1,2,4-oxadiazol-5-yl)azetidine-1-carboxylate (40 mg, 0.075 mmol, 1.0 eq) in MeCN/H₂O (15 mL/3 mL) was added acetic anhydride (38.2 mg, 0.37 mmol, 5.0 eq) and NaHCO₃ (62.9 mg, 0.75 mmol, 10.0 eq). The solution was stirred at rt for 15 h, and then partitioned between EtOAc (20 mL) and saturated NaHCO₃ solution (20 mL). The aqueous layer was extracted with EtOAc (2×20 mL). The combined organic phases were concentrated under vacuum. The resulting residue was purified by prep-HPLC to afford methyl 3-(3-(3-(6-(4-acetylpiperazin-1-yl)imidazo[1,2-a]pyridine-3-carboxamido)-5-fluoro-4-methylphenyl)-1,2,4-oxadiazol-5-yl)azetidine-1-carboxylate as a white solid (1.8 mg, 4.2%). LCMS (M+H⁺) m/z calculated 577.2, found 577.1. ¹H NMR (DMSO-d₆, 400 MHz) δ 10.13 (s, 1H), 8.95 (d, 1H), 8.51 (s, 1H), 7.98 (s, 1H), 7.68 (t, 2H), 7.58 (d, 1H), 4.19-4.38 (m, 5H), 3.59-3.62 (m, 7H), 3.04-3.12 (m, 4H), 2.25 (s, 3H), 2.04 (s, 3H).

### Example 37: Preparation of Methyl 3-(5-(3-fluoro-5-(imidazo[1,2-a]pyridine-3-carboxamido)-4-methylphenyl)-1,2,4-oxadiazol-3-yl)azetidine-1-carboxylate

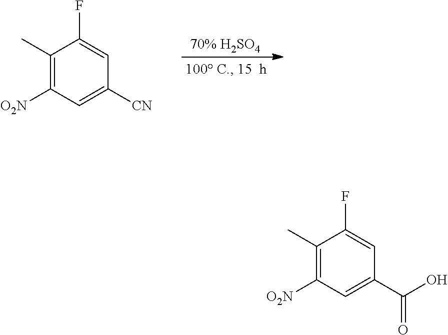

The mixture of 3-fluoro-4-methyl-5-nitrobenzonitrile (5.0 g, 27.8 mmol, 1.0 eq) in 50 mL H₂SO₄ (70% in H₂O) was stirred at 100° C. for 15 h. The mixture was poured into ice water (180 mL) and adjusted to pH=5 by adding saturated aqueous Na₂CO₃. The formed precipitate was filtered and dired to afford 3-fluoro-4-methyl-5-nitrobenzoic acid (3.1 g, 56.3%) as light grey solid, LCMS (M+H⁺) m/z calculated 200.0, found 199.9.

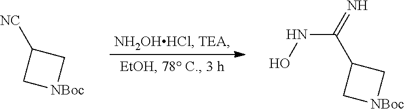 [SMILES: CC(C)(C)OC(=O)N1CC(C#N)C1 ; CC(C)(C)OC(=O)N1CC(C(=N)NO)C1]

A solution of tert-butyl 3-cyanoazetidine-1-carboxylate (5.0 mg, 27.5 mmol, 1.0 eq), NH2OH·HCl (2.9 g, 41.2 mmol, 1.5 eq) and TEA (17.6 mL, 54.9 mmol, 2.0 eq) in EtOH (30 mL) was stirred at 78° C. for 3 h, and the solvent was partially removed under vacuum. The crude product was diluted with EtOAc (200 mL). The organic layer was washed with water (200 mL) and brine (200 mL). The mixture was concentrated under vacuum to afford tert-butyl 3-(N-hydroxycarbamimidoyl)azetidine-1-carboxylate as a yellow solid (5.5 g, 93.2%). LCMS (M+H⁺) m/z calculated 216.1, found 216.0.

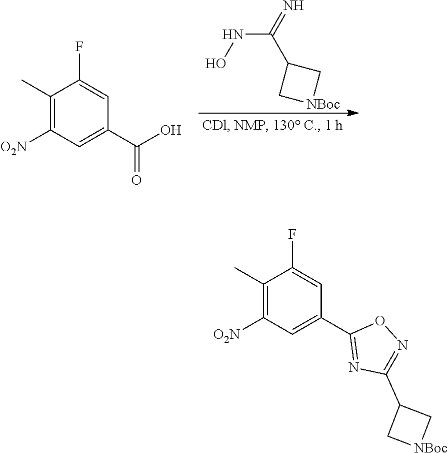 [SMILES: Cc1c(F)cc(C(=O)O)cc1[N+](=O)[O-] ; CC(C)(C)OC(=O)N1CC(C(=N)NO)C1 ; Cc1c(F)cc(-c2nc(C3CN(C(=O)OC(C)(C)C)C3)no2)cc1[N+](=O)[O-]]

To a solution of 3-fluoro-4-methyl-5-nitrobenzoic acid (2.0 g, 10.1 mmol, 1.0 eq) in anhydrous NMP (80 mL) was added CDI (2.5 g, 15.1 mmol, 1.5 eq) at rt. The reaction was stirred for 15 minutes, and tert-butyl 3-(N-hydroxycarbamimidoyl)azetidine-1-carboxylate (2.2 g, 10.1 mmol, 1.0 eq) was added. The reaction was stirred for 25 minutes, and then stirred at 130° C. for 1 h. The crude product was diluted with EtOAc (200 mL). The organic layer was washed with water (200 mL), brine (200 mL), fitered, and concentrated. The resulting residue was purified by column chromatography on silica gel (PE/EA=5/1, v/v) to afford tert-butyl 3-(5-(3-fluoro-4-methyl-5-nitrophenyl)-1,2,4-oxadiazol-3-yl)azetidine-1-carboxylate as a yellow solid (1.2 g, 31.6%). LCMS (M+H⁺) m/z calculated 379.1, found 379.0.

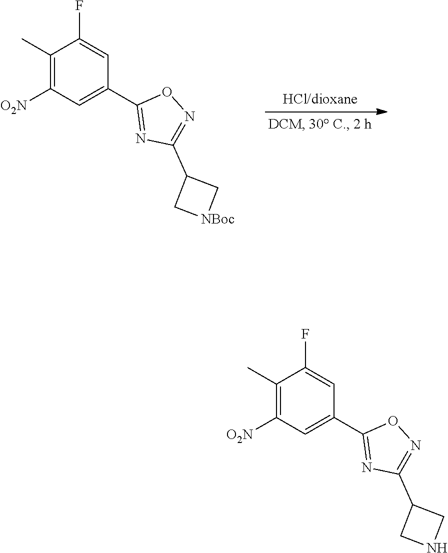 [SMILES: Cc1c(F)cc(-c2nc(C3CN(C(=O)OC(C)(C)C)C3)no2)cc1[N+](=O)[O-] ; Cc1c(F)cc(-c2nc(C3CNC3)no2)cc1[N+](=O)[O-]]

To a stirring solution of tert-butyl 3-(5-(3-fluoro-4-methyl-5-nitrophenyl)-1,2,4-oxadiazol-3-yl)azetidine-1-carboxylate (1.2 g, 3.2 mmol, 1.0 eq) in DCM (10 mL) was added 4 N HCl in dioxane (5 mL). The reaction was stirred at 30° C. for 2 h. The mixture was concentrated to afford 3-(azetidin-3-yl)-5-(3-fluoro-4-methyl-5-nitrophenyl)-1,2,4-oxadiazole as a yellow solid (1.2 g, quant.). LCMS (M+H⁺) m/z calculated 279.1, found 279.0.

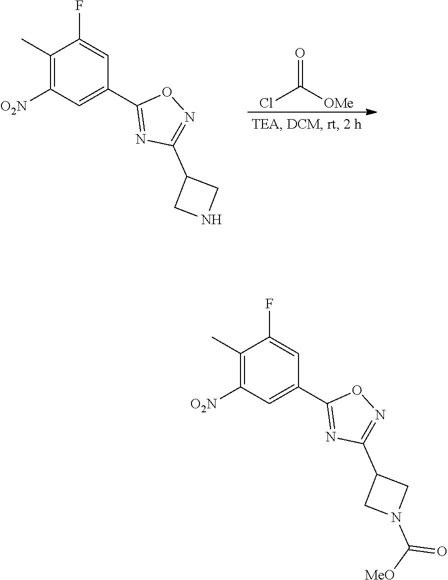 [SMILES: Cc1c(F)cc(-c2nc(C3CNC3)no2)cc1[N+](=O)[O-] ; COC(=O)Cl ; COC(=O)N1CC(c2noc(-c3cc(F)c(C)c([N+](=O)[O-])c3)n2)C1]

To a solution of 3-(azetidin-3-yl)-5-(3-fluoro-4-methyl-5-nitrophenyl)-1,2,4-oxadiazole (1.2 g, 4.3 mmol, 1.0 eq) in DCM (50 mL) was added methyl carbonochloridate (486.9 mg, 5.2 mmol, 1.2 eq) and TEA (3.0 mL, 21.6 mmol, 5.0 eq). The solution was stirred at rt for 2 h, and then partitioned between DCM (20 mL) and saturated NaHCO₃ solution (20 mL). The aqueous layer was extracted with DCM (20 mL×2), and the organic layers were concentrated to afford methyl 3-(5-(3-fluoro-4-methyl-5-nitrophenyl)-1,2,4-oxadiazol-3-yl)azetidine-1-carboxylate as a yellow solid (900.0 mg, 60.0%). LCMS (M+H⁺) m/z calculated 337.1, found 337.0.

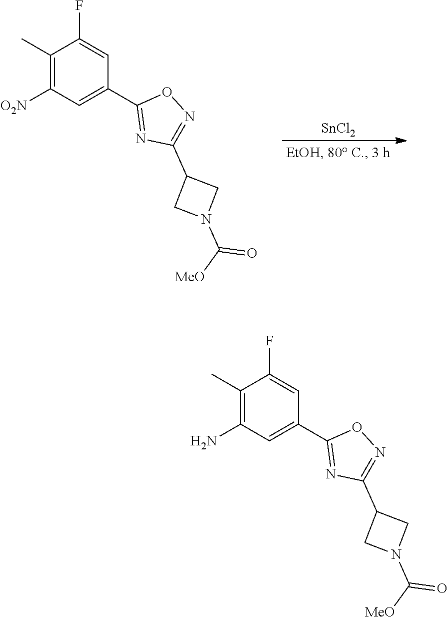 [SMILES: COC(=O)N1CC(c2noc(-c3cc(F)c(C)c([N+](=O)[O-])c3)n2)C1 ; "SnCl2" ; COC(=O)N1CC(c2noc(-c3cc(N)c(C)c(F)c3)n2)C1]

To a stirring suspension of methyl 3-(5-(3-fluoro-4-methyl-5-nitrophenyl)-1,2,4-oxadiazol-3-yl)azetidine-1-carboxylate (900 mg, 2.7 mmol, 1.0 eq) in EtOH (50 mL) was added SnCl₂ (1.5 g, 8.1 mmol, 3.0 eq). The reaction mixture was heated at 80° C. for 3 h. The reaction was cooled to rt and the solvent was partially concentrated. The pH was adjusted to slightly basic with saturated solution of sodium bicarbonate (90 mL). The resulting white suspension was filtered and washed with water (90 mL) and EtOAc (90 mL). The aqueous layer was extracted with EtOAc (150 mL×2). The combined organic layers were washed with brine (150 mL), dried over anhydrous sodium sulfate, filtered concentrated. The resulting residue was purified by column chromatography on silica gel (PE/EA=5/1, v/v) to afford methyl 3-(5-(3-amino-5-fluoro-4-methylphenyl)-1,2,4-oxadiazol-3-yl)azetidine-1-carboxylate as a white solid (550 mg. 63.5%). LCMS (M+H⁺) m/z calculated 307.1, found 307.0.

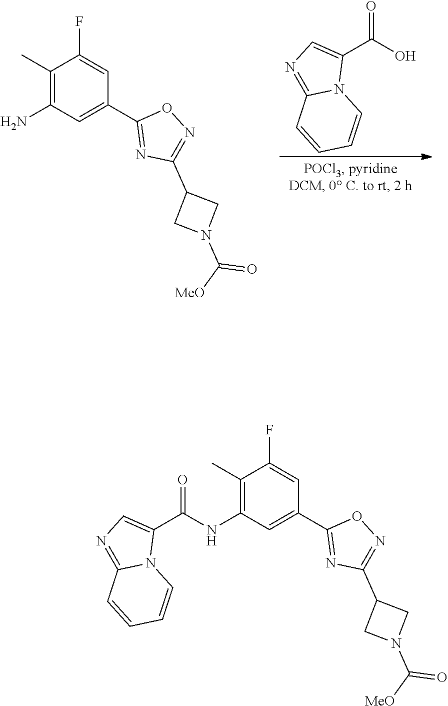 [SMILES: COC(=O)N1CC(c2noc(-c3cc(N)c(C)c(F)c3)n2)C1 ; O=C(O)c1cnc2ccccn12 ; COC(=O)N1CC(c2noc(-c3cc(F)c(C)c(NC(=O)c4cnc5ccccn45)c3)n2)C1]

To a solution of methyl 3-(5-(3-amino-5-fluoro-4-methylphenyl)-1,2,4-oxadiazol-3-yl)azetidine-1-carboxylate (200 mg, 0.65 mmol, 1.0 eq), imidazo[1,2-a]pyridine-3-carboxylic acid (116.5 mg, 0.72 mmol, 1.1 eq) and pyridine (0.3 mL, 1.3 mmol, 5.0 eq) in DCM (30 mL) was added POCl₃ (0.2 mL, 2.0 mmol, 3.0 eq) at 0° C. The reaction mixture was stirred at rt for 2 h. The reaction mixture was diluted with water (50 mL). The aqueous layer was extracted with DCM (50 mL×2). The combined organic layers were washed with brine (50 mL), dried over anhydrous sodium sulfate, filtered, and concentrated. The resulting residue was purified by prep-HPLC to afford methyl 3-(5-(3-fluoro-5-(imidazo[1,2-a]pyridine-3-carboxamido)-4-methylphenyl)-1,2,4-oxadiazol-3-yl)azetidine-1-carboxylate as a white solid (33.4 mg. 11.2%). LCMS (M+H⁺) m/z calculated 451.1, found 451.1. ¹H NMR (DMSO-d₆, 400 MHz) δ 9.45 (d, 1H), 8.61 (s, 1H), 8.12 (s, 1H), 7.79-7.83 (m, 2H), 7.54 (dd, 1H), 7.19 (t, 1H), 4.12-4.36 (m, 5H), 3.60 (s, 3H), 2.29 (s, 3H).

### Example 38: Preparation of 6-(4-acetylpiperazin-1-yl)-N-(5-(5-(3,3-difluorocyclobutyl)-1,2,4-oxadiazol-3-yl)-3-fluoro-2-methylphenyl)imidazo[1,2-a]pyridine-3-carboxamide

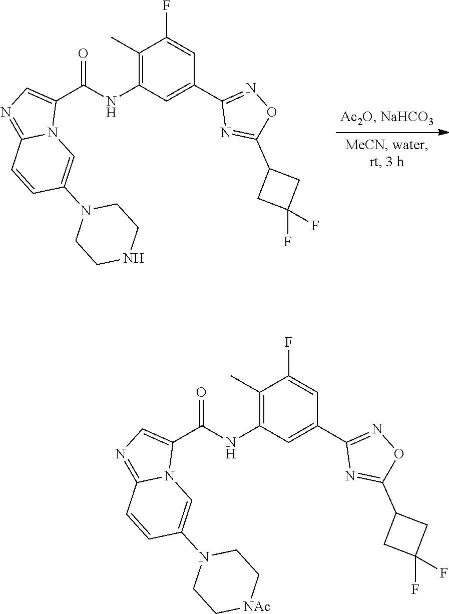

To a solution of N-(5-(5-(3,3-difluorocyclobutyl)-1,2,4-oxadiazol-3-yl)-3-fluoro-2-methylphenyl)-6-(piperazin-1-yl)imidazo[1,2-a]pyridine-3-carboxamide (40 mg, 0.078 mmol, 1.0 eq) in MeCN/H₂O (15 mL/3 mL) was added acetic anhydride (39.9 mg, 0.39 mmol, 5.0 eq) and NaHCO₃ (65.7 mg, 0.78 mmol, 10.0 eq). The solution was stirred at rt for 15 h, and then partitioned between EtOAc (20 mL) and saturated NaHCO₃ solution (20 mL). The aqueous layer was extracted with EtOAc (20 mL×2) concentrated. The resulting residue was purified by prep-HPLC to afford 6-(4-acetylpiperazin-1-yl)-N-(5-(5-(3,3-difluorocyclobutyl)-1,2,4-oxadiazol-3-yl)-3-fluoro-2-methylphenyl)imidazo[1,2-a]pyridine-3-carboxamide as a white solid (17.6 mg, 40.6%). LCMS (M+H⁺) m/z calculated 554.2, found 554.1. ¹H NMR (CDCl₃, 400 MHz) δ 9.18 (s, 1H), 8.75 (s, 1H), 8.59 (s, 1H), 8.25 (s, 1H), 7.77 (d, 1H), 7.67 (d, 1H), 7.44 (d, 1H), 3.64-3.82 (m, 5H), 3.08-3.19 (m, 8H), 2.36 (s, 3H), 2.15 (s, 3H).

### Example 39: Preparation of 6-(4-acetylpiperazin-1-yl)-N-(3-fluoro-5-(5-((1R,2S)-2-fluorocyclopropyl)-1,2,4-oxadiazol-3-yl)-2-methylphenyl)imidazo[1,2-a]pyridine-3-carboxamide

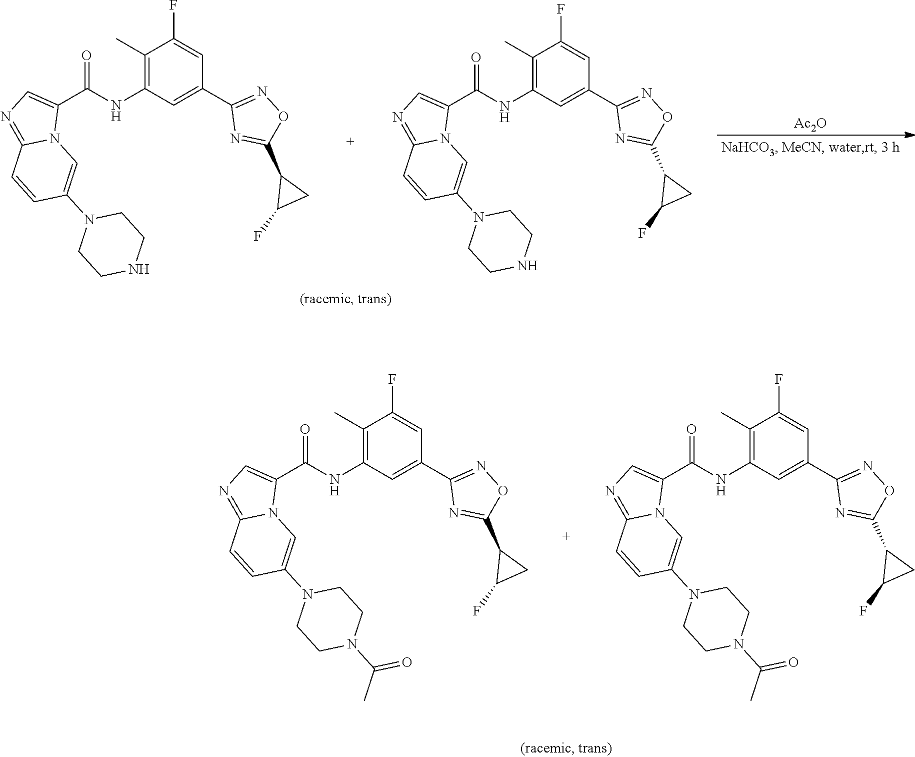 [SMILES: "Ac2O" ; Cc1c(F)cc(-c2noc([C@H]3C[C@@H]3F)n2)cc1NC(=O)c1cnc2ccc(N3CCNCC3)cn12 ; Cc1c(F)cc(-c2noc([C@@H]3C[C@H]3F)n2)cc1NC(=O)c1cnc2ccc(N3CCNCC3)cn12 ; CC(=O)N1CCN(c2ccc3ncc(C(=O)Nc4cc(-c5noc([C@H]6C[C@@H]6F)n5)cc(F)c4C)n3c2)CC1 ; CC(=O)N1CCN(c2ccc3ncc(C(=O)Nc4cc(-c5noc([C@@H]6C[C@H]6F)n5)cc(F)c4C)n3c2)CC1]

To a solution of N-(3-fluoro-5-(5-((1R,2S)-2-fluorocyclopropyl)-1,2,4-oxadiazol-3-yl)-2-methylphenyl)-6-(piperazin-1-yl)imidazo[1,2-a]pyridine-3-carboxamide and N-(3-fluoro-5-(5-((1S,2R)-2-fluorocyclopropyl)-1,2,4-oxadiazol-3-yl)-2-methylphenyl)-6-(piperazin-1-yl)imidazo[1,2-a]pyridine-3-carboxamide (racemic, trans, 100 mg, 0.21 mmol, 1.0 eq) in MeCN/H₂O (15 mL/3 mL) was added acetic anhydride (106.5 mg, 1.0 mmol, 5.0 eq) and NaHCO₃ (175.4 mg, 2.1 mmol, 10.0 eq). The solution was stirred at rt for 3 h, and then partitioned between EtOAc (20 mL) and saturated NaHCO₃ solution (20 mL). The aqueous layer was extracted with EtOAc (2×20 mL), and the combinded organic layers were concentrated under vacuum. The resulting residue was purified by prep-HPLC to afford 6-(4-acetylpiperazin-1-yl)-N-(3-fluoro-5-(5-((1R,2S)-2-fluorocyclopropyl)-1,2,4-oxadiazol-3-yl)-2-methylphenyl)imidazo[1,2-a]pyridine-3-carboxamide and 6-(4-acetylpiperazin-1-yl)-N-(3-fluoro-5-(5-((1S,2R)-2-fluorocyclopropyl)-1,2,4-oxadiazol-3-yl)-2-methylphenyl)imidazo[1,2-a]pyridine-3-carboxamide (racemic, trans) as a white solid (43.3 mg, 39.8%). LCMS (M+H⁺) m/z calculated 522.2, found 522.1. ¹H NMR (CDCl₃, 400 MHz) δ 9.06 (d, 1H), 8.30 (s, 1H), 8.15 (s, 1H), 7.59-7.68 (m, 3H), 7.33 (dd, 1H), 4.97-5.15 (m, 1H), 3.63-3.82 (m, 5H), 3.12-3.17 (m, 4H), 2.70-2.73 (m, 1H), 2.32 (d, 3H), 2.15 (s, 3H), 1.82-1.89 (m, 1H), 1.59-1.67 (m, 1H).

### Example 40: Preparation of Methyl 3-(3-(3-(6-carbamoylimidazo[1,2-a]pyridine-3-carboxamido)-5-fluoro-4-methylphenyl)-1,2,4-oxadiazol-5-yl)azetidine-1-carboxylate

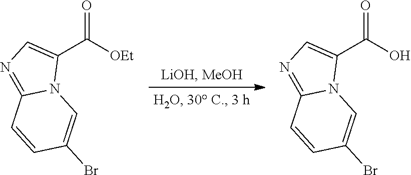 [SMILES: CCOC(=O)c1cnc2ccc(Br)cn12 ; O=C(O)c1cnc2ccc(Br)cn12]

A solution of ethyl ethyl 6-bromoimidazo[1,2-a]pyridine-3-carboxylate (1.0 g, 3.7 mmol, 1.0 eq) and LiOH (178.4 mg, 7.4 mmol, 2.0 eq) in MeOH/H₂O (30 mL/5 mL) was stirred at 30° C. for 3 h, and the solvent was partially removed under vacuum. The pH was adjusted to 5 with conc. HCl, then the solid was collected by vacuum filtration and dried under vacuum to afford 6-bromoimidazo[1,2-a]pyridine-3-carboxylic acid as a white solid (950.0 mg, quant.). LCMS (M+H⁺) m/z calculated 242.0, found 241.9.

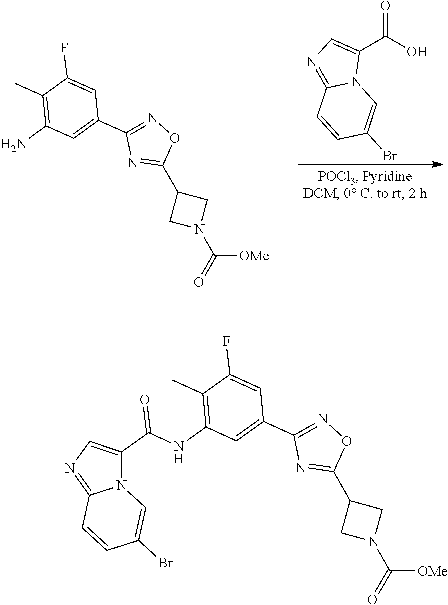 [SMILES: COC(=O)N1CC(c2nc(-c3cc(N)c(C)c(F)c3)no2)C1 ; O=C(O)c1cnc2ccc(Br)cn12 ; COC(=O)N1CC(c2nc(-c3cc(F)c(C)c(NC(=O)c4cnc5ccc(Br)cn45)c3)no2)C1]

To a solution of methyl 3-(3-(3-amino-5-fluoro-4-methylphenyl)-1,2,4-oxadiazol-5-yl)azetidine-1-carboxylate (1.2 g, 4.0 mmol, 1.0 eq), 6-bromoimidazo[1,2-a]pyridine-3-carboxylic acid (950.0 mg, 4.0 mmol, 1.0 eq) and pyridine (1.6 mL, 19.8 mmol, 5.0 eq) in DCM (30 mL) was added POCl₃ (1.1 mL, 11.9 mmol, 3.0 eq) at 0° C. The reaction mixture was stirred at rt for 2 h, and diluted with water (50 mL). The aqueous layer was extracted with DCM (50 mL×2). The combined organic layers were washed with brine (50 mL), dried over anhydrous sodium sulfate, filtered and concentrated. The resulting residue was purified by column chromatography on silica gel (DCM/MeOH=50/1) to afford methyl 3-(3-(3-(6-bromoimidazo[1,2-a]pyridine-3-carboxamido)-5-fluoro-4-methylphenyl)-1,2,4-oxadiazol-5-yl)azetidine-1-carboxylate as a yellow solid (800.0 mg, 38.3%). LCMS (M+H⁺) m/z calculated 529.1, found 529.0.

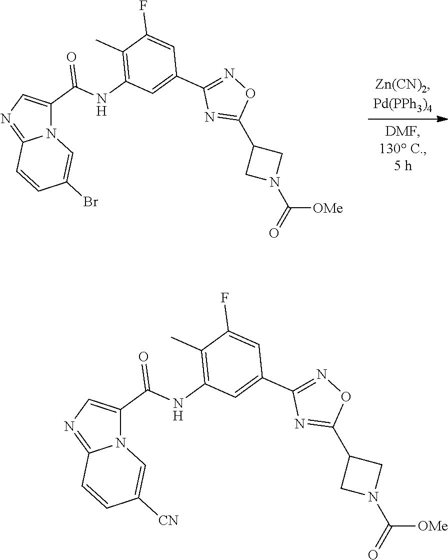 [SMILES: COC(=O)N1CC(c2nc(-c3cc(F)c(C)c(NC(=O)c4cnc5ccc(Br)cn45)c3)no2)C1 ; COC(=O)N1CC(c2nc(-c3cc(F)c(C)c(NC(=O)c4cnc5ccc(C#N)cn45)c3)no2)C1]

To a solution of methyl 3-(3-(3-(6-bromoimidazo[1,2-a]pyridine-3-carboxamido)-5-fluoro-4-methylphenyl)-1,2,4-oxadiazol-5-yl)azetidine-1-carboxylate (300 mg, 0.57 mmol, 1.0 eq) in DMF (20 mL) were added Zn(CN)₂ (100.1 mg, 0.85 mmol. 1.5 eq) and Pd(PPh₃)₄ (65.7 mg, 0.056 mmol, 0.1 eq). The mixture was stirred at 130° C. for 5 h, then the reaction mixture was diluted with water (100 mL) and extracted with EtOAc (100 mL×3). The combined organic layers were washed with brine (100 mL×2), dried with anhydrous sodium sulfate, filtered, and concentrated. The resulting residue was purified by prep-HPLC to afford methyl 3-(3-(3-(6-cyanoimidazo[1,2-a]pyridine-3-carboxamido)-5-fluoro-4-methylphenyl)-1,2,4-oxadiazol-5-yl)azetidine-1-carboxylate as a white solid (54.6 mg, 20.4%). LCMS (M+H⁺) m/z calculated 476.1, found 476.0.

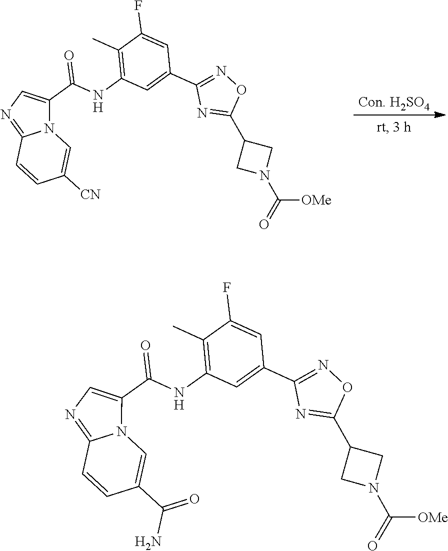 [SMILES: COC(=O)N1CC(c2nc(-c3cc(F)c(C)c(NC(=O)c4cnc5ccc(C#N)cn45)c3)no2)C1 ; COC(=O)N1CC(c2nc(-c3cc(F)c(C)c(NC(=O)c4cnc5ccc(C(N)=O)cn45)c3)no2)C1]

The mixture of methyl 3-(3-(3-(6-cyanoimidazo[1,2-a]pyridine-3-carboxamido)-5-fluoro-4-methylphenyl)-1,2,4-oxadiazol-5-yl)azetidine-1-carboxylate (40.0 mg, 0.084 mmol, 1.0 eq) in 5 mL H₂SO₄ (98% in H₂O) was stirred at rt for 3 h. The mixture was poured into ice water (30 mL) and adjusted to pH 9 by adding saturated aqueous Na₂CO₃. The aqueous layer was extracted with EtOAc (50 mL×2). The combined organic layers were washed with brine (20 mL), dried over anhydrous sodium sulfate, filered and concentrated. The resulting residue was purified by prep-HPLC to afford methyl 3-(3-(3-(6-carbamoylimidazo[1,2-a]pyridine-3-carboxamido)-5-fluoro-4-methylphenyl)-1,2,4-oxadiazol-5-yl)azetidine-1-carboxylate as a white solid (1.0 mg, 2.4%). LCMS (M+H⁺) m/z calculated 494.2, found 494.0. ¹H NMR (DMSO-d₆, 400 MHz) δ 10.31 (s, 1H), 9.96 (s, 1H), 8.66 (s, 1H), 8.24 (s, 1H), 8.01 (s, 1H), 7.95 (d, 1H), 7.83 (d, 1H), 7.68 (d, 1H), 7.62 (s, 1H), 4.20-4.38 (m, 5H), 3.60 (s, 3H), 2.27 (s, 3H).

### Example 41: Preparation of 2-aminoethyl 3-(3-(3-fluoro-5-(imidazo[1,2-a]pyridine-3-carboxamido)-4-methylphenyl)-1,2,4-oxadiazol-5-yl)azetidine-1-carboxylate

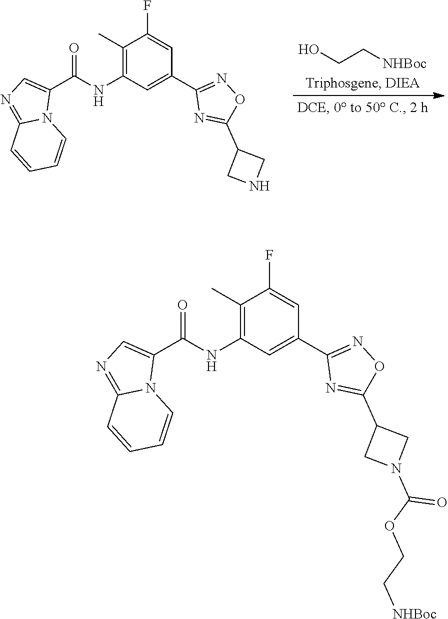 [SMILES: Cc1c(F)cc(-c2noc(C3CNC3)n2)cc1NC(=O)c1cnc2ccccn12 ; CC(C)(C)OC(=O)NCCO ; Cc1c(F)cc(-c2noc(C3CN(C(=O)OCCNC(=O)OC(C)(C)C)C3)n2)cc1NC(=O)c1cnc2ccccn12]

To a solution of tert-butyl (2-hydroxyethyl)carbamate (82.1 mg, 0.51 mmol, 1.0 eq), TEA (515.3 mg, 5.1 mmol, 10.0 eq) in DCE (10 mL) was added triphosgene (151.5 mg, 0.51 mmol, 1.0 eq) at 0° C. The solution was warmed to rt and stirred for 1 h, and then N-(5-(5-(azetidin-3-yl)-1,2,4-oxadiazol-3-yl)-3-fluoro-2-methylphenyl)imidazo[1,2-a]pyridine-3-carboxamide (200.0 mg, 0.51 mmol, 1.0 eq) was added. The resulting solution was heated at 50° C. for 2 h. After cooling to rt, the solution was diluted with DCM (50 mL). The resulting solution was washed with brine (30 mL). The organic layer was dried over anhydrous sodium sulfate, fitered, concentrated. The resulting residue was purified by column chromatography on silica gel (DCM/MeOH=100/1, v/v) to afford 2-((tert-butoxycarbonyl)amino)ethyl 3-(3-(3-fluoro-5-(imidazo[1,2-a]pyridine-3-carboxamido)-4-methylphenyl)-1,2,4-oxadiazol-5-yl)azetidine-1-carboxylate (100.0 mg, 33.9%) as a yellow solid. LCMS (M+H⁺) m/z calculated 580.2, found 580.1.

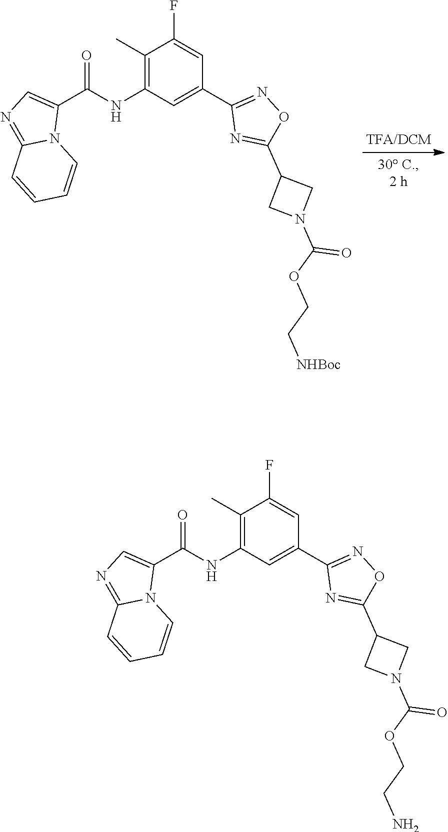 [SMILES: Cc1c(F)cc(-c2noc(C3CN(C(=O)OCCNC(=O)OC(C)(C)C)C3)n2)cc1NC(=O)c1cnc2ccccn12 ; Cc1c(F)cc(-c2noc(C3CN(C(=O)OCCN)C3)n2)cc1NC(=O)c1cnc2ccccn12]

To a stirring solution of 2-((tert-butoxycarbonyl)amino)ethyl 3-(3-(3-fluoro-5-(imidazo[1,2-a]pyridine-3-carboxamido)-4-methylphenyl)-1,2,4-oxadiazol-5-yl)azetidine-1-carboxylate (100 mg, 0.17 mmol, 1.0 eq) in DCM (10 mL) was added TFA (5 mL). The reaction was stirred at 30° C. for 2 h. The mixture was concentrated and purified by prep-HPLC to afford 2-aminoethyl 3-(3-(3-fluoro-5-(imidazo[1,2-a]pyridine-3-carboxamido)-4-methylphenyl)-1,2,4-oxadiazol-5-yl)azetidine-1-carboxylate as a white solid (22.1 mg, 26.8%). LCMS (M+H⁺) m/z calculated 480.2, found 480.0. ¹H NMR (DMSO-d₆, 400 MHz) δ 9.45 (d, 1H), 8.61 (s, 1H), 7.99 (s, 1H), 7.79 (d, 1H), 7.67 (dd, 1H), 7.51-7.56 (m, 1H), 7.19 (t, 1H), 4.19-4.38 (m, 5H), 3.93-3.99 (m, 2H), 3.15-3.23 (m, 2H), 2.55-2.72 (m, 2H), 2.24 (d, 3H).

### Example 42: Preparation of 2-methoxyethyl 3-(3-(3-fluoro-5-(imidazo[1,2-a]pyridine-3-carboxamido)-4-methylphenyl)-1,2,4-oxadiazol-5-yl)azetidine-1-carboxylate

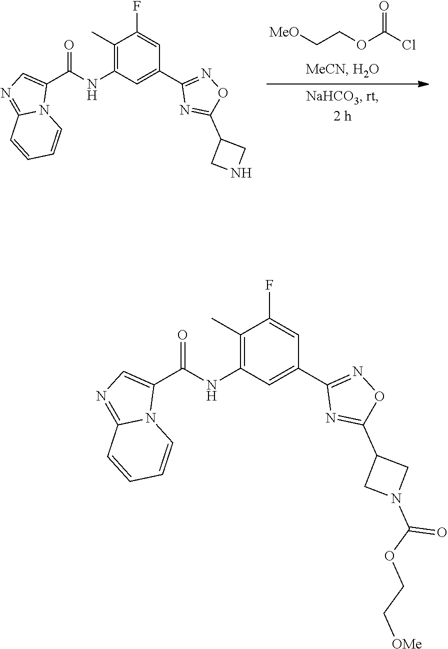 [SMILES: Cc1c(F)cc(-c2noc(C3CNC3)n2)cc1NC(=O)c1cnc2ccccn12 ; COCCOC(=O)Cl ; COCCOC(=O)N1CC(c2nc(-c3cc(F)c(C)c(NC(=O)c4cnc5ccccn45)c3)no2)C1]

To a solution of N-(5-(5-(azetidin-3-yl)-1,2,4-oxadiazol-3-yl)-3-fluoro-2-methylphenyl)imidazo[1,2-a]pyridine-3-carboxamide (100 mg, 0.26 mmol, 1.0 eq) in MeCN/H₂O (15 mL/3 mL) was added 2-methoxyethyl carbonochloridate (52.8 mg, 0.38 mmol, 1.5 eq) and NaHCO₃ (214.2 mg, 2.6 mmol, 10.0 eq). The solution was stirred at rt for 2 h, and then partitioned between EtOAc (20 mL) and saturated NaHCO₃ solution (20 mL). The aqueous layer was extracted with EtOAc (20 mL×2). The organic layers were concentrated and purified by prep-HPLC to afford 2-methoxyethyl 3-(3-(3-fluoro-5-(imidazo[1,2-a]pyridine-3-carboxamido)-4-methylphenyl)-1,2,4-oxadiazol-5-yl)azetidine-1-carboxylate as a white solid (18.1 mg, 14.4%). LCMS (M+H⁺) m/z calculated 495.2, found 495.2. ¹H NMR (DMSO-d₆, 400 MHz) δ 10.26 (s, 1H), 9.54 (d, 1H), 8.55 (s, 1H), 8.06 (s, 1H), 7.76 (d, 1H), 7.59 (d, 1H), 7.51 (t, 1H), 7.15 (t, 1H), 4.10-4.38 (m, 7H), 3.49-3.52 (m, 2H), 3.26 (s, 3H), 2.25 (d, 3H).

### Example 43: Preparation of Methyl 3-(3-(3-(7-carbamoylimidazo[1,2-a]pyridine-3-carboxamido)-5-fluoro-4-methylphenyl)-1,2,4-oxadiazol-5-yl)azetidine-1-carboxylate

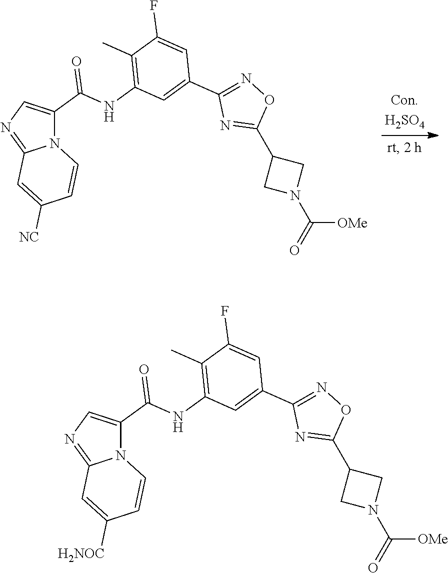 [SMILES: COC(=O)N1CC(c2nc(-c3cc(F)c(C)c(NC(=O)c4cnc5cc(C#N)ccn45)c3)no2)C1 ; COC(=O)N1CC(c2nc(-c3cc(F)c(C)c(NC(=O)c4cnc5cc(C(N)=O)ccn45)c3)no2)C1]

The mixture of methyl 3-(3-(3-(7-cyanoimidazo[1,2-a]pyridine-3-carboxamido)-5-fluoro-4-methylphenyl)-1,2,4-oxadiazol-5-yl)azetidine-1-carboxylate (40.0 mg, 0.084 mmol, 1.0 eq) in 5 mL H₂SO₄ (98% in H₂O) was stirred at rt for 2 h. The mixture was poured into ice water (30 mL) and adjusted to pH 9 by adding saturated aqueous Na₂CO₃. The aqueous layer was extracted with EtOAc (50 mL×2). The combined organic layers were washed with brine (20 mL), dried over anhydrous sodium sulfate, filtered and concentrated. The resulting residue was purified by prep-HPLC to afford methyl 3-(3-(3-(7-carbamoylimidazo[1,2-a]pyridine-3-carboxamido)-5-fluoro-4-methylphenyl)-1,2,4-oxadiazol-5-yl)azetidine-1-carboxylate as a white solid (15.4 mg, 36.5%). LCMS (M+H⁺) m/z calculated 494.2, found 494.0. ¹H NMR (DMSO-d₆, 400 MHz) δ 10.32 (s, 1H), 9.45 (d, 1H), 8.70 (s, 1H), 8.30-8.33 (m, 2H), 8.00 (s, 1H), 7.40-7.70 (m, 3H), 4.20-4.38 (m, 5H), 3.60 (s, 3H), 2.26 (s, 3H).

### Example 44: Preparation of Methyl 3-(5-(3-fluoro-5-(imidazo[1,2-a]pyridine-3-carboxamido)-4-methylphenyl)-1,3,4-oxadiazol-2-yl)azetidine-1-carboxylate

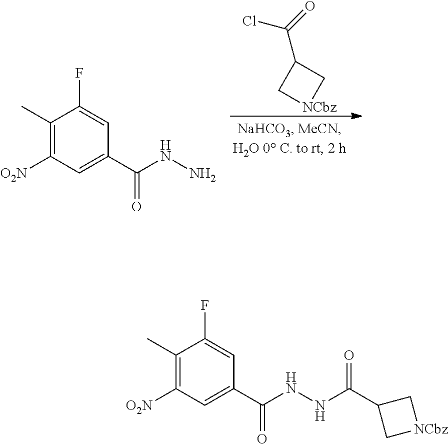 [SMILES: Cc1c(F)cc(C(=O)NN)cc1[N+](=O)[O-] ; O=C(Cl)C1CN(C(=O)OCc2ccccc2)C1 ; Cc1c(F)cc(C(=O)NNC(=O)C2CN(C(=O)OCc3ccccc3)C2)cc1[N+](=O)[O-]]

To the solution of 3-fluoro-4-methyl-5-nitrobenzohydrazide (800.0 mg, 3.8 mmol, 1.0 eq) and NaHCO₃ (957.6 mg, 11.4 mmol, 3.0 eq) in MeCN (9.0 mL) and H₂O (3.0 mL) was added benzyl 3-(chlorocarbonyl)azetidine-1-carboxylate (1.9 g, 7.6 mmol, 2.0 eq) in MeCN (3.0 mL) at 0° C. and stirred at rt for 2 h. The reaction was diluted with water (50.0 mL) and the aqueous layer was extracted with DCM (50.0 mL×3). The combined organic layers were washed with brine (50.0 mL), dried over anhydrous sodium sulfate, filtered, and concentrated. The resulting residue was purified by column chromatography on silica gel (PE/EtOAc=1/1, v/v) to afford benzyl 3-(2-(3-fluoro-4-methyl-5-nitrobenzoyl)hydrazine-1-carbonyl)azetidine-1-carboxylate as a pale yellow solid (961.0 mg, 60.0%). LCMS (M+H⁺) m/z calculated 431.1, found 431.1.

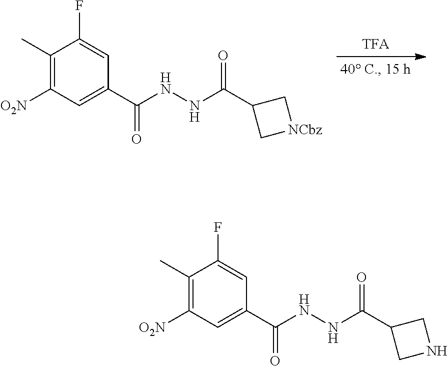 [SMILES: Cc1c(F)cc(C(=O)NNC(=O)C2CN(C(=O)OCc3ccccc3)C2)cc1[N+](=O)[O-] ; Cc1c(F)cc(C(=O)NNC(=O)C2CNC2)cc1[N+](=O)[O-]]

The mixture of benzyl 3-(2-(3-fluoro-4-methyl-5-nitrobenzoyl)hydrazine-1-carbonyl)azetidine-1-carboxylate (961.0 mg, 2.2 mmol, 1.0 eq) in TFA (2.0 mL) was stirred at 40° C. for 15 h. The mixture was concentrated and the residue was obtained as crude N′-(3-fluoro-4-methyl-5-nitrobenzoyl)azetidine-3-carbohydrazide as pale yellow solid (1.2 g, quant.), which was used in the next step without further purification. LCMS (M+H⁺) m/z calculated 297.1, found 297.1.

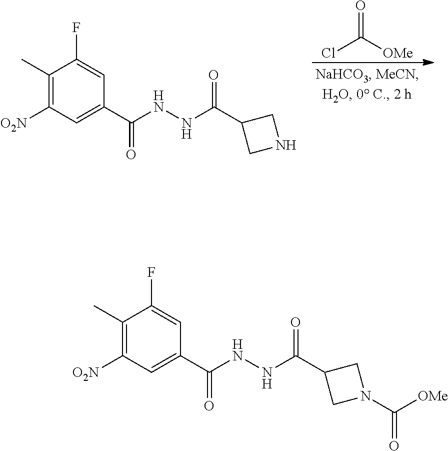 [SMILES: Cc1c(F)cc(C(=O)NNC(=O)C2CNC2)cc1[N+](=O)[O-] ; COC(=O)Cl ; COC(=O)N1CC(C(=O)NNC(=O)c2cc(F)c(C)c([N+](=O)[O-])c2)C1]

To the solution of N′-(3-fluoro-4-methyl-5-nitrobenzoyl)azetidine-3-carbohydrazide (1.2 g, 4.1 mmol, 1.0 eq) and NaHCO₃ (1.0 g, 12.3 mmol, 3.0 eq) in MeCN (15.0 mL) and H₂O (5.0 mL) was added methyl carbonochloridate (789.6 mg, 8.4 mmol, 2.0 eq) in MeCN (5.0 mL) at 0° C., and stirred at rt for 2.0 h. The reaction was diluted with water (30 mL) and the aqueous layer was extracted with DCM (30 mL×3). The combined organic layers were washed with brine (30 mL), dried over anhydrous sodium sulfate, filtered, and concentrated. The resulting residue was purified by column chromatography on silica gel (PE/EtOAc=1/1, v/v) to afford methyl 3-(2-(3-fluoro-4-methyl-5-nitrobenzoyl)hydrazine-1-carbonyl)azetidine-1-carboxylate as a pale yellow solid (370.0 mg, 26.4%). LCMS (M+H⁺) m/z calculated 355.1, found 355.1.

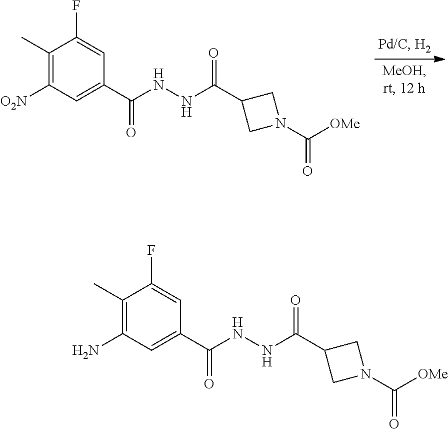 [SMILES: COC(=O)N1CC(C(=O)NNC(=O)c2cc(F)c(C)c([N+](=O)[O-])c2)C1 ; COC(=O)N1CC(C(=O)NNC(=O)c2cc(N)c(C)c(F)c2)C1]

The mixture of methyl 3-(2-(3-fluoro-4-methyl-5-nitrobenzoyl)hydrazine-1-carbonyl)azetidine-1-carboxylate (370.0 mg, 1.0 mmol, 1.0 eq) and 50.0 mg Pd/C in MeOH (10.0 mL) was stirred in the charge of H₂ at rt for 12 h. The mixture was filtered to remove Pd/C and the filtrate was concentrated. The resulting residue was purified by column chromatography on silica gel (PE/EtOAc=1/1, v/v) to afford methyl 3-(2-(3-amino-5-fluoro-4-methylbenzoyl)hydrazine-1-carbonyl)azetidine-1-carboxylate as a yellow solid (200.0 mg, 54.2%). LCMS (M+H⁺) m/z calculated 325.1, found 325.1.

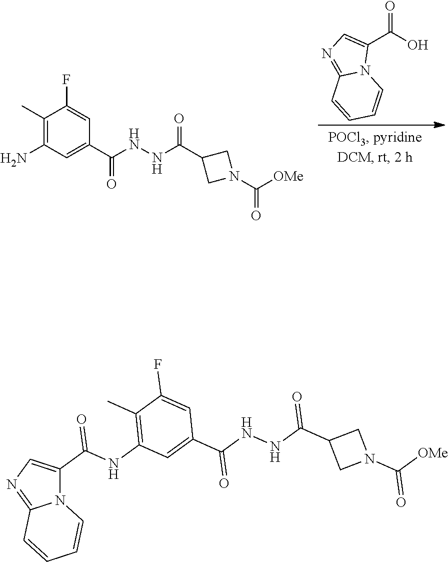 [SMILES: COC(=O)N1CC(C(=O)NNC(=O)c2cc(N)c(C)c(F)c2)C1 ; O=C(O)c1cnc2ccccn12 ; COC(=O)N1CC(C(=O)NNC(=O)c2cc(F)c(C)c(NC(=O)c3cnc4ccccn34)c2)C1]

To a stirring solution of imidazo[1,2-a]pyridine-3-carboxylic acid (100.4 mg, 0.62 mmol, 1.0 eq), methyl 3-(2-(3-amino-5-fluoro-4-methylbenzoyl)hydrazine-1-carbonyl)azetidine-1-carboxylate (200.0 mg, 0.62 mmol, 1.0 eq) and pyridine (244.9 mg, 3.1 mmol, 5.0 eq) in DCM (5.0 mL) was added POCl₃ (284.6 mg, 1.9 mmol, 3.0 eq) at rt. The reaction mixture was stirred at rt for 2 h, diluted with water (10 mL). The aqueous layer was extracted with DCM (20 mL×3) and the combined organic layers were washed with brine (30 mL), dried over anhydrous sodium sulfate, filtered, and concentrated. The resulting residue was purified by column chromatography on silica gel (PE/EtOAc=1/2, v/v) to afford methyl 3-(2-(3-fluoro-5-(imidazo[1,2-a]pyridine-3-carboxamido)-4-methylbenzoyl)hydrazine-1-carbonyl)azetidine-1-carboxylate (120.0 mg, 45.3%). LCMS (M+H⁺) m/z calculated 469.2, found 469.2.

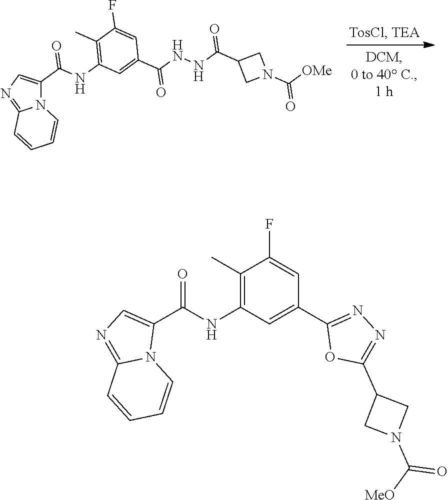 [SMILES: COC(=O)N1CC(C(=O)NNC(=O)c2cc(F)c(C)c(NC(=O)c3cnc4ccccn34)c2)C1 ; COC(=O)N1CC(c2nnc(-c3cc(F)c(C)c(NC(=O)c4cnc5ccccn45)c3)o2)C1]

To a stirring solution of methyl 3-(2-(3-fluoro-5-(imidazo[1,2-a]pyridine-3-carboxamido)-4-methylbenzoyl)hydrazine-1-carbonyl)azetidine-1-carboxylate (120.0 mg, 0.26 mmol, 1.0 eq), TEA (131.3 mg, 1.3 mmol, 5.0 eq) in DCM (5.0 mL) was added TosCl (148.2 mg, 0.78 mmol, 3.0 eq) at 0° C. The reaction mixture was stirred at 40° C. for 1 h. The reaction mixture was diluted with water (10 mL) and extracted with DCM (20 mL×3). The combined organic layers were washed with brine (30 mL), dried over anhydrous sodium sulfate, filtered, and concentrated. The resulting residue was purified by prep-HPLC to afford methyl 3-(5-(3-fluoro-5-(imidazo[1,2-a]pyridine-3-carboxamido)-4-methylphenyl)-1,3,4-oxadiazol-2-yl)azetidine-1-carboxylate (11.3 mg, 9.8%). LCMS (M+H⁺) m/z calculated 451.1, found 451.2. ¹H NMR (MeOD-d₄, 400 MHz) δ 9.50-9.52 (d, 1H), 8.49 (s, 1H), 8.00 (s, 1H), 7.68-7.76 (m, 2H), 7.56-7.60 (m, 1H), 7.16-7.20 (m, 1H), 4.43-4.47 (m, 2H), 4.31-4.34 (m, 2H), 4.25-4.25 (m, 1H), 3.69 (s, 3H), 2.32-2.33 (d, 3H).

### Example 45: Preparation of Methyl 3-(4-(3-fluoro-5-(imidazo[1,2-a]pyridine-3-carboxamido)-4-methylphenyl)oxazol-2-yl)azetidine-1-carboxylate

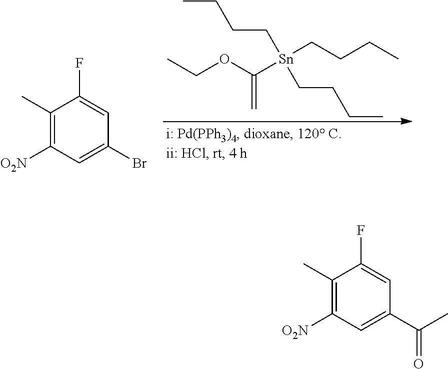 [SMILES: Cc1c(F)cc(Br)cc1[N+](=O)[O-] ; C=[C](OCC)[Sn]([CH2]CCC)([CH2]CCC)[CH2]CCC ; CC(=O)c1cc(F)c(C)c([N+](=O)[O-])c1]

The solution of 5-bromo-1-fluoro-2-methyl-3-nitrobenzene (10.0 g, 42.9 mmol, 1.0 eq), tributyl(1-ethoxyvinyl)stannane (31.1 g, 85.8 mmol, 2.0 eq) and Pd(PPh₃)₄ (4.9 g, 4.3 mmol, 0.1 eq) in dioxane (500.0 mL) was stirred at 120° C. for 12 h. 1N HCl (50 mL) was added and was stirred at rt for 4.0 h. The reaction mixture was poured into H₂O (500 mL) and extracted with EtOAc (500 mL×3). The combined organic layers were washed with brine (500 mL), dried over anhydrous sodium sulfate, filtered, and concentrated. The resulting residue was purified by column chromatography on silica gel (PE/EtOAc=5/1, v/v) to afford 1-(3-fluoro-4-methyl-5-nitrophenyl)ethan-1-one as a pale yellow solid (9.0 g, 99.0%).

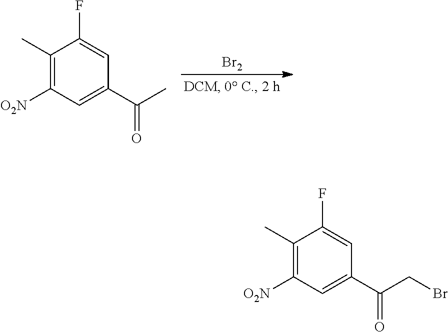

To a solution of 1-(3-fluoro-4-methyl-5-nitrophenyl)ethan-1-one (9.0 g, 45.7 mmol, 1.0 eq) in DCM (100.0 mL) was added dibromine (7.9 g, 50.3 mmol, 1.1 eq) at 0° C. and the mixture was stirred at 0° C. for 2 h. The mixture was concentrated and purified by column chromatography on silica gel (PE/EtOAc=3/1, v/v) to afford 2-bromo-1-(3-fluoro-4-methyl-5-nitrophenyl)ethan-1-one as a yellow solid (4.7 g, 37.6%).

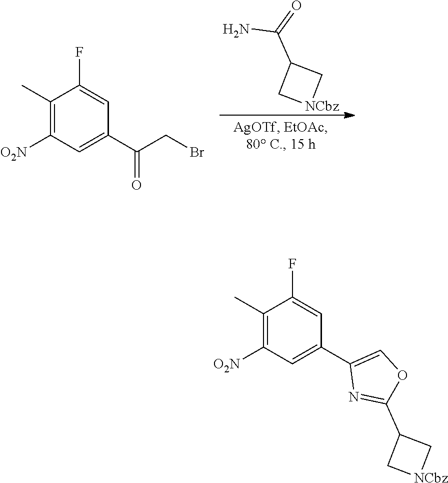 [SMILES: Cc1c(F)cc(C(=O)CBr)cc1[N+](=O)[O-] ; NC(=O)C1CN(C(=O)OCc2ccccc2)C1 ; Cc1c(F)cc(-c2coc(C3CN(C(=O)OCc4ccccc4)C3)n2)cc1[N+](=O)[O-]]

The solution of 2-bromo-1-(3-fluoro-4-methyl-5-nitrophenyl)ethan-1-one (500.0 mg, 1.8 mmol, 1.0 eq), benzyl 3-carbamoylazetidine-1-carboxylate (842.4 mg, 3.6 mmol, 2.0 eq) and Ag(SO₃CF₃) (466.2 mg, 1.8 mmol, 1.0 eq) in EtOAc (10.0 mL) was stirred at 80° C. for 15 h. The reaction mixture was poured into H₂O (30.0 mL) and extracted with EtOAc (50.0 mL×3). The combined organic layers were washed with brine (50.0 mL), dried over anhydrous sodium sulfate, filtered, and concentrated. The resulting residue was purified by column chromatography on silica gel (PE/EtOAc=5/1, v/v) to afford benzyl 3-(4-(3-fluoro-4-methyl-5-nitrophenyl)oxazol-2-yl)azetidine-1-carboxylate as a pale yellow solid (200.0 mg, 27.0%). LCMS (M+H⁺) m/z calculated 412.1, found 412.1.

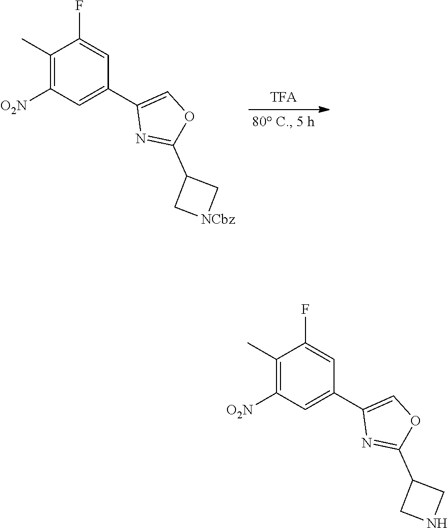 [SMILES: Cc1c(F)cc(-c2coc(C3CN(C(=O)OCc4ccccc4)C3)n2)cc1[N+](=O)[O-] ; Cc1c(F)cc(-c2coc(C3CNC3)n2)cc1[N+](=O)[O-]]

The mixture of benzyl 3-(4-(3-fluoro-4-methyl-5-nitrophenyl)oxazol-2-yl)azetidine-1-carboxylate (200.0 mg, 0.49 mmol, 1.0 eq) in TFA (2.0 mL) was stirred at 80° C. for 5 h. The mixture was concentrated to afford crude 2-(azetidin-3-yl)-4-(3-fluoro-4-methyl-5-nitrophenyl)oxazole as pale yellow solid (120.0 mg, 89.6%), which was used in the next step without further purification. LCMS (M+H⁺) m/z calculated 278.1, found 278.1.

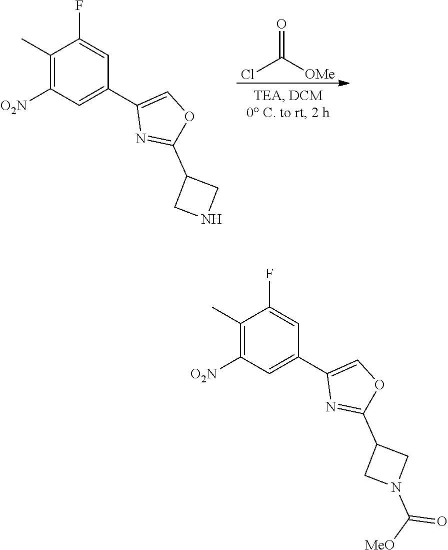 [SMILES: Cc1c(F)cc(-c2coc(C3CNC3)n2)cc1[N+](=O)[O-] ; COC(=O)Cl ; COC(=O)N1CC(c2nc(-c3cc(F)c(C)c([N+](=O)[O-])c3)co2)C1]

To a solution of 2-(azetidin-3-yl)-4-(3-fluoro-4-methyl-5-nitrophenyl)oxazole (120.0 mg, 0.43 mmol, 1.0 eq) and TEA (131.3 mg, 1.3 mmol, 3.0 eq) in DCM (5.0 mL) was added methyl carbonochloridate (80.9 mg, 0.86 mmol, 2.0 eq) at 0° C. and the mixture was stirred at rt for 2 h. The mixture was concentrated and purified by column chromatography on silica gel (PE/EtOAc=1/1, v/v) to afford methyl 3-(4-(3-fluoro-4-methyl-5-nitrophenyl)oxazol-2-yl)azetidine-1-carboxylate a yellow solid (120.0 mg, 82.8%). LCMS (M+H⁺) m/z calculated 336.1, found 336.1.

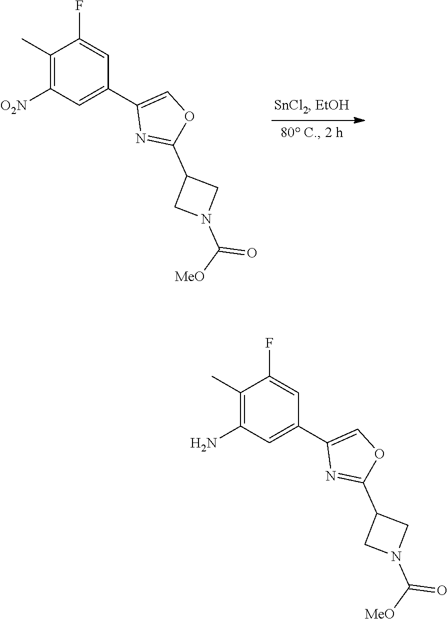 [SMILES: COC(=O)N1CC(c2nc(-c3cc(F)c(C)c([N+](=O)[O-])c3)co2)C1 ; COC(=O)N1CC(c2nc(-c3cc(N)c(C)c(F)c3)co2)C1]

To a suspension of methyl 3-(4-(3-fluoro-4-methyl-5-nitrophenyl)oxazol-2-yl)azetidine-1-carboxylate (120.0 mg, 0.36 mmol, 1.0 eq) in EtOH (5.0 mL) was added SnCl₂ (204.2 mg, 1.1 mmol, 3.0 eq). The reaction mixture was stirred at 80° C. for 2 h. The reaction was cooled to rt and the solvent was partially removed. The pH was adjusted to slightly basic with saturated solution of sodium bicarbonate. The resulting white suspension was filtered and washed with water (30 mL) and EtOAc (30 mL). The aqueous layer was extracted with EtOAc (30 mL×3). The combined organic layers were washed with brine (30.0 mL), dried over anhydrous sodium sulfate, filtered, and concentrated. The resulting residue was purified by column chromatography on silica gel (PE/EtOAc=1/1, v/v) to afford methyl 3-(4-(3-amino-5-fluoro-4-methylphenyl)oxazol-2-yl)azetidine-1-carboxylate as a yellow solid (80.0 mg, 73.4%). LCMS (M+H⁺) m/z calculated 306.1, found 306.1.

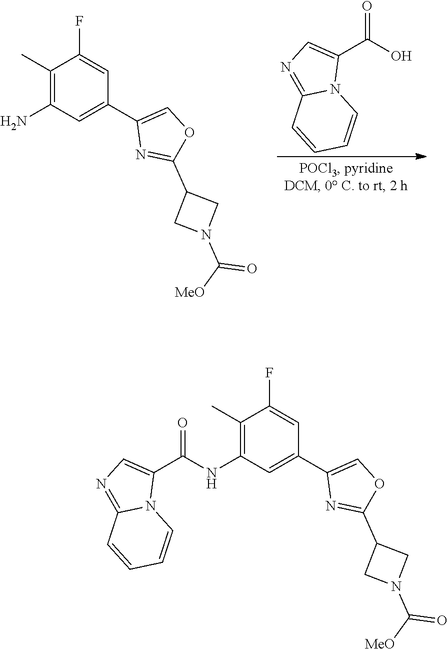 [SMILES: COC(=O)N1CC(c2nc(-c3cc(N)c(C)c(F)c3)co2)C1 ; O=C(O)c1cnc2ccccn12 ; COC(=O)N1CC(c2nc(-c3cc(F)c(C)c(NC(=O)c4cnc5ccccn45)c3)co2)C1]

To a stirring solution of imidazo[1,2-a]pyridine-3-carboxylic acid (42.5 mg, 0.26 mmol, 1.0 eq), methyl 3-(4-(3-amino-5-fluoro-4-methylphenyl)oxazol-2-yl)azetidine-1-carboxylate (80.0 mg, 0.26 mmol, 1.0 eq) and pyridine (102.7 mg, 1.3 mmol, 5.0 eq) in DCM (5.0 mL) was added POCl₃ (119.3 mg, 0.78 mmol, 3.0 eq) at 0° C. The reaction mixture was stirred at rt for 2 h. The reaction mixture was diluted with water (10.0 mL). The aqueous layer was extracted with DCM (20 mL×3) and the combined organic layers were washed with brine (30 mL), dried over anhydrous sodium sulfate, filtered, and concentrated. The resulting residue was purified by prep-HPLC to afford methyl 3-(4-(3-fluoro-5-(imidazo[1,2-a]pyridine-3-carboxamido)-4-methylphenyl)oxazol-2-yl)azetidine-1-carboxylate (23.3 mg, 19.7%). LCMS (M+H⁺) m/z calculated 450.1, found 450.2. ¹H NMR (DMSO-d₆, 400 MHz) δ 10.15 (s, 1H), 9.44-9.46 (m, 1H), 8.66 (s, 1H), 8.59 (s, 1H), 7.77-7.79 (m, 1H), 7.69 (s, 1H), 7.50-7.54 (m, 2H), 7.16-7.19 (m, 1H), 4.28-4.31 (m, 2H), 4.04-4.13 (m, 3H), 3.58 (s, 3H), 2.19 (s, 3H).

### Example 46: Preparation of Methyl 3-(5-(3-fluoro-5-(imidazo[1,2-a]pyridine-3-carboxamido)-4-methylphenyl)oxazol-2-yl)azetidine-1-carboxylate

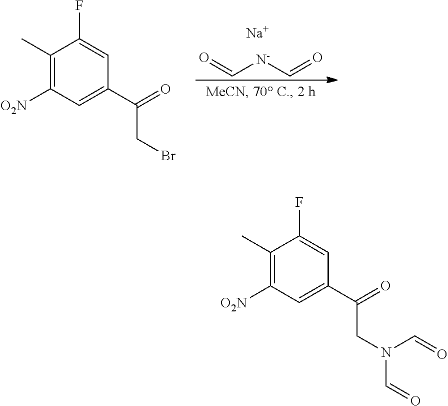 [SMILES: Cc1c(F)cc(C(=O)CBr)cc1[N+](=O)[O-] ; O=C[N-]C=O ; [Na+] ; Cc1c(F)cc(C(=O)CN(C=O)C=O)cc1[N+](=O)[O-]]

The solution of 2-bromo-1-(3-fluoro-4-methyl-5-nitrophenyl)ethan-1-one (1.0 g, 3.6 mmol, 1.0 eq) and sodium N-formylformamide (959.5 mg, 10.1 mmol, 3.0 eq) in MeCN (10.0 mL) was stirred at 70° C. for 2 h. The mixture was concentrated and purified by column chromatography on silica gel (PE/EtOAc=3/1, v/v) to afford N-(2-(3-fluoro-4-methyl-5-nitrophenyl)-2-oxoethyl)-N-formylformamide as a yellow solid (348.0 mg, 35.7%). LCMS (M+H⁺) m/z calculated 269.1, found 269.1.

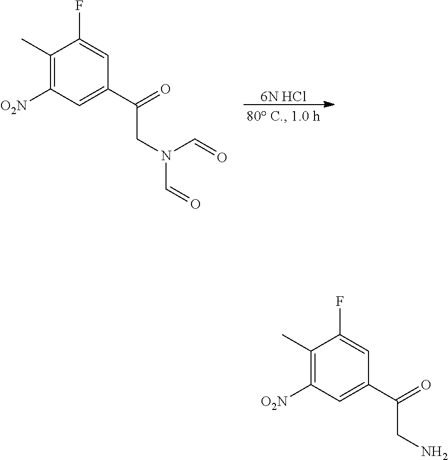

The solution of N-(2-(3-fluoro-4-methyl-5-nitrophenyl)-2-oxoethyl)-N-formylformamide (348.0 mg, 1.3 mmol, 1.0 eq) in 6N HCl (5.0 mL) was stirred at 80° C. for 1.0 h. The mixture was concentrated and purified by column chromatography on silica gel (PE/EtOAc=1/1, v/v) to afford 2-amino-1-(3-fluoro-4-methyl-5-nitrophenyl)ethan-1-one as a yellow solid (400.0 mg, quant.). LCMS (M+H⁺) m/z calculated 213.1, found 213.1.

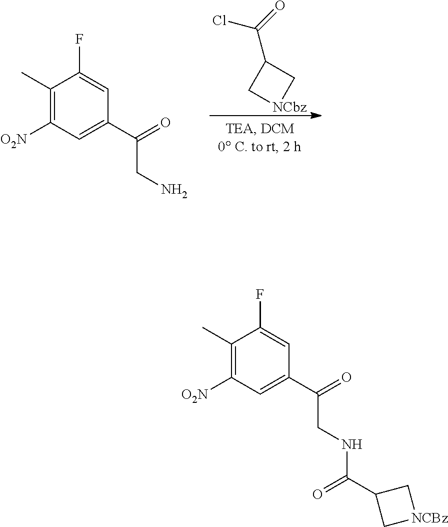

To a solution of 2-amino-1-(3-fluoro-4-methyl-5-nitrophenyl)ethan-1-one (400.0 mg, 1.9 mmol, 1.0 eq) and TEA (575.7 mg, 5.7 mmol, 3.0 eq) in DCM (5.0 mL) was added benzyl 3-(chlorocarbonyl)azetidine-1-carboxylate (961.4 mg, 3.8 mmol, 2.0 eq) at 0° C. and the mixture was stirred at rtrt for 2 h. The mixture was concentrated and purified by column chromatography on silica gel (PE/EtOAc=1/1, v/v) to afford benzyl 3-((2-(3-fluoro-4-methyl-5-nitrophenyl)-2-oxoethyl)carbamoyl)azetidine-1-carboxylate as a yellow solid (438.0 mg, 61.8%). LCMS (M+H⁺) m/z calculated 430.1, found 430.1.

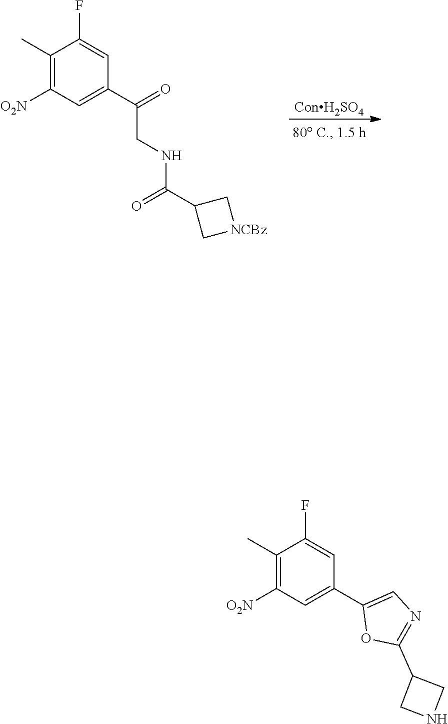

The mixture of benzyl 3-((2-(3-fluoro-4-methyl-5-nitrophenyl)-2-oxoethyl)carbamoyl)azetidine-1-carboxylate (438.0 mg, 1.0 mmol, 1.0 eq) in Con·H₂SO₄ (2.0 mL) was stirred at 80° C. for 1.5 h. The mixture was poured into ice water, and the pH was adjusted to 10.0 by adding saturated aqueous Na₂CO₃. The mixture was extracted with DCM/MeOH (30 mL×3). The combined organic layers were washed with brine (50 mL), dried over anhydrous sodium sulfate, filtered, and concentrated. The resulting residue was purified by column chromatography on silica gel (DCM/MeOH=10/1, v/v) to afford 2-(azetidin-3-yl)-5-(3-fluoro-4-methyl-5-nitrophenyl)oxazole as pale yellow solid (180.0 mg, 64.2%). LCMS (M+H⁺) m/z calculated 278.1, found 278.1.

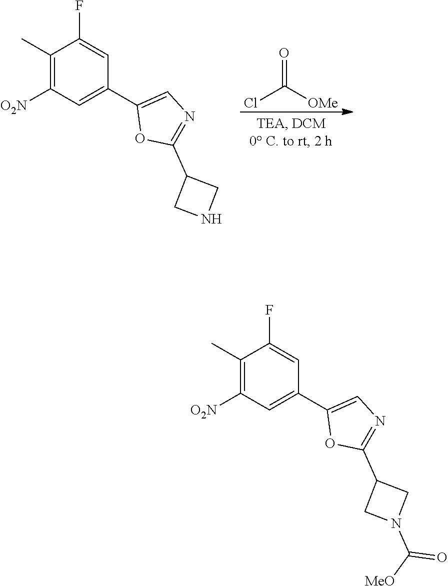 [SMILES: Cc1c(F)cc(-c2cnc(C3CNC3)o2)cc1[N+](=O)[O-] ; COC(=O)Cl ; COC(=O)N1CC(c2ncc(-c3cc(F)c(C)c([N+](=O)[O-])c3)o2)C1]

To a solution of 2-(azetidin-3-yl)-5-(3-fluoro-4-methyl-5-nitrophenyl)oxazole (180.0 mg, 0.65 mmol, 1.0 eq) and TEA (196.9 mg, 1.9 mmol, 3.0 eq) in DCM (5.0 mL) was added methyl carbonochloridate (122.2 mg, 1.3 mmol, 2.0 eq) at 0° C., and the mixture was stirred at rtrt for 2 h. The mixture was concentrated and purified by column chromatography on silica gel (PE/EtOAc=1/1, v/v) to afford methyl 3-(5-(3-fluoro-4-methyl-5-nitrophenyl)oxazol-2-yl)azetidine-1-carboxylate as a yellow solid (180.0 mg, 79.2%). LCMS (M+H⁺) m/z calculated 336.1, found 336.1.

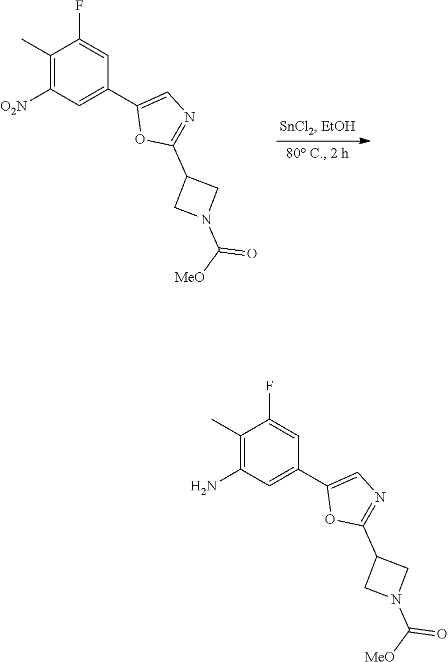 [SMILES: COC(=O)N1CC(c2ncc(-c3cc(F)c(C)c([N+](=O)[O-])c3)o2)C1 ; COC(=O)N1CC(c2ncc(-c3cc(N)c(C)c(F)c3)o2)C1]

To a suspension of methyl 3-(5-(3-fluoro-4-methyl-5-nitrophenyl)oxazol-2-yl)azetidine-1-carboxylate (180.0 mg, 0.54 mmol, 1.0 eq) in EtOH (5.0 mL) was added SnCl₂ (306.3 mg, 1.6 mmol, 3.0 eq). The reaction mixture was stirred at 80° C. for 2 h. The reaction was cooled to rt and the solvent was partially removed under vacuum. The pH was adjusted to slightly basic with saturated solution of sodium bicarbonate. The resulting white suspension was filtered and washed with water (30 mL) and EtOAc (30 mL). The aqueous layer was extracted with EtOAc (30 mL×3). The combined organic layers were washed with brine (30.0 mL), dried over anhydrous sodium sulfate, filtered, and concentrated. The resulting residue was purified by column chromatography on silica gel (PE/EtOAc=1/1, v/v) to afford methyl 3-(5-(3-amino-5-fluoro-4-methylphenyl)oxazol-2-yl)azetidine-1-carboxylate as a yellow solid (82.0 mg, 49.0%). LCMS (M+H⁺) m/z calculated 306.1, found 306.1.

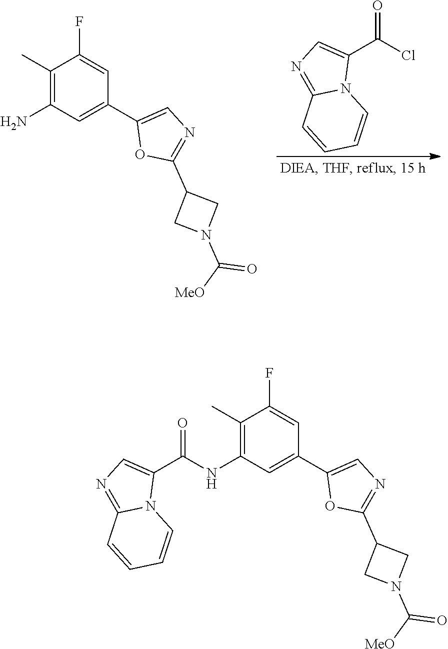 [SMILES: COC(=O)N1CC(c2ncc(-c3cc(N)c(C)c(F)c3)o2)C1 ; O=C(Cl)c1cnc2ccccn12 ; COC(=O)N1CC(c2ncc(-c3cc(F)c(C)c(NC(=O)c4cnc5ccccn45)c3)o2)C1]

To a solution of methyl 3-(5-(3-amino-5-fluoro-4-methylphenyl)oxazol-2-yl)azetidine-1-carboxylate (82.0 mg, 0.26 mmol, 1.0 eq) and DIEA (101.5 mg, 0.79 mmol, 3.0 eq) in THF (5.0 mL) was added imidazo[1,2-a]pyridine-3-carbonyl chloride (93.6 mg, 0.52 mmol, 2.0 eq) at 0° C. and the mixture was stirred at reflux for 15 h. The mixture was concentrated and purified by prep-HPLC to afford methyl 3-(5-(3-fluoro-5-(imidazo[1,2-a]pyridine-3-carboxamido)-4-methylphenyl)oxazol-2-yl)azetidine-1-carboxylate as a white solid (51.1 mg, 42.0%). LCMS (M+H⁺) m/z calculated 450.1, found 450.2. ¹H NMR (DMSO-d₆, 400 MHz) δ 10.17 (s, 1H), 9.45-9.46 (d, 1H), 8.59 (s, 1H), 7.77-7.80 (d, 1H), 7.73 (s, 1H), 7.60 (s, 1H), 7.19-7.54 (m, 2H), 7.16-7.19 (m, 1H), 4.29-4.31 (m, 2H), 4.06-4.15 (m, 3H), 3.58 (s, 3H), 2.19 (s, 3H).

### Example 47: Preparation of Methyl 3-(2-(3-fluoro-5-(imidazo[1,2-a]pyridine-3-carboxamido)-4-methylphenyl)oxazol-4-yl)azetidine-1-carboxylate

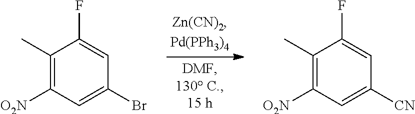

The solution of 5-bromo-1-fluoro-2-methyl-3-nitrobenzene (15.0 g, 64.3 mmol, 1.0 eq), Zn(CN)₂ (15.0 g, 128.6 mmol, 2.0 eq) and Pd(PPh₃)₄ (7.4 g, 6.4 mmol, 0.1 eq) in DMF (300.0 mL) was stirred at 130° C. for 15 h. The reaction mixture was poured into brine (2000 mL) and extracted with EtOAc (500 mL×3). The combined organic layers were washed with brine (1000 mL), dried over anhydrous sodium sulfate, filtered, and concentrated. The resulting residue was purified by column chromatography on silica gel (PE/EtOAc=5/1, v/v) to afford 3-fluoro-4-methyl-5-nitrobenzonitrile as a pale yellow solid (10.0 g, 86.0%).

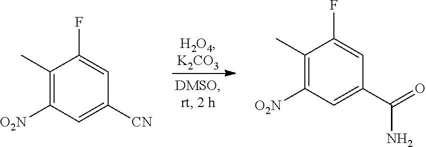

To the mixture of 3-fluoro-4-methyl-5-nitrobenzonitrile (10.0 g, 55.6 mmol, 1.0 eq), K₂CO₃ (15.3 g, 111.2 mmol, 2.0 eq) in DMSO (200.0 mL) was added 20.0 mL H₂O₂ and stirred at rt for 2.0 h. The reaction mixture was poured into brine (2000 mL) and extracted with EtOAc (500 mL×3). The combined organic layers were washed with brine (1000 mL), dried over anhydrous sodium sulfate, filtered, and concentrated. The resulting residue was purified by column chromatography on silica gel (PE/EtOAc=1/1, v/v) to afford 3-fluoro-4-methyl-5-nitrobenzamide as a pale yellow solid (5.2 g, 47.3%). LCMS (M+H⁺) m/z calculated 199.1, found 199.1.

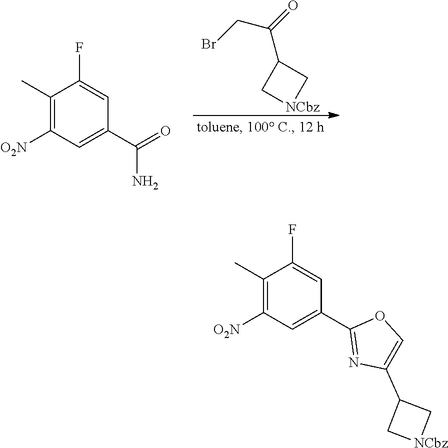 [SMILES: Cc1c(F)cc(C(N)=O)cc1[N+](=O)[O-] ; O=C(CBr)C1CN(C(=O)OCc2ccccc2)C1 ; Cc1c(F)cc(-c2nc(C3CN(C(=O)OCc4ccccc4)C3)co2)cc1[N+](=O)[O-]]

The solution of 3-fluoro-4-methyl-5-nitrobenzamide (890.0 mg, 4.5 mmol, 1.0 eq) and benzyl 3-(2-bromoacetyl)azetidine-1-carboxylate (1.7 g, 5.4 mmol, 1.2 eq) in toluene (10.0 mL) was stirred at 100° C. for 12 h. The mixture was concentrated and purified by column chromatography on silica gel (PE/EtOAc=2/1, v/v) to afford benzyl 3-(2-(3-fluoro-4-methyl-5-nitrophenyl)oxazol-4-yl)azetidine-1-carboxylate as a yellow solid (120.0 mg, 5.0%). LCMS (M+H⁺) m/z calculated 412.1, found 412.1.

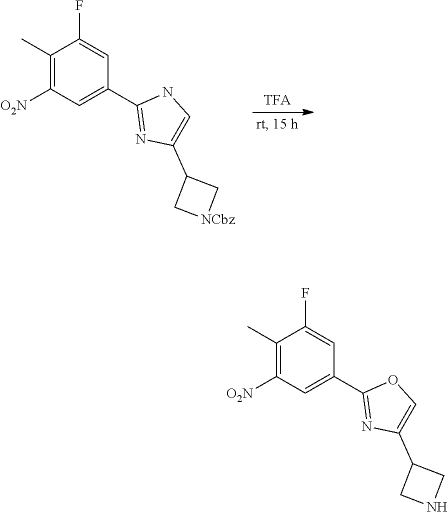 [SMILES: Cc1c(F)cc(-c2nc(C3CN(C(=O)OCc4ccccc4)C3)c[nH]2)cc1[N+](=O)[O-] ; Cc1c(F)cc(-c2nc(C3CNC3)co2)cc1[N+](=O)[O-]]

The mixture of benzyl 3-(2-(3-fluoro-4-methyl-5-nitrophenyl)oxazol-4-yl)azetidine-1-carboxylate (120.0 mg, 0.29 mmol, 1.0 eq) in TFA (2.0 mL) was stirred at rt for 15.0 h. The mixture was concentrated and the residue was obtained as crude 4-(azetidin-3-yl)-2-(3-fluoro-4-methyl-5-nitrophenyl)oxazole as a pale yellow solid (150.0 mg, quant.), which was used in the next step without further purification. LCMS (M+H⁺) m/z calculated 278.1, found 278.1.

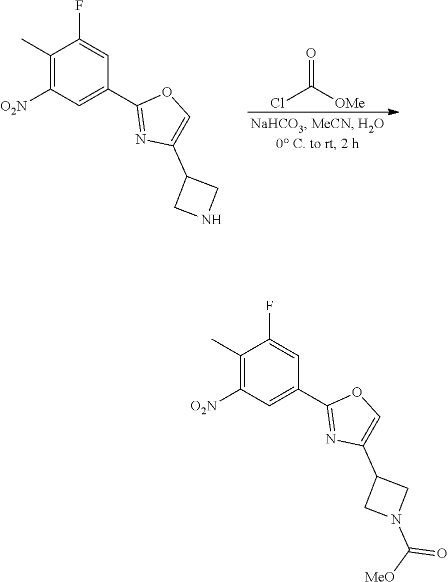 [SMILES: Cc1c(F)cc(-c2nc(C3CNC3)co2)cc1[N+](=O)[O-] ; COC(=O)Cl ; COC(=O)N1CC(c2coc(-c3cc(F)c(C)c([N+](=O)[O-])c3)n2)C1]

To the solution of 4-(azetidin-3-yl)-2-(3-fluoro-4-methyl-5-nitrophenyl)oxazole (150.0 mg, 0.54 mmol, 1.0 eq) and NaHCO₃ (136.1 mg, 1.6 mmol, 3.0 eq) in MeCN (3.0 mL) and H₂O (1.0 mL) was added methyl carbonochloridate (103.4 mg, 1.1 mmol, 2.0 eq) in MeCN (1.0 mL) at 0° C. and stirred at rt for 2.0 h. The reaction was diluted with water (10 mL) and extracted with DCM (20 mL×3). The combined organic layers were washed with brine (20 mL), dried over anhydrous sodium sulfate, filtered, and concentrated. The resulting residue was purified by column chromatography on silica gel (PE/EtOAc=1/1, v/v) to afford methyl 3-(2-(3-fluoro-4-methyl-5-nitrophenyl)oxazol-4-yl)azetidine-1-carboxylate as a pale yellow solid (80.0 mg, 44.0%). LCMS (M+H⁺) m/z calculated 336.1, found 336.1.

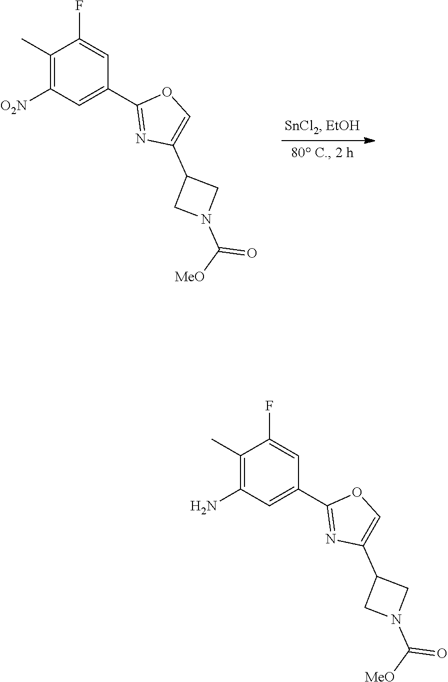 [SMILES: COC(=O)N1CC(c2coc(-c3cc(F)c(C)c([N+](=O)[O-])c3)n2)C1 ; COC(=O)N1CC(c2coc(-c3cc(N)c(C)c(F)c3)n2)C1]

To a suspension of methyl 3-(2-(3-fluoro-4-methyl-5-nitrophenyl)oxazol-4-yl)azetidine-1-carboxylate (80.0 mg, 0.24 mmol, 1.0 eq) in EtOH (50.0 mL) was added SnCl₂ (136.8 mg, 0.72 mmol, 3.0 eq). The reaction mixture was stirred at 70° C. for 3 h. The reaction was cooled to rt and the solvent was partially removed under vacuum. The pH was adjusted to slightly basic with saturated solution of sodium bicarbonate. The resulting white suspension was filtered and washed with water (30.0 mL) and EtOAc (30 mL). The aqueous layer was extracted with EtOAc (30 mL×3). The combined organic layers were washed with brine (30 mL), dried over anhydrous sodium sulfate, filtered, and concentrated. The resulting residue was purified by column chromatography on silica gel (PE/EtOAc=1/1, v/v) to afford methyl 3-(2-(3-amino-5-fluoro-4-methylphenyl)oxazol-4-yl)azetidine-1-carboxylate as a yellow solid (60.0 mg, 83.0%). LCMS (M+H⁺) m/z calculated 306.1, found 306.1.

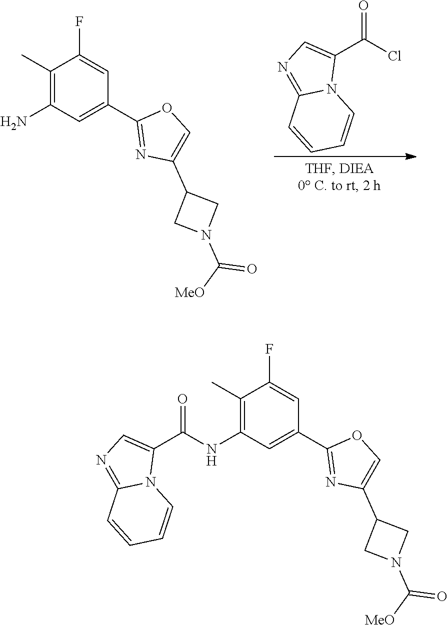 [SMILES: COC(=O)N1CC(c2coc(-c3cc(N)c(C)c(F)c3)n2)C1 ; O=C(Cl)c1cnc2ccccn12 ; COC(=O)N1CC(c2coc(-c3cc(F)c(C)c(NC(=O)c4cnc5ccccn45)c3)n2)C1]

To a solution of methyl 3-(2-(3-amino-5-fluoro-4-methylphenyl)oxazol-4-yl)azetidine-1-carboxylate (60.0 mg, 0.2 mmol, 1.0 eq) and DIEA (77.4 mg, 0.6 mmol, 3.0 eq) in THF (5.0 mL) was added imidazo[1,2-a]pyridine-3-carbonyl chloride (72.0 mg, 0.4 mmol, 2.0 eq) at 0° C. The reaction was stirred at rt for 2 h. The mixture was concentrated and purified by prep-HPLC to afford methyl 3-(2-(3-fluoro-5-(imidazo[1,2-a]pyridine-3-carboxamido)-4-methylphenyl)oxazol-4-yl)azetidine-1-carboxylate as a white solid (4.3 mg, 4.9%). LCMS (M+H⁺) m/z calculated 450.1, found 450.2. ¹H NMR (DMSO-d₆, 400 MHz) δ 10.18 (s, 1H), 9.44-9.46 (d, 1H), 8.50 (s, 1H), 8.20 (s, 1H), 7.94 (s, 1H), 7.78-7.80 (d, 1H), 7.60-7.63 (d, 1H), 7.51-7.55 (m, 2H), 7.16-7.20 (t, 1H), 4.23-4.25 (m, 2H), 3.96-3.99 (m, 2H), 3.86-3.88 (m, 1H), 3.58 (s, 3H), 2.32 (s, 3H).

### Example 48: Preparation of Methyl 3-(5-(3-fluoro-5-(imidazo[1,2-a]pyridine-3-carboxamido)-4-methylphenyl)-4H-1,2,4-triazol-3-yl)azetidine-1-carboxylate

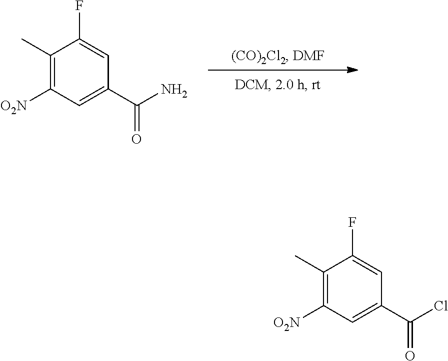

To the solution of 3-fluoro-4-methyl-5-nitrobenzoic acid (3.8 g, 19.1 mmol, 1.0 eq) in DCM (50.0 mL) were added 5 drops DMF and oxalyl chloride (12.1 g, 95.5 mmol, 5.0 eq). The reaction was stirred at rt for 2.0 h. The mixture was concentrated to afford crude 3-fluoro-4-methyl-5-nitrobenzoyl chloride as a pale yellow solid (4.1 g, quant.), which was used in the next step without further purification.

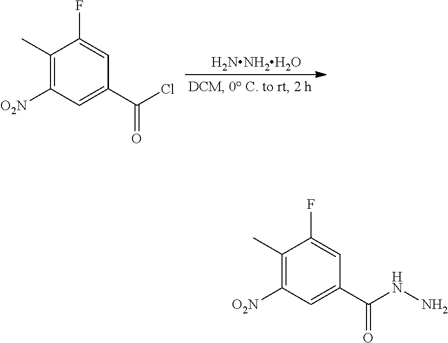

To the solution of hydrazine hydrate (40.0 mL) was added 3-fluoro-4-methyl-5-nitrobenzoyl chloride (4.1 g, 18.9 mmol, 1.0 eq) in DCM (30.0 mL) at 0° C. The mixture was then warmed to rt and stirred for 2 h. The mixture was concentrated and diluted by H₂O (100 mL). The precipitate was formed, and was filtered. The filtrate cake was dried to give 3-fluoro-4-methyl-5-nitrobenzohydrazide as a pale yellow solid (3.4 g, 85.0%). LCMS (M+H⁺) m/z calculated 214.1, found 214.1.

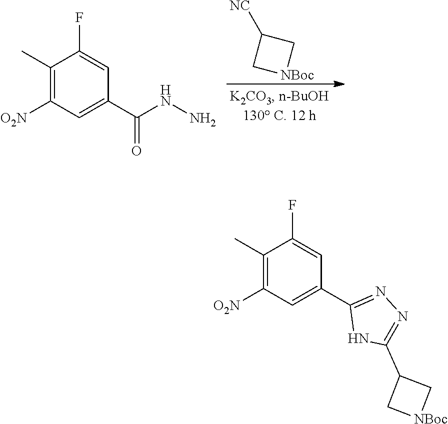 [SMILES: Cc1c(F)cc(C(=O)NN)cc1[N+](=O)[O-] ; CC(C)(C)OC(=O)N1CC(C#N)C1 ; Cc1c(F)cc(-c2nnc(C3CN(C(=O)OC(C)(C)C)C3)[nH]2)cc1[N+](=O)[O-]]

The solution of 3-fluoro-4-methyl-5-nitrobenzohydrazide (2.3 g, 10.8 mmol, 1.0 eq), tert-butyl 3-cyanoazetidine-1-carboxylate (3.9 g, 21.6 mmol, 2.0 eq) and K₂CO₃ (4.5 g, 32.4 mmol, 3.0 eq) in n-BuOH (40.0 mL) was stirred at 130° C. for 12 h. The reaction mixture was poured into H₂O (100 mL) and extracted with EtOAc (100 mL×3). The combined organic layers were washed with brine (100 mL),dried over anhydrous sodium sulfate, filtered, and concentrated. The resulting residue was purified by column chromatography on silica gel (PE/EtOAc=1/1, v/v) to afford tert-butyl 3-(5-(3-fluoro-4-methyl-5-nitrophenyl)-4H-1,2,4-triazol-3-yl)azetidine-1-carboxylate as a pale yellow solid (1.4 g, 44.0%). LCMS (M+H⁺) m/z calculated 378.2, found 378.2.

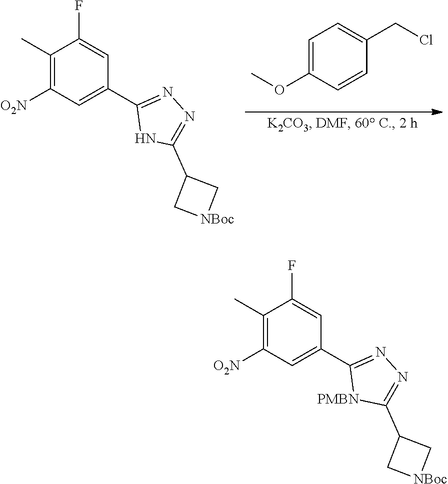

The solution of tert-butyl 3-(5-(3-fluoro-4-methyl-5-nitrophenyl)-4H-1,2,4-triazol-3-yl)azetidine-1-carboxylate (900.0 mg, 2.4 mmol, 1.0 eq), 1-(chloromethyl)-4-methoxybenzene (748.8 mg, 4.8 mmol, 2.0 eq) and K₂CO₃ (993.6 mg, 7.2 mmol, 3.0 eq) in DMF (10.0 mL) was stirred at 60° C. for 2.0 h. The reaction mixture was poured into brine (100 mL) and extracted with EtOAc (100 mL×3). The combined organic layers were washed with brine (100 mL), dried over anhydrous sodium sulfate, filtered, and concentrated. The resulting residue was purified by column chromatography on silica gel (PE/EtOAc=1/1, v/v) to afford tert-butyl 3-(5-(3-fluoro-4-methyl-5-nitrophenyl)-4-(4-methoxybenzyl)-4H-1,2,4-triazol-3-yl)azetidine-1-carboxylate as a pale yellow solid (800.0 mg, 67%). LCMS (M+H⁺) m/z calculated 498.2, found 498.2.

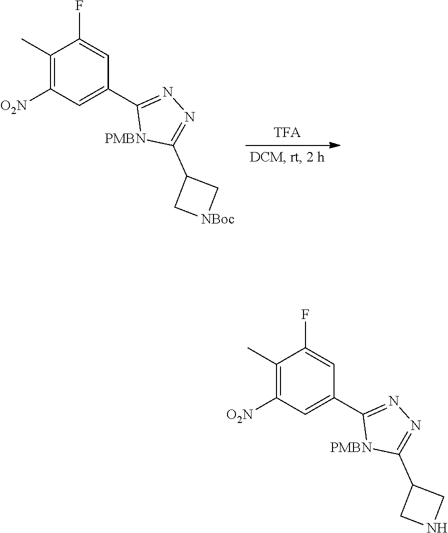

The mixture of tert-butyl 3-(5-(3-fluoro-4-methyl-5-nitrophenyl)-4-(4-methoxybenzyl)-4H-1,2,4-triazol-3-yl)azetidine-1-carboxylate (350.0 mg, 0.7 mmol, 1.0 eq) and TFA (400.0 mg, 3.5 mmol, 5.0 eq) in DCM (5.0 mL) was stirred at rt for 2 h. The mixture was concentrated to afford crude 3-(azetidin-3-yl)-5-(3-fluoro-4-methyl-5-nitrophenyl)-4-(4-methoxybenzyl)-4H-1,2,4-triazole as a pale yellow solid (200.0 mg, 71.0%), which was used in the next step without further purification. LCMS (M+H⁺) m/z calculated 398.2, found 398.2.

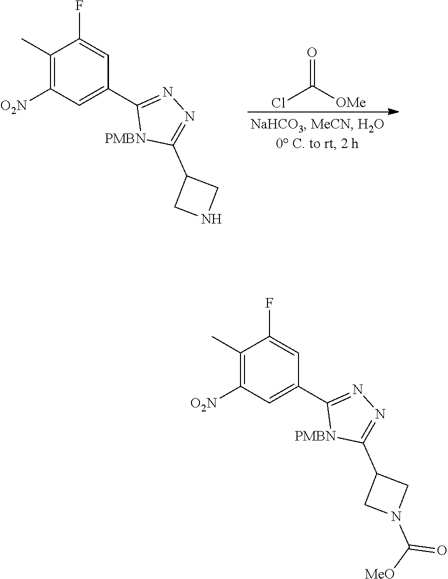

To the solution of 3-(azetidin-3-yl)-5-(3-fluoro-4-methyl-5-nitrophenyl)-4-(4-methoxybenzyl)-4H-1,2,4-triazole (200.0 mg, 0.5 mmol, 1.0 eq) and NaHCO₃ (126.0 mg, 1.5 mmol, 3.0 eq) in MeCN (3.0 mL) and H₂O (1.0 mL) was added methyl carbonochloridate (94.0 mg, 1.0 mmol, 2.0 eq) in MeCN (1.0 mL) at 0° C. The reaction was stirred at rt for 2.0 h. The reaction mixture was diluted with water (10.0 mL) and the aqueous layer was extracted with DCM (20 mL×3). The combined organic layers were washed with brine (20 mL), dried over anhydrous sodium sulfate, filtered, and concentrated. The resulting residue was purified by column chromatography on silica gel (PE/EtOAc=1/1, v/v) to afford methyl 3-(5-(3-fluoro-4-methyl-5-nitrophenyl)-4-(4-methoxybenzyl)-4H-1,2,4-triazol-3-yl)azetidine-1-carboxylate as a pale yellow solid (220.0 mg, quant.). LCMS (M+H⁺) m/z calculated 456.2, found 456.2.

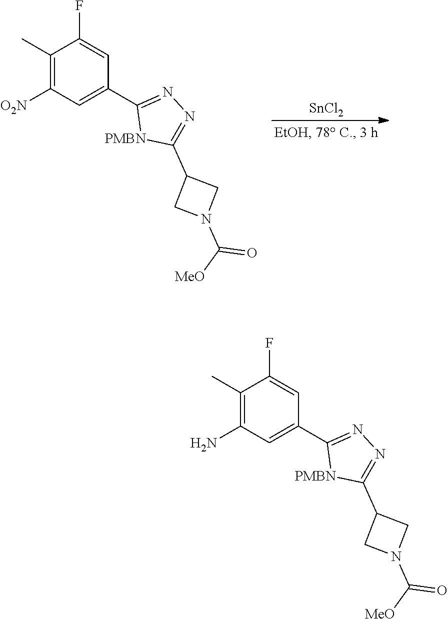

To a suspension of methyl 3-(5-(3-fluoro-4-methyl-5-nitrophenyl)-4-(4-methoxybenzyl)-4H-1,2,4-triazol-3-yl)azetidine-1-carboxylate (220.0 mg, 0.48 mmol, 1.0 eq) in EtOH (10.0 mL) was added SnCl₂ (273.6 mg, 1.4 mmol, 3.0 eq). The reaction mixture was stirred at 78° C. for 3 h. The reaction was cooled to rt and the solvent was partially removed under vacuum. The pH was adjusted to slightly basic with saturated solution of sodium bicarbonate. The resulting white suspension was filtered and washed with water (50 mL) and EtOAc (50 mL). The aqueous layer was extracted with EtOAc (50 mL×3). The combined organic layers were washed with brine (50 mL), dried over anhydrous sodium sulfate, filtered, and concentrated. The resulting residue was purified by column chromatography on silica gel (PE/EtOAc=1/1, v/v) to afford methyl 3-(5-(3-amino-5-fluoro-4-methylphenyl)-4-(4-methoxybenzyl)-4H-1,2,4-triazol-3-yl)azetidine-1-carboxylate as a yellow solid (150.0 mg, 73.2%). LCMS (M+H⁺) m/z calculated 426.2, found 426.2.

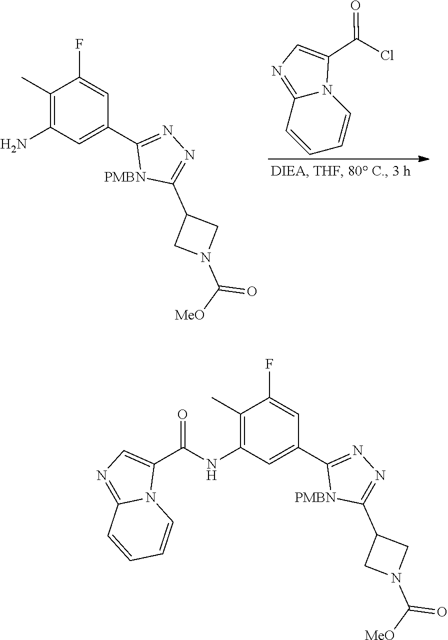

To a solution of methyl 3-(5-(3-amino-5-fluoro-4-methylphenyl)-4-(4-methoxybenzyl)-4H-1,2,4-triazol-3-yl)azetidine-1-carboxylate (150.0 mg, 0.35 mmol, 1.0 eq) and DIEA (136.6 mg, 10.5 mmol, 3.0 eq) in THF (5.0 mL) was added imidazo[1,2-a]pyridine-3-carbonyl chloride (126.0 mg, 0.7 mmol, 2.0 eq) at 0° C., and the mixture was stirred at 80° C. for 3 h. The mixture was concentrated and purified by column chromatography on silica gel (PE/EtOAc=1/1, v/v) to afford methyl 3-(5-(3-fluoro-5-(imidazo[1,2-a]pyridine-3-carboxamido)-4-methylphenyl)-4-(4-methoxybenzyl)-4H-1,2,4-triazol-3-yl)azetidine-1-carboxylate as a yellow solid (150.0 mg, 75%). LCMS (M+H⁺) m/z calculated 570.2, found 570.2.

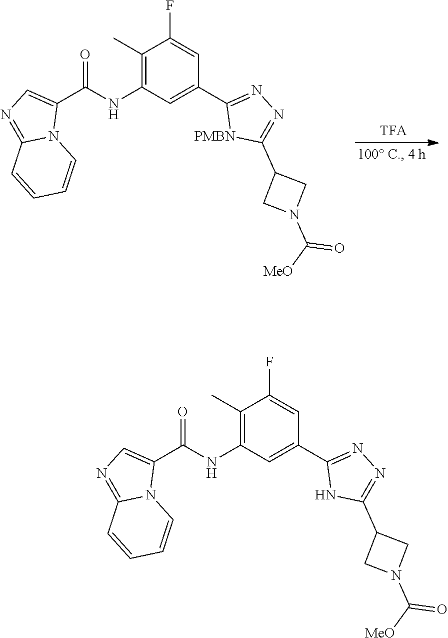

The mixture of methyl 3-(5-(3-fluoro-5-(imidazo[1,2-a]pyridine-3-carboxamido)-4-methylphenyl)-4-(4-methoxybenzyl)-4H-1,2,4-triazol-3-yl)azetidine-1-carboxylate (150.0 mg, 0.26 mmol, 1.0 eq) in TFA (2.0 mL) was stirred at 100° C. for 4 h. The mixture was concentrated under vacuum and purified by prep-HPLC to afford methyl 3-(5-(3-fluoro-5-(imidazo[1,2-a]pyridine-3-carboxamido)-4-methylphenyl)-4H-1,2,4-triazol-3-yl)azetidine-1-carboxylate as a white solid (77.6 mg, 65.8%). LCMS (M+H⁺) m/z calculated 450.2, found 450.2. ¹H NMR (DMSO-d₆, 400 MHz) δ 10.17 (s, 1H), 9.43-9.46 (m, 1H), 8.59-8.61 (d, 1H), 7.92 (s, 1H), 7.78-7.80 (d, 1H), 7.63-7.67 (m, 1H), 7.50-7.55 (m, 1H), 7.16-7.20 (m, 1H), 4.26-4.31 (m, 2H), 3.95-4.10 (m, 3H), 3.56-3.58 (d, 3H), 2.18-2.20 (d, 3H).

### Example 49: Preparation of methyl (R)-3-(3-(3-fluoro-5-(imidazo[1,2-a]pyridine-3-carboxamido)-4-methylphenyl)-1,2,4-oxadiazol-5-yl)piperidine-1-carboxylate

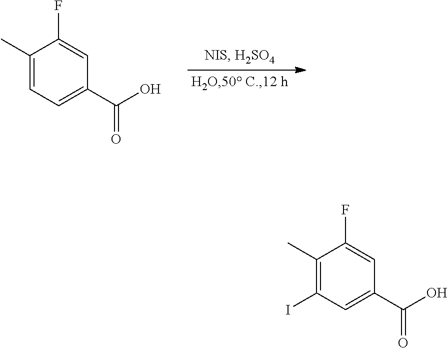

To the mixture of 3-fluoro-4-methylbenzoic acid (10.0 g, 64.9 mmol, 1.0 eq) in Con·H₂SO₄ (50.0 mL) and H₂O (50.0 mL) was added N-Iodosuccinimide (17.5 g, 77.9 mmol, 1.2 eq) and the mixture was stirred at 50° C. for 12 h. The mixture was poured into ice water. The precipitate was filtered and dried to give 3-fluoro-5-iodo-4-methylbenzoic acid as a pale yellow solid (7.1 g, 40.0%). LCMS (M−H⁺) m/z calculated 278.9, found 279.1.

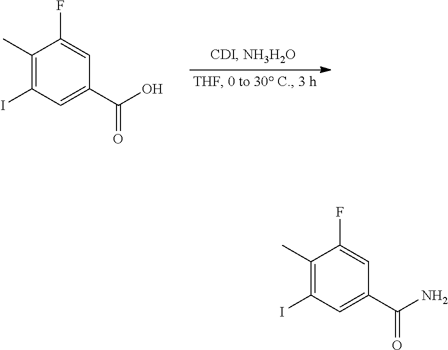

To the solution of 3-fluoro-5-iodo-4-methylbenzoic acid (7.1 g, 25.5 mmol, 1.0 eq) in THF (100.0 mL) was added 1,1′-Carbonyldiimidazole (6.2 g, 38.3 mmol, 1.5 eq) and the mixture was stirred at 30° C. for 3.0 h. The reaction mixture was added to 26% NH₃·H₂O (200 mL) at 0° C. and stirred at 30° C. for 3.0 h. The mixture was poured into ice water and precipitate was formed. The precipitate was filtered and dried to give 3-fluoro-5-iodo-4-methylbenzamide as a pale yellow solid (6.0 g, 86.0%). LCMS (M+H⁺) m/z calculated 280.0, found 280.1.

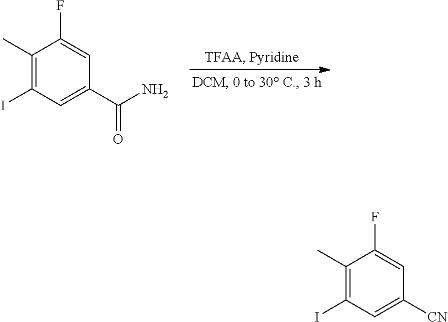

To the mixture of 3-fluoro-5-iodo-4-methylbenzamide (6.0 g, 21.5 mmol, 1.0 eq) and pyridine (8.5 g, 107.5 mmol, 5.0 eq) was added trifluoroacetic anhydride (9.0 g, 43.0 mmol, 2.0 eq) in DCM (200.0 mL) at 0° C. and the mixture was stirred at 30° C. for 3 h. The mixture was concentrated and purified by column chromatography on silica gel (PE/EtOAc=5/1, v/v) to afford 3-fluoro-5-iodo-4-methylbenzonitrile as a pale yellow solid (3.5 g, 62.5%).

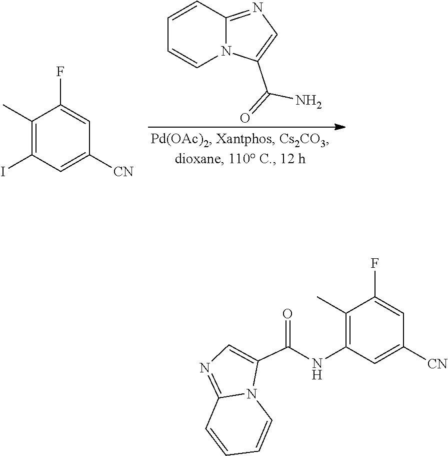 [SMILES: Cc1c(F)cc(C#N)cc1I ; NC(=O)c1cnc2ccccn12 ; Cc1c(F)cc(C#N)cc1NC(=O)c1cnc2ccccn12]

The mixture of 3-fluoro-5-iodo-4-methylbenzonitrile (3.5 g, 13.4 mmol, 1.0 eq), imidazo[1,2-a]pyridine-3-carboxamide (2.2 g, 13.4 mmol, 1.0 eq), palladium (II) acetate (216.0 mg, 1.3 mmol, 0.1 eq), Xantphos (776.0 mg, 1.3 mmol, 0.1 eq) and Cs₂CO₃ (8.7 g, 26.8 mmol, 2.0 eq) in dioxane (100.0 mL) was stirred at 110° C. for 12 h. The reaction was quenched by adding H₂O (100 mL) and the mixture was extracted with EtOAc (150 mL×2). The combined organic layers were washed with brine (150 mL), dried over anhydrous sodium sulfate, filtered, and concentrated. The resulting residue was purified by column chromatography on silica gel (PE/EtOAc=2/1, v/v) to afford N-(5-cyano-3-fluoro-2-methylphenyl)imidazo[1,2-a]pyridine-3-carboxamide as a pale yellow solid (1.9 g, 48.7%). LCMS (M+H⁺) m/z calculated 295.1, found 295.1.

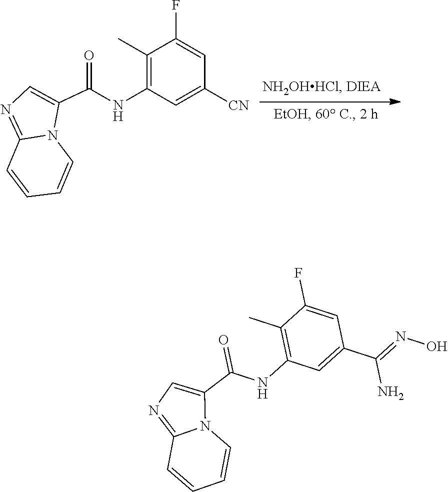 [SMILES: Cc1c(F)cc(C#N)cc1NC(=O)c1cnc2ccccn12 ; Cc1c(F)cc(/C(N)=N/O)cc1NC(=O)c1cnc2ccccn12]

The mixture of N-(5-cyano-3-fluoro-2-methylphenyl)imidazo[1,2-a]pyridine-3-carboxamide (1.9 g, 6.4 mmol, 1.0 eq), hydroxylamine hydrochloride (892.0 mg, 12.8 mmol, 2.0 eq) and N,N-Diisopropylethylamine (4.1 g, 12.0 mmol, 5.0 eq) in EtOH (30.0 mL) was stirred at 60° C. for 2 h. The reaction was quenched by adding H₂O (100 mL) and extracted with EtOAc (100 mL×2). The combined organic layers were washed with brine (100 mL), dried over anhydrous sodium sulfate, filtered, and concentrated. The resulting residue was purified by column chromatography on silica gel (PE/EtOAc=2/1, v/v) to afford (Z)-N-(3-fluoro-5-(N′-hydroxycarbamimidoyl)-2-methylphenyl)imidazo[1,2-a]pyridine-3-carboxamide as a pale yellow solid (700.0 mg, 42.0%). LCMS (M+H⁺) m/z calculated 328.1, found 328.1.

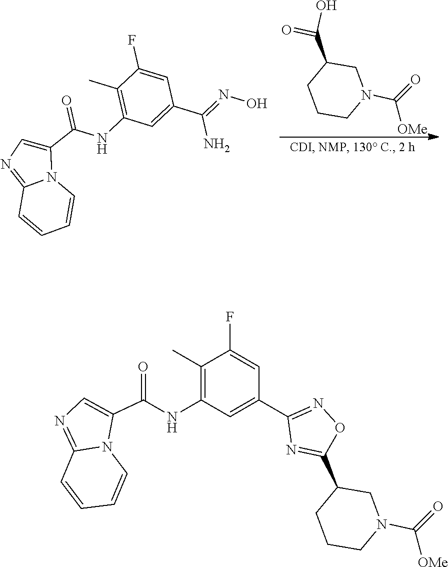 [SMILES: Cc1c(F)cc(/C(N)=N/O)cc1NC(=O)c1cnc2ccccn12 ; COC(=O)N1CCC[C@@H](C(=O)O)C1 ; COC(=O)N1CCC[C@@H](c2nc(-c3cc(F)c(C)c(NC(=O)c4cnc5ccccn45)c3)no2)C1]

To the mixture of (R)-1-(methoxycarbonyl)piperidine-3-carboxylic acid (299.0 mg, 1.6 mmol, 1.5 eq) in NMP (10.0 mL) was added 1,1′-Carbonyldiimidazole (260.0 mg, 1.6 mmol, 1.5 eq) and the mixture was stirred at 40° C. for 2 h. (Z)-N-(3-fluoro-5-(N′-hydroxycarbamimidoyl)-2-methylphenyl)imidazo[1,2-a]pyridine-3-carboxamide (350.0 mg, 1.1 mmol, 1.0 eq) was added to the above mixture and stirred at 130° C. for 2 h. The reaction was quenched by adding H₂O (100 mL) and the mixture was extracted with EtOAc (50 mL×3). The combined organic layers were washed with brine (100 mL), dried over anhydrous sodium sulfate, filtered, and concentrated. The resulting residue was purified by prep-HPLC to afford methyl (R)-3-(3-(3-fluoro-5-(imidazo[1,2-a]pyridine-3-carboxamido)-4-methylphenyl)-1,2,4-oxadiazol-5-yl)piperidine-1-carboxylate as a white solid (148.9 mg, 29.1%). LCMS (M+H⁺) m/z calculated 479.2, found 479.2. ¹H NMR (DMSO-d₆, 400 MHz) δ 10.22 (s, 1H), 9.43-9.45 (d, 1H), 8.60 (s, 1H), 7.95 (s, 1H), 7.78-7.80 (d, 1H), 7.63-7.65 (d, 1H), 7.51-7.55 (t, 1H), 7.17-7.20 (t, 1H), 4.12-4.16 (m, 1H), 3.74-3.77 (m, 1H), 3.59 (s, 3H), 3.36-3.37 (m, 2H), 3.05-3.27 (m, 1H), 2.31-2.32 (d, 3H), 2.18-2.25 (m, 1H), 1.85-1.88 (m, 1H), 1.57-1.72 (m, 1H), 1.50-1.57 (m, 1H).

### Example 50: Preparation of N-(3-fluoro-5-(5-((1S,2R)-2-fluorocyclopropyl)-1,2,4-oxadiazol-3-yl)-2-methylphenyl)-7-morpholinoimidazo[1,2-a]pyridine-3-carboxamide

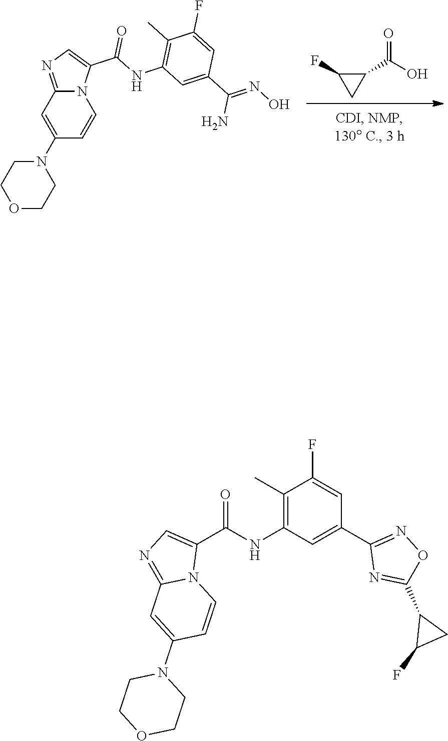 [SMILES: Cc1c(F)cc(/C(N)=N/O)cc1NC(=O)c1cnc2cc(N3CCOCC3)ccn12 ; O=C(O)[C@@H]1C[C@H]1F ; Cc1c(F)cc(-c2noc([C@@H]3C[C@H]3F)n2)cc1NC(=O)c1cnc2cc(N3CCOCC3)ccn12]

To the mixture of (1S,2R)-2-fluorocyclopropane-1-carboxylic acid (47.3 mg, 0.45 mmol, 1.5 eq) in NMP (5.0 mL) was added 1,1′-Carbonyldiimidazole (72.9 mg, 0.45 mmol, 1.5 eq) and the mixture was stirred at 40° C. for 2.0 h. (Z)-N-(3-fluoro-5-(N′-hydroxycarbamimidoyl)-2-methylphenyl)imidazo[1,2-a]pyridine-3-carboxamide (125.0 mg, 0.3 mmol, 1.0 eq) was added to the above mixture and stirred at 130° C. for 3 h. The reaction was quenched by adding H₂O (50 mL) and the mixture was extracted with EtOAc (50 mL×3). The combined organic layers were washed with brine (50 mL), dried over anhydrous sodium sulfate, filtered, and concentrated. The resulting residue was purified by prep-HPLC to afford N-(3-fluoro-5-(5-((1S,2R)-2-fluorocyclopropyl)-1,2,4-oxadiazol-3-yl)-2-methylphenyl)-7-morpholinoimidazo[1,2-a]pyridine-3-carboxamide as a white solid (28.3 mg, 19.5%). LCMS (M+H⁺) m/z calculated 481.2, found 481.2. ¹H NMR (DMSO-d₆, 400 MHz) δ 9.96 (s, 1H), 9.14-9.16 (d, 1H), 8.40 (s, 1H), 7.90 (s, 1H), 7.56-7.58 (d, 1H), 7.08-7.10 (m, 1H), 6.90-6.91 (d, 1H), 5.20-5.21 (m, 1H), 3.74-3.77 (t, 4H), 3.31-3.32 (t, 4H), 3.05-3.09 (m, 1H), 2.23-2.24 (d, 3H), 1.92-1.98 (m, 1H), 1.57-1.61 (m, 1H).

### Example 51: Preparation of Methyl 3-(2-(3-fluoro-5-(imidazo[1,2-a]pyridine-3-carboxamido)-4-methylphenyl)oxazol-5-yl)azetidine-1-carboxylate

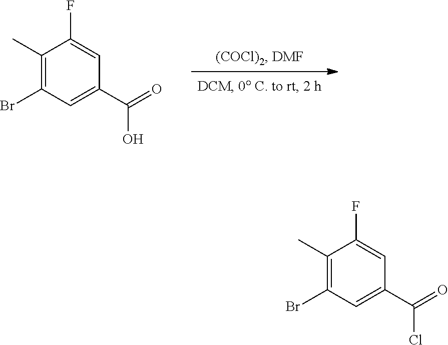

To the solution of 3-bromo-5-fluoro-4-methylbenzoic acid (200.0 mg, 0.86 mmol, 1.0 eq) and 2 drops DMF in DCM (5.0 mL) was added oxalyl chloride (546.0 mg, 4.3 mmol, 5.0 eq).The mixture was stirred at rt for 2 h, and concentrated to afford crude 3-bromo-5-fluoro-4-methylbenzoyl chloride as pale yellow solid (250.0 mg, quant.), which was used in the next step without further purification.

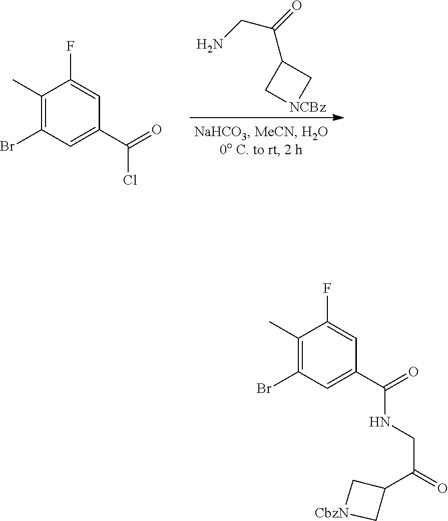

To the solution of benzyl 3-glycylazetidine-1-carboxylate (248.0 mg, 1.0 mmol, 1.0 eq) and NaHCO₃ (252.0 mg, 3.0 mmol, 3.0 eq) in MeCN (3.0 mL) and H₂O (1.0 mL) was added 3-bromo-5-fluoro-4-methylbenzoyl chloride (250.0 mg, 1.0 mmol, 1.0 eq) in MeCN (3.0 mL) at 0° C. and stirred at rt for 2 h. The reaction was diluted with water (20 mL) and the aqueous layer was extracted with DCM (50 mL×3). The combined organic layers were washed with brine (20 mL), dried over anhydrous sodium sulfate, filtered, and concentrated. The resulting residue was purified by column chromatography on silica gel (PE/EtOAc=2/1, v/v) to afford benzyl 3-((3-bromo-5-fluoro-4-methylbenzoyl)glycyl)azetidine-1-carboxylate as white solid (110.0 mg, 50.0%). LCMS (M+H⁺) m/z calculated 463.1, found 463.1.

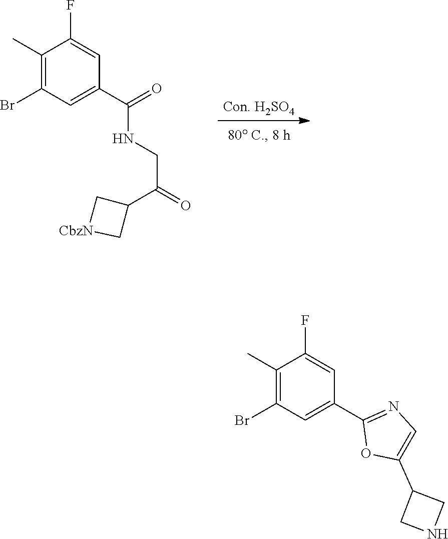 [SMILES: Cc1c(F)cc(C(=O)NCC(=O)C2CN(C(=O)OCc3ccccc3)C2)cc1Br ; Cc1c(F)cc(-c2ncc(C3CNC3)o2)cc1Br]

The mixture of benzyl 3-((3-bromo-5-fluoro-4-methylbenzoyl)glycyl)azetidine-1-carboxylate (110.0 mg, 0.24 mmol, 1.0 eq) in Con·H₂SO₄ (2.0 mL) was stirred at 80° C. for 8 h. The mixture was poured into ice water and the pH was adjusted to 10.0 by adding saturated aqueous Na₂CO₃. The mixture was extracted with DCM/MeOH (30 mL×3). The combined organic layers were washed with brine (50 mL), dried over anhydrous sodium sulfate, filtered, and concentrated. The resulting residue was purified by column chromatography on silica gel (DCM/MeOH=10/1, v/v) to afford 5-(azetidin-3-yl)-2-(3-bromo-5-fluoro-4-methylphenyl)oxazole as pale yellow solid (60.0 mg, 83.0%). LCMS (M+H⁺) m/z calculated 311.0, found 311.1.

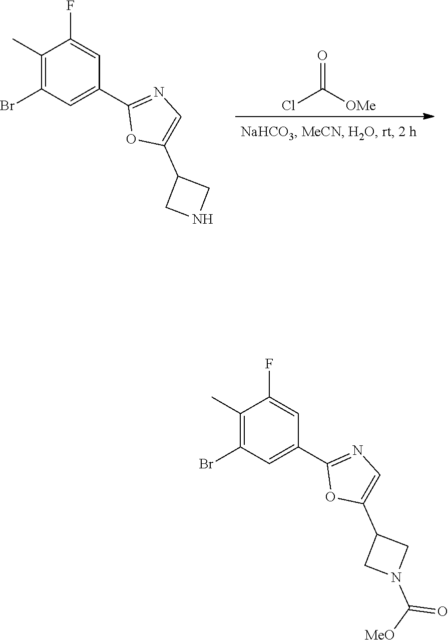 [SMILES: Cc1c(F)cc(-c2ncc(C3CNC3)o2)cc1Br ; COC(=O)Cl ; COC(=O)N1CC(c2cnc(-c3cc(F)c(C)c(Br)c3)o2)C1]

To the solution of 5-(azetidin-3-yl)-2-(3-bromo-5-fluoro-4-methylphenyl)oxazole (60.0 mg, 0.19 mmol, 1.0 eq) and NaHCO₃ (47.0 mg, 0.58 mmol, 3.0 eq) in MeCN (1.0 mL) and H₂O (0.5 mL) was added methyl carbonochloridate (54.5 mg, 0.58 mmol, 1.0 eq) in MeCN (1.0 mL) at 0° C. and stirred at rt for 2 h. The reaction was diluted with water (10 mL) and the aqueous layer was extracted with DCM (20 mL×3). The combined organic layers were washed with brine (20 mL),dried over anhydrous sodium sulfate, filtered, and concentrated. The resulting residue was purified by column chromatography on silica gel (PE/EtOAc=1/1, v/v) to afford methyl 3-(2-(3-bromo-5-fluoro-4-methylphenyl)oxazol-5-yl)azetidine-1-carboxylate as a pale yellow solid (50.0 mg, 53.0%). LCMS (M+H⁺) m/z calculated 369.0, found 369.1.

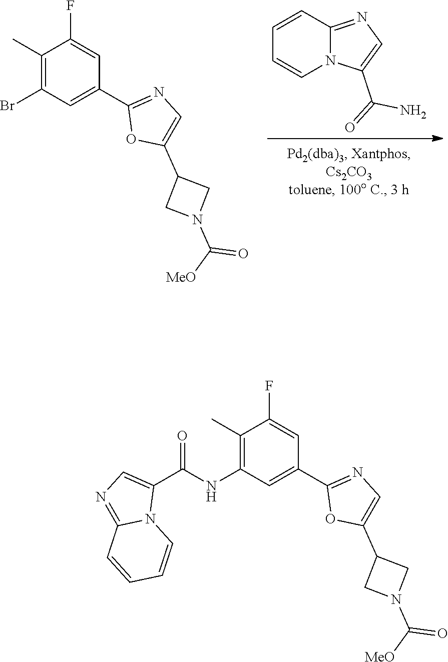 [SMILES: COC(=O)N1CC(c2cnc(-c3cc(F)c(C)c(Br)c3)o2)C1 ; NC(=O)c1cnc2ccccn12 ; COC(=O)N1CC(c2cnc(-c3cc(F)c(C)c(NC(=O)c4cnc5ccccn45)c3)o2)C1]

The mixture of methyl 3-(2-(3-bromo-5-fluoro-4-methylphenyl)oxazol-5-yl)azetidine-1-carboxylate (50.0 mg, 0.14 mmol, 1.0 eq), imidazo[1,2-a]pyridine-3-carboxamide (21.8 mg, 0.14 mmol, 1.0 eq), Pa₂(dba)₃ (12.8 mg, 0.0.014 mmol, 0.1 eq), Xantphos (8.1 mg, 0.014 mmol, 0.1 eq) and Cs₂CO₃ (106.4 mg, 0.28 mmol, 2.0 eq) in toluene (2.0 mL) was stirred at 100° C. for 3 h. The reaction was quenched by adding H₂O (5.0 mL), and the mixture was extracted with EtOAc (20 mL×2). The combined organic layers were washed with brine (20 mL), dried over anhydrous sodium sulfate, filtered, and concentrated. The resulting residue was purified by prep-HPLC to afford methyl 3-(2-(3-fluoro-5-(imidazo[1,2-a]pyridine-3-carboxamido)-4-methylphenyl)oxazol-5-yl)azetidine-1-carboxylate as a white solid (1.0 mg, 1.6%). LCMS (M+H⁺) m/z calculated 450.1, found 450.2. ¹H NMR (DMSO-d₆, 400 MHz) δ 10.21 (s, 1H), 9.44-9.45 (d, 1H), 8.59 (s, 1H), 7.88 (s, 1H), 7.78-7.80 (d, 1H), 7.61-7.64 (t, 1H), 7.51-7.55 (m, 1H), 7.31 (s, 1H), 7.16-7.18 (t, 1H), 4.27-4.29 (m, 2H), 4.07-4.09 (m, 3H), 3.58 (s, 3H), 2.32-2.33 (d, 3H).

### Example 52: Preparation of Methyl 3-(3-(2,5-difluoro-3-(imidazo[1,2-a]pyridine-3-carboxamido)-4-methylphenyl)-1,2,4-oxadiazol-5-yl)azetidine-1-carboxylate

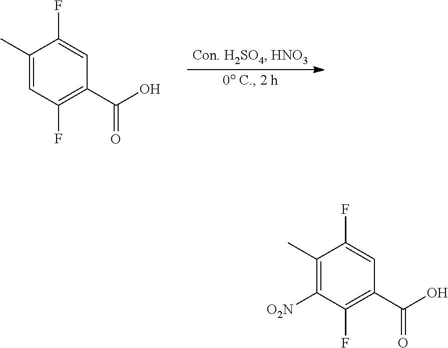

To the mixture of 2,5-difluoro-4-methylbenzoic acid (10.0 g, 58.1 mmol, 1.0 eq) in Con·H₂SO₄ (50.0 mL) was added fuming HNO₃ (10.0 mL) at 0° C. and the mixture was stirred for 2.0 h. The mixture was poured into ice water and precipitate was formed. The mixture was filtered and the solid was dried to give 2,5-difluoro-4-methyl-3-nitrobenzoic acid as a pale yellow solid (8.0 g, 63.0%). LCMS (M−H⁺) m/z calculated 216.1, found 216.1.

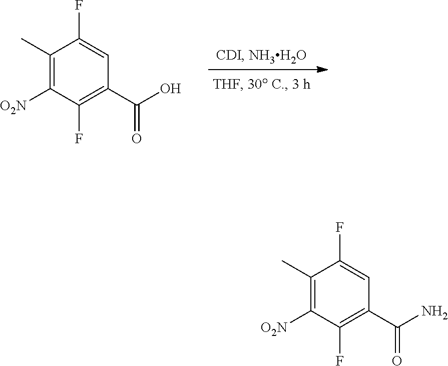

To the mixture of 2,5-difluoro-4-methyl-3-nitrobenzoic acid (8.0 g, 47.1 mmol, 1.0 eq) in THF (200.0 mL) was added 1,1′-Carbonyldiimidazole (8.9 g, 55.2 mmol, 1.5 eq) and the mixture was stirred at 30° C. for 3.0 h. The reaction mixture was added to 26% NH₃·H₂O (200.0 mL) at 0° C. and stirred at 0° C. for 3.0 h. The mixture was poured into ice water. The precipitate was filtered and dried to give 2,5-difluoro-4-methyl-3-nitrobenzamide as a pale yellow solid (5.2 g, 65.8%). LCMS (M+H⁺) m/z calculated 217.1, found 217.1.

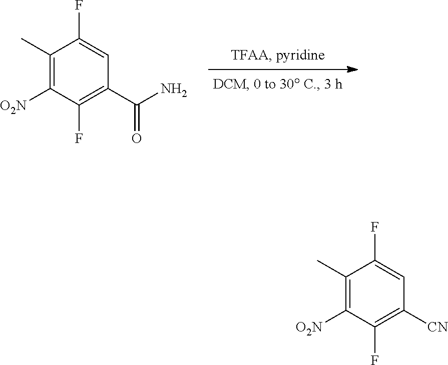

To the mixture of 2,5-difluoro-4-methyl-3-nitrobenzamide (5.2 g, 24.1 mmol, 1.0 eq) and pyridine (9.5 g, 120.5 mmol, 5.0 eq) in DCM (200.0 mL) was added trifluoroacetic anhydride (10.1 g, 48.2 mmol, 2.0 eq) at 0° C., and the mixture was stirred at 30° C. for 3 h. The mixture was concentrated and purified by column chromatography on silica gel (PE/EtOAc=5/1, v/v) to afford 2,5-difluoro-4-methyl-3-nitrobenzonitrile as a pale yellow solid (4.2 g, 87.5%).

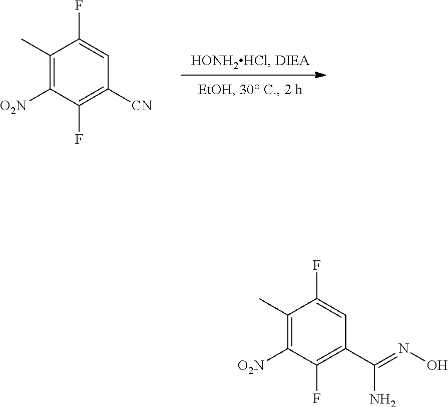 [SMILES: Cc1c(F)cc(C#N)c(F)c1[N+](=O)[O-] ; Cc1c(F)cc(/C(N)=N/O)c(F)c1[N+](=O)[O-]]

The mixture of 2,5-difluoro-4-methyl-3-nitrobenzonitrile (2.0 g, 10.1 mmol, 1.0 eq), hydroxylamine hydrochloride (1.4 g, 20.2 mmol, 2.0 eq) and N,N-Diisopropylethylamine (6.5 g, 50.5 mmol, 5.0 eq) in EtOH (40.0 mL) was stirred at 30° C. for 2 h. The reaction was quenched by adding H₂O (100.0 mL) and the mixture was extracted with EtOAc (100 mL×2). The combined organic layers were washed with brine (100.0 mL), dried over anhydrous sodium sulfate, filtered, and concentrated. The resulting residue was purified by column chromatography on silica gel (PE/EtOAc=2/1, v/v) to afford (Z)-2,5-difluoro-N′-hydroxy-4-methyl-3-nitrobenzimidamide as a pale yellow solid (1.5 g, 65.2%). LCMS (M+H⁺) m/z calculated 232.1, found 232.1.

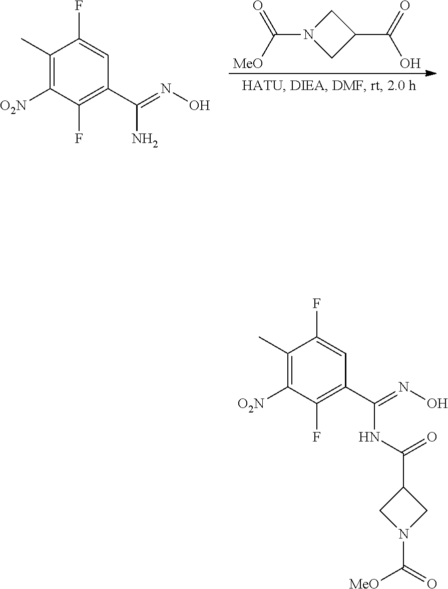 [SMILES: Cc1c(F)cc(/C(N)=N/O)c(F)c1[N+](=O)[O-] ; COC(=O)N1CC(C(=O)O)C1 ; COC(=O)N1CC(C(=O)N/C(=N\O)c2cc(F)c(C)c([N+](=O)[O-])c2F)C1]

To the mixture of (Z)-2,5-difluoro-N′-hydroxy-4-methyl-3-nitrobenzimidamide (1.4 g, 6.1 mmol, 1.0 eq), 1-(methoxycarbonyl)azetidine-3-carboxylic acid (356 mg, 6.1 mmol, 1.0 eq) and HATU (4.6 g, 12.2 mmol, 2.0 eq) in DMF (20.0 mL) was added DIEA (2.3 g, 18.3 mmol, 3.0 eq) at 0° C. and the mixture was stirred at rt for 2.0 h. The reaction was quenched by adding H₂O (100 mL) and the mixture was extracted with EtOAc (100 mL×3). The combined organic layers were washed with brine (100 mL), dried over anhydrous sodium sulfate, filtered, and concentrated. The resulting residue was purified by column chromatography on silica gel (PE/EtOAc=1/1, v/v) to afford methyl (Z)-3-(((2,5-difluoro-4-methyl-3-nitrophenyl)(hydroxyimino)methyl)carbamoyl)azetidine-1-carboxylate as a pale yellow solid (1.5 g, 65.2%). LCMS (M+H⁺) m/z calculated 373.1, found 373.1.

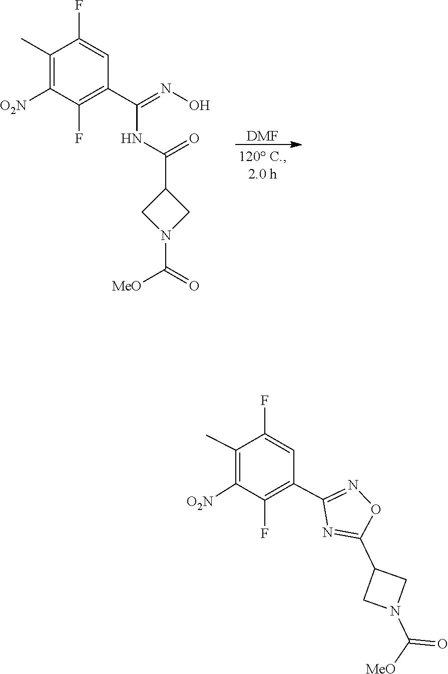

The mixture of methyl (Z)-3-(((2,5-difluoro-4-methyl-3-nitrophenyl)(hydroxyimino)methyl)carbamoyl)azetidine-1-carboxylate (1.5 g, 4.0 mmol, 1.0 eq) in DMF (20.0 mL) was stirred at 120° C. for 2.0 h. The mixture was poured into brine (200.0 mL) and extracted with EtOAc (100.0 mL×3). The combined organic layers were washed with brine (100.0 mL), dried over anhydrous sodium sulfate, filtered, and concentrated. The resulting residue was purified by column chromatography on silica gel (PE/EtOAc=1/1, v/v) to afford methyl 3-(3-(2,5-difluoro-4-methyl-3-nitrophenyl)-1,2,4-oxadiazol-5-yl)azetidine-1-carboxylate as pale yellow oil (1.1 g, 78.5%). LCMS (M+H⁺) m/z calculated 355.1, found 355.1.

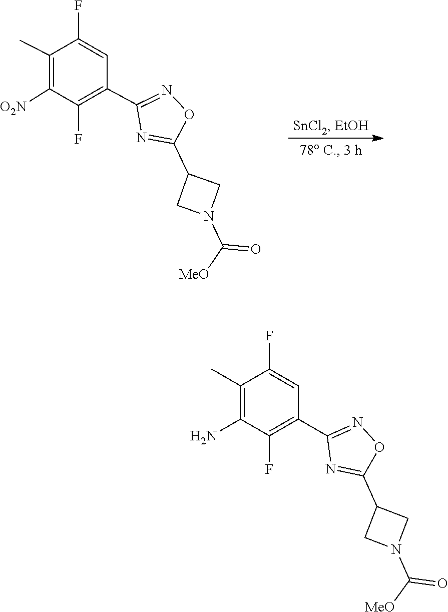 [SMILES: COC(=O)N1CC(c2nc(-c3cc(F)c(C)c([N+](=O)[O-])c3F)no2)C1 ; COC(=O)N1CC(c2nc(-c3cc(F)c(C)c(N)c3F)no2)C1]

To a suspension of methyl 3-(3-(2,5-difluoro-4-methyl-3-nitrophenyl)-1,2,4-oxadiazol-5-yl)azetidine-1-carboxylate (1.1 g, 3.1 mmol, 1.0 eq) in EtOH (20.0 mL) was added SnCl₂ (1.8 g, 9.3 mmol, 3.0 eq). The reaction mixture was stirred at 78° C. for 3 h. The reaction was cooled to rt and the solvent was partially removed under vacuum. The pH was adjusted to slightly basic with saturated solution of sodium bicarbonate. The resulting white suspension was filtered and washed with water (30 mL) and EtOAc (30 mL). The aqueous layer was extracted with EtOAc (100 mL×2). The combined organic layers were washed with brine (100 mL), dried over anhydrous sodium sulfate, filtered, and concentrated. The resulting residue was purified by column chromatography on silica gel (PE/EtOAc=1/1, v/v) to afford methyl 3-(3-(3-amino-2,5-difluoro-4-methylphenyl)-1,2,4-oxadiazol-5-yl)azetidine-1-carboxylate as a yellow solid (800.0 mg. 80.0%). LCMS (M+H⁺) m/z calculated 325.1, found 325.1.

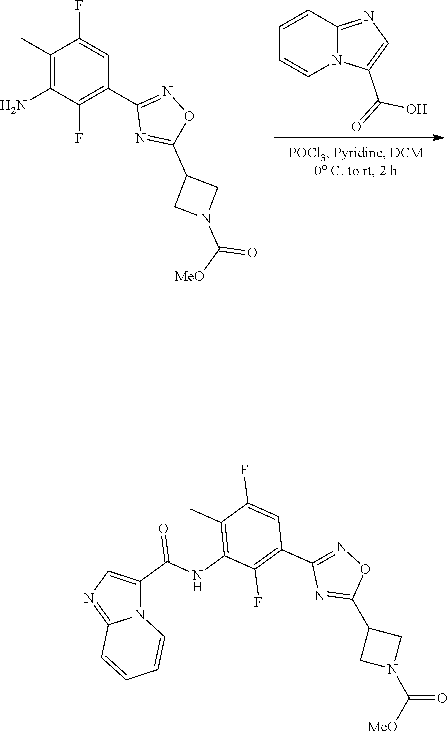 [SMILES: COC(=O)N1CC(c2nc(-c3cc(F)c(C)c(N)c3F)no2)C1 ; O=C(O)c1cnc2ccccn12 ; COC(=O)N1CC(c2nc(-c3cc(F)c(C)c(NC(=O)c4cnc5ccccn45)c3F)no2)C1]

To a stirring solution of imidazo[1,2-a]pyridine-3-carboxylic acid (275.0 mg, 1.7 mmol, 1.0 eq), methyl 3-(3-(3-amino-2,5-difluoro-4-methylphenyl)-1,2,4-oxadiazol-5-yl)azetidine-1-carboxylate (550.0 mg, 1.7 mmol, 1.0 eq) and pyridine (829.5 mg, 10.5 mmol, 5.0 eq) in DCM (15.0 mL) was added POCl₃ (780.3 mg, 5.1 mmol, 3.0 eq) at 0° C. The reaction mixture was stirred at rt for 2 h, and diluted with water (30 mL). The aqueous layer was extracted with DCM (30 mL×3). The combined organic layers were washed with brine (50 mL), dried over anhydrous sodium sulfate, filtered, and concentrated. The resulting residue was purified by prep-HPLC to afford methyl 3-(3-(2,5-difluoro-3-(imidazo[1,2-a]pyridine-3-carboxamido)-4-methylphenyl)-1,2,4-oxadiazol-5-yl)azetidine-1-carboxylate (170.0 mg, 21.4%). LCMS (M+H⁺) m/z calculated 469.1, found 469.1. ¹H NMR (DMSO-d₆, 400 MHz) δ 10.30 (s, 1H), 9.40-9.42 (d, 1H), 8.63 (s, 1H), 7.77-7.82 (m, 2H), 7.53-7.57 (m, 1H), 7.18-7.21 (t, 1H), 4.34-4.38 (m, 2H), 4.30-4.33 (m, 1H), 4.21-4.22 (m, 2H), 3.60 (s, 3H), 2.26-2.27 (d, 3H).

### Example 53: Preparation of methyl (R)-3-(3-(2,5-difluoro-3-(imidazo[1,2-a]pyridine-3-carboxamido)-4-methylphenyl)-1,2,4-oxadiazol-5-yl)pyrrolidine-1-carboxylate

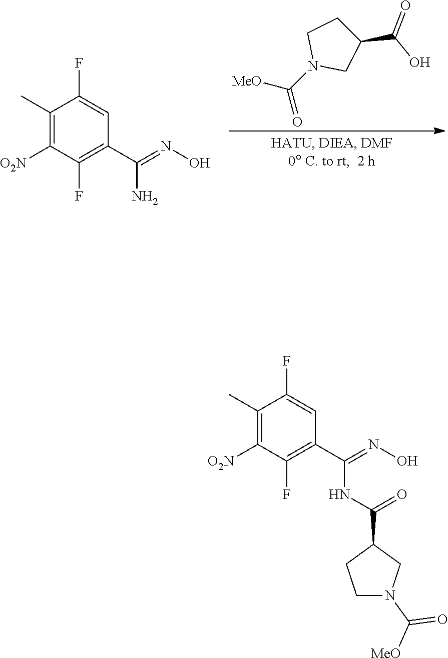 [SMILES: Cc1c(F)cc(/C(N)=N/O)c(F)c1[N+](=O)[O-] ; COC(=O)N1CC[C@@H](C(=O)O)C1 ; COC(=O)N1CC[C@@H](C(=O)N/C(=N\O)c2cc(F)c(C)c([N+](=O)[O-])c2F)C1]

To the mixture of (Z)-2,5-difluoro-N′-hydroxy-4-methyl-3-nitrobenzimidamide (900.0 mg, 3.9 mmol, 1.0 eq), 1-(methoxycarbonyl)azetidine-3-carboxylic acid (674.0 mg, 3.9 mmol, 1.0 eq) and HATU (2.9 g, 7.8 mmol, 2.0 eq) in DMF (20.0 mL) was added DIEA (1.1 g, 8.7 mmol, 3.0 eq) at 0° C. and the mixture was stirred at rt for 2 h. The reaction was quenched by adding H₂O (100 mL) and the mixture was extracted with EtOAc (100 mL×3). The combined organic layers were washed with brine (100 mL), dried over anhydrous sodium sulfate, filtered, and concentrated. The resulting residue was purified by column chromatography on silica gel (PE/EtOAc=1/1, v/v) to afford methyl (R,Z)-3-(((2,5-difluoro-4-methyl-3-nitrophenyl)(hydroxyimino)methyl)carbamoyl)pyrrolidine-1-carboxylate as a pale yellow solid (900.0 mg, 60.0%). LCMS (M+H⁺) m/z calculated 387.1, found 387.1.

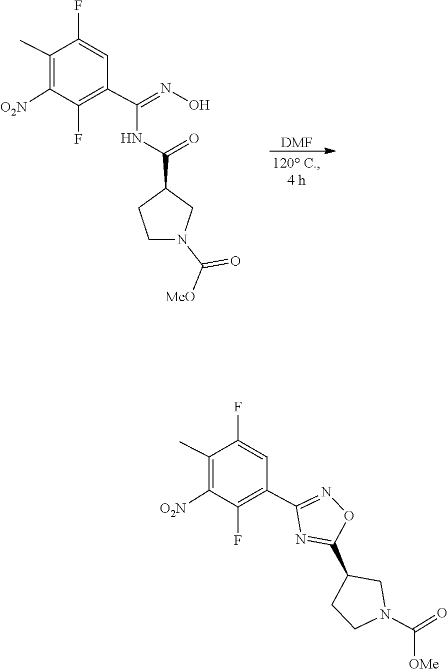

The mixture of methyl (R,Z)-3-(((2,5-difluoro-4-methyl-3-nitrophenyl)(hydroxyimino)methyl)carbamoyl)pyrrolidine-1-carboxylate (900.0 mg, 2.3 mmol, 1.0 eq) in DMF (20.0 mL) was stirred at 120° C. for 4 h. The mixture was poured into brine (200 mL) and extracted with EtOAc (100 mL×3). The combined organic layers were concentrated, and the resulting residue was purified by column chromatography on silica gel (PE/EtOAc=1/1, v/v) to afford methyl (R)-3-(3-(2,5-difluoro-4-methyl-3-nitrophenyl)-1,2,4-oxadiazol-5-yl)pyrrolidine-1-carboxylate as pale yellow oil (700.0 mg, 81.6%). LCMS (M+H⁺) m/z calculated 369.1, found 369.1.

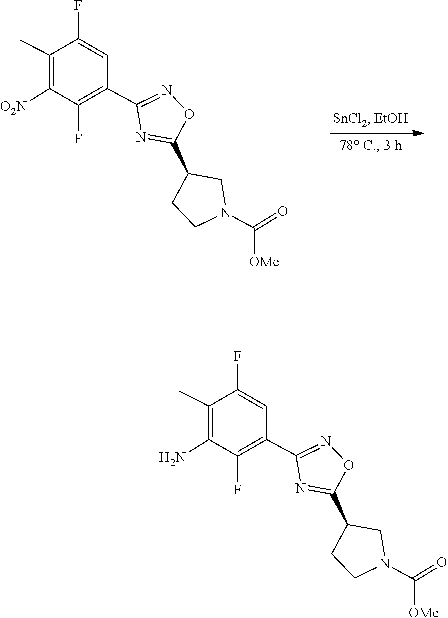 [SMILES: COC(=O)N1CC[C@@H](c2nc(-c3cc(F)c(C)c([N+](=O)[O-])c3F)no2)C1 ; COC(=O)N1CC[C@@H](c2nc(-c3cc(F)c(C)c(N)c3F)no2)C1]

To a suspension of methyl (R)-3-(3-(2,5-difluoro-4-methyl-3-nitrophenyl)-1,2,4-oxadiazol-5-yl)pyrrolidine-1-carboxylate (700.0 mg, 1.9 mmol, 1.0 eq) in EtOH (20.0 mL) was added SnCl₂ (1.1 g, 5.7 mmol, 3.0 eq). The reaction mixture was stirred at 78° C. for 3 h. The reaction was cooled to rt and the solvent was partially removed under vacuum. The pH was adjusted to slightly basic with saturated solution of sodium bicarbonate. The resulting white suspension was filtered and washed with water (30 mL) and EtOAc (30 mL). The aqueous layer was extracted with EtOAc (100 mL×2). The combined organic layers were washed with brine (100 mL), dried over anhydrous sodium sulfate, filtered, and concentrated. The resulting residue was purified by column chromatography on silica gel (PE/EtOAc=1/1, v/v) to afford methyl (R)-3-(3-(3-amino-2,5-difluoro-4-methylphenyl)-1,2,4-oxadiazol-5-yl)pyrrolidine-1-carboxylate as a yellow solid (500.0 mg, 77.8%). LCMS (M+H⁺) m/z calculated 339.1, found 339.1.

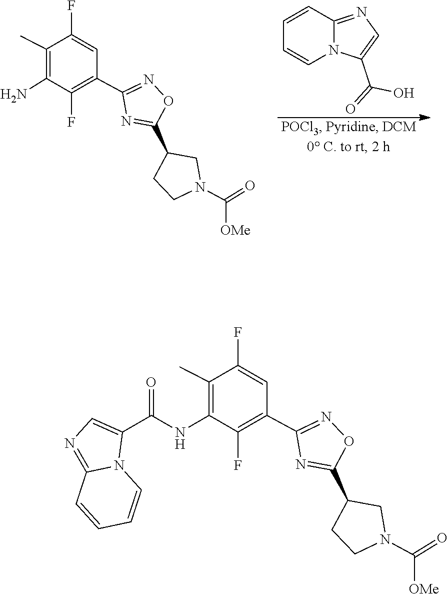

To a stirring solution of imidazo[1,2-a]pyridine-3-carboxylic acid (240.0 mg, 1.5 mmol, 1.0 eq), methyl (R)-3-(3-(3-amino-2,5-difluoro-4-methylphenyl)-1,2,4-oxadiazol-5-yl)pyrrolidine-1-carboxylate (500.0 mg, 1.5 mmol, 1.0 eq) and pyridine (592.5 mg, 7.5 mmol, 5.0 eq) in DCM (15.0 mL) was added POCl₃ (688.5 mg, 4.5 mmol, 3.0 eq) at 0° C. The reaction mixture was stirred at rt for 2 h. The reaction mixture was diluted with water (30 mL). The aqueous layer was extracted with DCM (30 mL×3). The combined organic layers were washed with brine (100 mL), dried over anhydrous sodium sulfate, filtered, and concentrated. The resulting residue was purified by prep-HPLC to afford methyl (R)-3-(3-(2,5-difluoro-3-(imidazo[1,2-a]pyridine-3-carboxamido)-4-methylphenyl)-1,2,4-oxadiazol-5-yl)pyrrolidine-1-carboxylate (94.6 mg, 13.3%). LCMS (M+H⁺) m/z calculated 483.2, found 483.2. ¹H NMR (DMSO-d₆, 400 MHz) δ 10.56 (s, 1H), 9.49-9.50 (d, 1H), 8.83 (s, 1H), 7.92-7.94 (d, 1H), 7.74-7.76 (t, 2H), 7.36-7.39 (t, 1H), 3.95-3.98 (m, 1H), 3.37-3.83 (m, 2H), 3.61 (s, 3H), 3.42-3.50 (m, 2H), 2.32-2.43 (m, 2H), 2.27-2.28 (d, 3H).

### Example 54: Preparation of methyl (R)-3-(3-(2,5-difluoro-3-(imidazo[1,2-a]pyridine-3-carboxamido)-4-methylphenyl)-1,2,4-oxadiazol-5-yl)piperidine-1-carboxylate 2

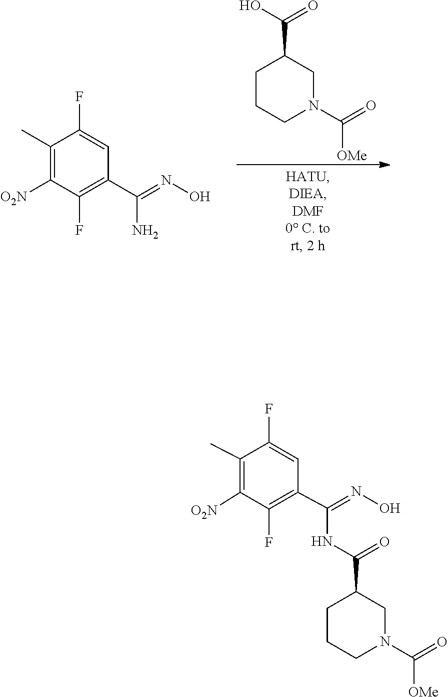 [SMILES: Cc1c(F)cc(/C(N)=N/O)c(F)c1[N+](=O)[O-] ; COC(=O)N1CCC[C@@H](C(=O)O)C1 ; COC(=O)N1CCC[C@@H](C(=O)N/C(=N\O)c2cc(F)c(C)c([N+](=O)[O-])c2F)C1]

To the mixture of (Z)-2,5-difluoro-N′-hydroxy-4-methyl-3-nitrobenzimidamide (300.0 mg, 1.3 mmol, 1.0 eq), (R)-1-(methoxycarbonyl)piperidine-3-carboxylic acid (242.0 mg, 1.3 mmol, 1.0 eq) and HATU (988 mg, 2.6 mmol, 2.0 eq) in DMF (10.0 mL) was added DIEA (503.0 mg, 3.9 mmol, 3.0 eq) at 0° C. and the mixture was stirred at rt for 2 h. The reaction was quenched by adding H₂O (50 mL) and the mixture was extracted with EtOAc (50 mL×3). The combined organic layers were washed with brine (100 mL), dried over anhydrous sodium sulfate, filtered, and concentrated. The resulting residue was purified by column chromatography on silica gel (PE/EtOAc=1/1, v/v) to afford methyl (R,Z)-3-(((2,5-difluoro-4-methyl-3-nitrophenyl)(hydroxyimino)methyl)carbamoyl)piperidine-1-carboxylate as a pale yellow solid (350.0 mg, 67.0%). LCMS (M+H⁺) m/z calculated 401.1, found 401.1.

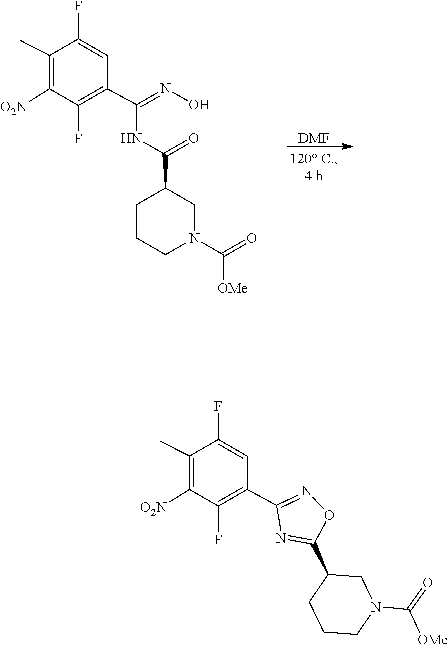

The mixture of methyl (R,Z)-3-(((2,5-difluoro-4-methyl-3-nitrophenyl)(hydroxyimino)methyl)carbamoyl)piperidine-1-carboxylate (350.0 mg, 0.87 mmol, 1.0 eq) in DMF (10.0 mL) was stirred at 120° C. for 4 h. The mixture was poured into brine (100 mL) and extracted with EtOAc (50 mL×3). The combined organic layers were washed with brine (100 mL), dried over anhydrous sodium sulfate, filtered, and concentrated. The resulting residue was purified by column chromatography on silica gel (PE/EtOAc=1/1, v/v) to afford methyl (R)-3-(3-(2,5-difluoro-4-methyl-3-nitrophenyl)-1,2,4-oxadiazol-5-yl)piperidine-1-carboxylate as a pale yellow oil (300.0 mg, 89.0%). LCMS (M+H⁺) m/z calculated 383.1, found 383.1.

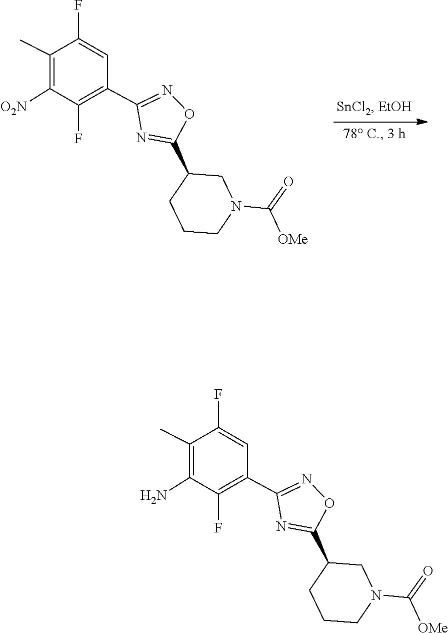 [SMILES: COC(=O)N1CCC[C@@H](c2nc(-c3cc(F)c(C)c([N+](=O)[O-])c3F)no2)C1 ; COC(=O)N1CCC[C@@H](c2nc(-c3cc(F)c(C)c(N)c3F)no2)C1]

To a suspension of methyl methyl (R)-3-(3-(2,5-difluoro-4-methyl-3-nitrophenyl)-1,2,4-oxadiazol-5-yl)piperidine-1-carboxylate (300.0 mg, 0.79 mmol, 1.0 eq) in EtOH (10.0 mL) was added SnCl₂ (447.6 mg, 2.4 mmol, 3.0 eq). The reaction mixture was stirred at 78° C. for 3 h. The reaction was cooled to rt and the solvent was partially removed under vacuum. The pH was adjusted to slightly basic with saturated solution of sodium bicarbonate. The resulting white suspension was filtered and washed with water (50 mL) and EtOAc (50 mL). The aqueous layer was extracted with EtOAc (50 mL×3). The combined organic layers were washed with brine (100 mL), dried over anhydrous sodium sulfate, filtered, and concentrated. The resulting residue was purified by column chromatography on silica gel (PE/EtOAc=1/1, v/v) to afford methyl (R)-3-(3-(3-amino-2,5-difluoro-4-methylphenyl)-1,2,4-oxadiazol-5-yl)piperidine-1-carboxylate as a yellow solid (230.0 mg, 83.0%). LCMS (M+H⁺) m/z calculated 353.1, found 353.1.

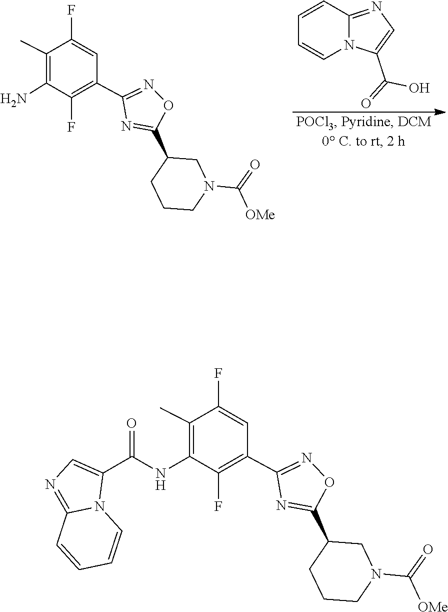

To a stirring solution of imidazo[1,2-a]pyridine-3-carboxylic acid (105.9 mg, 0.65 mmol, 1.0 eq), methyl (R)-3-(3-(3-amino-2,5-difluoro-4-methylphenyl)-1,2,4-oxadiazol-5-yl)piperidine-1-carboxylate (230.0 mg, 0.65 mmol, 1.0 eq) and pyridine (256.8 mg, 3.3 mmol, 5.0 eq) in DCM (10.0 mL) was added POCl₃ (298.4 mg, 1.9 mmol, 3.0 eq) at 0° C. The reaction mixture was stirred at rt for 2 h. The reaction mixture was diluted with water (50 mL). The aqueous layer was extracted with DCM (50 mL×3), the combined organic layers were washed with brine (100 mL), dried over anhydrous sodium sulfate, filtered, and concentrated. The resulting residue was purified by prep-HPLC to afford methyl (R)-3-(3-(2,5-difluoro-3-(imidazo[1,2-a]pyridine-3-carboxamido)-4-methylphenyl)-1,2,4-oxadiazol-5-yl)piperidine-1-carboxylate (31.6 mg, 9.8%). LCMS (M+H⁺) m/z calculated 497.2, found 497.2. ¹H NMR (DMSO-d₆, 400 MHz) δ 10.37 (s, 1H), 9.50-9.52 (d, 1H), 8.55 (s, 1H), 7.75-7.78 (d, 1H), 7.61-7.64 (m, 1H), 7.47-7.51 (t, 1H), 7.13-7.16 (t, 1H), 4.14-4.15 (m, 1H), 3.73-3.76 (m, 1H), 3.60 (s, 3H), 3.41-3.45 (m, 2H), 3.07-3.14 (m, 1H), 2.23-2.24 (d, 3H), 2.17-2.20 (m, 1H), 1.77-1.91 (m, 1H), 1.71-1.75 (m, 1H), 1.49-1.58 (m, 1H).

### Example 55: Preparation of methyl (S)-3-(3-(2,5-difluoro-3-(imidazo[1,2-a]pyridine-3-carboxamido)-4-methylphenyl)-1,2,4-oxadiazol-5-yl)pyrrolidine-1-carboxylate

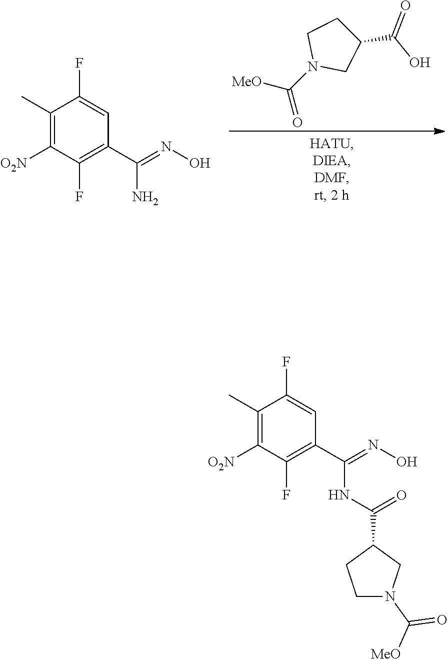 [SMILES: Cc1c(F)cc(/C(N)=N/O)c(F)c1[N+](=O)[O-] ; COC(=O)N1CC[C@H](C(=O)O)C1 ; COC(=O)N1CC[C@H](C(=O)N/C(=N\O)c2cc(F)c(C)c([N+](=O)[O-])c2F)C1]

To the mixture of (Z)-2,5-difluoro-N′-hydroxy-4-methyl-3-nitrobenzimidamide (2.0 g, 8.7 mmol, 1.0 eq), 1-(methoxycarbonyl)azetidine-3-carboxylic acid (1.5 g, 8.7 mmol, 1.0 eq) and HATU (6.6 g, 17.4 mmol, 2.0 eq) in DMF (40.0 mL) was added DIEA (3.4 g, 26.1 mmol, 3.0 eq) at 0° C. The mixture was stirred at rt for 2 h. The reaction was quenched by adding H₂O (100 mL) and the mixture was extracted with EtOAc (150 mL×3). The combined organic layers were washed with brine (100 mL), dried over anhydrous sodium sulfate, filtered, and concentrated. The resulting residue was purified by column chromatography on silica gel (PE/EtOAc=1/1, v/v) to afford methyl (S,Z)-3-(((2,5-difluoro-4-methyl-3-nitrophenyl)(hydroxyimino)methyl)carbamoyl)pyrrolidine-1-carboxylate as a pale yellow solid (2.4 g, 72.0%). LCMS (M+H⁺) m/z calculated 387.1, found 387.1.

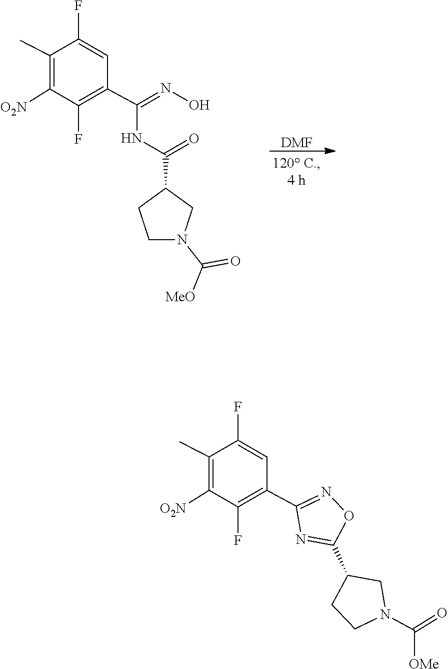

The mixture of methyl (S,Z)-3-(((2,5-difluoro-4-methyl-3-nitrophenyl)(hydroxyimino)methyl)carbamoyl)pyrrolidine-1-carboxylate (2.4 g, 6.2 mmol, 1.0 eq) in DMF (30.0 mL) was stirred at 120° C. for 4 h. The mixture was poured into brine (300 mL) and extracted with EtOAc (150 mL×3). The combined organic layers were washed with brine (100 mL), dried over anhydrous sodium sulfate, filtered, and concentrated. The resulting residue was purified by column chromatography on silica gel (PE/EtOAc=1/1, v/v) to afford methyl (S)-3-(3-(2,5-difluoro-4-methyl-3-nitrophenyl)-1,2,4-oxadiazol-5-yl)pyrrolidine-1-carboxylate as pale yellow oil (2.0 g, 86.0%). LCMS (M+H⁺) m/z calculated 369.1, found 369.1.

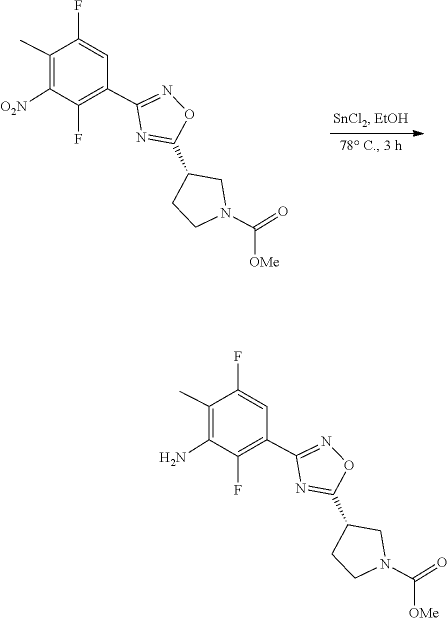 [SMILES: COC(=O)N1CC[C@H](c2nc(-c3cc(F)c(C)c([N+](=O)[O-])c3F)no2)C1 ; COC(=O)N1CC[C@H](c2nc(-c3cc(F)c(C)c(N)c3F)no2)C1]

To a suspension of methyl (S)-3-(3-(2,5-difluoro-4-methyl-3-nitrophenyl)-1,2,4-oxadiazol-5-yl)pyrrolidine-1-carboxylate (2.0 g, 5.4 mmol, 1.0 eq) in EtOH (20.0 mL) was added SnCl₂ (3.1 g, 16.2 mmol, 3.0 eq). The reaction mixture was stirred at 78° C. for 3 h. The reaction was cooled to rt and the solvent was partially removed under vacuum. The pH was adjusted to slightly basic with saturated solution of sodium bicarbonate. The resulting white suspension was filtered and washed with water (50.0 mL) and EtOAc (50.0 mL). The aqueous layer was extracted with EtOAc (100 mL×3). The combined organic layers were washed with brine (100 mL), dried over anhydrous sodium sulfate, filtered, and concentrated. The resulting residue was purified by column chromatography on silica gel (PE/EtOAc=1/1, v/v) to afford methyl (S)-3-(3-(3-amino-2,5-difluoro-4-methylphenyl)-1,2,4-oxadiazol-5-yl)pyrrolidine-1-carboxylate as a yellow solid (1.4 g, 77.0%). LCMS (M+H⁺) m/z calculated 339.1, found 339.1.

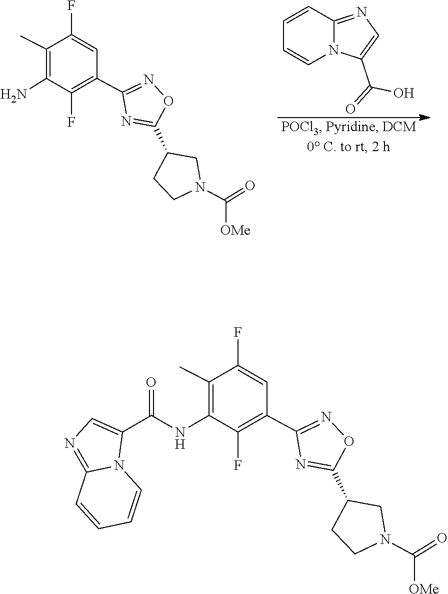

To a stirring solution of imidazo[1,2-a]pyridine-3-carboxylic acid (664.2 mg, 4.1 mmol, 1.0 eq), methyl (R)-3-(3-(3-amino-2,5-difluoro-4-methylphenyl)-1,2,4-oxadiazol-5-yl)pyrrolidine-1-carboxylate (1.4 g, 4.1 mmol, 1.0 eq) and pyridine (1.6 g, 20.5 mmol, 5.0 eq) in DCM (20.0 mL) was added POCl₃ (1.9 g, 12.3 mmol, 3.0 eq) at 0° C. The reaction mixture was stirred at rt for 2 h. The reaction mixture was diluted with water (50.0 mL). The aqueous layer was extracted with DCM (50 mL×3) and the combined organic layers were washed with brine (100 mL), dried over anhydrous sodium sulfate, filtered, and concentrated. The resulting residue was purified by prep-HPLC to afford methyl (S)-3-(3-(2,5-difluoro-3-(imidazo[1,2-a]pyridine-3-carboxamido)-4-methylphenyl)-1,2,4-oxadiazol-5-yl)pyrrolidine-1-carboxylate (744.6 mg, 37.2%). LCMS (M+H⁺) m/z calculated 483.2, found 483.2. ¹H NMR (DMSO-d₆, 400 MHz) δ 10.29 (s, 1H), 9.40-9.42 (d, 1H), 8.63 (s, 1H), 7.79-7.82 (d, 1H), 7.72-7.76 (t, 1H), 7.52-7.56 (m, 1H), 7.18-7.21 (t, 1H), 3.93-3.98 (m, 1H), 3.79-3.84 (m, 1H), 3.66-3.70 (m, 1H), 3.61 (s, 3H), 3.42-3.53 (m, 2H), 2.39-2.44 (m, 2H), 2.07-2.08 (d, 3H).

### Example 56: Preparation of methyl (S)-3-(3-(2,5-difluoro-3-(imidazo[1,2-a]pyridine-3-carboxamido)-4-methylphenyl)-1,2,4-oxadiazol-5-yl)piperidine-1-carboxylate

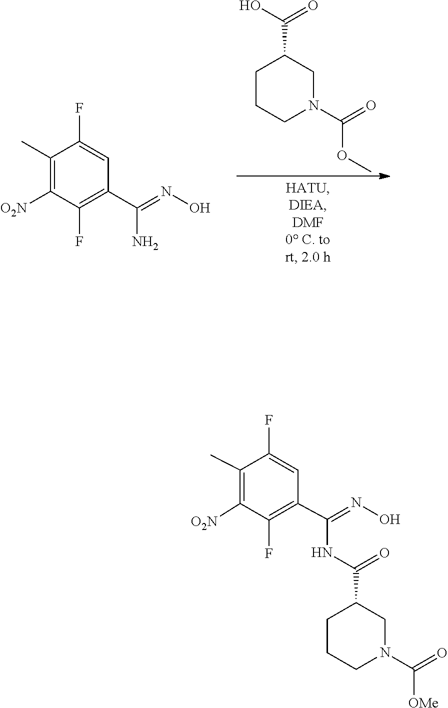 [SMILES: Cc1c(F)cc(/C(N)=N/O)c(F)c1[N+](=O)[O-] ; COC(=O)N1CCC[C@H](C(=O)O)C1 ; COC(=O)N1CCC[C@H](C(=O)N/C(=N\O)c2cc(F)c(C)c([N+](=O)[O-])c2F)C1]

To the mixture of (Z)-2,5-difluoro-N′-hydroxy-4-methyl-3-nitrobenzimidamide (500.0 mg, 2.2 mmol, 1.0 eq), (S)-1-(methoxycarbonyl)piperidine-3-carboxylic acid (411.4 mg, 2.2 mmol, 1.0 eq) and HATU (1.7 g, 4.4 mmol, 2.0 eq) in DMF (10.0 mL) was added DIEA (851.4 mg, 6.6 mmol, 3.0 eq) at 0° C. The mixture was stirred at rt for 2 h. The reaction was quenched by adding H₂O (50 mL) and the mixture was extracted with EtOAc (50 mL×3). The combined organic layers were washed with brine (100 mL), dried over anhydrous sodium sulfate, filtered, and concentrated. The resulting residue was purified by column chromatography on silica gel (PE/EtOAc=1/1, v/v) to afford methyl (S,Z)-3-(((2,5-difluoro-4-methyl-3-nitrophenyl)(hydroxyimino)methyl)carbamoyl)piperidine-1-carboxylate as a pale yellow solid (600.0 mg, 69.4%). LCMS (M+H⁺) m/z calculated 401.1, found 401.1.

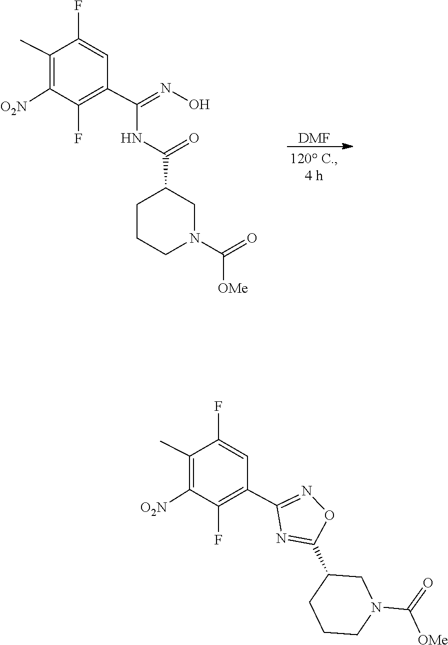

The mixture of methyl (S,Z)-3-(((2,5-difluoro-4-methyl-3-nitrophenyl)(hydroxyimino)methyl)carbamoyl)piperidine-1-carboxylate (600.0 mg, 1.5 mmol, 1.0 eq) in DMF (10.0 mL) was stirred at 120° C. for 4 h. The mixture was poured into brine (100 mL) and extracted with EtOAc (50 mL×3). The combined organic layers were washed with brine (100 mL), dried over anhydrous sodium sulfate, filtered, and concentrated. The resulting residue was purified by column chromatography on silica gel (PE/EtOAc=1/1, v/v) to afford methyl (S)-3-(3-(2,5-difluoro-4-methyl-3-nitrophenyl)-1,2,4-oxadiazol-5-yl)piperidine-1-carboxylate as a pale yellow oil (500.0 mg, 87.3%). LCMS (M+H⁺) m/z calculated 383.1, found 383.1.

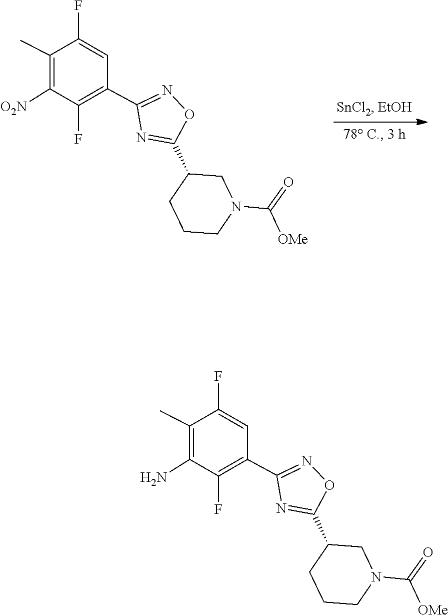 [SMILES: COC(=O)N1CCC[C@H](c2nc(-c3cc(F)c(C)c([N+](=O)[O-])c3F)no2)C1 ; COC(=O)N1CCC[C@H](c2nc(-c3cc(F)c(C)c(N)c3F)no2)C1]

To a suspension of methyl methyl (S)-3-(3-(2,5-difluoro-4-methyl-3-nitrophenyl)-1,2,4-oxadiazol-5-yl)piperidine-1-carboxylate (500.0 mg, 1.3 mmol, 1.0 eq) in EtOH (10.0 mL) was added SnCl₂ (741.0 mg, 3.9 mmol, 3.0 eq). The reaction mixture was stirred at 78° C. for 3 h. The reaction was cooled to rt and the solvent was partially removed under vacuum. The pH was adjusted to slightly basic with saturated solution of sodium bicarbonate. The resulting white suspension was filtered and washed with water (50 mL) and EtOAc (50 mL). The aqueous layer was extracted with EtOAc (50 mL×3). The combined organic layers were washed with brine (100 mL), dried over anhydrous sodium sulfate, filtered, and concentrated. The resulting residue was purified by column chromatography on silica gel (PE/EtOAc=1/1, v/v) to afford methyl (S)-3-(3-(3-amino-2,5-difluoro-4-methylphenyl)-1,2,4-oxadiazol-5-yl)piperidine-1-carboxylate as a yellow solid (500.0 mg, quant.). LCMS (M+H⁺) m/z calculated 353.1, found 353.1.

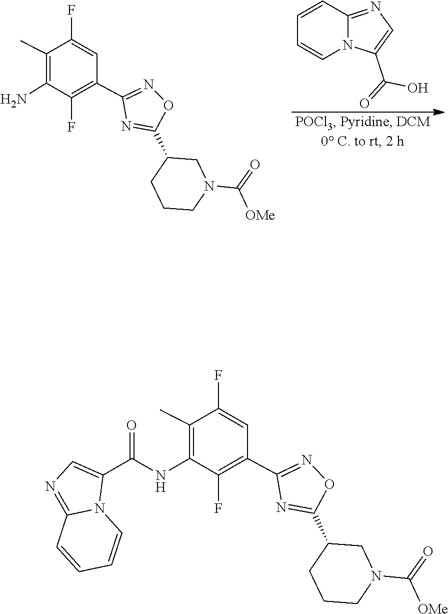

To a stirring solution of imidazo[1,2-a]pyridine-3-carboxylic acid (230.0 mg, 1.4 mmol, 1.0 eq), methyl (S)-3-(3-(3-amino-2,5-difluoro-4-methylphenyl)-1,2,4-oxadiazol-5-yl)piperidine-1-carboxylate (500.0 mg, 1.4 mmol, 1.0 eq) and pyridine (553.0 mg, 7.0 mmol, 5.0 eq) in DCM (10.0 mL) was added POCl₃ (642.6 mg, 4.2 mmol, 3.0 eq) at 0° C. The reaction mixture was stirred at rt for 2.0 h, and diluted with water (50 mL). The aqueous layer was extracted with DCM (50 mL×3), the combined organic layers were washed with brine (100 mL), dried over anhydrous sodium sulfate, filtered, and concentrated. The resulting residue was purified by prep-HPLC to afford methyl (S)-3-(3-(2,5-difluoro-3-(imidazo[1,2-a]pyridine-3-carboxamido)-4-methylphenyl)-1,2,4-oxadiazol-5-yl)piperidine-1-carboxylate (77.0 mg, 10.9%). LCMS (M+H⁺) m/z calculated 497.2, found 497.2. ¹H NMR (DMSO-d₆, 400 MHz) δ 10.41 (s, 1H), 9.45-9.46 (d, 1H), 8.71 (s, 1H), 7.86-7.89 (d, 1H), 7.73-7.75 (t, 1H), 7.64-7.68 (t, 1H), 7.28-7.31 (t, 1H), 4.13-4.16 (m, 1H), 3.75-3.76 (m, 1H), 3.60 (s, 3H), 3.33-3.35 (m, 2H), 3.08-3.14 (m, 1H), 2.32-2.33 (d, 3H), 2.17-2.21 (m, 1H), 1.86-1.89 (m, 1H), 1.73-1.76 (m, 1H), 1.52-1.56 (m, 1H).

### II. Biological Evaluation

### Example 1. Biochemical Assay

The inhibitory activity against c-KIT was measured using ADP-Glo assay. The percent (%) inhibition at each concentration of compound is calculated based on and relative to the luminescence signal in the Max and Min control wells contained within each assay plate. The Max control wells contain enzyme and substrate as 0% inhibition, and the Min control wells only contain substrate without enzyme as 100% inhibition. The concentrations and % inhibition values for tested compounds are plotted and the concentration of compound required for 50% inhibition (IC₅₀) is determined with a four-parameter logistic dose response equation. The ability of the compounds in Table 1 to inhibit c-KIT was determined.

TABLE 1

| Compound | |
|---|---|
| No. | Chemical Name |
| C001 | N-(5-(5-(3,3-difluorocyclobutyl)-1,2,4-oxadiazol-3-yl)-3-fluoro-2- |
| | methylphenyl)imidazo[1,2-a]pyridine-3-carboxamide |
| C002 | N-(3-fluoro-5-(5-((1R,2S)-2-fluorocyclopropyl)-1,2,4-oxadiazol-3-yl)-2- |
| | methylphenyl)imidazo[1,2-a]pyridine-3-carboxamide |
| C003 | methyl 3-(3-(3-fluoro-5-(imidazo[1,2-a]pyridine-3-carboxamido)-4- |
| | methylphenyl)-1,2,4-oxadiazol-5-yl)azetidine-1-carboxylate |
| C004 | N-(5-(5-(3,3-difluorocyclobutyl)-1,2,4-oxadiazol-3-yl)-3-fluoro-2- |
| | methylphenyl)pyrazolo[1,5-a]pyridine-3-carboxamide |
| C005 | N-(3-fluoro-5-(5-((1R,2S)-2-fluorocyclopropyl)-1,2,4-oxadiazol-3-yl)-2- |
| | methylphenyl)pyrazolo[1,5-a]pyridine-3-carboxamide |
| C006 | methyl 3-(3-(3-fluoro-4-methyl-5-(pyrazolo[1,5-a]pyridine-3- |
| | carboxamido)phenyl)-1,2,4-oxadiazol-5-yl)azetidine-1-carboxylate |
| C007 | N-(5-(5-(3,3-difluorocyclobutyl)-1,2,4-oxadiazol-3-yl)-3-fluoro-2- |
| | methylphenyl)-6-morpholinoimidazo[1,2-a]pyridine-3-carboxamide |
| C008 | N-(3-fluoro-5-(5-((1R,2S)-2-fluorocyclopropyl)-1,2,4-oxadiazol-3-yl)-2- |
| | methylphenyl)-6-morpholinoimidazo[1,2-a]pyridine-3-carboxamide |
| C009 | methyl 3-(3-(3-fluoro-4-methyl-5-(6-morpholinoimidazo[1,2-a]pyridine-3- |
| | carboxamido)phenyl)-1,2,4-oxadiazol-5-yl)azetidine-1-carboxylate |
| C010 | N-(5-(5-(3,3-difluorocyclobutyl)-1,2,4-oxadiazol-3-yl)-3-fluoro-2- |
| | methylphenyl)-7-(piperazin-1-yl)imidazo[1,2-a]pyridine-3-carboxamide |
| C011 | N-(5-(5-(3,3-difluorocyclobutyl)-1,2,4-oxadiazol-3-yl)-3-fluoro-2- |
| | methylphenyl)-7-(4-methylpiperazin-1-yl)imidazo[1,2-a]pyridine-3- |
| | carboxamide |
| C012 | methyl 3-(3-(3-(6-chloroimidazo[1,2-a]pyridine-3-carboxamido)-5-fluoro-4- |
| | methylphenyl)-1,2,4-oxadiazol-5-yl)azetidine-1-carboxylate |
| C013 | N-(5-(5-(3,3-difluorocyclobutyl)-1,2,4-oxadiazol-3-yl)-3-fluoro-2- |
| | methylphenyl)-7-morpholinoimidazo[1,2-a]pyridine-3-carboxamide |
| C014 | N-(3-fluoro-5-(5-((1R,2S)-2-fluorocyclopropyl)-1,2,4-oxadiazol-3-yl)-2- |
| | methylphenyl)-7-morpholinoimidazo[1,2-a]pyridine-3-carboxamide |
| C015 | methyl 3-(3-(3-fluoro-4-methyl-5-(7-morpholinoimidazo[1,2-a]pyridine-3- |
| | carboxamido)phenyl)-1,2,4-oxadiazol-5-yl)azetidine-1-carboxylate |
| C016 | methyl 3-(3-(3-(7-chloroimidazo[1,2-a]pyridine-3-carboxamido)-5-fluoro-4- |
| | methylphenyl)-1,2,4-oxadiazol-5-yl)azetidine-1-carboxylate |
| C017 | methyl 3-(3-(3-fluoro-4-methyl-5-(7-methylimidazo[1,2-a]pyridine-3- |
| | carboxamido)phenyl)-1,2,4-oxadiazol-5-yl)azetidine-1-carboxylate |
| C018 | 7-(4-acetylpiperazin-1-yl)-N-(5-(5-(3,3-difluorocyclobutyl)-1,2,4-oxadiazol-3- |
| | yl)-3-fluoro-2-methylphenyl)imidazo[1,2-a]pyridine-3-carboxamide |
| C019 | methyl 3-(3-(3-fluoro-5-(6-fluoroimidazo[1,2-a]pyridine-3-carboxamido)-4- |
| | methylphenyl)-1,2,4-oxadiazol-5-yl)azetidine-1-carboxylate |
| C020 | methyl 3-(3-(3-fluoro-4-methyl-5-(6-(piperazin-1-yl)imidazo[1,2-a]pyridine- |
| | 3-carboxamido)phenyl)-1,2,4-oxadiazol-5-yl)azetidine-1-carboxylate |
| C021 | methyl 3-(3-(3-fluoro-4-methyl-5-(6-methylimidazo[1,2-a]pyridine-3- |
| | carboxamido)phenyl)-1,2,4-oxadiazol-5-yl)azetidine-1-carboxylate |
| C022 | N-(3-fluoro-5-(5-((1R,2S)-2-fluorocyclopropyl)-1,2,4-oxadiazol-3-yl)-2- |
| | methylphenyl)-7-(piperazin-1-yl)imidazo[1,2-a]pyridine-3-carboxamide |
| C023 | ethyl 3-(3-(3-fluoro-5-(imidazo[1,2-a]pyridine-3-carboxamido)-4- |
| | methylphenyl)-1,2,4-oxadiazol-5-yl)azetidine-1-carboxylate |
| C024 | methyl 3-(3-(3-fluoro-5-(7-fluoroimidazo[1,2-a]pyridine-3-carboxamido)-4- |
| | methylphenyl)-1,2,4-oxadiazol-5-yl)azetidine-1-carboxylate |
| C025 | N-(5-(5-(3,3-difluorocyclobutyl)-1,2,4-oxadiazol-3-yl)-3-fluoro-2- |
| | methylphenyl)-6-(piperazin-1-yl)imidazo[1,2-a]pyridine-3-carboxamide |
| C026 | N-(3-fluoro-5-(5-((1R,2S)-2-fluorocyclopropyl)-1,2,4-oxadiazol-3-yl)-2- |
| | methylphenyl)-6-(piperazin-1-yl)imidazo[1,2-a]pyridine-3-carboxamide |
| C027 | methyl 3-(3-(3-fluoro-5-(6-methoxyimidazo[1,2-a]pyridine-3-carboxamido)- |
| | 4-methylphenyl)-1,2,4-oxadiazol-5-yl)azetidine-1-carboxylate |
| C028 | methyl 3-(3-(3-fluoro-4-methyl-5-(6-(4-methylpiperazin-1-yl)imidazo[1,2- |
| | a]pyridine-3-carboxamido)phenyl)-1,2,4-oxadiazol-5-yl)azetidine-1- |
| | carboxylate |
| C029 | methyl 3-(3-(3-fluoro-5-(7-methoxyimidazo[1,2-a]pyridine-3-carboxamido)- |
| | 4-methylphenyl)-1,2,4-oxadiazol-5-yl)azetidine-1-carboxylate |
| C030 | methyl 3-(3-(3-fluoro-4-methyl-5-(7-(piperazin-1-yl)imidazo[1,2-a]pyridine- |
| | 3-carboxamido)phenyl)-1,2,4-oxadiazol-5-yl)azetidine-1-carboxylate |
| C031 | cyclopropyl 3-(3-(3-fluoro-5-(imidazo[1,2-a]pyridine-3-carboxamido)-4- |
| | methylphenyl)-1,2,4-oxadiazol-5-yl)azetidine-1-carboxylate |
| C032 | N-(3-fluoro-2-methyl-5-(5-(1-(methylcarbamoyl)azetidin-3-yl)-1,2,4- |
| | oxadiazol-3-yl)phenyl)imidazo[1,2-a]pyridine-3-carboxamide |
| C033 | N-(5-(5-(3,3-difluorocyclobutyl)-1,2,4-oxadiazol-3-yl)-3-fluoro-2- |
| | methylphenyl)-6-(4-methylpiperazin-1-yl)imidazo[1,2-a]pyridine-3- |
| | carboxamide |
| C034 | N-(3-fluoro-5-(5-((1R,2S)-2-fluorocyclopropyl)-1,2,4-oxadiazol-3-yl)-2- |
| | methylphenyl)-6-(4-methylpiperazin-1-yl)imidazo[1,2-a]pyridine-3- |
| | carboxamide |
| C035 | methyl 3-(3-(3-(7-cyanoimidazo[1,2-a]pyridine-3-carboxamido)-5-fluoro-4- |
| | methylphenyl)-1,2,4-oxadiazol-5-yl)azetidine-1-carboxylate |
| C036 | methyl 3-(3-(3-(6-(4-acetylpiperazin-1-yl)imidazo[1,2-a]pyridine-3- |
| | carboxamido)-5-fluoro-4-methylphenyl)-1,2,4-oxadiazol-5-yl)azetidine-1- |
| | carboxylate |
| C037 | methyl 3-(5-(3-fluoro-5-(imidazo[1,2-a]pyridine-3-carboxamido)-4- |
| | methylphenyl)-1,2,4-oxadiazol-3-yl)azetidine-1-carboxylate |
| C038 | 6-(4-acetylpiperazin-1-yl)-N-(5-(5-(3,3-difluorocyclobutyl)-1,2,4-oxadiazol-3- |
| | yl)-3-fluoro-2-methylphenyl)imidazo[1,2-a]pyridine-3-carboxamide |
| C039 | 6-(4-acetylpiperazin-1-yl)-N-(3-fluoro-5-(5-((1R,2S)-2-fluorocyclopropyl)- |
| | 1,2,4-oxadiazol-3-yl)-2-methylphenyl)imidazo[1,2-a]pyridine-3-carboxamide |
| C040 | methyl 3-(3-(3-(6-carbamoylimidazo[1,2-a]pyridine-3-carboxamido)-5-fluoro- |
| | 4-methylphenyl)-1,2,4-oxadiazol-5-yl)azetidine-1-carboxylate |
| C041 | 2-aminoethyl 3-(3-(3-fluoro-5-(imidazo[1,2-a]pyridine-3-carboxamido)-4- |
| | methylphenyl)-1,2,4-oxadiazol-5-yl)azetidine-1-carboxylate |
| C042 | 2-methoxyethyl 3-(3-(3-fluoro-5-(imidazo[1,2-a]pyridine-3-carboxamido)-4- |
| | methylphenyl)-1,2,4-oxadiazol-5-yl)azetidine-1-carboxylate |
| C043 | methyl 3-(3-(3-(7-carbamoylimidazo[1,2-a]pyridine-3-carboxamido)-5-fluoro- |
| | 4-methylphenyl)-1,2,4-oxadiazol-5-yl)azetidine-1-carboxylate |
| C044 | methyl 3-(5-(3-fluoro-5-(imidazo[1,2-a]pyridine-3-carboxamido)-4- |
| | methylphenyl)-1,3,4-oxadiazol-2-yl)azetidine-1-carboxylate |
| C045 | methyl 3-(4-(3-fluoro-5-(imidazo[1,2-a]pyridine-3-carboxamido)-4- |
| | methylphenyl)oxazol-2-yl)azetidine-1-carboxylate |
| C046 | methyl 3-(5-(3-fluoro-5-(imidazo[1,2-a]pyridine-3-carboxamido)-4- |
| | methylphenyl)oxazol-2-yl)azetidine-1-carboxylate |
| C047 | methyl 3-(2-(3-fluoro-5-(imidazo[1,2-a]pyridine-3-carboxamido)-4- |
| | methylphenyl)oxazol-4-yl)azetidine-1-carboxylate |
| C048 | methyl 3-(5-(3-fluoro-5-(imidazo[1,2-a]pyridine-3-carboxamido)-4- |
| | methylphenyl)-4H-1,2,4-triazol-3-yl)azetidine-1-carboxylate |
| C049 | methyl (R)-3-(3-(3-fluoro-5-(imidazo[1,2-a]pyridine-3-carboxamido)-4- |
| | methylphenyl)-1,2,4-oxadiazol-5-yl)piperidine-1-carboxylate |
| C050 | N-(3-fluoro-5-(5-((1S,2R)-2-fluorocyclopropyl)-1,2,4-oxadiazol-3-yl)-2- |
| | methylphenyl)-7-morpholinoimidazo[1,2-a]pyridine-3-carboxamide |
| C051 | methyl 3-(2-(3-fluoro-5-(imidazo[1,2-a]pyridine-3-carboxamido)-4- |
| | methylphenyl)oxazol-5-yl)azetidine-1-carboxylate |
| C052 | methyl 3-(3-(2,5-difluoro-3-(imidazo[1,2-a]pyridine-3-carboxamido)-4- |
| | methylphenyl)-1,2,4-oxadiazol-5-yl)azetidine-1-carboxylate |
| C053 | methyl (R)-3-(3-(2,5-difluoro-3-(imidazo[1,2-a]pyridine-3-carboxamido)-4- |
| | methylphenyl)-1,2,4-oxadiazol-5-yl)pyrrolidine-1-carboxylate |
| C054 | methyl (R)-3-(3-(2,5-difluoro-3-(imidazo[1,2-a]pyridine-3-carboxamido)-4- |
| | methylphenyl)-1,2,4-oxadiazol-5-yl)piperidine-1-carboxylate |
| C055 | methyl (S)-3-(3-(2,5-difluoro-3-(imidazo[1,2-a]pyridine-3-carboxamido)-4- |
| | methylphenyl)-1,2,4-oxadiazol-5-yl)pyrrolidine-1-carboxylate |
| C056 | methyl (S)-3-(3-(2,5-difluoro-3-(imidazo[1,2-a]pyridine-3-carboxamido)-4- |
| | methylphenyl)-1,2,4-oxadiazol-5-yl)piperidine-1-carboxylate |

Table 2 shows IC₅₀ data of several compounds of the invention against c-KIT using ADP-Glo assay. The IC₅₀ data are designated within the following ranges: A: ≤0.10 μM, B: >0.10 μM to ≥1 μM, C: >1 μM to ≤20 μM

TABLE 2

| | c-KIT |
|---|---|
| Compounds | IC₅₀ |
| C003, C015, C029, C036, C038, C039, C040, C041, C043, | A |
| C052 | |
| C005, C006, C007, C009, C010, C011, C012, C013, C014, | B |
| C016, C017, C018, C019, C021, C023, C024, C025, C026, | |
| C027, C028, C030, C033, C034, C037, C042, C045, C047, | |
| C048, C049, C050, C054, C055, C056 | |
| C001, C002, C004, C008, C020, C022, C031, C032, C035, | C |
| C044, C046, C051, C053 | |

Table 3 provides comparative data in the c-KIT ADP-Glo assay between two prior art compounds (WO 2013/033167) and example 3 (C003) of the present disclosure.

TABLE 3

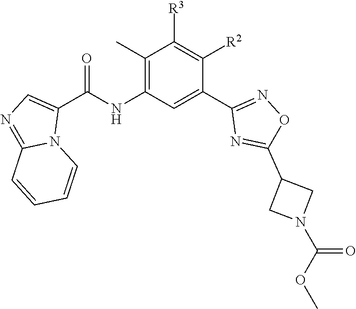

| Chemical Name | R² | R³ | c-KIT IC₅₀ (nM) |
|---|---|---|---|
| methyl 3-(3-(2-fluoro-5-(imidazo[1,2- | F | H | 4952.0 |
| a]pyridine-3-carboxamido)-4-methylphenyl)- | | | |
| 1,2,4-oxadiazol-5-yl)azetidine-1-carboxylate | | | |
| methyl 3-(3-(3-(imidazo[1,2-a]pyridine-3- | H | H | 171.6 |
| carboxamido)-4-methylphenyl)-1,2,4- | | | |
| oxadiazol-5-yl)azetidine-1-carboxylate | | | |
| methyl 3-(3-(3-fluoro-5-(imidazo[1,2- | H | F | 43.0 |
| a]pyridine-3-carboxamido)-4-methylphenyl)- | | | |
| 1,2,4-oxadiazol-5-yl)azetidine-1-carboxylate | | | |

### III. Preparation of Pharmaceutical Dosage Forms

### Example 1: Oral Capsule

The active ingredient is a compound of Formula (I) or Table 1, or a pharmaceutically acceptable salt or solvate thereof. A capsule for oral administration is prepared by mixing 1-1000 mg of active ingredient with starch or other suitable powder blend. The mixture is incorporated into an oral dosage unit such as a hard gelatin capsule, which is suitable for oral administration.

### Example 2: Solution for Injection

The active ingredient is a compound of Formula (I) or Table 1, or a pharmaceutically acceptable salt or solvate thereof, and is formulated as a solution in sesame oil at a concentration of 50 mg-eq/mL.

The examples and embodiments described herein are for illustrative purposes only and various modifications or changes suggested to persons skilled in the art are to be included within the spirit and purview of this application and scope of the appended claims.

We claim:

1. A compound, or a pharmaceutically acceptable salt or solvate thereof, having the structure of Formula (I): 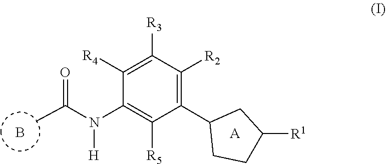
wherein,
Ring A is an optionally substituted 5-membered nitrogen-containing heteroaryl;
Ring B is an optionally substituted 9- or 10-membered bicyclic nitrogen-containing heteroaryl;
R¹ is optionally substituted alkyl, optionally substituted carbocyclyl, optionally substituted carbocyclylalkyl, optionally substituted heterocyclyl, optionally substituted heterocyclylalkyl, optionally substituted aryl, optionally substituted aralkyl, optionally substituted heteroaryl, or optionally substituted heteroaralkyl.
R² is hydrogen, halo, cyano, or optionally substituted C1-C6 alkyl;
R³ is halo;
R⁴ is optionally substituted C1-C6 alkyl; and
R⁵ is hydrogen, halo, cyano, or optionally substituted C1-C6 alkyl.

2. The compound of claim 1, or a pharmaceutically acceptable salt or solvate thereof, wherein ring A is N

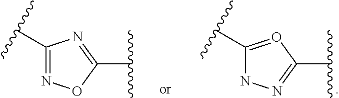

3. The compound of claim 1, or a pharmaceutically acceptable salt or solvate thereof, wherein ring A is

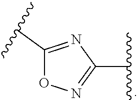 [SMILES: *c1noc(*)n1]

4. The compound of claim 1, or a pharmaceutically acceptable salt or solvate thereof, wherein ring A is

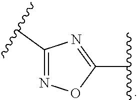 [SMILES: *c1noc(*)n1]

5. The compound of claim 1, or a pharmaceutically acceptable salt or solvate thereof, wherein ring A is

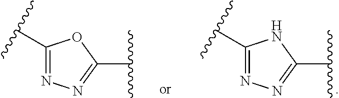 [SMILES: *c1nnc(*)o1 ; *c1nnc(*)[nH]1]

6. The compound of claim 1, or a pharmaceutically acceptable salt or solvate thereof, wherein ring A is

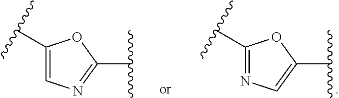 [SMILES: *c1cnc(*)o1 ; *c1cnc(*)o1]

7. The compound of claim 1, or a pharmaceutically acceptable salt or solvate thereof, wherein ring A is

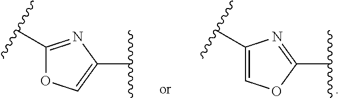 [SMILES: *c1coc(*)n1 ; *c1coc(*)n1]

8. The compound of any one of claims 1-7, or a pharmaceutically acceptable salt or solvate thereof, wherein ring B is an optionally substituted 9-membered bicyclic nitrogen-containing heteroaryl.

9. The compound of any one of claims 1-7, or a pharmaceutically acceptable salt or solvate thereof, wherein ring B is an optionally substituted 10-membered bicyclic nitrogen-containing heteroaryl.

10. The compound of claim 8, or a pharmaceutically acceptable salt or solvate thereof, wherein ring B is 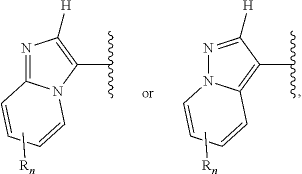
wherein n is 0-4; and each R is independently selected from the group consisting of hydrogen, cyano, halo, hydroxy, azido, amino, nitro, —CO₂H, —S(O)—R¹⁰, —S—R¹⁰, —S(O)₂—R¹⁰, optionally substituted C1-C6 alkoxy, optionally substituted C1-C6 alkyl, optionally substituted C2-C6 alkynyl, optionally substituted carbocyclyl, optionally substituted C2-C6 alkenyl, optionally substituted heterocyclyl, —N(R¹¹)₂, —CO—R¹⁰, —CO₂—R¹⁰, —CON(R¹¹)₂, —NR¹¹CO—R¹⁰, —NR¹¹CO₂—R¹⁰, —SO₂N(R¹¹)₂, —C(═NR¹²)—N(R¹¹)₂, —NR¹¹CO—R¹⁰, —NR¹¹CO₂—R¹⁰, —NR¹¹CO—N(R¹⁰)₂, and —NR¹¹SO₂—N(R¹⁰)₂;
each R¹⁰ is independently selected from the group consisting of optionally substituted C1-C6 alkyl, optionally substituted C3-C8 carbocyclyl, optionally substituted heterocyclyl, optionally substituted aryl, and optionally substituted heteroaryl;
each R¹¹ is independently selected from the group consisting of hydrogen, optionally substituted C1-C6 alkyl, optionally substituted C3-C8 carbocyclyl, optionally substituted heterocyclyl, optionally substituted aryl, and optionally substituted heteroaryl; and
R¹² is H or optionally substituted C1-C6 alkyl.

11. The compound of claim 10, or a pharmaceutically acceptable salt or solvate thereof, wherein ring B is 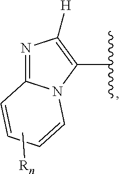
wherein n is 0-4; and each R is independently selected from the group consisting of hydrogen, cyano, halo, hydroxy, azido, amino, nitro, —CO₂H, —S(O)—R¹⁰, —S—R¹⁰, —S(O)₂—R¹⁰, optionally substituted C1-C6 alkoxy, optionally substituted C1-C6 alkyl, optionally substituted C1-C6 alkynyl, optionally substituted carbocyclyl, optionally substituted C1-C6 alkenyl, optionally substituted heterocyclyl, —N(R¹¹)₂, —CO—R¹⁰, —CO₂—R¹⁰, —CON(R¹¹)₂, —NR¹¹CO—R¹⁰, —NR¹¹CO₂—R¹⁰, —SO₂N(R¹¹)₂, —C(═NR¹²)—N(R¹¹)₂, —NR¹¹CO—R¹⁰, —NR¹¹CO₂—R¹⁰, —NR¹¹CO—N(R¹⁰)₂, and —NR¹¹SO₂—N(R¹⁰)₂;
each R¹⁰ is independently selected from the group consisting of optionally substituted C1-C6 alkyl, optionally substituted C3-C8 carbocyclyl, optionally substituted heterocyclyl, optionally substituted aryl, and optionally substituted heteroaryl;
each R¹¹ is independently selected from the group consisting of hydrogen, optionally substituted C1-C6 alkyl, optionally substituted C3-C8 carbocyclyl, optionally substituted heterocyclyl, optionally substituted aryl, and optionally substituted heteroaryl; and
R¹² is H or optionally substituted C1-C6 alkyl.

12. The compound of claim 10, or a pharmaceutically acceptable salt or solvate thereof, wherein ring B is 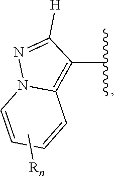
wherein n is 0-4; and each R is independently selected from the group consisting of hydrogen, cyano, halo, hydroxy, azido, amino, nitro, —CO₂H, —S(O)—R¹⁰, —S—R¹⁰, —S(O)₂—R¹⁰, optionally substituted C1-C6 alkoxy, optionally substituted C1-C6 alkyl, optionally substituted C1-C6 alkynyl, optionally substituted carbocyclyl, optionally substituted C1-C6 alkenyl, optionally substituted heterocyclyl, —N(R¹¹)₂, —CO—R¹⁰, —CO₂—R¹⁰, —CON(R¹¹)₂, —NR¹¹CO—R¹⁰, —NR¹¹CO₂—R¹⁰, —SO₂N(R¹¹)₂, —C(═NR¹²)—N(R¹¹)₂, —NR¹¹CO—R¹⁰, —NR¹¹CO₂—R¹⁰, —NR¹¹CO—N(R¹⁰)₂, and —NR¹¹SO₂—N(R¹⁰)₂;
each R¹⁰ is independently selected from the group consisting of optionally substituted C1-C6 alkyl, optionally substituted C3-C8 carbocyclyl, optionally substituted heterocyclyl, optionally substituted aryl, and optionally substituted heteroaryl;
each R¹¹ is independently selected from the group consisting of hydrogen, optionally substituted C1-C6 alkyl, optionally substituted C3-C8 carbocyclyl, optionally substituted heterocyclyl, optionally substituted aryl, and optionally substituted heteroaryl; and
R¹² is H or optionally substituted C1-C6 alkyl.

13. The compound of claim 10, or a pharmaceutically acceptable salt or solvate thereof, wherein ring B is 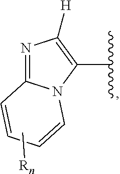
wherein n is 0.

14. The compound of claim 10, or a pharmaceutically acceptable salt or solvate thereof, wherein ring B is 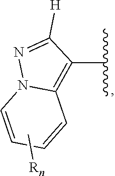
wherein n is 0.

15. The compound of claim 11 or 12, or a pharmaceutically acceptable salt or solvate thereof, wherein R is hydrogen, halo, cyano, —CONH₂, or heterocyclyl.

16. The compound of claim 11 or 12, or a pharmaceutically acceptable salt or solvate thereof, wherein R is hydrogen or halo.

17. The compound of claim 11 or 12, or a pharmaceutically acceptable salt or solvate thereof, wherein n is 1 or 2; and each R is independently selected from the group consisting of cyano, halo, hydroxy, —CO₂H, optionally substituted C1-C6 alkoxy, optionally substituted C1-C6 alkyl, optionally substituted heterocyclyl, or —CON(R¹¹)₂.

18. The compound of claim 11 or 12, or a pharmaceutically acceptable salt or solvate thereof, wherein n is 1; and R is optionally substituted heterocyclyl.

19. The compound of claim 18, or a pharmaceutically acceptable salt or solvate thereof, wherein the optionally substituted heterocyclyl is an optionally substituted morpholinyl or piperazinyl.

20. The compound of any one of claims 18 or 19, or a pharmaceutically acceptable salt or solvate thereof, wherein the R group is placed to provide the regioisomer indicated below:

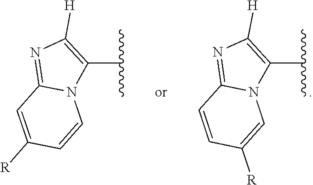

21. The compound of any one of claims 1-20, or a pharmaceutically acceptable salt or solvate thereof, wherein R¹ is optionally substituted carbocyclyl.

22. The compound of claim 21, or a pharmaceutically acceptable salt or solvate thereof, wherein the optionally substituted carbocyclyl is a 3- or 4-membered optionally substituted carbocyclyl.

23. The compound of any one of claims 1-20, or a pharmaceutically acceptable salt or solvate thereof, wherein R¹ is optionally substituted heterocyclyl.

24. The compound of claim 23, or a pharmaceutically acceptable salt or solvate thereof, wherein R¹ is an optionally substituted azetidine, or an optionally substituted oxetane.

25. The compound of claim 23, or a pharmaceutically acceptable salt or solvate thereof, wherein R¹ is an optionally substituted piperazine, or an optionally substituted morpholine.

26. The compound of claim 23, or a pharmaceutically acceptable salt or solvate thereof, wherein R¹ is an optionally substituted pyrrolidine.

27. The compound of claim 24, or a pharmaceutically acceptable salt or solvate thereof, wherein the optionally substituted azetidine is optionally substituted with one or more substituents selected from optionally substituted alkyl, halo, fluoroalkyl, oxo, thioxo, cyano, nitro, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); where each R^{a} is independently hydrogen, alkyl (optionally substituted with halogen, hydroxy, methoxy, amino or trifluoromethyl), fluoroalkyl, cycloalkyl (optionally substituted with halogen, hydroxy, methoxy, amino or trifluoromethyl), cycloalkylalkyl (optionally substituted with halogen, hydroxy, methoxy, amino or trifluoromethyl), aryl (optionally substituted with halogen, hydroxy, methoxy, amino or trifluoromethyl), aralkyl (optionally substituted with halogen, hydroxy, methoxy, amino or trifluoromethyl), heterocyclyl (optionally substituted with halogen, hydroxy, methoxy, amino or trifluoromethyl), heterocyclylalkyl (optionally substituted with halogen, hydroxy, methoxy, amino or trifluoromethyl), heteroaryl (optionally substituted with halogen, hydroxy, methoxy, amino or trifluoromethyl), or heteroarylalkyl (optionally substituted with halogen, hydroxy, methoxy, amino or trifluoromethyl), or optionally, for a —N(R^{a})₂ group, both R^{a} groups may join to form a nitrogen-containing heterocyclyl; each R^{b} is independently a direct bond or a straight or branched alkylene or alkenylene chain; R^{c} is a straight or branched alkylene or alkenylene chain; and where each of the R^{a}, R^{b}, or R^{c} substituents is unsubstituted unless otherwise indicated.

28. The compound of claim 24, or a pharmaceutically acceptable salt or solvate thereof, wherein the optionally substituted azetidine is optionally substituted with one or more substituents selected from —R^{b}—C(O)OR^{a}, —R^{b}—C(O)N(R^{a})₂; where each R^{a} is independently hydrogen, alkyl (optionally substituted with halogen, hydroxy, methoxy, amino or trifluoromethyl), fluoroalkyl, cycloalkyl (optionally substituted with halogen, hydroxy, methoxy, amino or trifluoromethyl), cycloalkylalkyl (optionally substituted with halogen, hydroxy, methoxy, amino or trifluoromethyl), aryl (optionally substituted with halogen, hydroxy, methoxy, amino or trifluoromethyl), aralkyl (optionally substituted with halogen, hydroxy, methoxy, amino or trifluoromethyl), heterocyclyl (optionally substituted with halogen, hydroxy, methoxy, amino or trifluoromethyl), heterocyclylalkyl (optionally substituted with halogen, hydroxy, methoxy, amino or trifluoromethyl), heteroaryl (optionally substituted with halogen, hydroxy, methoxy, amino or trifluoromethyl), or heteroarylalkyl (optionally substituted with halogen, hydroxy, methoxy, amino or trifluoromethyl), or optionally, for a —N(R^{a})₂ group, both R^{a} groups may join to form a nitrogen-containing heterocyclyl; each R^{b} is independently a direct bond or a straight or branched alkylene or alkenylene chain; and where each of the R^{a} or R^{b} substituents is unsubstituted unless otherwise indicated.

29. The compound of claim 24, or a pharmaceutically acceptable salt or solvate thereof, wherein the optionally substituted azetidine is optionally substituted with one or more substituents selected from —C(O)OR^{a}, —C(O)N(R^{a})₂; where each R^{a} is independently hydrogen, alkyl (optionally substituted with halogen, hydroxy, methoxy, amino or trifluoromethyl), or cycloalkyl (optionally substituted with halogen, hydroxy, methoxy, amino or trifluoromethyl).

30. The compound of any one of claims 23-29, or a pharmaceutically acceptable salt or solvate thereof, wherein the optional substituent is bonded to the nitrogen of the heterocyclyl group.

31. The compound of any one of claims 27, 28, 29 or 30, or a pharmaceutically acceptable salt or solvate thereof, wherein R¹ is

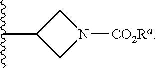

32. The compound of any one of claims 1-20, or a pharmaceutically acceptable salt or solvate thereof, wherein R¹ is optionally substituted alkyl, optionally substituted carbocyclylalkyl, optionally substituted heterocyclylalkyl, optionally substituted heteroaryl, or optionally substituted heteroaralkyl.

33. The compound of claim 21 or 22, or a pharmaceutically acceptable salt or solvate thereof, wherein the optionally substituted carbocyclyl is substituted with at least a halogen.

34. The compound of any one of claims 1-33, or a pharmaceutically acceptable salt or solvate thereof, wherein R² is hydrogen, or halo.

35. The compound of any one of claims 1-33, or a pharmaceutically acceptable salt or solvate thereof, wherein R² is hydrogen.

36. The compound of any one of claims 1-35, or a pharmaceutically acceptable salt or solvate thereof, wherein R³ is fluoro.

37. The compound of any one of claims 1-36, or a pharmaceutically acceptable salt or solvate thereof, wherein R⁴ is optionally substituted C1-C2 alkyl.

38. The compound of any one of claims 1-36, or a pharmaceutically acceptable salt or solvate thereof, wherein R⁴ is optionally substituted C1 alkyl.

39. The compound of any one of claims 1-36, or a pharmaceutically acceptable salt or solvate thereof, wherein R⁴ is CH₃.

40. The compound of any one of claims 1-39, or a pharmaceutically acceptable salt or solvate thereof, wherein R⁵ is hydrogen, or halo.

41. The compound of any one of claims 1-39, or a pharmaceutically acceptable salt or solvate thereof, wherein R⁵ is hydrogen.

42. The compound of any one of claims 1-39, or a pharmaceutically acceptable salt or solvate thereof, wherein R⁵ is fluoro.

43. The compound of claim 1, or a pharmaceutically acceptable salt or solvate thereof, wherein R² is hydrogen, R³ is fluoro, R⁴ is CH₃, and R⁵ is fluoro.

44. The compound of any one of claims 4, 10, 11, 13, 20, 24 or 31, or a pharmaceutically acceptable salt or solvate thereof, wherein R² is hydrogen, R³ is fluoro, R⁴ is CH₃, and R⁵ is fluoro.

45. The compound of claim 4, or a pharmaceutically acceptable salt or solvate thereof, wherein R² is hydrogen, R³ is fluoro, R⁴ is CH₃, and R⁵ is fluoro.

46. The compound of claim 45, or a pharmaceutically acceptable salt or solvate thereof, wherein R¹ is a azetidine substituted with a —C(O)OR^{a}, wherein R^{a} is alkyl (optionally substituted with halogen, hydroxy, methoxy, amino or trifluoromethyl).

47. The compound of claim 45 or 46, or a pharmaceutically acceptable salt or solvate thereof, wherein ring B is 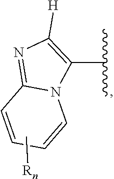
wherein n is 0.

48. A compound, or a pharmaceutically acceptable salt or solvate thereof, selected from the group consisting of:
N-(5-(5-(3,3-difluorocyclobutyl)-1,2,4-oxadiazol-3-yl)-3-fluoro-2-methylphenyl)imidazo[1,2-a]pyridine-3-carboxamide;
N-(3-fluoro-5-(5-((1R,2S)-2-fluorocyclopropyl)-1,2,4-oxadiazol-3-yl)-2-methylphenyl)imidazo[1,2-a]pyridine-3-carboxamide;
methyl 3-(3-(3-fluoro-5-(imidazo[1,2-a]pyridine-3-carboxamido)-4-methylphenyl)-1,2,4-oxadiazol-5-yl)azetidine-1-carboxylate;
N-(5-(5-(3,3-difluorocyclobutyl)-1,2,4-oxadiazol-3-yl)-3-fluoro-2-methylphenyl)pyrazolo[1,5-a]pyridine-3-carboxamide;
N-(3-fluoro-5-(5-((1R,2S)-2-fluorocyclopropyl)-1,2,4-oxadiazol-3-yl)-2-methylphenyl)pyrazolo[1,5-a]pyridine-3-carboxamide;
methyl 3-(3-(3-fluoro-4-methyl-5-(pyrazolo[1,5-a]pyridine-3-carboxamido)phenyl)-1,2,4-oxadiazol-5-yl)azetidine-1-carboxylate;
N-(5-(5-(3,3-difluorocyclobutyl)-1,2,4-oxadiazol-3-yl)-3-fluoro-2-methylphenyl)-6-morpholinoimidazo[1,2-a]pyridine-3-carboxamide;
N-(3-fluoro-5-(5-((1R,2S)-2-fluorocyclopropyl)-1,2,4-oxadiazol-3-yl)-2-methylphenyl)-6-morpholinoimidazo[1,2-a]pyridine-3-carboxamide;
methyl 3-(3-(3-fluoro-4-methyl-5-(6-morpholinoimidazo[1,2-a]pyridine-3-carboxamido)phenyl)-1,2,4-oxadiazol-5-yl)azetidine-1-carboxylate;
N-(5-(5-(3,3-difluorocyclobutyl)-1,2,4-oxadiazol-3-yl)-3-fluoro-2-methylphenyl)-7-(piperazin-1-yl)imidazo[1,2-a]pyridine-3-carboxamide;
N-(5-(5-(3,3-difluorocyclobutyl)-1,2,4-oxadiazol-3-yl)-3-fluoro-2-methylphenyl)-7-(4-methylpiperazin-1-yl)imidazo[1,2-a]pyridine-3-carboxamide;
methyl 3-(3-(3-(6-chloroimidazo[1,2-a]pyridine-3-carboxamido)-5-fluoro-4-methylphenyl)-1,2,4-oxadiazol-5-yl)azetidine-1-carboxylate;
N-(5-(5-(3,3-difluorocyclobutyl)-1,2,4-oxadiazol-3-yl)-3-fluoro-2-methylphenyl)-7-morpholinoimidazo[1,2-a]pyridine-3-carboxamide;
N-(3-fluoro-5-(5-((1R,2S)-2-fluorocyclopropyl)-1,2,4-oxadiazol-3-yl)-2-methylphenyl)-7-morpholinoimidazo[1,2-a]pyridine-3-carboxamide;
methyl 3-(3-(3-fluoro-4-methyl-5-(7-morpholinoimidazo[1,2-a]pyridine-3-carboxamido)phenyl)-1,2,4-oxadiazol-5-yl)azetidine-1-carboxylate;
methyl 3-(3-(3-(7-chloroimidazo[1,2-a]pyridine-3-carboxamido)-5-fluoro-4-methylphenyl)-1,2,4-oxadiazol-5-yl)azetidine-1-carboxylate;
methyl 3-(3-(3-fluoro-4-methyl-5-(7-methylimidazo[1,2-a]pyridine-3-carboxamido)phenyl)-1,2,4-oxadiazol-5-yl)azetidine-1-carboxylate;
7-(4-acetylpiperazin-1-yl)-N-(5-(5-(3,3-difluorocyclobutyl)-1,2,4-oxadiazol-3-yl)-3-fluoro-2-methylphenyl)imidazo[1,2-a]pyridine-3-carboxamide;
methyl 3-(3-(3-fluoro-5-(6-fluoroimidazo[1,2-a]pyridine-3-carboxamido)-4-methylphenyl)-1,2,4-oxadiazol-5-yl)azetidine-1-carboxylate;
methyl 3-(3-(3-fluoro-4-methyl-5-(6-(piperazin-1-yl)imidazo[1,2-a]pyridine-3-carboxamido)phenyl)-1,2,4-oxadiazol-5-yl)azetidine-1-carboxylate;
methyl 3-(3-(3-fluoro-4-methyl-5-(6-methylimidazo[1,2-a]pyridine-3-carboxamido)phenyl)-1,2,4-oxadiazol-5-yl)azetidine-1-carboxylate;
N-(3-fluoro-5-(5-((1R,2S)-2-fluorocyclopropyl)-1,2,4-oxadiazol-3-yl)-2-methylphenyl)-7-(piperazin-1-yl)imidazo[1,2-a]pyridine-3-carboxamide;
ethyl 3-(3-(3-fluoro-5-(imidazo[1,2-a]pyridine-3-carboxamido)-4-methylphenyl)-1,2,4-oxadiazol-5-yl)azetidine-1-carboxylate;
methyl 3-(3-(3-fluoro-5-(7-fluoroimidazo[1,2-a]pyridine-3-carboxamido)-4-methylphenyl)-1,2,4-oxadiazol-5-yl)azetidine-1-carboxylate;
N-(5-(5-(3,3-difluorocyclobutyl)-1,2,4-oxadiazol-3-yl)-3-fluoro-2-methylphenyl)-6-(piperazin-1-yl)imidazo[1,2-a]pyridine-3-carboxamide;
N-(3-fluoro-5-(5-((1R,2S)-2-fluorocyclopropyl)-1,2,4-oxadiazol-3-yl)-2-methylphenyl)-6-(piperazin-1-yl)imidazo[1,2-a]pyridine-3-carboxamide;
methyl 3-(3-(3-fluoro-5-(6-methoxyimidazo[1,2-a]pyridine-3-carboxamido)-4-methylphenyl)-1,2,4-oxadiazol-5-yl)azetidine-1-carboxylate;
methyl 3-(3-(3-fluoro-4-methyl-5-(6-(4-methylpiperazin-1-yl)imidazo[1,2-a]pyridine-3-carboxamido)phenyl)-1,2,4-oxadiazol-5-yl)azetidine-1-carboxylate;
methyl 3-(3-(3-fluoro-5-(7-methoxyimidazo[1,2-a]pyridine-3-carboxamido)-4-methylphenyl)-1,2,4-oxadiazol-5-yl)azetidine-1-carboxylate;
methyl 3-(3-(3-fluoro-4-methyl-5-(7-(piperazin-1-yl)imidazo[1,2-a]pyridine-3-carboxamido)phenyl)-1,2,4-oxadiazol-5-yl)azetidine-1-carboxylate;
cyclopropyl 3-(3-(3-fluoro-5-(imidazo[1,2-a]pyridine-3-carboxamido)-4-methylphenyl)-1,2,4-oxadiazol-5-yl)azetidine-1-carboxylate;
N-(3-fluoro-2-methyl-5-(5-(1-(methylcarbamoyl)azetidin-3-yl)-1,2,4-oxadiazol-3-yl)phenyl)imidazo[1,2-a]pyridine-3-carboxamide;
N-(5-(5-(3,3-difluorocyclobutyl)-1,2,4-oxadiazol-3-yl)-3-fluoro-2-methylphenyl)-6-(4-methylpiperazin-1-yl)imidazo[1,2-a]pyridine-3-carboxamide;
N-(3-fluoro-5-(5-((1R,2S)-2-fluorocyclopropyl)-1,2,4-oxadiazol-3-yl)-2-methylphenyl)-6-(4-methylpiperazin-1-yl)imidazo[1,2-a]pyridine-3-carboxamide;
methyl 3-(3-(3-(7-cyanoimidazo[1,2-a]pyridine-3-carboxamido)-5-fluoro-4-methylphenyl)-1,2,4-oxadiazol-5-yl)azetidine-1-carboxylate;
methyl 3-(3-(3-(6-(4-acetylpiperazin-1-yl)imidazo[1,2-a]pyridine-3-carboxamido)-5-fluoro-4-methylphenyl)-1,2,4-oxadiazol-5-yl)azetidine-1-carboxylate;
methyl 3-(5-(3-fluoro-5-(imidazo[1,2-a]pyridine-3-carboxamido)-4-methylphenyl)-1,2,4-oxadiazol-3-yl)azetidine-1-carboxylate;
6-(4-acetylpiperazin-1-yl)-N-(5-(5-(3,3-difluorocyclobutyl)-1,2,4-oxadiazol-3-yl)-3-fluoro-2-methylphenyl)imidazo[1,2-a]pyridine-3-carboxamide;
6-(4-acetylpiperazin-1-yl)-N-(3-fluoro-5-(5-((1R,2S)-2-fluorocyclopropyl)-1,2,4-oxadiazol-3-yl)-2-methylphenyl)imidazo[1,2-a]pyridine-3-carboxamide;
methyl 3-(3-(3-(6-carbamoylimidazo[1,2-a]pyridine-3-carboxamido)-5-fluoro-4-methylphenyl)-1,2,4-oxadiazol-5-yl)azetidine-1-carboxylate;
2-aminoethyl 3-(3-(3-fluoro-5-(imidazo[1,2-a]pyridine-3-carboxamido)-4-methylphenyl)-1,2,4-oxadiazol-5-yl)azetidine-1-carboxylate;
2-methoxyethyl 3-(3-(3-fluoro-5-(imidazo[1,2-a]pyridine-3-carboxamido)-4-methylphenyl)-1,2,4-oxadiazol-5-yl)azetidine-1-carboxylate;
methyl 3-(3-(3-(7-carbamoylimidazo[1,2-a]pyridine-3-carboxamido)-5-fluoro-4-methylphenyl)-1,2,4-oxadiazol-5-yl)azetidine-1-carboxylate;
isopropyl 3-(3-(3-fluoro-5-(imidazo[1,2-a]pyridine-3-carboxamido)-4-methylphenyl)-1,2,4-oxadiazol-5-yl)azetidine-1-carboxylate;
N-(5-(5-(1-(ethylcarbamoyl)azetidin-3-yl)-1,2,4-oxadiazol-3-yl)-3-fluoro-2-methylphenyl)imidazo[1,2-a]pyridine-3-carboxamide;
N-(3-fluoro-5-(5-(1-(isopropylcarbamoyl)azetidin-3-yl)-1,2,4-oxadiazol-3-yl)-2-methylphenyl)imidazo[1,2-a]pyridine-3-carboxamide;
N-(3-fluoro-5-(5-(1-((2-hydroxyethyl)carbamoyl)azetidin-3-yl)-1,2,4-oxadiazol-3-yl)-2-methylphenyl)imidazo[1,2-a]pyridine-3-carboxamide;
N-(5-(5-(1-(cyclopropylcarbamoyl)azetidin-3-yl)-1,2,4-oxadiazol-3-yl)-3-fluoro-2-methylphenyl)imidazo[1,2-a]pyridine-3-carboxamide;
N-(3-fluoro-5-(5-(1-((2-methoxyethyl)carbamoyl)azetidin-3-yl)-1,2,4-oxadiazol-3-yl)-2-methylphenyl)imidazo[1,2-a]pyridine-3-carboxamide;
N-(5-(5-(1-((2-aminoethyl)carbamoyl)azetidin-3-yl)-1,2,4-oxadiazol-3-yl)-3-fluoro-2-methylphenyl)imidazo[1,2-a]pyridine-3-carboxamide;
N-(3-fluoro-2-methyl-5-(5-(1-(morpholine-4-carbonyl)azetidin-3-yl)-1,2,4-oxadiazol-3-yl)phenyl)imidazo[1,2-a]pyridine-3-carboxamide;
N-(3-fluoro-2-methyl-5-(5-(1-(piperazine-1-carbonyl)azetidin-3-yl)-1,2,4-oxadiazol-3-yl)phenyl)imidazo[1,2-a]pyridine-3-carboxamide;
N-(3-fluoro-2-methyl-5-(5-(1-(4-methylpiperazine-1-carbonyl)azetidin-3-yl)-1,2,4-oxadiazol-3-yl)phenyl)imidazo[1,2-a]pyridine-3-carboxamide;
methyl 3-(3-(3-(6-cyanoimidazo[1,2-a]pyridine-3-carboxamido)-5-fluoro-4-methylphenyl)-1,2,4-oxadiazol-5-yl)azetidine-1-carboxylate;
methyl 3-(3-(3-fluoro-4-methyl-5-(6-(trifluoromethyl)imidazo[1,2-a]pyridine-3-carboxamido)phenyl)-1,2,4-oxadiazol-5-yl)azetidine-1-carboxylate;
methyl 3-(3-(3-fluoro-5-(6-isopropylimidazo[1,2-a]pyridine-3-carboxamido)-4-methylphenyl)-1,2,4-oxadiazol-5-yl)azetidine-1-carboxylate;
methyl 3-(3-(3-(6-cyclopropylimidazo[1,2-a]pyridine-3-carboxamido)-5-fluoro-4-methylphenyl)-1,2,4-oxadiazol-5-yl)azetidine-1-carboxylate;
methyl 3-(3-(3-fluoro-5-(7-isopropylimidazo[1,2-a]pyridine-3-carboxamido)-4-methylphenyl)-1,2,4-oxadiazol-5-yl)azetidine-1-carboxylate;
methyl 3-(3-(3-(7-cyclopropylimidazo[1,2-a]pyridine-3-carboxamido)-5-fluoro-4-methylphenyl)-1,2,4-oxadiazol-5-yl)azetidine-1-carboxylate;
methyl 3-(3-(3-fluoro-4-methyl-5-(7-(4-methylpiperazin-1-yl)imidazo[1,2-a]pyridine-3-carboxamido)phenyl)-1,2,4-oxadiazol-5-yl)azetidine-1-carboxylate;
methyl 4-(3-(3-fluoro-5-(imidazo[1,2-a]pyridine-3-carboxamido)-4-methylphenyl)-1,2,4-oxadiazol-5-yl)piperazine-1-carboxylate;
methyl 4-(3-(3-fluoro-5-(imidazo[1,2-a]pyridine-3-carboxamido)-4-methylphenyl)-1,2,4-oxadiazol-5-yl)-2-methylpiperazine-1-carboxylate;
methyl 4-(3-(3-fluoro-5-(imidazo[1,2-a]pyridine-3-carboxamido)-4-methylphenyl)-1,2,4-oxadiazol-5-yl)-2-isopropylpiperazine-1-carboxylate;
methyl 4-(3-(3-fluoro-5-(imidazo[1,2-a]pyridine-3-carboxamido)-4-methylphenyl)-1,2,4-oxadiazol-5-yl)-3-methylpiperazine-1-carboxylate;
methyl 4-(3-(3-fluoro-5-(imidazo[1,2-a]pyridine-3-carboxamido)-4-methylphenyl)-1,2,4-oxadiazol-5-yl)-3-isopropylpiperazine-1-carboxylate;
N-(5-(5-(4-acetylpiperazin-1-yl)-1,2,4-oxadiazol-3-yl)-3-fluoro-2-methylphenyl)imidazo[1,2-a]pyridine-3-carboxamide;
N-(5-(5-(4-carbamoylpiperazin-1-yl)-1,2,4-oxadiazol-3-yl)-3-fluoro-2-methylphenyl)imidazo[1,2-a]pyridine-3-carboxamide;
N-(3-fluoro-2-methyl-5-(5-(4-methylpiperazin-1-yl)-1,2,4-oxadiazol-3-yl)phenyl)imidazo[1,2-a]pyridine-3-carboxamide;
N-(3-fluoro-2-methyl-5-(5-(piperazin-1-yl)-1,2,4-oxadiazol-3-yl)phenyl)imidazo[1,2-a]pyridine-3-carboxamide;
N-(3-fluoro-2-methyl-5-(5-morpholino-1,2,4-oxadiazol-3-yl)phenyl)imidazo[1,2-a]pyridine-3-carboxamide;
6-fluoro-N-(3-fluoro-5-(5-((1R,2S)-2-fluorocyclopropyl)-1,2,4-oxadiazol-3-yl)-2-methylphenyl)imidazo[1,2-a]pyridine-3-carboxamide;
6-chloro-N-(3-fluoro-5-(5-((1R,2S)-2-fluorocyclopropyl)-1,2,4-oxadiazol-3-yl)-2-methylphenyl)imidazo[1,2-a]pyridine-3-carboxamide;
6-cyano-N-(3-fluoro-5-(5-((1R,2S)-2-fluorocyclopropyl)-1,2,4-oxadiazol-3-yl)-2-methylphenyl)imidazo[1,2-a]pyridine-3-carboxamide;
N3-(3-fluoro-5-(5-((1R,2S)-2-fluorocyclopropyl)-1,2,4-oxadiazol-3-yl)-2-methylphenyl)imidazo[1,2-a]pyridine-3,6-dicarboxamide;
N-(3-fluoro-5-(5-((1R,2S)-2-fluorocyclopropyl)-1,2,4-oxadiazol-3-yl)-2-methylphenyl)-6-(trifluoromethyl)imidazo[1,2-a]pyridine-3-carboxamide;
N-(3-fluoro-5-(5-((1R,2S)-2-fluorocyclopropyl)-1,2,4-oxadiazol-3-yl)-2-methylphenyl)-6-methylimidazo[1,2-a]pyridine-3-carboxamide;
N-(3-fluoro-5-(5-((1R,2S)-2-fluorocyclopropyl)-1,2,4-oxadiazol-3-yl)-2-methylphenyl)-6-isopropylimidazo[1,2-a]pyridine-3-carboxamide;
6-cyclopropyl-N-(3-fluoro-5-(5-((1R,2S)-2-fluorocyclopropyl)-1,2,4-oxadiazol-3-yl)-2-methylphenyl)imidazo[1,2-a]pyridine-3-carboxamide;
7-fluoro-N-(3-fluoro-5-(5-((1R,2S)-2-fluorocyclopropyl)-1,2,4-oxadiazol-3-yl)-2-methylphenyl)imidazo[1,2-a]pyridine-3-carboxamide;
7-chloro-N-(3-fluoro-5-(5-((1R,2S)-2-fluorocyclopropyl)-1,2,4-oxadiazol-3-yl)-2-methylphenyl)imidazo[1,2-a]pyridine-3-carboxamide;
N-(3-fluoro-5-(5-((1R,2S)-2-fluorocyclopropyl)-1,2,4-oxadiazol-3-yl)-2-methylphenyl)-7-(trifluoromethyl)imidazo[1,2-a]pyridine-3-carboxamide;
7-cyano-N-(3-fluoro-5-(5-((1R,2S)-2-fluorocyclopropyl)-1,2,4-oxadiazol-3-yl)-2-methylphenyl)imidazo[1,2-a]pyridine-3-carboxamide;
N3-(3-fluoro-5-(5-((1R,2S)-2-fluorocyclopropyl)-1,2,4-oxadiazol-3-yl)-2-methylphenyl)imidazo[1,2-a]pyridine-3,7-dicarboxamide;
N-(3-fluoro-5-(5-((1R,2S)-2-fluorocyclopropyl)-1,2,4-oxadiazol-3-yl)-2-methylphenyl)-7-methylimidazo[1,2-a]pyridine-3-carboxamide;
N-(3-fluoro-5-(5-((1R,2S)-2-fluorocyclopropyl)-1,2,4-oxadiazol-3-yl)-2-methylphenyl)-7-isopropylimidazo[1,2-a]pyridine-3-carboxamide;
7-cyclopropyl-N-(3-fluoro-5-(5-((1R,2S)-2-fluorocyclopropyl)-1,2,4-oxadiazol-3-yl)-2-methylphenyl)imidazo[1,2-a]pyridine-3-carboxamide;
N-(3-fluoro-5-(5-((1R,2S)-2-fluorocyclopropyl)-1,2,4-oxadiazol-3-yl)-2-methylphenyl)-7-(4-methylpiperazin-1-yl)imidazo[1,2-a]pyridine-3-carboxamide;
methyl 3-(3-(3-fluoro-4-methyl-5-(7-(trifluoromethyl)imidazo[1,2-a]pyridine-3-carboxamido)phenyl)-1,2,4-oxadiazol-5-yl)azetidine-1-carboxylate;
N-(3-fluoro-5-(5-((1S,2R)-2-fluorocyclopropyl)-1,2,4-oxadiazol-3-yl)-2-methylphenyl)imidazo[1,2-a]pyridine-3-carboxamide;
N-(3-fluoro-5-(5-((1R,2R)-2-fluorocyclopropyl)-1,2,4-oxadiazol-3-yl)-2-methylphenyl)imidazo[1,2-a]pyridine-3-carboxamide;
N-(3-fluoro-5-(5-((1S,2S)-2-fluorocyclopropyl)-1,2,4-oxadiazol-3-yl)-2-methylphenyl)imidazo[1,2-a]pyridine-3-carboxamide;
methyl 3-(3-(3-fluoro-5-(6-fluoropyrazolo[1,5-a]pyridine-3-carboxamido)-4-methylphenyl)-1,2,4-oxadiazol-5-yl)azetidine-1-carboxylate;
methyl 3-(3-(3-(6-chloropyrazolo[1,5-a]pyridine-3-carboxamido)-5-fluoro-4-methylphenyl)-1,2,4-oxadiazol-5-yl)azetidine-1-carboxylate;
methyl 3-(3-(3-(6-cyanopyrazolo[1,5-a]pyridine-3-carboxamido)-5-fluoro-4-methylphenyl)-1,2,4-oxadiazol-5-yl)azetidine-1-carboxylate;
methyl 3-(3-(3-(6-carbamoylpyrazolo[1,5-a]pyridine-3-carboxamido)-5-fluoro-4-methylphenyl)-1,2,4-oxadiazol-5-yl)azetidine-1-carboxylate;
methyl 3-(3-(3-fluoro-4-methyl-5-(6-(trifluoromethyl)pyrazolo[1,5-a]pyridine-3-carboxamido)phenyl)-1,2,4-oxadiazol-5-yl)azetidine-1-carboxylate;
methyl 3-(3-(3-fluoro-5-(6-methoxypyrazolo[1,5-a]pyridine-3-carboxamido)-4-methylphenyl)-1,2,4-oxadiazol-5-yl)azetidine-1-carboxylate;
methyl 3-(3-(3-fluoro-5-(6-isopropylpyrazolo[1,5-a]pyridine-3-carboxamido)-4-methylphenyl)-1,2,4-oxadiazol-5-yl)azetidine-1-carboxylate;
methyl 3-(3-(3-(6-cyclopropylpyrazolo[1,5-a]pyridine-3-carboxamido)-5-fluoro-4-methylphenyl)-1,2,4-oxadiazol-5-yl)azetidine-1-carboxylate;
methyl 3-(3-(3-fluoro-4-methyl-5-(6-methylpyrazolo[1,5-a]pyridine-3-carboxamido)phenyl)-1,2,4-oxadiazol-5-yl)azetidine-1-carboxylate;
methyl 3-(3-(3-fluoro-4-methyl-5-(5-methylpyrazolo[1,5-a]pyridine-3-carboxamido)phenyl)-1,2,4-oxadiazol-5-yl)azetidine-1-carboxylate;
methyl 3-(3-(3-(5-cyclopropylpyrazolo[1,5-a]pyridine-3-carboxamido)-5-fluoro-4-methylphenyl)-1,2,4-oxadiazol-5-yl)azetidine-1-carboxylate;
methyl 3-(3-(3-fluoro-5-(5-isopropylpyrazolo[1,5-a]pyridine-3-carboxamido)-4-methylphenyl)-1,2,4-oxadiazol-5-yl)azetidine-1-carboxylate;
methyl 3-(3-(3-fluoro-5-(5-fluoropyrazolo[1,5-a]pyridine-3-carboxamido)-4-methylphenyl)-1,2,4-oxadiazol-5-yl)azetidine-1-carboxylate;
methyl 3-(3-(3-(5-chloropyrazolo[1,5-a]pyridine-3-carboxamido)-5-fluoro-4-methylphenyl)-1,2,4-oxadiazol-5-yl)azetidine-1-carboxylate;
methyl 3-(3-(3-(5-cyanopyrazolo[1,5-a]pyridine-3-carboxamido)-5-fluoro-4-methylphenyl)-1,2,4-oxadiazol-5-yl)azetidine-1-carboxylate;
methyl 3-(3-(3-(5-carbamoylpyrazolo[1,5-a]pyridine-3-carboxamido)-5-fluoro-4-methylphenyl)-1,2,4-oxadiazol-5-yl)azetidine-1-carboxylate;
methyl 3-(3-(3-fluoro-4-methyl-5-(5-(trifluoromethyl)pyrazolo[1,5-a]pyridine-3-carboxamido)phenyl)-1,2,4-oxadiazol-5-yl)azetidine-1-carboxylate;
methyl 3-(3-(3-fluoro-4-methyl-5-(5-morpholinopyrazolo[1,5-a]pyridine-3-carboxamido)phenyl)-1,2,4-oxadiazol-5-yl)azetidine-1-carboxylate;
methyl 3-(3-(3-fluoro-4-methyl-5-(5-(piperazin-1-yl)pyrazolo[1,5-a]pyridine-3-carboxamido)phenyl)-1,2,4-oxadiazol-5-yl)azetidine-1-carboxylate;
methyl 3-(3-(3-fluoro-4-methyl-5-(5-(4-methylpiperazin-1-yl)pyrazolo[1,5-a]pyridine-3-carboxamido)phenyl)-1,2,4-oxadiazol-5-yl)azetidine-1-carboxylate;
methyl 3-(3-(3-fluoro-4-methyl-5-(6-morpholinopyrazolo[1,5-a]pyridine-3-carboxamido)phenyl)-1,2,4-oxadiazol-5-yl)azetidine-1-carboxylate;
methyl 3-(3-(3-fluoro-4-methyl-5-(6-(piperazin-1-yl)pyrazolo[1,5-a]pyridine-3-carboxamido)phenyl)-1,2,4-oxadiazol-5-yl)azetidine-1-carboxylate;
methyl 3-(3-(3-fluoro-4-methyl-5-(6-(4-methylpiperazin-1-yl)pyrazolo[1,5-a]pyridine-3-carboxamido)phenyl)-1,2,4-oxadiazol-5-yl)azetidine-1-carboxylate;
N-(5-(3-(3,3-difluorocyclobutyl)-1,2,4-oxadiazol-5-yl)-3-fluoro-2-methylphenyl)imidazo[1,2-a]pyridine-3-carboxamide;
N-(3-fluoro-5-(3-((1R,2S)-2-fluorocyclopropyl)-1,2,4-oxadiazol-5-yl)-2-methylphenyl)imidazo[1,2-a]pyridine-3-carboxamide;
isopropyl 3-(5-(3-fluoro-5-(imidazo[1,2-a]pyridine-3-carboxamido)-4-methylphenyl)-1,2,4-oxadiazol-3-yl)azetidine-1-carboxylate;
cyclopropyl 3-(5-(3-fluoro-5-(imidazo[1,2-a]pyridine-3-carboxamido)-4-methylphenyl)-1,2,4-oxadiazol-3-yl)azetidine-1-carboxylate;
N-(3-fluoro-2-methyl-5-(3-(1-(methylcarbamoyl)azetidin-3-yl)-1,2,4-oxadiazol-5-yl)phenyl)imidazo[1,2-a]pyridine-3-carboxamide;
N-(3-fluoro-2-methyl-5-(3-(1-(morpholine-4-carbonyl)azetidin-3-yl)-1,2,4-oxadiazol-5-yl)phenyl)imidazo[1,2-a]pyridine-3-carboxamide;
N-(3-fluoro-2-methyl-5-(3-(1-(piperazine-1-carbonyl)azetidin-3-yl)-1,2,4-oxadiazol-5-yl)phenyl)imidazo[1,2-a]pyridine-3-carboxamide;
methyl 3-(5-(3-fluoro-5-(6-fluoroimidazo[1,2-a]pyridine-3-carboxamido)-4-methylphenyl)-1,2,4-oxadiazol-3-yl)azetidine-1-carboxylate;
methyl 3-(5-(3-(6-chloroimidazo[1,2-a]pyridine-3-carboxamido)-5-fluoro-4-methylphenyl)-1,2,4-oxadiazol-3-yl)azetidine-1-carboxylate;
methyl 3-(5-(3-(6-cyanoimidazo[1,2-a]pyridine-3-carboxamido)-5-fluoro-4-methylphenyl)-1,2,4-oxadiazol-3-yl)azetidine-1-carboxylate;
methyl 3-(5-(3-(6-carbamoylimidazo[1,2-a]pyridine-3-carboxamido)-5-fluoro-4-methylphenyl)-1,2,4-oxadiazol-3-yl)azetidine-1-carboxylate;
methyl 3-(5-(3-fluoro-4-methyl-5-(6-(trifluoromethyl)imidazo[1,2-a]pyridine-3-carboxamido)phenyl)-1,2,4-oxadiazol-3-yl)azetidine-1-carboxylate;
methyl 3-(5-(3-fluoro-4-methyl-5-(6-methylimidazo[1,2-a]pyridine-3-carboxamido)phenyl)-1,2,4-oxadiazol-3-yl)azetidine-1-carboxylate;
methyl 3-(5-(3-fluoro-5-(6-isopropylimidazo[1,2-a]pyridine-3-carboxamido)-4-methylphenyl)-1,2,4-oxadiazol-3-yl)azetidine-1-carboxylate;
methyl 3-(5-(3-(6-cyclopropylimidazo[1,2-a]pyridine-3-carboxamido)-5-fluoro-4-methylphenyl)-1,2,4-oxadiazol-3-yl)azetidine-1-carboxylate;
methyl 3-(5-(3-fluoro-4-methyl-5-(6-morpholinoimidazo[1,2-a]pyridine-3-carboxamido)phenyl)-1,2,4-oxadiazol-3-yl)azetidine-1-carboxylate;
methyl 3-(5-(3-fluoro-4-methyl-5-(6-(piperazin-1-yl)imidazo[1,2-a]pyridine-3-carboxamido)phenyl)-1,2,4-oxadiazol-3-yl)azetidine-1-carboxylate;
methyl 3-(5-(3-fluoro-4-methyl-5-(6-(4-methylpiperazin-1-yl)imidazo[1,2-a]pyridine-3-carboxamido)phenyl)-1,2,4-oxadiazol-3-yl)azetidine-1-carboxylate;
methyl 3-(5-(3-fluoro-5-(7-fluoroimidazo[1,2-a]pyridine-3-carboxamido)-4-methylphenyl)-1,2,4-oxadiazol-3-yl)azetidine-1-carboxylate;
methyl 3-(5-(3-fluoro-4-methyl-5-(7-methylimidazo[1,2-a]pyridine-3-carboxamido)phenyl)-1,2,4-oxadiazol-3-yl)azetidine-1-carboxylate;
methyl 3-(5-(3-(7-chloroimidazo[1,2-a]pyridine-3-carboxamido)-5-fluoro-4-methylphenyl)-1,2,4-oxadiazol-3-yl)azetidine-1-carboxylate;
methyl 3-(5-(3-(7-cyanoimidazo[1,2-a]pyridine-3-carboxamido)-5-fluoro-4-methylphenyl)-1,2,4-oxadiazol-3-yl)azetidine-1-carboxylate;
methyl 3-(5-(3-(7-carbamoylimidazo[1,2-a]pyridine-3-carboxamido)-5-fluoro-4-methylphenyl)-1,2,4-oxadiazol-3-yl)azetidine-1-carboxylate;
methyl 3-(5-(3-fluoro-4-methyl-5-(7-(trifluoromethyl)imidazo[1,2-a]pyridine-3-carboxamido)phenyl)-1,2,4-oxadiazol-3-yl)azetidine-1-carboxylate;
methyl 3-(5-(3-fluoro-4-methyl-5-(7-morpholinoimidazo[1,2-a]pyridine-3-carboxamido)phenyl)-1,2,4-oxadiazol-3-yl)azetidine-1-carboxylate;
methyl 3-(5-(3-fluoro-4-methyl-5-(7-(piperazin-1-yl)imidazo[1,2-a]pyridine-3-carboxamido)phenyl)-1,2,4-oxadiazol-3-yl)azetidine-1-carboxylate;
methyl 3-(5-(3-fluoro-4-methyl-5-(7-(4-methylpiperazin-1-yl)imidazo[1,2-a]pyridine-3-carboxamido)phenyl)-1,2,4-oxadiazol-3-yl)azetidine-1-carboxylate;
N-(3-fluoro-2-methyl-5-(3-morpholino-1,2,4-oxadiazol-5-yl)phenyl)imidazo[1,2-a]pyridine-3-carboxamide;
N-(3-fluoro-2-methyl-5-(3-(piperazin-1-yl)-1,2,4-oxadiazol-5-yl)phenyl)imidazo[1,2-a]pyridine-3-carboxamide;
methyl 4-(5-(3-fluoro-5-(imidazo[1,2-a]pyridine-3-carboxamido)-4-methylphenyl)-1,2,4-oxadiazol-3-yl)piperazine-1-carboxylate;
methyl 4-(5-(3-fluoro-5-(imidazo[1,2-a]pyridine-3-carboxamido)-4-methylphenyl)-1,2,4-oxadiazol-3-yl)-2-methylpiperazine-1-carboxylate;
methyl 4-(5-(3-fluoro-5-(imidazo[1,2-a]pyridine-3-carboxamido)-4-methylphenyl)-1,2,4-oxadiazol-3-yl)-2-isopropylpiperazine-1-carboxylate;
methyl 4-(5-(3-fluoro-5-(imidazo[1,2-a]pyridine-3-carboxamido)-4-methylphenyl)-1,2,4-oxadiazol-3-yl)-3-methylpiperazine-1-carboxylate;
methyl 4-(5-(3-fluoro-5-(imidazo[1,2-a]pyridine-3-carboxamido)-4-methylphenyl)-1,2,4-oxadiazol-3-yl)-3-isopropylpiperazine-1-carboxylate;
methyl 3-(5-(3-fluoro-5-(imidazo[1,2-a]pyridine-3-carboxamido)-4-methylphenyl)-1,3,4-oxadiazol-2-yl)azetidine-1-carboxylate;
methyl 3-(4-(3-fluoro-5-(imidazo[1,2-a]pyridine-3-carboxamido)-4-methylphenyl)oxazol-2-yl)azetidine-1-carboxylate;
methyl 3-(5-(3-fluoro-5-(imidazo[1,2-a]pyridine-3-carboxamido)-4-methylphenyl)oxazol-2-yl)azetidine-1-carboxylate;
methyl 3-(2-(3-fluoro-5-(imidazo[1,2-a]pyridine-3-carboxamido)-4-methylphenyl)oxazol-4-yl)azetidine-1-carboxylate;
methyl 3-(5-(3-fluoro-5-(imidazo[1,2-a]pyridine-3-carboxamido)-4-methylphenyl)-4H-1,2,4-triazol-3-yl)azetidine-1-carboxylate;
methyl (R)-3-(3-(3-fluoro-5-(imidazo[1,2-a]pyridine-3-carboxamido)-4-methylphenyl)-1,2,4-oxadiazol-5-yl)piperidine-1-carboxylate;
N-(3-fluoro-5-(5-((1S,2R)-2-fluorocyclopropyl)-1,2,4-oxadiazol-3-yl)-2-methylphenyl)-7-morpholinoimidazo[1,2-a]pyridine-3-carboxamide;
methyl 3-(2-(3-fluoro-5-(imidazo[1,2-a]pyridine-3-carboxamido)-4-methylphenyl)oxazol-5-yl)azetidine-1-carboxylate;
methyl 3-(3-(2,5-difluoro-3-(imidazo[1,2-a]pyridine-3-carboxamido)-4-methylphenyl)-1,2,4-oxadiazol-5-yl)azetidine-1-carboxylate;
methyl (R)-3-(3-(2,5-difluoro-3-(imidazo[1,2-a]pyridine-3-carboxamido)-4-methylphenyl)-1,2,4-oxadiazol-5-yl)pyrrolidine-1-carboxylate;
methyl (R)-3-(3-(2,5-difluoro-3-(imidazo[1,2-a]pyridine-3-carboxamido)-4-methylphenyl)-1,2,4-oxadiazol-5-yl)piperidine-1-carboxylate;
methyl (S)-3-(3-(2,5-difluoro-3-(imidazo[1,2-a]pyridine-3-carboxamido)-4-methylphenyl)-1,2,4-oxadiazol-5-yl)pyrrolidine-1-carboxylate;
methyl (S)-3-(3-(2,5-difluoro-3-(imidazo[1,2-a]pyridine-3-carboxamido)-4-methylphenyl)-1,2,4-oxadiazol-5-yl)piperidine-1-carboxylate;
N-(3-(5-(3,3-difluorocyclobutyl)-1,2,4-oxadiazol-3-yl)-2,5-difluoro-6-methylphenyl)imidazo[1,2-a]pyridine-3-carboxamide;
N-(3-(5-(3,3-difluorocyclobutyl)-1,2,4-oxadiazol-3-yl)-2,5-difluoro-6-methylphenyl)-7-morpholinoimidazo[1,2-a]pyridine-3-carboxamide;
N-(3-(5-(3,3-difluorocyclobutyl)-1,2,4-oxadiazol-3-yl)-2,5-difluoro-6-methylphenyl)-7-(piperazin-1-yl)imidazo[1,2-a]pyridine-3-carboxamide;
N-(2,5-difluoro-3-(5-((1R,2S)-2-fluorocyclopropyl)-1,2,4-oxadiazol-3-yl)-6-methylphenyl)imidazo[1,2-a]pyridine-3-carboxamide;
N-(2,5-difluoro-3-(5-((1S,2R)-2-fluorocyclopropyl)-1,2,4-oxadiazol-3-yl)-6-methylphenyl)imidazo[1,2-a]pyridine-3-carboxamide;
N-(2,5-difluoro-3-(5-((1R,2S)-2-fluorocyclopropyl)-1,2,4-oxadiazol-3-yl)-6-methylphenyl)-7-morpholinoimidazo[1,2-a]pyridine-3-carboxamide;
N-(2,5-difluoro-3-(5-((1S,2R)-2-fluorocyclopropyl)-1,2,4-oxadiazol-3-yl)-6-methylphenyl)-7-morpholinoimidazo[1,2-a]pyridine-3-carboxamide;
N-(2,5-difluoro-3-(5-((1S,2R)-2-fluorocyclopropyl)-1,2,4-oxadiazol-3-yl)-6-methylphenyl)-7-methylimidazo[1,2-a]pyridine-3-carboxamide;
N-(2,5-difluoro-3-(5-((1S,2R)-2-fluorocyclopropyl)-1,2,4-oxadiazol-3-yl)-6-methylphenyl)-7-methoxyimidazo[1,2-a]pyridine-3-carboxamide;
methyl 3-(3-(2,5-difluoro-4-methyl-3-(7-methylimidazo[1,2-a]pyridine-3-carboxamido)phenyl)-1,2,4-oxadiazol-5-yl)azetidine-1-carboxylate;
methyl 3-(3-(2,5-difluoro-3-(7-methoxyimidazo[1,2-a]pyridine-3-carboxamido)-4-methylphenyl)-1,2,4-oxadiazol-5-yl)azetidine-1-carboxylate;
methyl 3-(3-(2,5-difluoro-4-methyl-3-(7-morpholinoimidazo[1,2-a]pyridine-3-carboxamido)phenyl)-1,2,4-oxadiazol-5-yl)azetidine-1-carboxylate;
methyl 3-(3-(2,5-difluoro-4-methyl-3-(7-(piperazin-1-yl)imidazo[1,2-a]pyridine-3-carboxamido)phenyl)-1,2,4-oxadiazol-5-yl)azetidine-1-carboxylate;
methyl 3-(3-(2,5-difluoro-4-methyl-3-(7-(4-methylpiperazin-1-yl)imidazo[1,2-a]pyridine-3-carboxamido)phenyl)-1,2,4-oxadiazol-5-yl)azetidine-1-carboxylate;
methyl 3-(3-(2,5-difluoro-4-methyl-3-(6-methylimidazo[1,2-a]pyridine-3-carboxamido)phenyl)-1,2,4-oxadiazol-5-yl)azetidine-1-carboxylate;
methyl 3-(3-(2,5-difluoro-3-(6-methoxyimidazo[1,2-a]pyridine-3-carboxamido)-4-methylphenyl)-1,2,4-oxadiazol-5-yl)azetidine-1-carboxylate;
methyl 3-(3-(2,5-difluoro-4-methyl-3-(6-morpholinoimidazo[1,2-a]pyridine-3-carboxamido)phenyl)-1,2,4-oxadiazol-5-yl)azetidine-1-carboxylate;
methyl 3-(3-(2,5-difluoro-4-methyl-3-(6-(piperazin-1-yl)imidazo[1,2-a]pyridine-3-carboxamido)phenyl)-1,2,4-oxadiazol-5-yl)azetidine-1-carboxylate;
methyl 3-(3-(2,5-difluoro-4-methyl-3-(6-(4-methylpiperazin-1-yl)imidazo[1,2-a]pyridine-3-carboxamido)phenyl)-1,2,4-oxadiazol-5-yl)azetidine-1-carboxylate;
N-(3-(5-(3,3-difluorocyclobutyl)-1,2,4-oxadiazol-3-yl)-2,5-difluoro-6-methylphenyl)pyrazolo[1,5-a]pyridine-3-carboxamide;
N-(2,5-difluoro-3-(5-((1R,2S)-2-fluorocyclopropyl)-1,2,4-oxadiazol-3-yl)-6-methylphenyl)pyrazolo[1,5-a]pyridine-3-carboxamide;
methyl 3-(3-(2,5-difluoro-4-methyl-3-(pyrazolo[1,5-a]pyridine-3-carboxamido)phenyl)-1,2,4-oxadiazol-5-yl)azetidine-1-carboxylate;
N-(3-(3-(3,3-difluorocyclobutyl)-1,2,4-oxadiazol-5-yl)-2,5-difluoro-6-methylphenyl)imidazo[1,2-a]pyridine-3-carboxamide;
N-(2,5-difluoro-3-(3-((1R,2S)-2-fluorocyclopropyl)-1,2,4-oxadiazol-5-yl)-6-methylphenyl)imidazo[1,2-a]pyridine-3-carboxamide;
methyl 3-(5-(2,5-difluoro-3-(imidazo[1,2-a]pyridine-3-carboxamido)-4-methylphenyl)-1,2,4-oxadiazol-3-yl)azetidine-1-carboxylate;
methyl 3-(5-(2,5-difluoro-4-methyl-3-(7-methylimidazo[1,2-a]pyridine-3-carboxamido)phenyl)-1,2,4-oxadiazol-3-yl)azetidine-1-carboxylate;
methyl 3-(5-(2,5-difluoro-3-(7-methoxyimidazo[1,2-a]pyridine-3-carboxamido)-4-methylphenyl)-1,2,4-oxadiazol-3-yl)azetidine-1-carboxylate;
methyl 3-(5-(2,5-difluoro-4-methyl-3-(7-morpholinoimidazo[1,2-a]pyridine-3-carboxamido)phenyl)-1,2,4-oxadiazol-3-yl)azetidine-1-carboxylate; and
methyl 3-(5-(2,5-difluoro-4-methyl-3-(7-(piperazin-1-yl)imidazo[1,2-a]pyridine-3-carboxamido)phenyl)-1,2,4-oxadiazol-3-yl)azetidine-1-carboxylate.

49. A pharmaceutical composition comprising a compound, or a pharmaceutically acceptable salt or solvate thereof, as described in any one of claims 1-48 and a pharmaceutically acceptable excipient.

50. A method of preparing a pharmaceutical composition comprising mixing a compound, or a pharmaceutically acceptable salt or solvate thereof, of any one of claims 1-48, and a pharmaceutically acceptable carrier.

51. A compound of any one of claims 1-48, or a pharmaceutically acceptable salt or solvate thereof, for use in a method of treatment of the human or animal body.

52. A compound of any one of claims 1-48, or a pharmaceutically acceptable salt or solvate thereof, for use in a method of treatment of cancer, inflammatory, allergic, or autoimmune disease.

53. Use of a compound of any one of claims 1-48, or a pharmaceutically acceptable salt or solvate thereof, in the manufacture of a medicament for the treatment of cancer, inflammatory, allergic, or autoimmune disease.

54. A compound of any one of claims 1-48, or a pharmaceutically acceptable salt or solvate thereof, for use in a method of treatment of c-KIT-mediated disease.

55. Use of a compound of any one of claims 1-48, or a pharmaceutically acceptable salt or solvate thereof, in the manufacture of a medicament for the treatment of c-KIT-mediated disease.

56. A method of treating cancer, inflammatory, allergic, or autoimmune disease in a patient in need thereof, comprising administering to the patient a compound as described in any one of claims 1-48, or a pharmaceutically acceptable salt or solvate thereof.

57. A method of treating cancer, inflammatory, allergic, or autoimmune disease in a patient in need thereof, comprising administering to the patient a pharmaceutical composition comprising a compound as described in any one of claims 1-48, or a pharmaceutically acceptable salt or solvate thereof, and a pharmaceutically acceptable excipient.

58. The method of claim 56 or 57, or the use of any one of claims 52-55, wherein the disease is selected from the group consisting of mastocytosis, asthma, chronic urticaria, rheumatoid arthritis, psoriasis, atopic dermatitis, neurofibromatosis, multiple sclerosis, and idiopathic pulmonary fibrosis.

59. A method of treating a c-KIT-mediated disease in a patient in need thereof, comprising administering to the patient a compound as described in any one of claims 1-48, or a pharmaceutically acceptable salt or solvate thereof.

60. A method of treating a c-KIT-mediated disease in a patient in need thereof, comprising administering to the patient a pharmaceutical composition comprising a compound as described in any one of claims 1-48, or a pharmaceutically acceptable salt or solvate thereof, and a pharmaceutically acceptable excipient.

61. A method of inhibiting a c-KIT kinase enzyme comprising contacting the enzyme with a compound of any one of claims 1-48, wherein the c-KIT kinase is contacted in an in vitro setting.

62. A method of inhibiting a c-KIT kinase enzyme comprising contacting the enzyme with a compound of any one of claims 1-48, wherein the c-KIT kinase is contacted in an in vivo setting.

63. A compound of any one of claims 1-48, or a pharmaceutically acceptable salt or solvate thereof, for use in a method of treating cancer.

64. Use of a compound of any one of claims 1-48, or a pharmaceutically acceptable salt or solvate thereof, in the manufacture of a medicament for the treatment of cancer.

65. A method of treating cancer in a patient in need thereof, comprising administering to the patient a compound as described in any one of claims 1-48, or a pharmaceutically acceptable salt or solvate thereof.

66. A method of treating cancer in a patient in need thereof, comprising administering to the patient a pharmaceutical composition comprising a compound as described in any one of claims 1-48, or a pharmaceutically acceptable salt or solvate thereof, and a pharmaceutically acceptable excipient.

67. The use of claim 63 or 64, or the method of claim 65 or 66, wherein the cancer is selected from the group consisting of leukemia, acute myeloid leukemia, chronic myeloid leukemia, acute lymphoblastic leukemia, lymphoma, germ cell tumor, hematopoietic cancers, gastrointestinal stromal tumor, uveal melanoma, colorectal carcinoma, breast carcinoma, small-cell lung carcinoma, neuroblastoma, gynecological tumor, malignant mesothelioma, papillary renal cell carcinoma, papillary renal carcinoma, mastocytosis, mast cell leukemia, thymic carcinoma, colorectal carcinoma, small-cell lung carcinoma, and neuroblastoma.

68. The use or method of claim 67, wherein the cancer is gastrointestinal stromal tumor.

69. The use or method of claim 67, wherein the cancer is acute myeloid leukemia.

70. The use or method of claim 67, wherein the cancer is melanoma.
